(19) United States
(12) Patent Application Publication
(10) Pub. No.: US 2025/0266125 A1
(43) Pub. Date: Aug. 21, 2025

(54) DE NOVO CHARACTERIZATION OF CELL-FREE DNA FRAGMENTATION HOTSPOTS IN HEALTHY AND EARLY-STAGE CANCERS

(71) Applicant: CHILDREN'S HOSPITAL MEDICAL CENTER, Cincinnati, OH (US)

(72) Inventors: Yaping LIU, Cincinnati, OH (US); Xionghui ZHOU, Wuhan City (CN); Haizi ZHENG, Cincinnati, OH (US)

(73) Assignee: CHILDREN'S HOSPITAL MEDICAL CENTER, Cincinnati, OH (US)

(21) Appl. No.: 18/069,469

(22) PCT Filed: Jun. 22, 2021
(86) PCT No.: PCT/US2021/038554

§ 371 Date: Dec. 21, 2022

Related U.S. Application Data
(60) Provisional application No. 63/042,116, filed on Jun. 22, 2020.
(60) Provisional application No. 63/051,752, filed on Jul. 14, 2020.

(51) Int. Cl. G16B 20/00; C12Q 1/6886; G16B 30/00; G16B 40/00; G16H 50/20

(52) U.S. Cl. CPC G16B 20/00; C12Q 1/6886; G16B 30/00; G16B 40/00; G16H 50/20; C12Q 2600/156

(57) ABSTRACT

A system and method for identifying genomic regions with higher fragmentation rates than the local and global backgrounds as part of diagnosing early stage cancer is provided. The method includes steps of: de-novo characterizing genome-wide cell-free DNA fragmentation regions with higher fragmentation rates than the local and global backgrounds from whole-genome sequencing by weighing the fragment coverages in each region by a ratio of average fragment sizes in the region versus that in the whole chromosome to generate a score; and identifying DNA fragmentation regions of interest based upon comparing the score with a threshold. The system and method can utilize identified DNA fragmentation hotspots for the detection and localization of multiple early-stage cancers (or certain other non-malignant disease).

### CROSS REFERENCE TO RELATED APPLICATIONS

The current application claims priority to U.S. provisional applications Ser. No. 63/042,116, filed Jun. 22, 2020 and Ser. No. 63/051,752, filed Jul. 14, 2020, the entire disclosures of which are incorporated herein by reference.

### BACKGROUND

Circulating cell-free DNA (cfDNA) from patients' plasma is a promising non-invasive biomarker for diagnosing and screening early-stage cancers[1]. The fragmentation patterns of cfDNA are not evenly distributed in the genome and associated with the local epigenetic backgrounds[2,3]. The cfDNA fragmentation patterns are altered in cancer, bringing enormous signals from both tumor and peripheral immune cells to detect early-stage cancers[4,5]. Recently, several patterns have been derived to capture the full spectrums of the cfDNA fragmentation in cancer, such as patterns near transcription start sites (TSS) and transcription factor binding sites (TFBS), orientation-aware cfDNA fragmentation (OCF), the preferred-ended position of cfDNA, motif diversity score (MDS), large-scale fragmentation patterns at mega-base level (DELFI), and nucleosome positioning (window protection score, WPS)[3,4,6-10]. However, the studies of fragmentation patterns at selected known regulatory elements, such as TSS[6], TFBS[9], and known open chromatin regions in immune cells (OCF)[8], limited their opportunities to unbiasedly characterize the genome-wide fragmentation aberrations on other regulatory regions in early-stage cancers. The preferred-ended position of cfDNA has not been associated with known gene-regulatory elements yet[7]. MDS[10] is a single summary statistic score for each patient that does not allow further explorations of its association with specific gene-regulatory elements. The large-scale fragmentation patterns at mega-bases level (DELFI)[4] are challenging to be associated with the fine-scale gene-regulatory elements, genes, pathways, and therefore further druggable targets for the interventions of early-stage cancers. These challenges limited their potential opportunity to characterize the underlying unknown gene-regulatory aberrations during the initiations of early-stage cancers.

To conquer these challenges, an unbiased genome-wide approach is needed to narrow down the regions of interest from cfDNA fragments directly. A previous study on cfDNA from healthy and late-stage cancers de novo characterize the regions with high WPS signals that are associated with nucleosome occupancies[3]. Nucleosome occupancies inside the cells are usually measured by MNase-seq, which is not comprehensively performed at various primary cell types across different human pathological conditions, such as cancer. Thus, the characterization of nucleosome occupied regions from cfDNA will still limit our scope to dissect the potential regulatory aberrations in cancer. However, reduced fragmentation process (“fragmentation coldspots”) at nucleosome-occupied regions, on the other side, indicates the potential existence of increased fragmentation process (“fragmentation hotspots”) at the open chromatin regions. Open chromatin regions have recently been comprehensively profiled by ATAC-seq and DNase-seq at many primary cell types across different physiological conditions, including cancer and immune cells[11,12]. Transcription factors usually bind the open chromatin regions rather than the nucleosome occupied regions[13]. Moreover, non-coding genetic variants associated with different complex diseases are enriched in the open chromatin regions from related cell types[14-16]. Therefore, instead of identifying “fragmentation coldspots” at nucleosome-occupied regions, we hypothesize that the characterization of cfDNA “fragmentation hotspots” at open chromatin regions will not only boost the power for the identification of nuanced pathological conditions, such as early-stage cancer, but also elucidate the unknown gene-regulatory mechanisms indicated by the fragmentation patterns from patients' plasma cfDNA.

### SUMMARY

The current disclosure provides an approach to de novo characterize the cell-free DNA fragmentation hotspots from whole-genome sequencing. In healthy, hotspots are enriched in gene-regulatory elements, including promoters, hematopoietic-specific enhancers, and 3′ end of transposons. In early-stage cancers, fragmentations are aberrant at hotspots near microsatellites, CTCF, and genes enriched in immune processes from peripheral immune cells, which indicated the aberrations of chromatin organizations and immune-gene expressions during cancer initiations. Utilizing these hotspots, we diagnosed eight early-stage cancers from two studies with high accuracy. Moreover, we identified the tissues-of-origin of multi-cancers with a median of 85% accuracy, which has not been shown by other fragmentation approaches. The results highlight the significance of de novo characterizing the cell-free DNA fragmentation hotspots for detecting early-stage cancers and dissection of gene-regulatory aberrations in cancers.

Embodiments of the current disclosure provide a computational approach, named Cell fRee dnA fraGmentation (CRAG), to de novo identify the genome-wide cfDNA fragmentation hotspots by utilizing the weighted fragment coverages from cfDNA paired-end WGS data. We analyzed the gene-regulatory potentials of these fragmentation hotspots in healthy individuals and patients with early-stage cancer, which revealed the previously unknown gene-regulatory aberrations from peripheral immune cells in cancers. Finally, we utilized these fragmentation hotspots for the detection and localization of multiple early-stage cancers.

In an aspect a method for identifying DNA fragmentation hotspots as part of diagnosing early stage cancer or certain other non-malignant disease includes steps of: de-novo characterizing genome-wide cell-free DNA fragmentation hotspots from whole-genome sequencing by integrating fragment size and coverage into a score; and identifying DNA fragmentation hotspots of interest based upon the score being below a threshold. In a further detailed embodiment, the score identifies regions with lower fragment coverage and smaller fragment size.

Alternatively, or in addition, the method further includes a step of scanning a chromosome with a sliding window of a first size and a step with a second size. In a detailed embodiment, the score is calculated by weighting fragment coverage based on a ratio of average fragment size in the sliding window versus that in the whole chromosome. In a further detailed embodiment, the score is calculated based upon the following equation wherein, in the iₜₕ window:

$${IFS}_{i} = {n}_{i} * ( 1 + \frac{{l}_{i}}{L} ) \qquad ( 1 )$$

$${C}_{i} = ⌊ {IFS}_{i} ⌋ \qquad ( 2 )$$

where Cᵢ is the IFS score round down to the nearest integer in the iₜₕ window, nᵢ is the number of fragments whose mid-points are located within the iₜₕ window, lᵢ is the average fragment size in the iₜₕ window, L is the average fragment size in the whole chromosome.

In an embodiment, the first size is 200 bp and the second size is 20 bp.

Alternatively, or in addition, the method may include a step of utilizing identified DNA fragmentation hotspots for the detection of early-stage cancer. In a further detailed embodiment, the detection step may include performing Gene Ontology (GO) analysis of the identified DNA fragmentation hotspots, or performing Motif analysis of the identified DNA fragmentation hotspots.

In an embodiment, the integrating step weighs fragment coverages with size information. In a further detailed embodiment, the integrating step weighs the fragment coverage based on a ratio of fragment size in a window versus that in the whole chromosome.

Another aspect provides a method for identifying genomic regions with higher fragmentation rates than the local and global backgrounds as part of diagnosing early stage cancer (or certain other non-malignant disease). The method includes steps of: de-novo characterizing genome-wide cell-free DNA fragmentation regions with higher fragmentation rates than the local and global backgrounds from whole-genome sequencing by weighing the fragment coverages in each region by a ratio of average fragment sizes in the region versus that in the whole chromosome to generate a score; and identifying DNA fragmentation regions of interest based upon comparing the score with a threshold. In an embodiment, the method further includes a step of scanning a chromosome with a sliding window of a first size and a step with a second size. In a further detailed embodiment, the score is calculated by weighting fragment coverage based on a ratio of average fragment size in the sliding window versus that in the whole chromosome. Alternatively, or in addition, the first size is 200 bp and the second size is 20 bp.

In an embodiment, the method further includes utilizing identified DNA fragmentation hotspots for the detection of early-stage cancer. In a more detailed embodiment, the detection step may include performing Gene Ontology (GO) analysis of the identified DNA fragmentation hotspots; or performing Motif analysis of the identified DNA fragmentation hotspots.

These and other aspects and advantages of the current disclosure will be apparent from the following description, the appended claims and the attached drawings.

### BRIEF DESCRIPTION OF THE DRAWINGS

FIGS. 1a-d. Illustrate a schematic of an exemplary CRAG approach.

FIG. 1a. Illustrates the overall workflow for the detection and localization of early-stage cancer.

FIG. 1b. Is a schematic of hotspot identification.

FIG. 1c. Is the Q-Q plot for the negative binomial modeling of IFS score distribution.

FIG. 1d. Is the distribution of IFS around the hotspots in the BH01 dataset.

FIG. 2a-2h. Provides charts illustrating CfDNA fragmentation hotspots are enriched at gene-regulatory regions in healthy.

FIG. 2a. Is the overlap of cfDNA fragmentation hotspots and CGI Transcription Starting Sites (TSSs), non-CGI TSSs, 5′ exon boundary (no TSS and CTCF within +/−2 kb), Transcription Termination Sites (TTSs) (no TSS and CTCF within +/−2 kb), CTCF transcription factor binding sites (no TSS within +/−4 kb), and random genomics regions.

FIG. 2b. Is the DNA accessibility levels from hematopoietic cells around the cfDNA fragmentation hotspots.

FIG. 2c. Is the histone modification levels from monocytes around the cfDNA fragmentation hotspots.

FIG. 2d. Is the H3K4me1 histone modification levels from hematopoietic (solid lines) and non-hematopoietic (dashed lines) cells around the cfDNA fragmentation hotspots.

FIG. 2e. Is the enrichment of hotspots at tissue-specific chromHMM states (TssA, TssFlank, and Enhancer, also overlapped with tissue-specific open chromatin regions). Odds ratio is compared with matched random regions (matched chromosome and length, repeated 10 times). Error bar is based on 95% confidence interval. P value is calculated based on Fisher exact test.

FIG. 2f. Is a ROC curve for the prediction of open chromatin regions by the linear SVM model on the IFS score and other features in the benchmark datasets.

FIG. 2g. Is the overlap of cfDNA fragmentation hotspots and 3′ end of transposons (Alu, L1, and LTR)

FIG. 2h. Is the cfDNA methylation level from healthy individuals around the 3′ end of Alu that overlapped or not overlapped with the cfDNA fragmentation hotspots.

FIGS. 3a-3g. Provide charts and graphs illustrating the aberrations of cfDNA fragmentation patterns at hotspots in early-stage cancers.

FIG. 3a. Is a volcano plot of z-score differences and p-value (two-way Mann-Whitney U test) for the aberration of IFS in cfDNA fragmentation hotspots between early-stage HCC and healthy.

FIG. 3b. Is unsupervised clustering on the Z-score of IFS at the top 10,000 most variable cfDNA fragmentation hotspots called from HCC and healthy samples.

FIG. 3c. Is receiver operator characteristics (ROC) for the detection of early-stage HCC by using IFS (after GC bias correction) from all the cfDNA fragmentation hotspots (red), copy number variations (brown), and mitochondrial genome copy number analysis (black).

FIG. 3d. Are scatter plots of z-score differences and feature importance (coefficient in linear SVM) split the cfDNA fragmentation hotspots into two groups: hypo-fragmented in cancer (Class I) and hyper-fragmented in cancer (Class II).

FIG. 3e. Is the fraction of Class I and Class II hotspots that are overlapped with microsatellite repeats, as well as their relative distance to the nearest TSS.

FIG. 3f. Is the top 10 motif enrichment at Class I and Class II hotspots.

FIG. 3g. Is the top 10 enrichment of Gene Ontology Biological Process at Class I and Class II hotspots.

FIG. 4a-d. Illustrates graphs and charts for the detection and localization of multiple early-stage cancers.

FIG. 4a. Is the t-SNE visualization on the Z-score of IFS (after GC bias correction) at the most variable cfDNA fragmentation hotspots (one-way ANOVA test with p value<0.01) across multiple different early-stage cancer types and healthy conditions.

FIG. 4b. Is unsupervised clustering on Z-score of IFS (after GC bias correction) at the top 40,000 most variable cfDNA fragmentation hotspots across multiple different early-stage cancer types and healthy conditions.

FIG. 4c. Is the sensitivity across different cancer stages at 100% specificity to distinguish cancer and healthy condition by using IFS (after GC bias correction) at cfDNA fragmentation hotspots. Error bars represent 95% confidence intervals.

FIG. 4d. Is percentages of patients correctly classified by one of the two most likely types (sum of orange and blue bars) or the most likely type (blue bar). Error bars represent 95% confidence intervals.

FIGS. S1a-b Represent fragmentation patterns near the cfDNA fragmentation hotspots.

FIG. S1a. The distribution of IFS from IH01.

FIG. S1b adjusted IFS (after k-mer correction) from BH01 around the fragmentation hotspots called at BH01 dataset.

FIG. S2a1-S2a12 are a representation of Genome browser tracking of cfDNA fragmentation hotspots. The first box is near promoter regions. The second box is at intergenic regions.

FIG. S3 is a graph presenting the enrichment of ATAC-see signals from neutrophils around the cfDNA fragmentation hotspots (BH01).

FIGS. S4a-b provide graphs illustrating epigenetic signals around cfDNA fragmentation hotspots (BH01).

FIG. S4a. The histone modification signal distributions (−log 10 P-value calculated by MACS2, downloaded from Roadmap Epigenomics Consortium) from neutrophil, B cell, and T cell around cfDNA fragmentation hotspots (BH01).

FIG. S4b. The enrichment of cfDNA hotspots from BH01 at tissues-specific chromHMM states (TssA, TssFlank, and Enhancer). The odds ratio is compared with matched random regions (matched chromosome and length, repeated 10 times). Error bar is based on the 95% confidence interval. P-value is calculated based on Fisher's exact test. BH01 cfDNA fragmentation hotspots are identified from GC-bias corrected IFS signals.

FIG. S5 provides a boxplot of the conservation score (PhastCons) within cfDNA fragmentation hotspots and matched random regions.

FIG. S6a-c. Illustrates CfDNA fragmentation hotspots and transposable elements (TE).

FIG. S6a. Is the mappability score distribution at 3′ end of TE.

FIG. S6b. Is the G+C % content distribution at 3′ end of TE.

FIG. S6c. The top 10 motif enrichment at hotspots after the 3′ end of TE.

FIG. S7 provides a graph illustrating the power estimation for the cfDNA fragmentation hotspots called by CRAG with different numbers of fragments.

FIG. S8. Illustrates unsupervised clustering on the Z-score of IFS at the top 10,000 most variable cfDNA fragmentation hotspots called from HCC and healthy samples (after GC bias correction).

FIGS. S9a-e. Illustrates unsupervised clustering on the Z-score of IFS at the most variable cfDNA fragmentation hotspots called from HCC and healthy samples.

FIG. S9a. Clustering on the euclidean distance metrics from the top 10,000 most variable hotspots.

FIG. S9b. Clustering on the spearman correlation distance metrics from the top 20,000 most variable hotspots.

FIG. S9c. Clustering on the euclidean distance metrics from the top 20,000 most variable hotspots.

FIG. S9d. Clustering on the spearman correlation distance metrics from the top 30,000 most variable hotspots.

FIG. S9e. Clustering on the euclidean distance metrics from the top 30,000 most variable hotspots.

FIGS. S10a-b. Provides graphs illustrating receiver operator characteristics (ROC) for the detection of early-stage HCC.

FIG. S10a. IFS from cfDNA fragmentation hotspots (after GC bias correction) and,

FIG. S10b. Using IFS signals but with different machine learning approaches.

FIGS. S11a-b. Provides charts illustrating the functional analysis of Class I hotspot and Class II hotspots in HCC and healthy controls.

FIG. S11a. The enrichment of silenced genes in PBMC (promoters are overlapped with Class I hotspots) from early-stage HCC comparing to that from healthy controls.

FIG. S11b. The cfDNA methylation level is significantly lower at HCC comparing to healthy controls in Class II hotspots (also overlapped with microsatellites).

FIG. S12a-c. Provides plots illustrating Principal Component Analysis (PCA) on the cfDNA fragmentation hotspots. PCA analysis on Z-score transformed IFS signals from

FIG. S12a. All hotspots from pooled HCC (red), chronic HBV infection (cyan), HBV-associated liver cirrhosis (green), and Healthy (blue) samples.

FIG. S12b. Matched random regions (matched chromosome and length with hotspots) from pooled HCC (red), chronic HBV infection (cyan), HBV-associated liver cirrhosis (green), and Healthy (blue) samples.

FIG. S12c. All hotspots from pooled random grouped samples, the sample sizes are matched with HCC, chronic HBV infection, HBV-associated liver cirrhosis, and Healthy.

FIG. S13. Illustrates unsupervised clustering on the Z-score of IFS at the top 10,000 most variable cfDNA fragmentation hotspots called from HCC (red), chronic HBV infection (cyan), HBV-associated liver cirrhosis (green), and Healthy (blue) samples (a). Before and (b). After GC bias correction.

FIG. S14a-i. Illustrates unsupervised clustering on the Z-score of IFS at the most variable cfDNA fragmentation hotspots called from HCC, HBV-associated liver cirrhosis, chronic HBV infection, and healthy individuals. \

FIG. S14a. Clustering on the euclidean distance metrics from the top 30,000 most variable hotspots.

FIG. S14b. Clustering on the spearman correlation distance metrics from the top 10,000 most variable hotspots.

FIG. S14c. Clustering on the euclidean distance metrics from the top 10,000 most variable hotspots.

FIG. S14d. Clustering on the spearman correlation distance metrics from the top 20,000 most variable hotspots.

FIG. S14e. Clustering on the euclidean distance metrics from the top 20,000 most variable hotspots.

FIG. S14f. Clustering on the spearman correlation distance metrics from the top 40,000 most variable hotspots.

FIG. S14g. Clustering on the euclidean distance metrics from the top 40,000 most variable hotspots.

FIG. S14h. Clustering on the spearman correlation distance metrics from the top 50,000 most variable hotspots.

FIG. S14 i. Clustering on the euclidean distance metrics from the top 50,000 most variable hotspots.

FIG. S15a-b. Provides graphs representing receiver operator characteristics (ROC) to distinguish early-stage HCC with benign conditions (HBV-associated liver cirrhosis and chronic HBV infection) by using IFS from cfDNA fragmentation hotspots

FIG. S15a. Before GC bias correction.

FIG. S15b. After GC bias correction.

FIG. S16a-c. Illustrates the aberrations of IFS (before GC bias correction) across multiple early-stage cancer and healthy.

FIG. S16a. t-SNE visualization on the Z-score of IFS (before GC bias correction) at the top 40,000 most variable cfDNA fragmentation hotspots across multiple different early-stage cancer types and healthy.

FIG. S16b. Unsupervised clustering (WPGMA method on spearman correlation distance) on Z-score of IFS (before GC bias correction) at the top 40,000 most variable cfDNA fragmentation hotspots across multiple different early-stage cancer types and healthy.

FIG. S16c. Unsupervised clustering (Ward's method on euclidean distance) on Z-score of IFS (before GC bias correction) at the top 40,000 most variable cfDNA fragmentation hotspots across multiple different early-stage cancer types and healthy.

FIG. S17a-g. Provides graphs illustrating receiver operator characteristics (ROC) for the detection of different early-stage cancers by using IFS from cfDNA fragmentation hotspots before (left panel) and after (right panel) GC bias correction.

FIG. S17a. Breast cancer.

FIG. S17b. Colorectal cancer.

FIG. S17c. Ovarian cancer.

FIG. S17d. Gastric cancer.

FIG. S17e. Lung cancer.

FIG. S17f. Pancreatic cancer.

FIG. S17g. Bile duct cancer.

FIG. S18a-g. Provides bar graphs illustrating the sensitivity across different cancer stages at 100% specificity for the detection of different early-stage cancers by using IFS from cfDNA fragmentation hotspots before (left panel) and after (right panel) GC bias correction. The sample size in each stage is at the bottom of each bar.

FIG. S18a. Breast cancer.

FIG. S18b. Colorectal cancer.

FIG. S18c. Ovarian cancer.

FIG. S18d. Gastric cancer.

FIG. S18e. Lung cancer.

FIG. S18f. Pancreatic cancer.

FIG. S18g. Bile duct cancer. Error bars represent 95% confidence intervals.

FIG. S19a-b. Provides bar graphs illustrating the sensitivity at 100% specificity for the detection of early-stage cancer across different tumor fractions.

FIG. S19a. Cristiano et al. data and

FIG. S19b. HCC vs. Healthy at Jiang et al. data. The tumor fraction is estimated by ichorCNA.

FIG. S20 Provides a bar graph illustrating tissues-of-origin prediction across six different cancer types. Percentages of patients correctly classified by one of the two most likely types (sum of orange and blue bars) or the most likely type (blue bar). Error bars represent 95% confidence intervals.

FIG. S21. Provides a bar graph illustrating tissues-of-origin prediction randomly by sample frequency across five cancer types. Percentages of patients correctly classified by one of the two most likely types (sum of orange and blue bars) or the most likely type (blue bar). Error bars represent 95% confidence intervals.

### DETAILED DESCRIPTION

### CRAG: A Probabilistic Model to Characterize the Cell-Free DNA Fragmentation Hotspots.

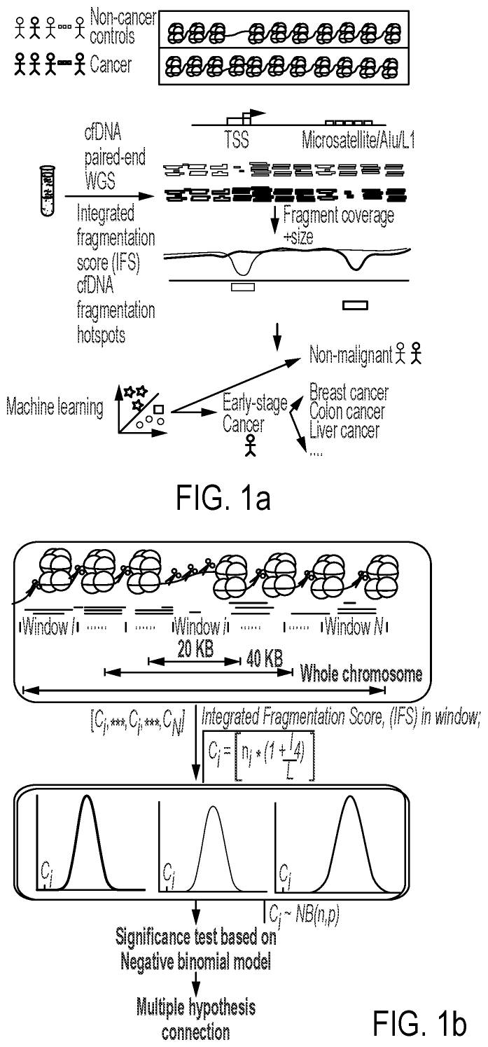

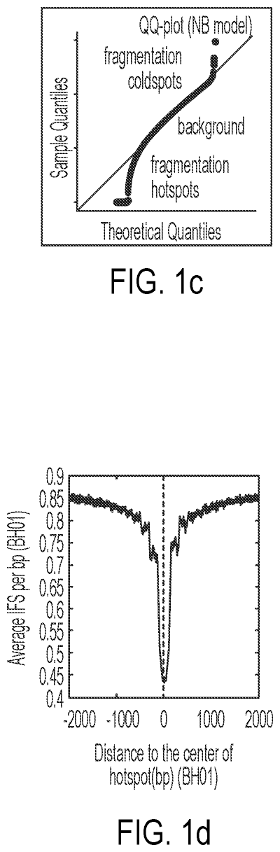

Embodiments of the current disclosure provide a computational approach to de novo characterize the fine-scale genomic regions with higher fragmentation rates than the local and global backgrounds, defined as cfDNA fragmentation hotspots (FIG. 1a-b). Since both fragment coverages and sizes are essential parts of evaluating the fragmentation process, we weighed the fragment coverages in each region by the ratio of average fragment sizes in the region versus that in the whole chromosome, named integrated fragmentation score (IFS) (Details in Methods). The negative binomial model we provided correctly captured the variation of IFS in the background and indicated the existence of cfDNA fragmentation hotspots (FIG. 1c, Details in Methods). Since sequencing coverages are usually affected by the G+C % content, we also normalized the IFS signals with the G+C % content within the regions (Details in Methods). We used the cfDNA deep WGS data (BH01, ˜100×)[3] from the healthy non-pregnant individuals as the primary data set to evaluate our approach in healthy individuals. In the BH01 dataset, we identified 277,109 cfDNA fragmentation hotspots. The IFS distributions in both BH01 and another independent dataset from a healthy individual (IH01, ˜100×) showed expected depletions at the center of BH01 hotspots (FIG. 1d, FIG. Sla).

Further, we normalized the IFS signals by k-mer composition (n=2) at BH01 hotspots (Details in Methods). We did not observe any change in the overall distribution of fragmentation patterns before and after the correction (FIG. S1b). These results suggested that our model robustly captured the cfDNA fragmentation hotspots in healthy individuals.

### Cell-Free DNA Fragmentation Hotspots are Highly Enriched in Gene-Regulatory Elements.

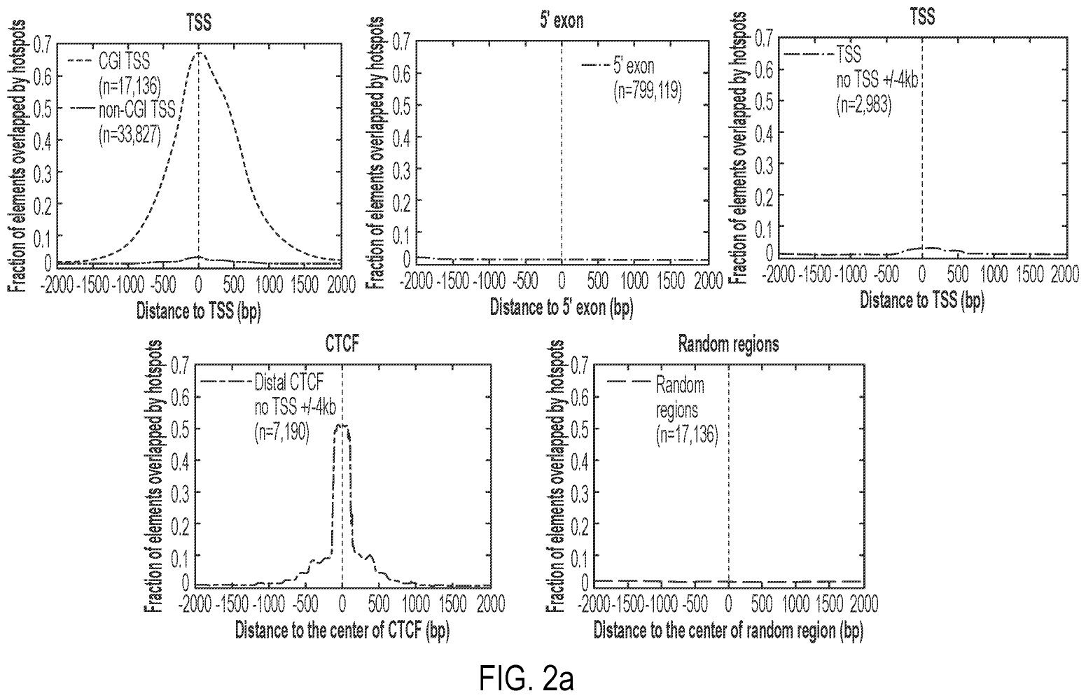

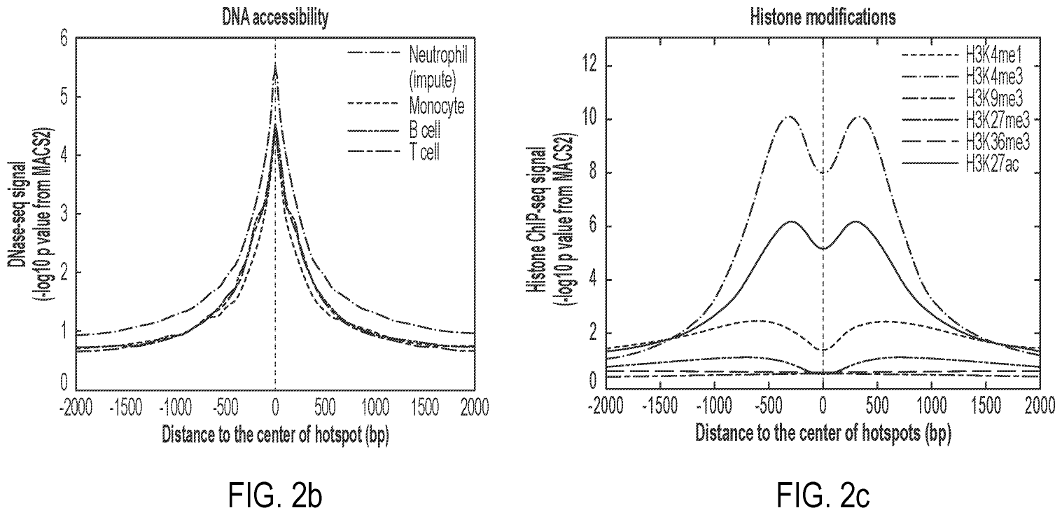

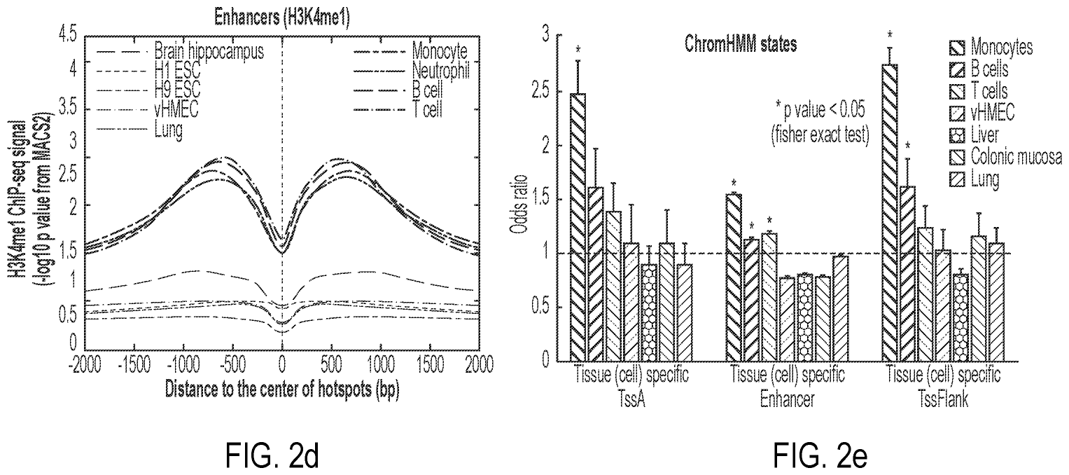

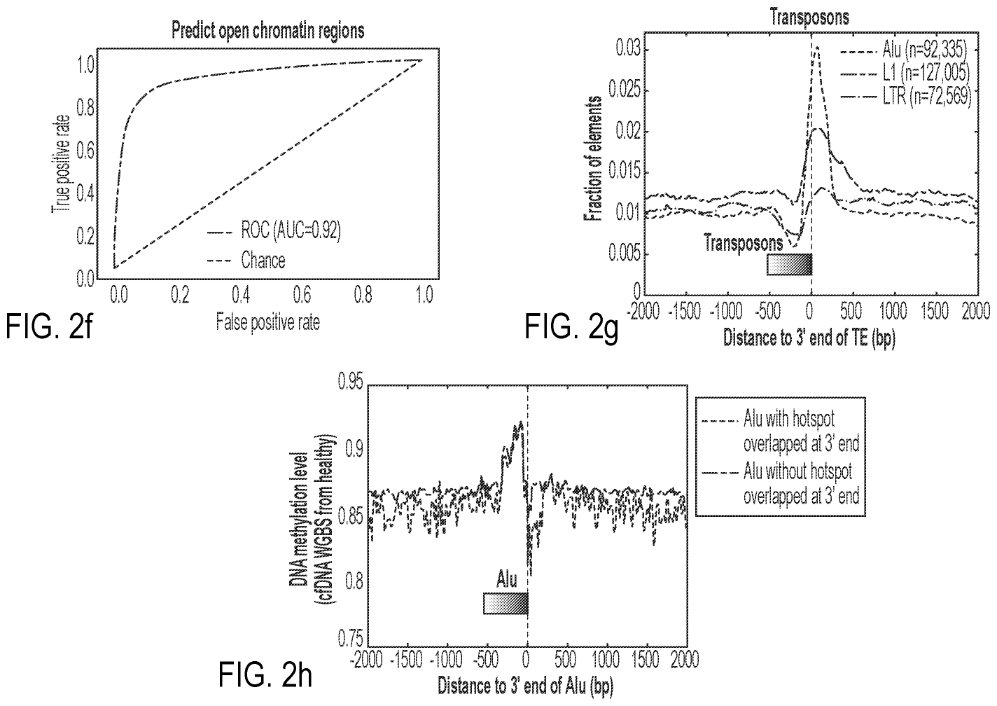

We next sought to characterize the genomic distributions of these fragmentation hotspots in healthy individuals (BH01). Similar to the previous studies on the open chromatin regions[17], the fragmentation hotspots are highly enriched at the CpG island (CGI) promoters and CTCF insulators, but not enriched at the non-CGI promoters, 5′ exon boundaries, transcription terminated sites (TTS), and random genomic regions (FIG. 2a). Since hematopoietic cells are the major contributors to cfDNA in healthy non-pregnant individuals[18], we plotted the distributions of DNA accessibility signals measured by different platforms at the major hematopoietic cell types in peripheral blood around the hotspots. We found the high enrichment patterns as expected (FIG. 2b, FIG. S2, FIG. S3). Also, we observed the high enrichment of active histone marks, such as H3K4me3 and H3K27ac. We found the depletion of repressive histone marks, such as H3K27me3, H3K9me3, as well as the gene-body histone mark H3K36me3. The enhancer mark H3K4me1, from hematopoietic cell types but not other cell types, showed the high enrichment around the hotspots (FIG. 2c-d, FIG. S2, FIG. S4a). To further understand the enrichment of fragmentation hotspots at different chromatin states, we utilized the 15-states chromHMM segmentation results across different cell types from the NIH Roadmap Epigenomics Mapping Consortium[19]. The hotspots mainly showed the enrichment in the tissue/cell-type-specific chromHMM states from hematopoietic cell types but not other cell types. (FIG. 2e, FIG. S4b). The evolutionary conservation score (phastCons) in hotspots is also significantly higher than matched random regions (two-sided Mann-Whitney U test, p<2.2e⁻¹⁶, FIG. S5)[20]. Finally, we utilized the constitutively active regions and repressive regions to benchmark the efficiency that we can detect the open chromatin regions by the fragmentation score, we achieved the 0.92 area under the curve (AUC) to predict the known open chromatin regions (FIG. 2f, Details in Methods).

To explore the unknown regulatory potentials of cfDNA fragmentation hotspots, we collected 523 public available open chromatin region datasets measured by DNase-seq or ATAC-seq across different cell types (Details in Table S1). These cell types are the major known contributors to cfDNA in healthy non-pregnant individuals, including liver and rest or activated immune cells from the Roadmap Epigenomics Consortium, ENCODE, BLUEPRINT, and other publications[12,19,21-23]. Interestingly, after excluding the potential overlap with these known open chromatin regions, we noticed a high enrichment of hotspots not within but right after the 3′ end of transposable elements (TEs), which are not the regions with the low mappability and high G+C % bias (FIG. 2g, FIG. S6a,b). The motif enrichment results at these hotspots right after the 3′ end of TEs further suggested the high enrichment of pioneer transcription factors, such as OCT (POU, Homeobox), which usually bind the nucleosome occupied regions (FIG. S6c)[24]. Moreover, we observed the differences of DNA methylation at the same regions (right after the 3′ end of Alu) with or without the overlap of hotspots, which indicates the potential functional association between hotspots and the local epigenetic status after the 3′ end of TEs (FIG. 2h).

TABLE S1
Table S1: list of public data we used

| Access id | cell type | assay type | publication | URL |
|---|---|---|---|---|
| gene annotation, | | | | https://www.gencodegenes.org/ |
| including TSS/exon | | | | human/release_30lift37.html |
| (Genecode Release | | | | |
| 30, GRCh37, | | | | |
| Comprehensive gene | | | | |
| annotation) | | | | |
| CTCF motif (hg19) | | | “Suresh Cuddapah | |
| | | | et al.““Global | |
| | | | Analysis of the | |
| | | | Insulator Binding | |
| | | | Protein CTCF in | |
| | | | Chromatin Barrier | |
| | | | Regions Reveals | |
| | | | Demarcation of | |
| | | | Active and | |
| | | | Repressive | |
| | | | Domains””. | |
| | | | Genome Res | |
| .2009 January; 19(1): 24-32. | | | | |
| doi: 10.1101/gr.082800.108. | | | | |
| Epub 2008 Dec. 3” | | | | |
| CpG island (UCSC Table | | | | https://genome.ucsc.edu/ |
| browser) | | | | cgi-bin/hgTables |
| repeats (hg19, | | | Smit AFA, Hubley | https://genome.ucsc.edu/ |
| RepeatMasker from | | | R, Green P. | cgi-bin/hgTables |
| UCSC table browser) | | | RepeatMasker | |
| | | | Open-3.0. | |
| | | | http://www.repeatmasker. | |
| | | | org.1996-2010. | |
| GC contents | | | | http://hgdownload.cse.ucsc.edu/ |
| | | | | goldenPath/hg19/gc5Base/ |
| Dark regions (merged | | | | https://genome.ucsc.edu/ |
| wgEncodeDacMapability | | | | cgi-bin/hgTables |
| ConsensusExcludable and | | | | |
| wgEncodeDukeMapabil- | | | | |
| ityRegionsExcludable | | | | |
| in hg19) | | | | |
| “phastCons scores for | | | | |
| multiple alignments | | | | |
| of 99 vertebrate | | | “Siepel A, | |
| genomes to the human | | | Bejerano G, | |
| genome(hg19)” | | | Pedersen J S, | |
| | | | Hinrichs A S, | |
| | | | Hou M, | |
| | | | Rosenbloom K, | |
| | | | Clawson | |
| H, Spieth J, Hillier L W, | | | | |
| Richards S, et al. | | | | |
| Evolutionarily conserved | | | | |
| elements | | | | |
| in vertebrate, insect, | | | | |
| worm, and yeast | | | | |
| genomes. Genome Res. | | | | |
| 2005 | | | | |
| August; 15(8): 1034-50. | http://hgdownload. | | | |
| (http://www.genome.org/ | cse.ucsc.edu/ | | | |
| cgi/doi/10.1101/ | goldenpath/hg19/ | | | |
| gr.3715005)” | phastCons100way/ | | | |
| | hg19.100way.phastCons.bw | | | |
| Merged data from 32 | Human | whole- | Sun et al. 2015 | |
| healthy controls | healthy | genome | PNAS. | |
| (EGAS00001001219) | plasma | bisulfite | https://doi.org/10.1073/ | |
| | | sequencing | pnas.1508736112 | |
| | | data | | |
| cfDNA WGBS data from | Human | whole- | Chan et al. 2013 | |
| early-stage HCC and | plasma | genome | PNAS. | |
| healthy controls | from HCC | bisulfite | https://doi.org/10.1073/ | |
| (EGAS00001000566) | and | sequencing | pnas.1313995110 | |
| | healthy | data | | |
| Gene expression data in | PBMC | gene | Shi et al. 2014 Eur | https://www.ncbi.nlm.nih.gov/ |
| PBMC from early-stage | | expression | J Cancer. | geo/query/acc.cgi?acc=GSE49515 |
| HCC and healthy | | microarray | https://doi.org/10.1016/ | |
| controls | | | j.ejca.2013.11.026 | |
| SRR2129993 (BH01) | Human | whole- | Snyder M W, | https://trace.ncbi.nlm.nih.gov/ |
| (SRA) | healthy | genome | Kircher M, Hill A J, | Traces/sra/?run=SRR2129993 |
| | plasma | sequencing | Daza R M et al. | |
| | | data | Cell-free DNA | |
| | | (Illumina | Comprises an In | |
| | | HiSeq 2000) | Vivo Nucleosome | |
| | | | Footprint that | |
| | | | Informs Its | |
| | | | Tissues-Of-Origin. | |
| | | | Cell 2016 Jan. | |
| | | | 14; 164(1-2): 57-68. | |
| | | | PMID: 26771485 | |
| SRR2130050 (IH01) | Human | whole- | Snyder M W, | https://trace.ncbi.nlm.nih.gov/ |
| (SRA) | healthy | genome | Kircher M, Hill A J, | Traces/sra/?run=SRR2130050 |
| | plasma | sequencing | Daza R M et al. | |
| | | data | Cell-free DNA | |
| | | (Illumina | Comprises an In | |
| | | HiSeq 2000) | Vivo Nucleosome | |
| | | | Footprint that | |
| | | | Informs Its | |
| | | | Tissues-Of-Origin. | |
| | | | Cell 2016 Jan. | |
| | | | 14; 164(1-2): 57-68. | |
| | | | PMID: 26771486 | |
| SRR2130051 (IH02) | Human | whole- | Snyder M W, | https://trace.ncbi.nlm.nih.gov/ |
| (SRA) | healthy | genome | Kircher M, Hill A J, | Traces/sra/?run=SRR2130051 |
| | plasma | sequencing | Daza R M et al. | |
| | | data | Cell-free DNA | |
| | | (Illumina | Comprises an In | |
| | | HiSeq 2000) | Vivo Nucleosome | |
| | | | Footprint that | |
| | | | Informs Its | |
| | | | Tissues-Of-Origin. | |
| | | | Cell 2016 Jan. | |
| | | | 14; 164(1-2): 57-68. | |
| | | | PMID: 26771487 | |
| 225 samples from PNAS | Human | whole- | Jiang P, Chan C W | |
| paper(32 healthy | plasma | genome | M, Chan K C A, et | |
| samples, 90 HCC | | sequencing | al. Lengthening | |
| samples, 67 HBV | | data | and shortening of | |
| samples and 36 | | (Illumina | plasma DNA in | |
| Cirrhosis samples) | | HiSeq 2000) | hepatocellular | |
| | | | carcinoma | |
| | | | patients[J]. | |
| | | | Proceedings of the | |
| | | | National Academy | |
| | | | of Sciences, 2015, | |
| | | | 112(11): E1317- | |
| | | | E1325. | |
| 423 samples from | Human | whole- | Cristiano S, Leal A, | |
| Nature paper (215 | plasma | genome | Phallen J, et al. | |
| healthy samples, 208 | | sequencing | Genome-wide | |
| cancer samples (54 | | data | cell-free DNA | |
| breast cancer samples, | | (Illumina | fragmentation in | |
| 26 bile duct samples, 27 | | HiSeq | patients with | |
| colorectal cancer | | 2000/2500) | cancer[J]. | |
| samples, 27 gastric | | | Nature, | |
| samples, 12 lung cancer | | | 2019, 570(7761): | |
| samples, 28 ovarian | | | 385-389. | |
| cancer samples and 34 | | | | |
| pancreatic samples)) | | | | |
| 196 peaks from 96 | White | DNase-Seq | | http://dcc.blueprint- |
| different samples | blood | | | epigenome.eu/#/files |
| (BLUEPRINT) | cells | | | |
| 14 peaks (narrow peaks | White | DNase-Seq | Bernstein B E, | https://egg2.wustl.edu/roadmap/ |
| and board peaks) from 7 | blood | | Stamatoyannopoulos J A, | data/byFileType/peaks/ |
| samples | cells | | Costello J F, | consolidated/ |
| (E29, E32, E33, E34, | | | et al. The NIH | |
| E46, E50, E51) (Roadmap) | | | roadmap | |
| | | | epigenomics | |
| | | | mapping | |
| | | | consortium[J]. | |
| | | | Nature | |
| | | | biotechnology, | |
| | | | 2010, 28(10): | |
| | | | 1045-1048. | |
| GSE118189 (NCBI) | 25 blood | ATAC-Seq | Diego Calderon, | https://www.ncbi.nlm.nih.gov/ |
| | cell types | | Michelle L. T. | geo/query/acc.cgi?acc=GSE118189 |
| | | | Nguyen, Anja | |
| | | | Mezger, Arwa | |
| | | | Kathiria, Vinh | |
| | | | Nguyen, Ninnia | |
| | | | Lescano, Beijing | |
| | | | Wu, John | |
| | | | Trombetta, Jessica | |
| | | | V. Ribado, David | |
| | | | A. Knowles, Ziyue | |
| | | | Gao, Audrey V. | |
| | | | Parent, Trevor D. | |
| | | | Burt, Mark S. | |
| | | | Anderson, Lindsey | |
| | | | A. Criswell, | |
| | | | William J. | |
| | | | Greenleaf, | |
| | | | Alexander | |
| | | | Marson, and | |
| | | | Jonathan K. | |
| | | | Pritchard. | |
| | | | Landscape of | |
| | | | stimulation- | |
| | | | responsive | |
| | | | chromatin across | |
| | | | diverse human | |
| | | | immune cells. | |
| | | | bioRxiv 409722; | |
| | | | doi: 10.1101/409722 | |
| GSE74912 (NCBI) | hematopoietic | ATAC-Seq | Corces M R, | https://www.ncbi.nlm.nih.gov/ |
| | and | | Buenrostro J D, | geo/query/acc.cgi?acc=GSE74912 |
| | leukemic | | Wu B, Greenside | |
| | cell types | | PG et al. Lineage- | |
| | | | specific and | |
| | | | single-cell | |
| | | | chromatin | |
| | | | accessibility | |
| | | | charts human | |
| | | | hematopoiesis | |
| | | | and leukemia | |
| | | | evolution. Nat | |
| | | | Genet 2016 | |
| | | | October; 48(10): | |
| | | | 1193-203. PMID: | |
| | | | 27526324 | |
| ENCFF461VKA and | liver featl | DNase-Seq | | https://www.encodeproject.org/ |
| ENCFF081JVT (ENCODE) | | | | experiments/ENCSR562FNN/ |
| GSM2400294 (Both | Homo sapiens | DNase-Seq | | https://www.ncbi.nlm.nih.gov/ |
| board peaks and narrow | right lobe | | | geo/query/acc.cgi?acc=GSM2400294 |
| peaks: NCBI) | of liver | | | |
| | tissue female | | | |
| | adult | | | |
| ENCFF957JFJ and | Homo sapiens | DNase-Seq | | https://www.encodeproject.org/ |
| ENCFF571RHF (ENcode) | HepG2 | | | experiments/ENCSR000EJV/ |
| E29 (macs2 −log10 p | Primary | DNase-Seq | | https://egg2.wustl.edu/roadmap/ |
| value signal files | monocytes | (signals), | | data/byFileType/signal/ |
| from Roadmap) | from | H3K4me1, | | consolidated/macs2signal/pval/ |
| | peripheral | H3K4me3, | | |
| | blood | H3K9me3, | | |
| | | H3K27me3, | | |
| | | H3K36me3, | | |
| | | H3K27ac | | |
| E30 (macs2 −log10 p | Primary | DNase-Seq | | https://egg2.wustl.edu/roadmap/ |
| value signal files | neutrophils | (signals, | | data/byFileType/signal/ |
| from Roadmap) | from | imputed | | consolidated/macs2signal/pval/; |
| | peripheral | data), | | https://egg2.wustl.edu/ |
| | blood | H3K4me1, | | roadmap/data/byFileType/ |
| | | H3K4me3, | | signal/consolidatedImputed/DNase/ |
| | | H3K9me3, | | E030-DNase.imputed.pval.signal.bigwig; |
| | | H3K27me3, | | https://egg2.wustl.edu/roadmap/ |
| | | H3K36me3, | | data/byFileType/signal/ |
| | | H3K27ac | | consolidatedImputed/H3K27ac/ |
| | | (imputed | | E030-H3K27ac.imputed.pval.signal.bigwig |
| | | data) | | |
| E32 (macs2 −log10 p | Primary | DNase-Seq | | https://egg2.wustl.edu/roadmap/ |
| value signal files | B cells | (signals), | | data/byFileType/signal/ |
| from Roadmap) | from | H3K4me1, | | consolidated/macs2signal/pval/ |
| | peripheral | H3K4me3, | | |
| | blood | H3K9me3, | | |
| | | H3K27me3, | | |
| | | H3K36me3, | | |
| | | H3K27ac | | |
| E34 (macs2 −log10 p | Primary T | DNase-Seq | | https://egg2.wustl.edu/roadmap/ |
| value signal files | cells | (signals), | | data/byFileType/signal/ |
| from Roadmap) | from | H3K4me1, | | consolidated/macs2signal/pval/ |
| | peripheral | H3K4me3, | | |
| | blood | H3K9me3, | | |
| | | H3K27me3, | | |
| | | H3K36me3, | | |
| | | H3K27ac | | |
| E71 (macs2 −log10 p | Brain | H3K4me1, | | https://egg2.wustl.edu/roadmap/ |
| value signal files | hippocampus | H3K4me3, | | data/byFileType/signal/ |
| from Roadmap) | | H3K9me3, | | consolidated/macs2signal/pval/ |
| | | H3K27me3, | | |
| | | H3K36me3, | | |
| | | H3K27ac | | |
| E03 (macs2 −log10 p | H1 Cell | H3K4me1 | Roadmap | https://egg2.wustl.edu/roadmap/ |
| value signal files | Line | | | data/byFileType/signal/ |
| from Roadmap) | | | | consolidated/macs2signal/pval/ |
| | | | | E003-H3K4me1.pval.signal.bigwig |
| E08 (macs2 −log10 p | H9 Cell | H3K4me1 | Roadmap | https://egg2.wustl.edu/roadmap/ |
| value signal files | Line | | | data/byFileType/signal/ |
| from Roadmap) | | | | consolidated/macs2signal/pval/ |
| | | | | E009-H3K4me1.pval.signal.bigwig |
| E27 (macs2 −log10 p | Breast | H3K4me2 | Roadmap | https://egg2.wustl.edu/roadmap/ |
| value signal files | Myoepithelial | | | data/byFileType/signal/ |
| from Roadmap) | Primary | | | consolidated/macs2signal/pval/ |
| | Cells | | | E027-H3K4me1.pval.signal.bigwig |
| E76 (macs2 −log10 p | Colon | H3K4me1 | Roadmap | https://egg2.wustl.edu/roadmap/ |
| value signal files | Smooth | | | data/byFileType/signal/ |
| from Roadmap) | Muscle | | | consolidated/macs2signal/pval/ |
| | | | | E076-H3K4me1.pval.signal.bigwig |
| E96 (macs2 −log10 p | Lung | H3K4me1 | Roadmap | https://egg2.wustl.edu/roadmap/ |
| value signal files | | | | data/byFileType/signal/ |
| from Roadmap) | | | | consolidated/macs2signal/pval/ |
| | | | | E096-H3K4me1.pval.signal.bigwig |
| 43_Hm05_BIMa_Ct | Macrophages | NOMe-seq | DEEP | https://epigenomesportal.ca/ |
| | | | | tracks/DEEP/hg19/63215.DEEP. |
| | | | | 43_Hm05_BIMa_Ct.NOMe- |
| | | | | Seq.accessibility_profile.bigWig |
| 43_Hm05_BIMa_TE | Macrophages | NOMe-seq | DEEP | https://epigenomesportal.ca/ |
| | | | | tracks/DEEP/hg19/63235.DEEP. |
| | | | | 43_Hm05_BIMa_TE.NOMe- |
| | | | | Seq.accessibility_profile.bigWig |
| 43_Hm05_BIMa_TO | Macrophages | NOMe-seq | DEEP | https://epigenomesportal.ca/ |
| | | | | tracks/DEEP/hg19/63255.DEEP. |
| | | | | 43_Hm05_BIMa_TO.NOMe- |
| | | | | Seq.accessibility_profile.bigWig |
| 43_Hm05_BIMo_Ct | Monocytes | NOMe-seq | DEEP | https://epigenomesportal.ca/ |
| | | | | tracks/DEEP/hg19/63275.DEEP. |
| | | | | 43_Hm05_BIMo_Ct.NOMe- |
| | | | | Seq.accessibility_profile.bigWig |
| 51_Hf01_BICM_Ct | Central | NOMe-seq | DEEP | https://epigenomesportal.ca/ |
| | memory | | | tracks/DEEP/hg19/63295.DEEP. |
| | T-cells | | | 51_Hf01_BICM_Ct.NOMe- |
| | | | | Seq.accessibility_profile.bigWig |
| 51_Hf03_BICM_Ct | Central | NOMe-seq | DEEP | https://epigenomesportal.ca/ |
| | memory | | | tracks/DEEP/hg19/63339.DEEP. |
| | T-cells | | | 51_Hf03_BICM_Ct.NOMe- |
| | | | | Seq.accessibility_profile.bigWig |
| 51_Hf03_BIEM_Ct | Effector | NOMe-seq | DEEP | https://epigenomesportal.ca/ |
| | memory | | | tracks/DEEP/hg19/63348.DEEP. |
| | T-cells | | | 51_Hf03_BIEM_Ct.NOMe- |
| | | | | Seq.accessibility_profile.bigWig |
| 51_Hf03_BITN_Ct | Naive | NOMe-seq | DEEP | https://epigenomesportal.ca/ |
| | T-cells | | | tracks/DEEP/hg19/63359.DEEP. |
| | | | | 51_Hf03_BITN_Ct.NOMe- |
| | | | | Seq.accessibility_profile.bigWig |
| 51_Hf04_BICM_Ct | Central | NOMe-seq | DEEP | https://epigenomesportal.ca/ |
| | memory | | | tracks/DEEP/hg19/63381.DEEP. |
| | T-cells | | | 51_Hf04_BICM_Ct.NOMe- |
| | | | | Seq.accessibility_profile.bigWig |
| 51_Hf04_BIEM_Ct | Effector | NOMe-seq | DEEP | https://epigenomesportal.ca/ |
| | memory | | | tracks/DEEP/hg19/63401.DEEP. |
| | T-cells | | | 51_Hf04_BIEM_Ct.NOMe- |
| | | | | Seq.accessibility_profile.bigWig |
| 51_Hf04_BITN_Ct | Naive | NOMe-seq | DEEP | https://epigenomesportal.ca/ |
| | T-cells | | | tracks/DEEP/hg19/63433.DEEP. |
| | | | | 51_Hf04_BITN_Ct.NOMe- |
| | | | | Seq.accessibility_profile.bigWig |
| GSM1972155 | human | ATAC-See | Chen X, Shen Y, | https://www.ncbi.nlm.nih.gov/geo/ |
| (NCBI GEO) | neutrophil | | Draper W, | query/acc.cgi?acc=GSM1972155 |
| | | | Buenrostro J D et | |
| | | | al. ATAC-see | |
| | | | reveals the | |
| | | | accessible | |
| | | | genome by | |
| | | | transposase- | |
| | | | mediated imaging | |
| | | | and sequencing. | |
| | | | Nat Methods 2016 | |
| | | | December; 13(12): | |
| | | | 1013-1020. PMID: | |
| | | | 27749837 | |
| HG00096 (1000 | | whole- | 1000 Genomes | ftp://ftp.1000genomes.ebi.ac.uk/vol1/ |
| Genomes) | | genome | Project | ftp/phase3/data/HG00096/alignment/ |
| | | sequencing | Consortium. A | HG00096.mapped.ILLUMINA.bwa.GBR. |
| | | data | global reference | low_coverage.20120522.bam |
| | | | for human genetic | |
| | | | variation[J]. | |
| | | | Nature, 2015, | |
| | | | 526(7571): 68-74. | |
| HG00097 (1000 | | whole- | 1000 Genomes | ftp://ftp.1000genomes.ebi.ac.uk/vol1/ |
| Genomes) | | genome | Project | ftp/phase3/data/HG00097/alignment/ |
| | | sequencing | Consortium. A | HG00097.mapped.ILLUMINA.bwa.GBR. |
| | | data | global reference | low_coverage.20130415.bam |
| | | | for human genetic | |
| | | | variation[J]. | |
| | | | Nature, 2015, | |
| | | | 526(7571): 68-74. | |
| HG00099 (1000 | | whole- | 1000 Genomes | ftp://ftp.1000genomes.ebi.ac.uk/vol1/ |
| Genomes) | | genome | Project | ftp/phase3/data/HG00099/alignment/ |
| | | sequencing | Consortium. A | HG00099.mapped.ILLUMINA.bwa.GBR. |
| | | data | global reference | low_coverage.20130415.bam |
| | | | for human genetic | |
| | | | variation[J]. | |
| | | | Nature, 2015, | |
| | | | 526(7571): 68-74. | |
| HG00100 (1000 | | whole- | 1000 Genomes | ftp://ftp.1000genomes.ebi.ac.uk/vol1/ |
| Genomes) | | genome | Project | ftp/phase3/data/HG00100/alignment/ |
| | | sequencing | Consortium. A | HG00100.mapped.ILLUMINA.bwa.GBR. |
| | | data | global reference | low_coverage.20130415.bam |
| | | | for human genetic | |
| | | | variation[J]. | |
| | | | Nature, 2015, | |
| | | | 526(7571): 68-74. | |
| HG00101 (1000 | | whole- | 1000 Genomes | ftp://ftp.1000genomes.ebi.ac.uk/vol1/ |
| Genomes) | | genome | Project | ftp/phase3/data/HG00101/alignment/ |
| | | sequencing | Consortium. A | HG00101.mapped.ILLUMINA.bwa.GBR. |
| | | data | global reference | low_coverage.20130415.bam |
| | | | for human genetic | |
| | | | variation[J]. | |
| | | | Nature, 2015, | |
| | | | 526(7571): 68-74. | |
| Chromatin state data | | | | https://egg2.wustl.edu/roadmap/data/ |
| (Core 15-state | | | | byFileType/chromhmmSegmentations/ |
| chromHMM model) of | | | | ChmmModels/coreMarks/ |
| 44 cell types (Roadmap) | | | | jointModel/final/ |

Taken together, in healthy individuals, these de novo characterized cfDNA fragmentation hotspots are highly enriched in the gene-regulatory elements.

Cell-Free DNA Fragmentation Hotspots Reveal the Potential Regulatory Aberrations of Microsatellites, CTCF, and Genes from Peripheral Immune Cells in Early-Stage Cancer.

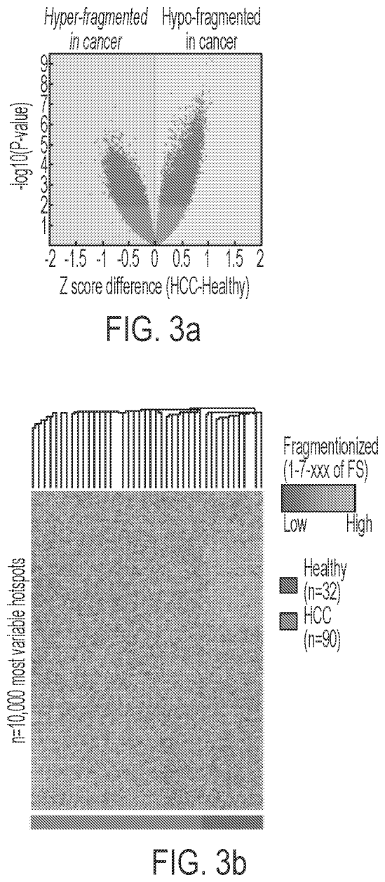

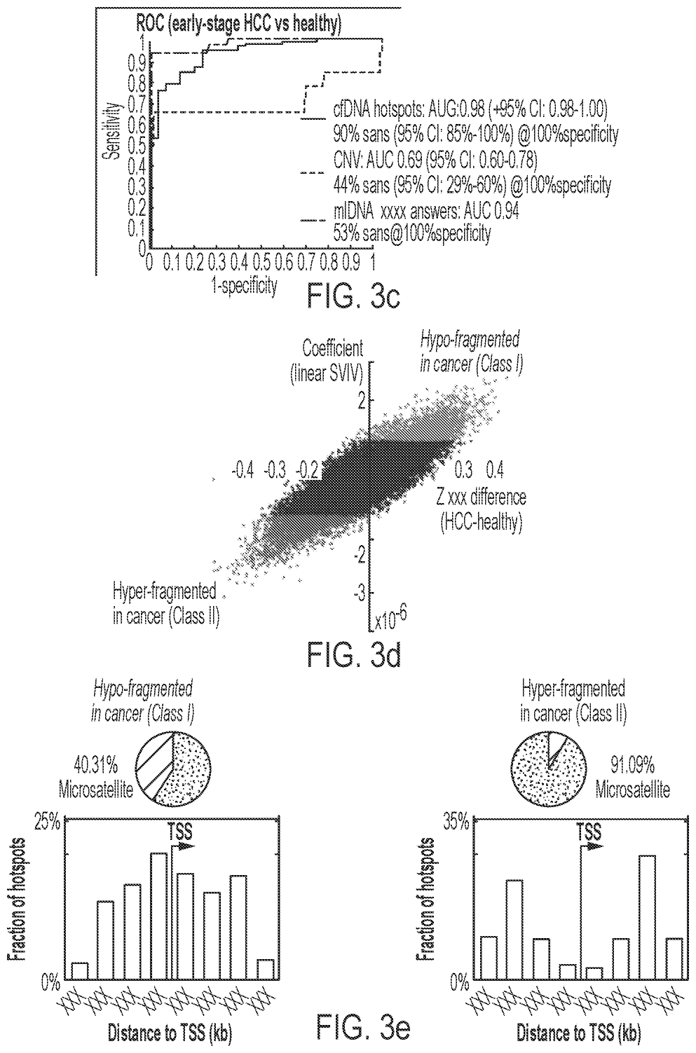

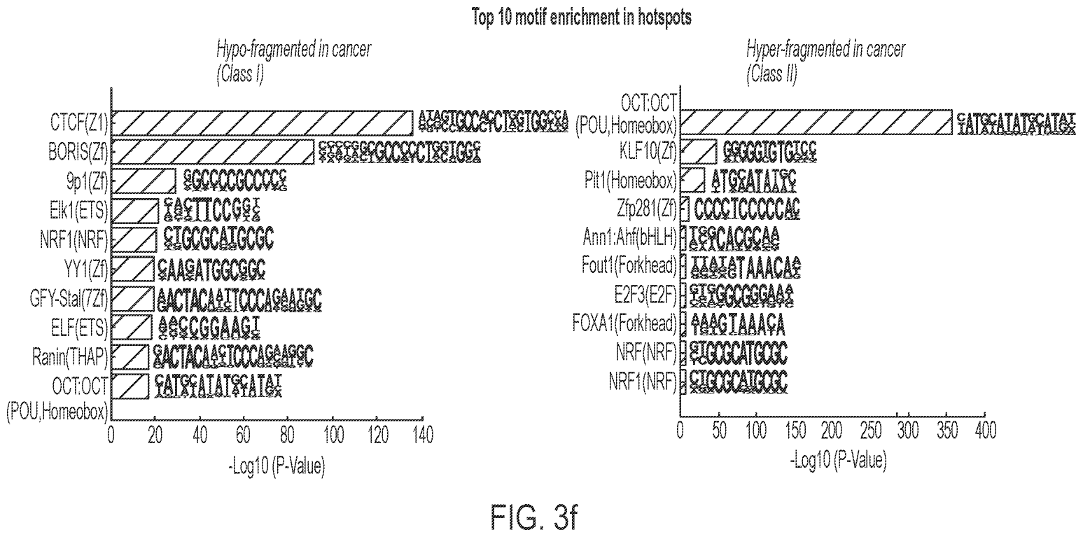

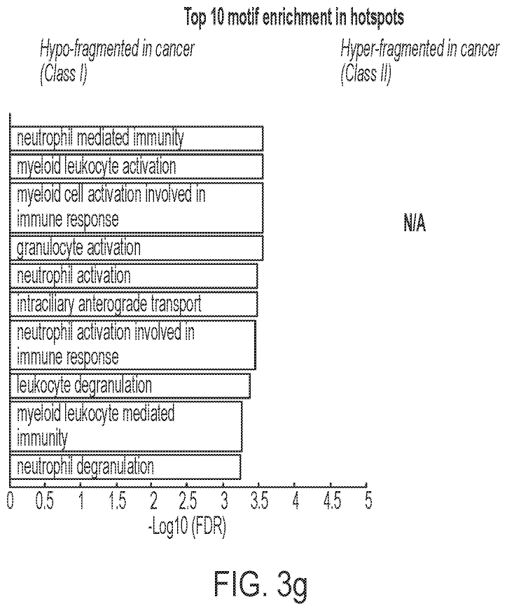

Further, characterized the cfDNA fragmentation hotspots in early-stage cancer. We collected the publicly available low-coverage cfDNA WGS (˜1×/sample) from 90 patients with early-stage hepatocellular carcinoma (HCC, 85 of them are Barcelona Clinic Liver Cancer stage A, 5 of them are stage B) and 32 healthy individuals from the same study[25]. We pooled the low-coverage cfDNA WGS to obtain enough fragments for the hotspot calling in each condition (>=400 million fragments, Details in Supplementary Methods, FIG. S7)[25]. The volcano plot of the p-value (two-sample t-test) and z-score difference of IFS between HCC and healthy across all the fragmentation hotspots showed more fractions of hypo-fragmented hotspots in early-stage HCC (FIG. 3a). Further, the unsupervised hierarchical clustering of the top 10,000 most variable hotspots showed a clear fragmentation dynamic between HCC and healthy (FIG. 3b, FIG. S8-9). Therefore, we utilized the IFS from the cfDNA fragmentation hotspots to classify the HCC and healthy individuals by a linear Support Vector Machine (SVM) approach (Details in Methods). By 10-fold cross-validation, we observed a much higher classification performance (93% sensitivity at 100% specificity) than that by using copy number variations (CNVs) with the same machine learning infrastructure and same data split (44% sensitivity at 100% specificity), mitochondria DNA (mtDNA)[25] (53% sensitivity at 100% specificity) (FIG. 3c, Table S2-3, FIG. S10a) and other previously developed fragmentation approaches [8,10,25]. We also applied other machine learning approaches with the same data split in cross-validation and observed overall good performances by using cfDNA fragmentation hotspots (FIG. S10b).

TABLE S2
Table S2: Performance comparison in HCC vs. healthy (before GC bias correction)

| | | Sensitivity | Sensitivity | Sensitivity | Sensitivity |
|---|---|---|---|---|---|
| | | @100% | @98% | @95% | @85% |
| | AUC | specificity | specificity | specificity | specificity |
| IFS | 0.9815 | 0.9333 | 0.9333 | 0.9333 | 0.9333 |
| hotspots | (0.95 CI: | (0.95 CI: | (0.95 CI: | (0.95 CI: | (0.95 CI: |
| | 0.9592-1.0000) | 0.8462-1.0000) | 0.8462-1.0000) | 0.8462-1.0000) | 0.8462-1.0000) |
| CNV | 0.6889 | 0.4444 | 0.6556 | 0.6556 | 0.6556 |
| | (0.95 CI: | (0.95 CI: | (0.95 CI: | (0.95 CI: | (0.95 CI: |
| | 0.5997-0.7781) | 0.2888-0.6001) | 0.5532-0.7580) | 0.5532-0.7580) | 0.5532-0.7580) |
| mtDNA | 0.9358 | 0.5333 | 0.5333 | 0.7556 | 0.8444 |

TABLE S3
Table S3: Performance in in HCC vs. healthy (after GC bias correction)

| | | Sensitivity | Sensitivity | Sensitivity | Sensitivity |
|---|---|---|---|---|---|
| | | @100% | @98% | @95% | @85% |
| | AUC | specificity | specificity | specificity | specificity |
| IFS | 0.9380 | 0.8778 | 0.8778 | 0.8778 | 0.8778 |
| hotspots | (0.95 CI: | (0.95 CI: | (0.95 CI: | (0.95 CI: | (0.95 CI: |
| | 0.8670-1.0000) | 0.7274-1.0000) | 0.7274-1.0000) | 0.7274-1.0000) | 0.7274-1.0000) |

We next asked why the IFS signals in fragmentation hotspots can boost the classification performance. We split the hotspots that significantly contributed to the classification model into two groups: Class I (Hypo-fragmented in cancer) and Class II (Hyper-fragmented in cancer) (FIG. 3d, Table S4). The Class I hotspots are mostly in promoter regions, which suggests the potential silencing of genes with the decreases of fragmentation (closed chromatin status). Further, these potential silenced genes were highly enriched in the immune-related gene ontology biological processes (GO BPs) from the peripheral immune cells, such as neutrophils and myeloid cells (FIG. 3e, g, Table S5). To confirm our observations by another dataset, we collected publicly available gene expression data in peripheral blood mononuclear cells (PBMC) from early-stage HCC patients and healthy individuals[26]. The results suggested that the significant fractions of genes, whose promoters are overlapped with Class I hotspots, are indeed silenced at peripheral immune cells in early-stage HCC patients compared to the global background (Fisher exact test, p=1.83e⁻⁵, FIG. S11a). Class II hotspots are mostly in microsatellites, which suggested the potential increases of fragmentation at microsatellites in early-stage cancer (FIG. 3e). Since the fragmentation process is known to be affected by DNA methylation[27], to validate this observation, we collected public available cfDNA methylation data measured by whole-genome bisulfite sequencing (WGBS) in early-stage HCC patients and healthy individuals[28]. The DNA methylation level in Class II hotspots showed hypomethylation in early-stage HCC patients compared to healthy individuals, which indeed suggested the potential changes of epigenetic environments near microsatellites that can affect the cfDNA fragmentation process (FIG. S11b). We further checked the enrichment of motifs at these two groups of hotspots. The results further suggested the differences of motif enrichment between two groups (FIG. 3f). Further experimental validations from the same patients are needed to make a solid conclusion.

TABLE S4
Most informative fragmentation hotspot location in between HCC and Healthy

| Chromosome | | | Coefficient | Mean IFS (z-score) | Mean IFS (z-score) |
|---|---|---|---|---|---|
| id | Start | End | in SVM | in healthy samples | in HCC samples |
| chr1 | 42127500 | 42128620 | −0.003092339 | −0.745976153 | −1.211270022 |
| chr6 | 1536280 | 1536520 | −0.00277068 | −0.899323728 | −1.274585773 |
| chr3 | 131723940 | 131724460 | −0.002745501 | −0.806179554 | −1.232007433 |
| chr12 | 16784660 | 16785180 | −0.002717536 | −0.977168066 | −1.378388792 |
| chr7 | 22601680 | 22602140 | −0.002670085 | −0.801244479 | −1.026692037 |
| chr4 | 172758540 | 172759080 | −0.002642817 | −0.830128732 | −1.279370094 |
| chr8 | 117284760 | 117285480 | 0.002625112 | −1.188765843 | −0.755657421 |
| chr4 | 62331240 | 62331720 | −0.002595125 | −0.956725388 | −1.232214749 |
| chr18 | 23410460 | 23410960 | −0.002566574 | −0.899173511 | −1.294271456 |
| chr8 | 96145560 | 96146820 | 0.002566441 | −1.280740188 | −0.919557716 |
| chr12 | 88793740 | 88794100 | −0.00255522 | −0.900556147 | −1.264688706 |
| chr13 | 114004660 | 114005120 | 0.002536307 | −1.179577214 | −0.78345726 |
| chr13 | 114004660 | 114005120 | 0.002536307 | −1.179577214 | −0.78345726 |
| chr1 | 37633700 | 37634060 | −0.002503515 | −0.770771142 | −1.190388787 |
| chr11 | 115271040 | 115271640 | −0.002498974 | −0.957313931 | −1.351390199 |
| chr6 | 72184260 | 72184660 | −0.002484748 | −0.884544501 | −1.319328215 |
| chr17 | 37048940 | 37049440 | 0.002464081 | −1.051891538 | −0.686862085 |
| chr11 | 64052060 | 64053720 | −0.002464024 | −0.811700068 | −1.145782704 |
| chr11 | 64052020 | 64053760 | −0.002464024 | −0.811700068 | −1.145782704 |
| chr6 | 40973940 | 40974140 | 0.002463092 | −1.301756354 | −0.9243507 |
| chr7 | 129697020 | 129697400 | −0.002441872 | −0.778470013 | −1.214812016 |
| chr10 | 119527440 | 119528100 | −0.002419826 | −0.685792918 | −1.18172942 |
| chr14 | 30132100 | 30132480 | −0.002400807 | −0.723720978 | −1.210908092 |
| chr6 | 74819240 | 74819800 | 0.002388047 | −1.192243304 | −0.920509929 |
| chr21 | 44189220 | 44190060 | 0.002386128 | −1.042645509 | −0.741092506 |
| chr2 | 207831820 | 207832300 | −0.002370527 | −0.565237499 | −0.992796478 |
| chr6 | 80051240 | 80051740 | −0.002359436 | −0.939575187 | −1.302910417 |
| chr16 | 7944320 | 7945420 | −0.00233685 | −0.67910391 | −1.133025032 |
| chr11 | 68608180 | 68608820 | 0.002334962 | −1.211278827 | −0.906214929 |
| chr9 | 125753080 | 125753300 | 0.002331956 | −1.100666842 | −0.775055031 |
| chr9 | 125753080 | 125753300 | 0.002331956 | −1.100666842 | −0.775055031 |
| chr3 | 185394240 | 185395300 | −0.002327637 | −0.885630637 | −1.363697683 |
| chr5 | 137617900 | 137618220 | −0.002323105 | −0.840614867 | −1.21701667 |
| chr22 | 40603220 | 40603620 | −0.002314718 | −0.705800753 | −0.960916681 |
| chr3 | 80453000 | 80453440 | 0.002312388 | −1.149766375 | −0.97064277 |
| chr5 | 27314780 | 27315160 | −0.002298809 | −0.831072591 | −1.159532797 |
| chr22 | 46511940 | 46513160 | 0.0022962 | −1.162368428 | −0.874595747 |
| chr1 | 155826720 | 155827260 | 0.002282698 | −1.157656741 | −0.77104378 |
| chr6 | 136605060 | 136605500 | −0.002272854 | −0.843049152 | −1.076637703 |
| chr15 | 75339480 | 75339800 | 0.002270929 | −1.099225098 | −0.833808561 |
| chr1 | 37633620 | 37634080 | −0.002268608 | −0.91823478 | −1.289763036 |
| chr10 | 63780620 | 63780960 | −0.00226826 | −0.804969567 | −1.130110254 |
| chr2 | 82047820 | 82048180 | −0.002266471 | −0.83849041 | −1.247244194 |
| chr2 | 51775780 | 51776260 | −0.002265467 | −0.898043667 | −1.320142328 |
| chr5 | 66748640 | 66749160 | −0.00226123 | −0.946135455 | −1.24939146 |
| chr5 | 84048300 | 84048780 | −0.002253323 | −0.904488238 | −1.259116765 |
| chr4 | 26929280 | 26929700 | −0.002250826 | −0.934122954 | −1.282166847 |
| chr3 | 185394260 | 185395300 | −0.002246469 | −0.930183547 | −1.368360953 |
| chr8 | 122389640 | 122389980 | 0.002238366 | −1.09654499 | −0.884019382 |
| chr11 | 5707740 | 5707960 | −0.002234188 | −0.803275988 | −1.149232478 |
| chr3 | 45931520 | 45932060 | −0.002233631 | −0.94409477 | −1.365151235 |
| chr5 | 35661220 | 35661520 | −0.002232496 | −0.937511406 | −1.2586665 |
| chr1 | 97373480 | 97373940 | −0.002229248 | −0.843481775 | −1.232304261 |
| chr16 | 18092480 | 18092980 | −0.002226695 | −0.710824591 | −1.082137048 |
| chr1 | 113741740 | 113742240 | −0.002222891 | −0.885269605 | −1.226255647 |
| chr15 | 75339460 | 75339780 | 0.002216959 | −1.172901618 | −0.87081135 |
| chr6 | 79117720 | 79118480 | −0.002210889 | −0.962347825 | −1.235986073 |
| chr1 | 39347780 | 39348380 | −0.002195442 | −0.765878752 | −1.102433981 |
| chr9 | 91056560 | 91056900 | −0.002189466 | −0.626590328 | −0.966183087 |
| chr19 | 16887700 | 16888880 | 0.002189206 | −1.149410214 | −0.819731392 |
| chr8 | 138742840 | 138744020 | 0.002187092 | −1.395477202 | −1.067514257 |
| chr8 | 138742820 | 138744040 | 0.002187092 | −1.395477202 | −1.067514257 |
| chr2 | 73297740 | 73299160 | 0.002180218 | −1.341321285 | −1.080881733 |
| chr8 | 117284760 | 117285420 | 0.002179818 | −1.194724848 | −0.819179875 |
| chr2 | 17315900 | 17316280 | −0.002176235 | −0.926659875 | −1.221121024 |
| chr6 | 48551900 | 48552440 | −0.002175686 | −0.749641475 | −1.246232536 |
| chr1 | 216496380 | 216496860 | −0.002169701 | −0.682337033 | −1.056119414 |
| chr5 | 37517820 | 37518140 | −0.002169195 | −0.766310186 | −1.146658833 |
| chr11 | 41491500 | 41491960 | 0.002163609 | −1.300432784 | −1.156103214 |
| chr6 | 72184160 | 72184780 | −0.00216248 | −0.884544501 | −1.265270993 |
| chr2 | 78117540 | 78118500 | 0.002161285 | −1.396284482 | −1.167468501 |
| chr4 | 106199100 | 106199560 | −0.002159953 | −0.907672149 | −1.189229624 |
| chr10 | 29042340 | 29042780 | −0.002157846 | −0.810947827 | −1.139366691 |
| chr16 | 18092480 | 18092920 | −0.002151984 | −0.796215616 | −1.192465801 |
| chr17 | 73936280 | 73937960 | 0.002149503 | −1.131490238 | −0.827364721 |
| chr2 | 112856920 | 112857280 | −0.002148878 | −0.894131991 | −1.1721262 |
| chr1 | 50099360 | 50099760 | −0.002147747 | −0.774491669 | −1.201886556 |
| chr15 | 75497040 | 75497460 | 0.002147105 | −1.19613241 | −0.88790135 |
| chr22 | 34896000 | 34896840 | 0.002143777 | −1.056089006 | −0.804089504 |
| chr6 | 38607280 | 38608160 | 0.002142368 | −1.257980376 | −0.982617816 |
| chr22 | 38749080 | 38749580 | 0.002137374 | −1.156192893 | −0.891676471 |
| chr13 | 61752060 | 61752560 | −0.002131038 | −0.932926362 | −1.201013005 |
| chr6 | 103306480 | 103306860 | 0.002129424 | −0.956523653 | −0.71096895 |
| chr3 | 45931580 | 45932020 | −0.002124758 | −0.981858347 | −1.388432933 |
| chr1 | 108092460 | 108092920 | 0.002124736 | −1.125125173 | −0.984037559 |
| chr17 | 2731780 | 2732040 | 0.002123727 | −1.21072564 | −0.84761373 |
| chr17 | 2731780 | 2732040 | 0.002123727 | −1.21072564 | −0.84761373 |
| chr1 | 98034780 | 98035200 | −0.002123482 | −0.656753725 | −0.994526857 |
| chr11 | 39607020 | 39607400 | −0.002120119 | −0.920310885 | −1.280187263 |
| chr1 | 36839460 | 36839780 | 0.00211989 | −1.118065861 | −0.762027957 |
| chr1 | 8635440 | 8635820 | −0.002118036 | −0.813576937 | −1.205300594 |
| chr1 | 8635380 | 8635880 | −0.002118036 | −0.813576937 | −1.205300594 |
| chr4 | 34988140 | 34988540 | −0.002115372 | −0.92124435 | −1.296923716 |
| chr9 | 118004600 | 118005020 | −0.002111585 | −0.448055744 | −0.736610843 |
| chr5 | 147762720 | 147764120 | 0.00211005 | −1.247801733 | −0.851898823 |
| chr1 | 244742000 | 244742280 | −0.002109047 | −0.680909314 | −0.988903933 |
| chr3 | 194338940 | 194339500 | −0.002107397 | −0.926289267 | −1.297815935 |
| chr11 | 74160440 | 74160700 | 0.002107094 | −1.256927884 | −0.980165326 |
| chr14 | 44908760 | 44909120 | −0.002105623 | −0.883430947 | −1.214042787 |
| chr19 | 3821820 | 3822360 | 0.002100105 | −1.160627889 | −0.900756484 |
| chr19 | 3821820 | 3822360 | 0.002100105 | −1.160627889 | −0.900756484 |
| chr13 | 94551060 | 94551400 | −0.00209945 | −0.815797236 | −1.18643113 |
| chr13 | 94551000 | 94551460 | −0.00209945 | −0.815797236 | −1.18643113 |
| chr5 | 100423060 | 100423260 | −0.002097332 | −0.934513265 | −1.241928494 |
| chr5 | 100423060 | 100423260 | −0.002097332 | −0.934513265 | −1.241928494 |
| chr14 | 78173840 | 78175040 | −0.002094919 | −0.847999108 | −1.111892702 |
| chr8 | 96145520 | 96147040 | 0.002093579 | −1.262956979 | −0.909594362 |
| chr20 | 48728900 | 48730520 | 0.002089984 | −1.387785571 | −1.090349593 |
| chr1 | 113741780 | 113742180 | −0.002088194 | −0.949498197 | −1.299581197 |
| chr7 | 2387680 | 2387960 | 0.002085878 | −1.038776035 | −0.771693755 |
| chr7 | 2387680 | 2387960 | 0.002085878 | −1.038776035 | −0.771693755 |
| chr3 | 97947980 | 97948440 | −0.002082591 | −0.901106932 | −1.183575775 |
| chr19 | 39137800 | 39138960 | 0.002081556 | −0.985959881 | −0.747786994 |
| chr11 | 114614420 | 114614800 | −0.002079925 | −0.751022389 | −1.08210914 |
| chr6 | 146568760 | 146569220 | −0.002074471 | −0.775337723 | −1.093809409 |
| chr9 | 137203080 | 137203600 | −0.002066086 | −0.77699018 | −1.157180879 |
| chr3 | 131723960 | 131724420 | −0.002064998 | −0.92287134 | −1.250774545 |
| chr17 | 46151740 | 46152040 | 0.002064131 | −1.240718001 | −0.973478313 |
| chr5 | 17400140 | 17400640 | −0.002063881 | −0.934465823 | −1.31043278 |
| chr2 | 54197400 | 54198460 | 0.002062867 | −1.345624484 | −0.99179228 |
| chr15 | 47076900 | 47077460 | −0.002061925 | −0.758983555 | −1.009633926 |
| chr2 | 107841300 | 107841800 | −0.002059263 | −0.886121025 | −1.220164855 |
| chr6 | 158552160 | 158552800 | −0.002057233 | −0.924685046 | −1.323796256 |
| chr1 | 6639540 | 6640660 | 0.00205701 | −1.280479873 | −0.948416654 |
| chr7 | 118731680 | 118732080 | 0.002056879 | −1.247623401 | −1.067267756 |
| chr9 | 130889860 | 130891760 | 0.002055939 | −1.055406619 | −0.774241955 |
| chr2 | 232574980 | 232575300 | 0.002051518 | −1.334994879 | −1.084166748 |
| chr3 | 10156860 | 10157660 | 0.002050067 | −1.363385293 | −1.124727708 |
| chr5 | 99901680 | 99902060 | −0.002046116 | −1.012013458 | −1.301140557 |
| chr10 | 116541220 | 116541680 | −0.002043776 | −0.793209616 | −1.088646672 |
| chr1 | 165775680 | 165776040 | 0.002043516 | −1.05608244 | −0.842175134 |
| chr4 | 132079620 | 132080200 | −0.002037348 | −0.900851695 | −1.225720573 |
| chr6 | 116851860 | 116852300 | −0.002034477 | −0.986899014 | −1.297578985 |
| chr13 | 101469260 | 101469860 | −0.002031506 | −1.009653953 | −1.275858695 |
| chr15 | 64530660 | 64530920 | −0.002031468 | −0.745286856 | −1.056675893 |
| chr17 | 4634000 | 4634260 | 0.00202941 | −1.054332539 | −0.707839773 |
| chr4 | 134085840 | 134086480 | 0.002026112 | −1.220803935 | −1.059390095 |
| chr7 | 119357280 | 119357640 | −0.00202504 | −0.966040167 | −1.273312416 |
| chr2 | 7302020 | 7302600 | −0.002024059 | −0.971934315 | −1.306649908 |
| chr16 | 80239520 | 80240120 | −0.002022756 | −0.771275912 | −1.154554522 |
| chr8 | 78755500 | 78756020 | −0.002021937 | −1.033161051 | −1.374139334 |
| chr4 | 90078060 | 90078620 | −0.002019676 | −1.025965094 | −1.33098645 |
| chr10 | 50356700 | 50357140 | −0.002019618 | −0.937032221 | −1.256237482 |
| chr7 | 20854340 | 20854580 | −0.002014372 | −0.740853496 | −1.068740906 |
| chr5 | 77655720 | 77657080 | 0.002013253 | −1.238977299 | −0.984212868 |
| chr5 | 173046000 | 173046260 | 0.002008371 | −1.289933021 | −1.001959848 |
| chr11 | 99191820 | 99192340 | −0.002006321 | −0.679196996 | −1.030112001 |
| chr12 | 8234640 | 8235100 | 0.002005278 | −1.266577406 | −0.966065513 |
| chr9 | 11725700 | 11726280 | −0.002005247 | −0.714949278 | −1.077955802 |
| chr11 | 6494280 | 6495480 | 0.002004683 | −1.230709397 | −0.874896625 |
| chr19 | 49630620 | 49631980 | 0.002002635 | −1.132108941 | −0.907694826 |
| chr6 | 83953360 | 83953760 | −0.002002063 | −1.040088202 | −1.355190294 |
| chr17 | 6569360 | 6569640 | 0.002000028 | −1.164509738 | −0.86335074 |
| chr4 | 168381920 | 168382340 | −0.001994842 | −0.909167318 | −1.249450734 |
| chr5 | 5705880 | 5706540 | −0.00199309 | −1.01918548 | −1.36628835 |
| chr2 | 204737760 | 204738340 | 0.001992478 | −1.171952813 | −1.024487387 |
| chr8 | 134465300 | 134466180 | 0.001990818 | −1.34156102 | −1.037791619 |
| chr19 | 42444160 | 42444780 | 0.001989685 | −1.165497572 | −0.80829194 |
| chr1 | 97373520 | 97373920 | −0.001989568 | −0.875738229 | −1.230132377 |
| chr8 | 30890020 | 30891460 | −0.001989522 | −1.054547947 | −1.253462818 |
| chr5 | 88679200 | 88679760 | −0.001988143 | −0.958925903 | −1.274862806 |
| chr4 | 84031600 | 84031880 | 0.001988017 | −1.295054845 | −1.023614201 |
| chr1 | 6639340 | 6640840 | 0.001987874 | −1.261363323 | −0.973966528 |
| chr20 | 6148300 | 6148700 | −0.001987138 | −0.824094249 | −1.130779993 |
| chr8 | 5646740 | 5647120 | −0.001986039 | −0.796030758 | −1.129834269 |
| chr8 | 16046360 | 16046580 | −0.001984397 | −0.895117567 | −1.14315055 |
| chr8 | 16046360 | 16046580 | −0.001984397 | −0.895117567 | −1.14315055 |
| chr22 | 39333400 | 39334040 | 0.001984134 | −1.042955385 | −0.726992713 |
| chr2 | 82047800 | 82048260 | −0.001982841 | −0.921720328 | −1.298913832 |
| chr11 | 40139400 | 40139960 | −0.001982104 | −0.795765352 | −1.201999879 |
| chr9 | 28584720 | 28585220 | 0.001976723 | −1.180014145 | −0.789915306 |
| chr6 | 137182840 | 137183380 | −0.001975408 | −1.025596874 | −1.317308565 |
| chr1 | 165775660 | 165776040 | 0.001974595 | −1.037521689 | −0.808647984 |
| chr9 | 100916440 | 100916880 | −0.001974439 | −0.744073719 | −1.082012409 |
| chr4 | 132079600 | 132080240 | −0.00197425 | −0.900851695 | −1.219174617 |
| chr2 | 207831840 | 207832260 | −0.001971008 | −0.602129851 | −0.998026073 |
| chr2 | 78117660 | 78118500 | 0.001967379 | −1.374041701 | −1.146776974 |
| chr14 | 61261180 | 61261600 | −0.001966973 | −0.855090081 | −1.126852743 |
| chr14 | 61261140 | 61261640 | −0.001966973 | −0.855090081 | −1.126852743 |
| chr10 | 23039620 | 23040080 | −0.00196649 | −0.838018352 | −1.138528966 |
| chr9 | 134722920 | 134723740 | 0.001965861 | −1.053479848 | −0.676054093 |
| chr21 | 16162080 | 16162520 | −0.001964712 | −0.822647934 | −1.110443439 |
| chr16 | 84764060 | 84764340 | 0.001964261 | −1.222874216 | −1.022661038 |
| chr2 | 22754880 | 22755220 | −0.00196286 | −0.955837177 | −1.215751512 |
| chr2 | 22754820 | 22755280 | −0.00196286 | −0.955837177 | −1.215751512 |
| chr5 | 17400200 | 17400560 | −0.001959929 | −0.914554078 | −1.28904379 |
| chr7 | 91268700 | 91269240 | −0.001959602 | −1.017278731 | −1.285241489 |
| chr17 | 79111440 | 79113000 | 0.00195755 | −1.163376165 | −0.906151214 |
| chr2 | 140850420 | 140850940 | −0.001956142 | −0.915955498 | −1.142394718 |
| chr17 | 54910600 | 54912420 | 0.001955458 | −1.17984244 | −0.967791609 |
| chr1 | 156115800 | 156116080 | 0.001950185 | −1.173452398 | −0.87626236 |
| chr6 | 131949040 | 131950040 | 0.001950158 | −1.288198899 | −0.950138449 |
| chr1 | 242607780 | 242608200 | −0.001949043 | −0.89202281 | −1.22619902 |
| chr3 | 195923540 | 195923800 | 0.001946723 | −1.314674999 | −1.018986704 |
| chr2 | 241505180 | 241505540 | 0.001944918 | −1.232223668 | −0.973024827 |
| chr3 | 121553720 | 121554240 | 0.001944536 | −1.405680828 | −1.046155111 |
| chr10 | 50213720 | 50214220 | −0.001943715 | −0.834165481 | −1.152543032 |
| chr6 | 66078920 | 66079440 | −0.001942752 | −0.964915561 | −1.294914938 |
| chr2 | 88957560 | 88957940 | −0.001936516 | −0.843589203 | −1.101117282 |
| chr11 | 82434860 | 82435300 | 0.001934117 | −1.163791173 | −0.792831769 |
| chr13 | 71719800 | 71720360 | −0.001934007 | −0.872561413 | −1.194090795 |
| chr18 | 30509620 | 30509980 | −0.001933271 | −0.890495236 | −1.162615214 |
| chr18 | 30509560 | 30510040 | −0.001933271 | −0.890495236 | −1.162615214 |
| chr9 | 30752940 | 30753380 | −0.001932736 | −0.695371627 | −1.050639567 |
| chr19 | 33616820 | 33617100 | 0.001932191 | −1.239927742 | −0.935625328 |
| chr10 | 25977260 | 25978140 | −0.001931915 | −0.976046062 | −1.273276488 |
| chr1 | 81729980 | 81730360 | −0.001930942 | −0.784806057 | −1.135012366 |
| chr6 | 134353820 | 134354060 | −0.00193054 | −0.881827394 | −1.254330697 |
| chr4 | 74987260 | 74987640 | −0.001928952 | −1.003049722 | −1.332136807 |
| chr19 | 49127000 | 49128180 | 0.0019278 | −1.15336219 | −0.845593144 |
| chr14 | 55493360 | 55494280 | 0.001926062 | −1.278830786 | −1.023621205 |
| chr5 | 84048320 | 84048720 | −0.001925184 | −0.996928649 | −1.306033651 |
| chr14 | 49501560 | 49502020 | 0.001924001 | −1.196049324 | −1.07740427 |
| chr18 | 19180040 | 19181560 | 0.001922927 | −1.359706295 | −1.045293622 |
| chr2 | 108579740 | 108580100 | −0.001922199 | −0.864038467 | −1.167398133 |
| chr1 | 32800780 | 32802320 | 0.001919181 | −1.202321443 | −0.991888931 |
| chr17 | 75494420 | 75494940 | 0.001916279 | −1.031250037 | −0.801578495 |
| chr1 | 235084740 | 235084980 | −0.001912758 | −0.644480109 | −0.983213515 |
| chr5 | 84006740 | 84007220 | −0.0019124 | −0.821731498 | −1.209265769 |
| chr1 | 97617860 | 97618420 | −0.001908367 | −0.784663192 | −1.074576847 |
| chr17 | 3795380 | 3796880 | 0.001908121 | −1.165111076 | −0.804202498 |
| chr12 | 124046460 | 124046880 | −0.001908049 | −0.868124155 | −1.151363872 |
| chr12 | 124046460 | 124046880 | −0.001908049 | −0.868124155 | −1.151363872 |
| chr16 | 5007640 | 5008980 | 0.001907648 | −1.146118674 | −0.836827197 |
| chr13 | 100993980 | 100994480 | −0.001906684 | −0.869523198 | −1.176655713 |
| chr9 | 130546680 | 130547080 | −0.001901626 | −0.676767187 | −0.87632373 |
| chr4 | 33132320 | 33132840 | −0.001894859 | −0.954191315 | −1.288774523 |
| chr4 | 134891040 | 134891420 | −0.001894633 | −0.846028503 | −1.168486243 |
| chr4 | 134891000 | 134891460 | −0.001894633 | −0.846028503 | −1.168486243 |
| chr1 | 154154740 | 154156460 | 0.001890937 | −1.181963889 | −0.930958681 |
| chr22 | 24382040 | 24382420 | 0.001889233 | −1.14981412 | −0.896701558 |
| chr21 | 46322340 | 46322680 | 0.001888972 | −1.023318947 | −0.714865189 |
| chr5 | 177977980 | 177978400 | −0.001887946 | −0.932529441 | −1.294480615 |
| chr4 | 144079740 | 144080100 | −0.001887937 | −0.794902204 | −1.154102186 |
| chr4 | 144079700 | 144080140 | −0.001887937 | −0.794902204 | −1.154102186 |
| chr6 | 151987680 | 151988100 | 0.001886421 | −1.261542839 | −0.97026494 |
| chr8 | 87631140 | 87631680 | 0.001883736 | −1.287820352 | −0.925681632 |
| chr6 | 11940980 | 11941500 | −0.001883691 | −1.02359292 | −1.280426423 |
| chr8 | 68813600 | 68814080 | −0.001879598 | −0.889711091 | −1.212107462 |
| chr10 | 73571440 | 73572320 | 0.001879212 | −1.183626149 | −0.971472561 |
| chr4 | 18741020 | 18741360 | −0.001875019 | −0.765255849 | −1.100649794 |
| chr17 | 39844780 | 39845900 | 0.001874206 | −1.298593414 | −0.965202572 |
| chr17 | 68079260 | 68079560 | −0.001872659 | −0.690347762 | −0.972242176 |
| chr5 | 131721140 | 131721360 | −0.001872325 | −0.871628502 | −1.072813992 |
| chr10 | 60234520 | 60234880 | −0.001871525 | −0.904869634 | −1.189110821 |
| chr7 | 105939560 | 105939760 | −0.001868739 | −0.77094356 | −1.00267862 |
| chr7 | 105939560 | 105939760 | −0.001868739 | −0.77094356 | −1.00267862 |
| chr19 | 12035680 | 12036020 | 0.001866059 | −1.272729988 | −1.052055652 |
| chr8 | 61564160 | 61565400 | 0.001865216 | −1.266092102 | −1.024918372 |
| chr12 | 9556980 | 9557240 | −0.001863546 | −0.973206987 | −1.198347579 |
| chr7 | 69467860 | 69468420 | −0.001861935 | −0.900391909 | −1.210527391 |
| chr6 | 7723280 | 7723600 | 0.001860389 | −1.214900128 | −0.8853237 |
| chr15 | 64890540 | 64891560 | −0.001859852 | −0.721168057 | −1.037749533 |
| chr4 | 110343600 | 110343880 | 0.001856663 | −1.280040079 | −0.953925839 |
| chr19 | 12885540 | 12886680 | 0.001849991 | −1.151520795 | −0.929255604 |
| chr1 | 229412580 | 229412980 | −0.001849841 | −0.873998841 | −1.135022665 |
| chr4 | 185376260 | 185376520 | −0.001849029 | −1.018481158 | −1.195657154 |
| chr19 | 1274680 | 1276140 | 0.001847047 | −1.26757334 | −1.03007361 |
| chr22 | 34896000 | 34896860 | 0.001846075 | −0.982958766 | −0.804746927 |
| chr7 | 52250580 | 52251000 | −0.001845175 | −0.898428905 | −1.17046736 |
| chr17 | 45776260 | 45776520 | 0.001844442 | −1.221769692 | −0.952590953 |
| chr7 | 136713120 | 136713540 | 0.001844311 | −1.077036246 | −0.895811526 |
| chr7 | 136713100 | 136713560 | 0.001844311 | −1.077036246 | −0.895811526 |
| chr20 | 6787340 | 6787900 | 0.001843887 | −1.149611904 | −1.038027896 |
| chr20 | 4434200 | 4434820 | −0.001843462 | −0.956199178 | −1.321297329 |
| chr2 | 108579660 | 108580160 | −0.001841815 | −0.901335835 | −1.187354346 |
| chr5 | 177978020 | 177978380 | −0.001841814 | −0.902175708 | −1.295429063 |
| chr16 | 60918560 | 60919180 | −0.00184142 | −0.891531736 | −1.186688099 |
| chr19 | 13215120 | 13215360 | 0.001840736 | −1.080958865 | −0.833853751 |
| chr8 | 132693600 | 132694180 | −0.001839751 | −0.892720962 | −1.219898906 |
| chr8 | 1949420 | 1951380 | −0.001839131 | −1.047404973 | −1.169564815 |
| chr6 | 158552100 | 158552840 | −0.001838476 | −0.945421021 | −1.330625834 |
| chr2 | 141730340 | 141730780 | −0.001837775 | −0.869173142 | −1.147677556 |
| chr13 | 46502640 | 46503120 | −0.001836885 | −1.074701158 | −1.353960765 |
| chr1 | 166161460 | 166161940 | −0.001836613 | −0.893237371 | −1.189644624 |
| chr5 | 150678580 | 150679160 | −0.001836514 | −0.96139621 | −1.197510092 |
| chr1 | 194181540 | 194182080 | −0.001835525 | −0.810657124 | −1.118916458 |
| chr4 | 55903800 | 55904340 | −0.001835414 | −0.882247298 | −1.257571533 |
| chr5 | 88679280 | 88679700 | −0.001830739 | −0.953338366 | −1.24928995 |
| chr14 | 31889340 | 31890220 | 0.001829299 | −1.410340596 | −1.145989199 |
| chr17 | 39844760 | 39845880 | 0.001826558 | −1.302187753 | −1.011380862 |
| chr11 | 41491420 | 41491960 | 0.001826273 | −1.209448312 | −1.11480952 |
| chr21 | 16097140 | 16098160 | −0.001825084 | −0.865352566 | −1.111299842 |
| chr1 | 74663300 | 74664540 | 0.001824807 | −1.156430284 | −0.859660201 |
| chr6 | 95667660 | 95668000 | −0.001822732 | −0.975451443 | −1.247323457 |
| chr3 | 3330760 | 3331300 | −0.001821952 | −1.107542754 | −1.323441626 |
| chr4 | 31014660 | 31015320 | 0.001821667 | −1.167954076 | −1.029593168 |
| chr9 | 74467240 | 74467500 | −0.001820974 | −0.797071498 | −1.080439763 |
| chr2 | 223590580 | 223590820 | −0.00182062 | −0.890646334 | −1.180985259 |
| chr20 | 38816380 | 38816780 | −0.001820484 | −0.867342906 | −1.160253087 |
| chr11 | 33060520 | 33061700 | 0.001819249 | −1.289308422 | −1.042945779 |
| chr3 | 152760860 | 152761120 | 0.001819056 | −1.228266992 | −1.043484356 |
| chr3 | 152760860 | 152761120 | 0.001819056 | −1.228266992 | −1.043484356 |
| chr19 | 39137880 | 39138760 | 0.001816547 | −1.109609538 | −0.891167108 |
| chr20 | 20300800 | 20301340 | −0.001814735 | −1.025281198 | −1.36077188 |
| chr6 | 31324740 | 31325180 | 0.001814206 | −1.29357914 | −0.996095078 |
| chr11 | 117817200 | 117818460 | 0.001812738 | −1.252515343 | −1.010349762 |
| chr2 | 192329240 | 192329640 | −0.001812133 | −0.988166375 | −1.245620225 |
| chr15 | 74753060 | 74754080 | 0.001811613 | −1.337152716 | −1.107402573 |
| chr6 | 12680120 | 12680600 | −0.001811591 | −1.012100779 | −1.245467031 |
| chr3 | 192875300 | 192875700 | 0.00181138 | −1.445056923 | −1.247174375 |
| chr3 | 128399400 | 128400140 | 0.001811323 | −1.371294175 | −1.066471895 |
| chr6 | 16070820 | 16071220 | −0.001810653 | −1.04535823 | −1.380084089 |
| chr19 | 13044100 | 13045000 | 0.00181024 | −1.207618924 | −1.04491129 |
| chr7 | 2490820 | 2491660 | 0.001807441 | −1.160102301 | −0.91871006 |
| chr1 | 155989960 | 155991340 | 0.001803773 | −1.150866957 | −0.877689454 |
| chr3 | 160282020 | 160283700 | 0.001803417 | −1.319931378 | −1.034191162 |
| chr7 | 55639260 | 55640720 | 0.00180304 | −1.202641744 | −0.928984511 |
| chr6 | 83953360 | 83953800 | −0.001801785 | −1.019426318 | −1.306365216 |
| chr6 | 153150560 | 153151020 | −0.001801748 | −0.894950521 | −1.263116453 |
| chr4 | 84453600 | 84454520 | −0.001799843 | −0.997078548 | −1.294739185 |
| chr2 | 175613640 | 175613920 | 0.001799382 | −1.282570363 | −0.982044975 |
| chr6 | 6108580 | 6109180 | −0.001798899 | −0.856776324 | −1.100207942 |
| chr5 | 2623160 | 2623680 | 0.001798234 | −1.166078723 | −1.032502533 |
| chr7 | 121180400 | 121180900 | 0.001797611 | −1.185286934 | −1.048168773 |
| chr3 | 151986280 | 151987880 | 0.001797108 | −1.235795903 | −0.987353124 |
| chr18 | 7021660 | 7022020 | −0.001795644 | −1.033381611 | −1.372516711 |
| chr10 | 87625480 | 87625920 | −0.001794808 | −0.898171333 | −1.129884428 |
| chr19 | 3985000 | 3986140 | 0.001794431 | −1.20318186 | −0.957074834 |
| chr17 | 54265700 | 54266180 | −0.001792861 | −0.996047914 | −1.181918678 |
| chr5 | 173045980 | 173046260 | 0.001792352 | −1.289601161 | −1.028839787 |
| chr15 | 35212480 | 35212760 | 0.001791998 | −1.149896496 | −0.88061882 |
| chr14 | 23235220 | 23236540 | 0.001790519 | −1.316081091 | −1.050051172 |
| chr10 | 30959960 | 30960260 | 0.001789428 | −1.141223925 | −0.889959216 |
| chr6 | 129577660 | 129578180 | −0.001789244 | −1.022929588 | −1.296476801 |
| chr12 | 110869660 | 110870040 | −0.001786508 | −0.974705003 | −1.276579815 |
| chr21 | 16162120 | 16162500 | −0.00178376 | −0.85005859 | −1.098942835 |
| chr5 | 145562080 | 145562560 | 0.001782785 | −1.299008385 | −1.023677488 |
| chr6 | 26596660 | 26597600 | 0.001782647 | −1.348347128 | −1.182107409 |
| chr6 | 123895820 | 123896200 | −0.001782231 | −0.964495978 | −1.239501762 |
| chr10 | 129651800 | 129653080 | 0.001781754 | −1.262666794 | −0.976993587 |
| chr5 | 5705840 | 5706620 | −0.001781534 | −1.031677331 | −1.35901961 |
| chr20 | 30864980 | 30866060 | 0.001777041 | −1.12957985 | −0.76334108 |
| chr1 | 237245620 | 237246080 | −0.001775549 | −0.876094016 | −1.087291364 |
| chr21 | 20231300 | 20231780 | 0.001773473 | −1.128272441 | −0.885918544 |
| chr21 | 20231300 | 20231780 | 0.001773473 | −1.128272441 | −0.885918544 |
| chr14 | 79653740 | 79654200 | 0.001772057 | −1.392981175 | −1.038848153 |
| chr2 | 27649940 | 27650400 | −0.001771784 | −1.018856367 | −1.285285496 |
| chr12 | 78982340 | 78982940 | 0.001771383 | −1.295624438 | −1.114862765 |
| chr4 | 84453500 | 84454640 | −0.001770829 | −0.994385148 | −1.278142937 |
| chr17 | 74117060 | 74118940 | 0.001770116 | −1.201780317 | −0.936350787 |
| chr1 | 172663140 | 172663400 | −0.001768812 | −0.993792317 | −1.232789091 |
| chr1 | 172663140 | 172663400 | −0.001768812 | −0.993792317 | −1.232789091 |
| chr9 | 1104920 | 1105520 | −0.001768092 | −0.611578364 | −1.023597246 |
| chr17 | 19647860 | 19648820 | 0.001767783 | −1.165707927 | −0.897877046 |
| chr21 | 46008500 | 46008720 | −0.001766549 | −0.730908746 | −1.067209279 |
| chr21 | 46008500 | 46008720 | −0.001766549 | −0.730908746 | −1.067209279 |
| chr2 | 167167720 | 167168060 | −0.001765014 | −1.009931771 | −1.244409402 |
| chr1 | 10532500 | 10532820 | 0.00176413 | −1.256777033 | −1.02024304 |
| chr3 | 42829040 | 42829320 | −0.001763178 | −0.890703282 | −1.173796372 |
| chr5 | 150632160 | 150633280 | −0.001762481 | −0.880561696 | −1.073022758 |
| chr11 | 110518720 | 110519180 | −0.00176193 | −1.050632739 | −1.315258747 |
| chr3 | 149526060 | 149526340 | 0.001761617 | −1.362245976 | −1.132139347 |
| chr6 | 7723280 | 7723580 | 0.001761529 | −1.288711661 | −0.950824528 |
| chr11 | 70048340 | 70050300 | 0.001760506 | −1.399675847 | −1.117597544 |
| chr6 | 16593820 | 16594560 | −0.001760267 | −0.945359005 | −1.285479954 |
| chr1 | 73435720 | 73436060 | −0.001759591 | −0.751495052 | −1.133330527 |
| chr13 | 77054320 | 77054580 | 0.001756602 | −1.241454974 | −1.047404335 |
| chr13 | 42429220 | 42429780 | −0.001756486 | −1.050334505 | −1.34474535 |
| chr7 | 80847140 | 80847680 | −0.001756106 | −0.836598874 | −1.152143238 |
| chr2 | 5856360 | 5856740 | −0.001751463 | −0.83597468 | −1.124499557 |
| chr6 | 68151720 | 68152160 | −0.001750467 | −1.021728072 | −1.259540327 |
| chr11 | 60927840 | 60929680 | 0.00175006 | −1.323171898 | −1.108509806 |
| chr6 | 151987660 | 151988100 | 0.001749569 | −1.278237632 | −1.00434993 |
| chr11 | 65626840 | 65627160 | 0.001746697 | −1.400438343 | −1.165384487 |
| chr17 | 6569360 | 6569660 | 0.001746608 | −1.144538957 | −0.864020538 |
| chr1 | 244553000 | 244553520 | 0.001746061 | −1.156244067 | −1.010300338 |
| chr8 | 19674320 | 19675800 | 0.001746 | −1.259330763 | −0.958927902 |
| chr2 | 192430580 | 192431740 | 0.001745801 | −1.241725258 | −0.89011032 |
| chr2 | 39643100 | 39643320 | −0.001745113 | −0.861266131 | −1.10534045 |
| chr6 | 40604280 | 40604940 | −0.001744313 | −0.879010153 | −1.172329832 |
| chr2 | 216973280 | 216974700 | 0.001743164 | −1.326584866 | −1.129055722 |
| chr4 | 143303560 | 143304060 | −0.001742047 | −1.063460069 | −1.344866457 |
| chr6 | 100335120 | 100335480 | −0.001741542 | −1.048063232 | −1.331089324 |
| chr17 | 76919280 | 76919620 | 0.001741477 | −1.111801004 | −0.972363198 |
| chr20 | 47804080 | 47805480 | 0.001738927 | −1.326170129 | −1.035640462 |
| chr12 | 14762280 | 14762800 | −0.001738903 | −0.893288218 | −1.211149445 |
| chr21 | 27905300 | 27905760 | −0.001738678 | −0.643528798 | −0.945033912 |
| chr1 | 75126160 | 75126540 | −0.001738135 | −0.761425205 | −1.051786736 |
| chr6 | 33128800 | 33129960 | 0.001737721 | −1.312484679 | −0.987068498 |
| chr16 | 68271380 | 68271860 | 0.001736829 | −1.161591467 | −0.820710301 |
| chr15 | 59312100 | 59312660 | −0.001735591 | −0.762275717 | −1.153328779 |
| chr20 | 23618120 | 23619520 | −0.001735261 | −0.947641183 | −1.099002751 |
| chr21 | 28895480 | 28895940 | −0.001734928 | −0.781910668 | −1.113819022 |
| chr21 | 45557880 | 45558160 | 0.001734693 | −1.124273086 | −0.778764573 |
| chr3 | 194338920 | 194339580 | −0.001734085 | −0.961465004 | −1.312051536 |
| chr19 | 6429140 | 6429340 | −0.00173406 | −0.858684978 | −1.195171502 |
| chr16 | 17849140 | 17849540 | −0.001731474 | −0.827805134 | −1.125462834 |
| chr8 | 20807100 | 20807580 | −0.001729889 | −1.090107369 | −1.338023403 |
| chr3 | 8051740 | 8052300 | −0.001729854 | −1.023701557 | −1.287743187 |
| chr16 | 85336660 | 85337480 | 0.00172983 | −1.079964428 | −0.798703969 |
| chr21 | 43092520 | 43093560 | 0.001729761 | −1.089626899 | −0.68305534 |
| chr21 | 39231640 | 39231960 | −0.001729405 | −0.819108796 | −1.059766945 |
| chr1 | 35703200 | 35703680 | −0.00172916 | −1.008184022 | −1.279741506 |
| chr2 | 55524240 | 55524560 | −0.00172847 | −0.882147997 | −1.173705257 |
| chr11 | 64878660 | 64878880 | 0.001727931 | −1.191267554 | −0.934874465 |
| chr17 | 56190120 | 56191060 | 0.001727558 | −1.165987886 | −0.944658718 |
| chr11 | 123984880 | 123985600 | −0.001724882 | −0.956087828 | −1.204894368 |
| chr4 | 70957640 | 70958120 | −0.001724235 | −1.048870702 | −1.270490777 |
| chr17 | 781400 | 782000 | −0.001724154 | −0.875705326 | −1.055358856 |
| chr1 | 35703260 | 35703640 | −0.001723328 | −0.988722861 | −1.271963656 |
| chr13 | 64549560 | 64549920 | −0.001721016 | −1.005887771 | −1.229575737 |
| chr13 | 64549520 | 64549960 | −0.001721016 | −1.005887771 | −1.229575737 |
| chr6 | 167061140 | 167061960 | −0.001720665 | −0.826858861 | −0.954227694 |
| chr5 | 81329440 | 81329980 | −0.001720664 | −0.985776945 | −1.301856776 |
| chr5 | 14351860 | 14352940 | 0.001719992 | −1.220917597 | −0.945499494 |
| chr10 | 129651980 | 129652880 | 0.001718821 | −1.223771891 | −0.958700177 |
| chr3 | 86036600 | 86037040 | −0.001717211 | −1.026491844 | −1.332356872 |
| chr12 | 125347700 | 125349340 | 0.001716106 | −1.348861442 | −1.136789015 |
| chr9 | 30752940 | 30753340 | −0.001715743 | −0.694047711 | −1.025655201 |
| chr2 | 77100400 | 77100800 | −0.0017142 | −0.854366259 | −1.167591256 |
| chr1 | 45262120 | 45262360 | −0.001714185 | −0.834196995 | −1.050385574 |
| chr2 | 229128160 | 229128500 | −0.001713136 | −0.916394002 | −1.191976206 |
| chr2 | 86041060 | 86041960 | 0.001711709 | −1.134735434 | −0.926637525 |
| chr3 | 156310600 | 156311040 | −0.001711465 | −0.996472958 | −1.37243759 |
| chr6 | 3852080 | 3852500 | −0.001710872 | −1.031718856 | −1.200633657 |
| chr7 | 25019060 | 25020420 | 0.001709465 | −1.132717218 | −0.894046798 |
| chr3 | 197519380 | 197519600 | −0.001707827 | −1.090936757 | −1.380632354 |
| chr17 | 26971520 | 26972460 | 0.001706283 | −1.156063571 | −0.867016715 |
| chr12 | 69550840 | 69551140 | 0.001705857 | −1.263667892 | −1.011940371 |
| chr12 | 7062980 | 7063300 | 0.001705247 | −1.314488286 | −1.017860338 |
| chr13 | 62654600 | 62655100 | −0.001705158 | −1.126667646 | −1.363631702 |
| chr9 | 136022980 | 136023180 | −0.001704601 | −0.906866869 | −1.02970209 |
| chr11 | 96105560 | 96106160 | −0.001703807 | −1.063057146 | −1.371390852 |
| chr15 | 75660120 | 75661180 | 0.00170334 | −1.138256196 | −0.888217117 |
| chr1 | 169304840 | 169305160 | −0.001700785 | −0.860621891 | −1.132814291 |
| chr2 | 37747060 | 37747460 | 0.001700254 | −1.160451951 | −0.993448778 |
| chr19 | 58513600 | 58514940 | 0.001699424 | −1.112306129 | −0.879981171 |
| chr4 | 52976620 | 52977060 | −0.001697189 | −0.904545709 | −1.218341753 |
| chr14 | 46973720 | 46974200 | 0.001695403 | −1.246281736 | −1.039818042 |
| chr19 | 58089680 | 58090980 | 0.001694225 | −1.089900508 | −0.855404993 |
| chr22 | 31503120 | 31504260 | 0.001693593 | −1.075100448 | −0.836750942 |
| chr1 | 117117260 | 117117560 | 0.001693353 | −1.361859533 | −1.069390233 |
| chr2 | 238581740 | 238582540 | 0.001692576 | −1.233049783 | −0.886686257 |
| chr6 | 68151700 | 68152200 | −0.001692412 | −1.046872273 | −1.297953681 |
| chr3 | 11313800 | 11314300 | 0.001691393 | −1.334284405 | −1.09279344 |
| chr17 | 58041760 | 58042560 | 0.001690054 | −1.250898652 | −1.072470864 |
| chr22 | 31627180 | 31627480 | 0.001689614 | −0.989139001 | −0.759994327 |
| chr2 | 1259400 | 1260040 | −0.00168858 | −1.009662749 | −1.25065727 |
| chr12 | 68725480 | 68727140 | 0.001685362 | −1.369584237 | −1.126726437 |
| chr7 | 135723100 | 135723480 | −0.001683674 | −0.827197135 | −1.119759947 |
| chr19 | 1089280 | 1089540 | 0.0016819 | −1.017523334 | −0.781426098 |
| chr17 | 2206160 | 2207460 | 0.001679437 | −1.124100687 | −0.847558428 |
| chr5 | 31292700 | 31293200 | −0.001678886 | −1.025157917 | −1.18164671 |
| chr11 | 46388900 | 46389780 | 0.001677774 | −1.235001314 | −0.971950386 |
| chr4 | 73395140 | 73395640 | −0.001676882 | −1.048203835 | −1.345566123 |
| chr12 | 89936760 | 89937020 | 0.001676621 | −1.254084726 | −1.011936134 |
| chr13 | 57458980 | 57459480 | −0.001676168 | −0.982350314 | −1.260000772 |
| chr7 | 78843480 | 78844460 | −0.00167616 | −0.968020636 | −1.233772483 |
| chr10 | 50356720 | 50357140 | −0.001676119 | −1.016517169 | −1.294223011 |
| chr3 | 103290480 | 103290900 | −0.001675934 | −0.998726485 | −1.294817608 |
| chr3 | 188240420 | 188240740 | −0.00167537 | −1.047325458 | −1.231163742 |
| chr1 | 153930260 | 153931820 | 0.001675349 | −1.212723567 | −1.014229284 |
| chr8 | 66274840 | 66275300 | −0.001674739 | −0.963460725 | −1.12509728 |
| chr15 | 67639960 | 67640420 | −0.001674313 | −0.820087292 | −1.13517581 |
| chr19 | 12035680 | 12036000 | 0.001673782 | −1.272729988 | −1.066418305 |
| chr2 | 175311960 | 175312800 | −0.001673324 | −0.861221172 | −1.11448268 |
| chr18 | 51883720 | 51884880 | 0.001670837 | −1.325131562 | −1.149232103 |
| chr7 | 157744760 | 157745300 | 0.00166978 | −1.106542763 | −0.735315622 |
| chr17 | 70556080 | 70557000 | 0.001669283 | −1.082245914 | −0.885462031 |
| chr14 | 105956340 | 105958320 | −0.001668235 | −0.985989602 | −1.132923133 |
| chr4 | 165676540 | 165676920 | −0.001668143 | −1.188994522 | −1.360187215 |
| chr14 | 27268640 | 27268920 | −0.001668096 | −0.914904525 | −1.27905516 |
| chr10 | 107431600 | 107432080 | −0.001666883 | −1.072688392 | −1.268020118 |
| chr1 | 247357660 | 247357980 | −0.001666693 | −0.92656584 | −1.168261754 |
| chr9 | 80509040 | 80509400 | −0.00166487 | −0.755518216 | −1.061514599 |
| chr1 | 68458620 | 68459180 | −0.001662997 | −0.886176957 | −1.201023011 |
| chr6 | 56410980 | 56411360 | −0.001662218 | −1.048235148 | −1.239251792 |
| chr12 | 49457760 | 49458020 | 0.001661632 | −1.294610017 | −1.049742962 |
| chr12 | 49457760 | 49458020 | 0.001661632 | −1.294610017 | −1.049742962 |
| chr3 | 7768060 | 7768560 | −0.001661077 | −1.053979809 | −1.318814564 |
| chr16 | 81756760 | 81757080 | 0.00166032 | −1.211849375 | −0.944712785 |
| chr8 | 79794780 | 79795360 | 0.001659996 | −1.261393801 | −1.165220012 |
| chr12 | 7063000 | 7063320 | 0.001659946 | −1.316058138 | −1.043993786 |
| chr12 | 62586480 | 62586780 | 0.001659735 | −1.143728478 | −0.915827913 |
| chr12 | 62586480 | 62586780 | 0.001659735 | −1.143728478 | −0.915827913 |
| chr7 | 111616220 | 111616500 | −0.001659653 | −0.905834879 | −1.198525099 |
| chr4 | 148814440 | 148814940 | −0.001659119 | −0.969385202 | −1.25234665 |
| chr16 | 55744820 | 55745360 | 0.00165839 | −1.115694033 | −1.069202302 |
| chr9 | 96755820 | 96756200 | −0.001657859 | −0.764365979 | −1.080295636 |
| chr7 | 148762880 | 148763220 | 0.001657589 | −1.253063946 | −1.035750432 |
| chr8 | 121310580 | 121311080 | −0.001657251 | −1.015432383 | −1.272619566 |
| chr6 | 129521080 | 129521840 | 0.001655127 | −1.192855995 | −1.050191286 |
| chr2 | 178299500 | 178299980 | −0.001653879 | −0.816526192 | −1.093640663 |
| chr13 | 87800200 | 87800580 | −0.001653092 | −0.995346712 | −1.226844566 |
| chr14 | 41362960 | 41363480 | −0.001652097 | −1.100282497 | −1.33875705 |
| chr22 | 41157080 | 41157600 | −0.001651765 | −0.755027366 | −1.155486288 |
| chr9 | 112170900 | 112171360 | −0.001651169 | −0.745388168 | −1.05627437 |
| chr15 | 52970340 | 52972260 | 0.001650084 | −1.045400322 | −0.756017521 |
| chr2 | 169833460 | 169833800 | −0.001649268 | −0.909290425 | −1.137861953 |
| chr9 | 11725740 | 11726180 | −0.001649267 | −0.797630046 | −1.086294568 |
| chr2 | 181278900 | 181279240 | −0.001649087 | −0.935627904 | −1.127640353 |
| chr2 | 181278900 | 181279240 | −0.001649087 | −0.935627904 | −1.127640353 |
| chr3 | 79898780 | 79899360 | −0.001647845 | −0.891628702 | −1.173954608 |
| chr14 | 55737780 | 55738800 | 0.001647584 | −1.31831064 | −1.00446761 |
| chr6 | 91788120 | 91788680 | −0.001647183 | −0.942568342 | −1.256917769 |
| chr6 | 153150560 | 153151040 | −0.001646725 | −0.880639547 | −1.250556264 |
| chr19 | 17530460 | 17531520 | 0.001646484 | −1.131241001 | −0.874950967 |
| chr12 | 87949960 | 87950460 | −0.001646237 | −1.104869052 | −1.35527986 |
| chr1 | 226312920 | 226313180 | 0.001645053 | −1.272567014 | −1.026719809 |
| chr2 | 38091320 | 38091780 | −0.001644507 | −0.897058668 | −1.209059159 |
| chr19 | 4606720 | 4607140 | 0.001644501 | −1.020703983 | −0.831550159 |
| chr9 | 75491200 | 75491560 | −0.001644003 | −0.750291667 | −1.07949311 |
| chr5 | 67045380 | 67045820 | −0.0016439 | −0.951134152 | −1.20744774 |
| chr16 | 1524440 | 1525860 | 0.001643408 | −1.085077662 | −0.779353372 |
| chr8 | 68813660 | 68814060 | −0.001641931 | −0.908575578 | −1.22988585 |
| chr10 | 130010200 | 130010520 | 0.00164136 | −1.270469574 | −0.967614448 |
| chr5 | 145562100 | 145562560 | 0.001641217 | −1.300470795 | −1.045935722 |
| chr20 | 37678440 | 37679340 | 0.001641169 | −1.341991547 | −1.106253475 |
| chr12 | 105580180 | 105580720 | −0.001641142 | −0.970544913 | −1.197268047 |
| chr8 | 36456600 | 36457020 | −0.00164048 | −0.937672334 | −1.199854976 |
| chr19 | 13210220 | 13210540 | 0.001640113 | −1.120635263 | −0.938994426 |
| chr18 | 63122300 | 63123160 | −0.001639573 | −0.924892376 | −1.322228651 |
| chr11 | 26223580 | 26224100 | −0.001638632 | −1.033593501 | −1.337980725 |
| chr6 | 156953440 | 156953960 | −0.001638246 | −0.888026954 | −1.136664864 |
| chr11 | 24650820 | 24651240 | 0.001637451 | −1.054629382 | −0.922398151 |
| chr18 | 70788460 | 70788740 | −0.001636764 | −0.891043114 | −1.214079035 |
| chr2 | 85154180 | 85154440 | 0.001636639 | −1.306900321 | −1.030822707 |
| chr19 | 40954380 | 40954660 | −0.001635953 | −0.759015349 | −1.089422743 |
| chr19 | 40954320 | 40954720 | −0.001635953 | −0.759015349 | −1.089422743 |
| chr21 | 19299220 | 19299680 | −0.001635097 | −0.786302515 | −1.07127293 |
| chr2 | 213256220 | 213256680 | −0.001634179 | −1.019662327 | −1.361173295 |
| chr4 | 68253440 | 68254000 | −0.001633697 | −1.057276455 | −1.299291853 |
| chr3 | 78685900 | 78686320 | −0.00163339 | −1.177863676 | −1.425974533 |
| chr19 | 10675980 | 10677320 | 0.001633184 | −1.04863403 | −0.842592423 |
| chr13 | 26722620 | 26723000 | −0.001632981 | −1.088615134 | −1.317713644 |
| chr13 | 26722560 | 26723060 | −0.001632981 | −1.088615134 | −1.317713644 |
| chr6 | 10412500 | 10412900 | 0.00163292 | −1.268422766 | −1.004447909 |
| chr7 | 150941460 | 150941700 | 0.001632136 | −1.14802976 | −0.957526521 |
| chr1 | 152055020 | 152055440 | −0.0016321 | −0.858829572 | −1.068072159 |
| chr18 | 28118720 | 28119000 | −0.001631578 | −0.949426462 | −1.142231884 |
| chr6 | 71275800 | 71277380 | 0.001631572 | −1.338425067 | −1.050318539 |
| chr2 | 202843440 | 202843860 | −0.001630077 | −0.915509456 | −1.205495194 |
| chr4 | 179446320 | 179446740 | −0.001629866 | −1.137753909 | −1.32774326 |
| chr4 | 179446280 | 179446780 | −0.001629866 | −1.137753909 | −1.32774326 |
| chr1 | 153721440 | 153722720 | 0.001629123 | −1.211772311 | −1.039691763 |
| chr2 | 144077820 | 144078220 | 0.001628538 | −1.129984855 | −0.909092392 |
| chr1 | 69737580 | 69737980 | −0.001626908 | −0.991285414 | −1.241454647 |
| chr1 | 69737520 | 69738040 | −0.001626908 | −0.991285414 | −1.241454647 |
| chr2 | 57779080 | 57779500 | −0.001626601 | −0.907732627 | −1.261871584 |
| chr1 | 249119700 | 249121340 | 0.001626517 | −1.288936759 | −1.05475076 |
| chr11 | 26223640 | 26224060 | −0.001626492 | −0.996056329 | −1.295634437 |
| chr3 | 8051760 | 8052260 | −0.001626301 | −1.046708748 | −1.260361129 |
| chr1 | 18001800 | 18003080 | 0.001625628 | −1.17531584 | −0.824571829 |
| chr1 | 18001760 | 18003120 | 0.001625628 | −1.17531584 | −0.824571829 |
| chr5 | 90850220 | 90850700 | −0.001625359 | −0.927967008 | −1.226042817 |
| chr5 | 90850220 | 90850700 | −0.001625359 | −0.927967008 | −1.226042817 |
| chr15 | 73414160 | 73414720 | −0.001623901 | −0.782172392 | −1.021552089 |
| chr8 | 132693560 | 132694180 | −0.001623893 | −0.946084036 | −1.200404221 |
| chr1 | 169304840 | 169305180 | −0.001623638 | −0.827976292 | −1.108414211 |
| chr10 | 27171000 | 27171260 | 0.001623443 | −1.209075078 | −1.017127802 |
| chr10 | 27170980 | 27171280 | 0.001623443 | −1.209075078 | −1.017127802 |
| chr9 | 30841780 | 30842300 | −0.001622919 | −0.744552338 | −1.041632524 |
| chr5 | 9710780 | 9711300 | −0.001622528 | −1.057346192 | −1.280181416 |
| chr6 | 83679600 | 83679980 | −0.001622244 | −0.884279549 | −1.120123507 |
| chr5 | 20242560 | 20242920 | −0.001621432 | −0.935016972 | −1.228392575 |
| chr6 | 56799180 | 56799580 | −0.001621303 | −1.025875306 | −1.233246639 |
| chr17 | 1390640 | 1390940 | 0.001621143 | −1.168140291 | −0.969734768 |
| chr19 | 54370600 | 54371200 | 0.001621077 | −1.061703058 | −0.883349758 |
| chr11 | 92026440 | 92026980 | −0.001620878 | −1.012961281 | −1.260178375 |
| chr5 | 99901660 | 99902100 | −0.001619393 | −1.05604558 | −1.300252962 |
| chr4 | 150264040 | 150264500 | −0.001618448 | −1.128667506 | −1.346126544 |
| chr4 | 150264020 | 150264520 | −0.001618448 | −1.128667506 | −1.346126544 |
| chr10 | 63780560 | 63781060 | −0.001617841 | −0.84208815 | −1.125652283 |
| chr2 | 224869580 | 224869980 | −0.001617308 | −1.076375934 | −1.374404261 |
| chr6 | 66169660 | 66170140 | −0.00161694 | −1.064454363 | −1.25300256 |
| chr17 | 75731660 | 75732140 | 0.001616451 | −0.968008964 | −0.756599012 |
| chr8 | 1683660 | 1683940 | 0.001615473 | −1.255054396 | −1.01912457 |
| chr2 | 70313760 | 70314980 | 0.001614536 | −1.292140119 | −1.114075244 |
| chr12 | 133356280 | 133356980 | 0.001614053 | −1.17549124 | −0.908154156 |
| chr10 | 126096660 | 126097120 | −0.001613715 | −0.956738249 | −1.215107737 |
| chr16 | 78744220 | 78744520 | −0.001613152 | −0.801707208 | −1.077826047 |
| chr15 | 40615600 | 40617080 | 0.001613065 | −1.121546454 | −0.855194144 |
| chr15 | 40615560 | 40617120 | 0.001613065 | −1.121546454 | −0.855194144 |
| chr15 | 53280240 | 53280940 | 0.001612461 | −0.99668246 | −0.918498723 |
| chr5 | 103240760 | 103241220 | −0.001611548 | −0.844886114 | −1.010625646 |
| chr1 | 28969160 | 28969920 | 0.001610667 | −1.141029428 | −0.916120346 |
| chr4 | 106199180 | 106199500 | −0.001610408 | −0.950294364 | −1.16387358 |
| chr13 | 77067040 | 77067440 | −0.001610261 | −1.11220931 | −1.357563354 |
| chr14 | 77338880 | 77339760 | 0.001610203 | −1.370092449 | −1.041847539 |
| chr1 | 194181600 | 194182080 | −0.001609339 | −0.971434624 | −1.216478939 |
| chr11 | 7166320 | 7166660 | −0.001607706 | −0.917151216 | −1.235231153 |
| chr3 | 37901520 | 37902560 | 0.001607693 | −1.359991245 | −1.094561792 |
| chr10 | 95545320 | 95546380 | 0.001606561 | −1.38669291 | −1.217112497 |
| chr19 | 10206380 | 10207320 | 0.001606264 | −1.178189621 | −0.978580908 |
| chr13 | 100994060 | 100994440 | −0.001605455 | −0.92487411 | −1.193256482 |
| chr2 | 159289360 | 159289960 | 0.001605021 | −0.958218445 | −0.803710037 |
| chr5 | 162132200 | 162132680 | −0.001604172 | −0.960612409 | −1.235544866 |
| chr18 | 44593080 | 44593460 | −0.001603385 | −1.060816684 | −1.2123037 |
| chr7 | 53760900 | 53761380 | −0.001602508 | −1.003954141 | −1.249764772 |
| chr9 | 132222460 | 132223000 | 0.001602438 | −1.187706327 | −0.965072321 |
| chr4 | 1713540 | 1715000 | 0.001601685 | −1.354817925 | −1.023663052 |
| chr3 | 158445580 | 158445860 | 0.001601371 | −1.372314433 | −1.080882552 |
| chr7 | 28776920 | 28777440 | −0.0016012 | −0.939330263 | −1.220214255 |
| chr4 | 74781140 | 74781580 | −0.0016011 | −0.940901781 | −1.18763956 |
| chr6 | 102473780 | 102474260 | −0.001601006 | −0.875018012 | −1.088294948 |
| chr10 | 14919800 | 14921480 | 0.001600893 | −1.222248692 | −0.996004968 |
| chr11 | 60928260 | 60929340 | 0.001600241 | −1.284231717 | −1.074888768 |
| chr16 | 67562080 | 67563200 | 0.001599976 | −1.158540618 | −0.898149352 |
| chr19 | 12750960 | 12751280 | 0.001597994 | −1.1387386 | −0.961839117 |
| chr15 | 46957840 | 46958380 | −0.00159619 | −0.920888826 | −1.284558745 |
| chr18 | 51883700 | 51884880 | 0.001595959 | −1.325131562 | −1.149077155 |
| chr7 | 38145520 | 38145820 | 0.001595938 | −1.156343129 | −0.944351043 |
| chr13 | 64232340 | 64232900 | −0.001595071 | −1.001909649 | −1.252382471 |
| chr15 | 90614540 | 90614800 | 0.001594572 | −1.154808842 | −0.8396283 |
| chr2 | 60837200 | 60837660 | 0.001593688 | −1.182460134 | −1.00131585 |
| chr15 | 66564120 | 66564600 | −0.001592848 | −0.755312829 | −1.040601274 |
| chr8 | 60417900 | 60418500 | −0.001592124 | −1.13762682 | −1.398494557 |
| chr8 | 60417900 | 60418500 | −0.001592124 | −1.13762682 | −1.398494557 |
| chr2 | 5856300 | 5856780 | −0.001590526 | −0.894171251 | −1.145369253 |
| chr1 | 1475120 | 1476160 | 0.001590243 | −1.111949016 | −0.924061792 |
| chr3 | 72094800 | 72095120 | −0.001590165 | −1.029451055 | −1.295134721 |
| chr11 | 62438940 | 62439260 | 0.001588103 | −1.337514775 | −1.005948152 |
| chr16 | 82164020 | 82164860 | 0.001587796 | −1.100356983 | −0.94937981 |
| chr7 | 150780260 | 150780780 | 0.001587795 | −1.222003542 | −0.954291242 |
| chr7 | 150780280 | 150780760 | 0.001587795 | −1.222003542 | −0.954291242 |
| chr1 | 67966140 | 67966460 | 0.001587162 | −1.164197394 | −0.844159857 |
| chr3 | 126001120 | 126001500 | −0.001586772 | −0.964061481 | −1.2341045 |
| chr17 | 13272100 | 13272660 | −0.001586627 | −0.829086888 | −1.195485854 |
| chr16 | 89652120 | 89652700 | 0.001585959 | −1.249306915 | −0.972431702 |
| chr10 | 119289420 | 119289820 | −0.001585779 | −0.865470472 | −1.049650509 |
| chr20 | 8497120 | 8497580 | −0.00158547 | −1.022481735 | −1.242417957 |
| chr20 | 54139380 | 54139880 | 0.001585161 | −1.205135954 | −1.062884522 |
| chr20 | 12817100 | 12817480 | −0.00158481 | −0.989711008 | −1.203432946 |
| chr5 | 66748640 | 66749060 | −0.001584096 | −0.980846966 | −1.196380008 |
| chr17 | 56409820 | 56410080 | 0.001583996 | −1.149443658 | −0.913326168 |
| chr10 | 96390860 | 96391220 | −0.00158368 | −0.920480795 | −1.236998778 |
| chr22 | 37882340 | 37882620 | 0.001583673 | −1.143144075 | −0.85530824 |
| chr19 | 10676020 | 10677060 | 0.001583294 | −1.0301869 | −0.78811203 |
| chr3 | 175387940 | 175388620 | −0.001581009 | −0.853371954 | −1.269444788 |
| chr10 | 70286980 | 70287500 | 0.001580904 | −1.237173833 | −0.963584649 |
| chr1 | 1274400 | 1274680 | 0.001580639 | −1.094449154 | −0.884375026 |
| chr1 | 1274420 | 1274660 | 0.001580639 | −1.094449154 | −0.884375026 |
| chr9 | 82358240 | 82358800 | −0.001579271 | −0.922655104 | −1.216113246 |
| chr8 | 78755580 | 78756000 | −0.001578983 | −1.101261792 | −1.398378597 |
| chr18 | 45893580 | 45893900 | −0.001578604 | −0.971920622 | −1.162311375 |
| chr10 | 88136540 | 88137780 | 0.001577935 | −1.278202397 | −0.980068184 |
| chr5 | 90896900 | 90897360 | −0.00157697 | −0.923720428 | −1.206023403 |
| chr5 | 90896880 | 90897380 | −0.00157697 | −0.923720428 | −1.206023403 |
| chr17 | 44018800 | 44019260 | 0.001575115 | −1.125241774 | −0.80826051 |
| chr5 | 176899760 | 176901160 | 0.001574524 | −1.195434044 | −0.964835491 |
| chr3 | 86082700 | 86083180 | 0.001574314 | −1.263062383 | −1.093777395 |
| chr3 | 86082640 | 86083240 | 0.001574314 | −1.263062383 | −1.093777395 |
| chr9 | 104876000 | 104876740 | −0.001574139 | −0.796786242 | −1.072749248 |
| chr4 | 160482060 | 160482380 | −0.001573523 | −0.976932615 | −1.211043446 |
| chr4 | 111405800 | 111406040 | −0.001573128 | −0.971989156 | −1.239298098 |
| chr4 | 111405800 | 111406040 | −0.001573128 | −0.971989156 | −1.239298098 |
| chr7 | 64411680 | 64411940 | 0.001571026 | −1.198219805 | −0.9534773 |
| chr17 | 65990500 | 65990740 | −0.001569725 | −0.986277328 | −1.185906006 |
| chr17 | 65990420 | 65990820 | −0.001569725 | −0.986277328 | −1.185906006 |
| chr5 | 21183280 | 21183680 | −0.001569423 | −0.939396931 | −1.1918402 |
| chr12 | 82041500 | 82041780 | −0.001568618 | −1.050086288 | −1.328603913 |
| chr7 | 52246600 | 52247140 | −0.001567939 | −0.86628472 | −1.264828649 |
| chr7 | 52246560 | 52247180 | −0.001567939 | −0.86628472 | −1.264828649 |
| chr10 | 14919740 | 14921560 | 0.001567503 | −1.292713977 | −1.060609026 |
| chr12 | 57030520 | 57030780 | 0.001567322 | −1.271993089 | −1.095855567 |
| chr13 | 88207660 | 88208300 | −0.001566885 | −1.068446972 | −1.318221192 |
| chr18 | 28226480 | 28227800 | −0.001566777 | −1.027671404 | −1.372043853 |
| chr3 | 26732320 | 26732840 | −0.001566648 | −1.026088133 | −1.273086704 |
| chr19 | 13961160 | 13961600 | 0.001565855 | −1.141230052 | −0.927119677 |
| chr10 | 57301780 | 57302340 | 0.00156567 | −1.133129027 | −0.997685885 |
| chr22 | 39925260 | 39925540 | 0.001565308 | −1.130737642 | −0.911547555 |
| chr22 | 39924960 | 39925840 | 0.001565308 | −1.130737642 | −0.911547555 |
| chr10 | 57109760 | 57110260 | −0.001565168 | −0.936651252 | −1.211837666 |
| chr12 | 84857160 | 84857600 | −0.001564681 | −1.039199465 | −1.252789124 |
| chr11 | 64512180 | 64513180 | 0.001564515 | −1.129419649 | −0.997515303 |
| chr1 | 201528820 | 201529120 | 0.001564246 | −1.226469072 | −1.024641927 |
| chr22 | 49770400 | 49770840 | −0.001563803 | −0.896685442 | −1.150092456 |
| chr21 | 19605040 | 19605500 | −0.001563769 | −0.875724356 | −1.102331571 |
| chr1 | 45262120 | 45262320 | −0.00156361 | −0.946455576 | −1.1801493 |
| chr19 | 42724180 | 42724560 | 0.001562713 | −1.157981781 | −0.911986881 |
| chr19 | 42724180 | 42724560 | 0.001562713 | −1.157981781 | −0.911986881 |
| chr21 | 18086360 | 18086820 | −0.001560396 | −0.795669945 | −1.044214393 |
| chr19 | 17530520 | 17531440 | 0.001560289 | −1.148153139 | −0.891563581 |
| chr1 | 80805020 | 80805460 | 0.001559374 | −1.069272978 | −0.939809899 |
| chr15 | 59821120 | 59821420 | 0.001558978 | −1.185907415 | −0.920596722 |
| chr19 | 7694040 | 7694800 | 0.001558631 | −1.03853981 | −0.854724275 |
| chr14 | 24603100 | 24603380 | −0.001558518 | −0.902027437 | −1.137827085 |
| chr17 | 66192540 | 66192860 | 0.001558416 | −1.114796727 | −0.903201533 |
| chr17 | 66192540 | 66192860 | 0.001558416 | −1.114796727 | −0.903201533 |
| chr3 | 195586220 | 195586500 | 0.00155795 | −1.326685732 | −1.028668869 |
| chr3 | 88710340 | 88710780 | −0.001557861 | −0.969055041 | −1.23695541 |
| chr12 | 97221740 | 97221940 | −0.001557575 | −1.016168676 | −1.282459858 |
| chr4 | 52795460 | 52795940 | −0.00155753 | −0.925454969 | −1.201482623 |
| chr4 | 52795420 | 52795980 | −0.00155753 | −0.925454969 | −1.201482623 |
| chr21 | 17592900 | 17593380 | −0.001557517 | −0.847361868 | −1.103072806 |
| chr2 | 120516680 | 120518080 | 0.00155652 | −1.230813383 | −1.01548829 |
| chr5 | 5778200 | 5778820 | −0.001556414 | −0.946680774 | −1.310027378 |
| chr5 | 84968640 | 84969760 | −0.001554712 | −0.903071743 | −1.23915503 |
| chr10 | 47664260 | 47665040 | 0.001554095 | −1.213820849 | −0.852073472 |
| chr10 | 47664240 | 47665060 | 0.001554095 | −1.213820849 | −0.852073472 |
| chr3 | 120343260 | 120343740 | −0.001553392 | −0.941425091 | −1.14060262 |
| chr19 | 51250960 | 51251440 | 0.001552475 | −1.075952503 | −0.99250022 |
| chr16 | 813640 | 813980 | 0.001551788 | −1.113202823 | −0.957864884 |
| chr3 | 174573380 | 174573920 | 0.001551281 | −1.174127584 | −0.994205333 |
| chr19 | 2307740 | 2308720 | 0.001551048 | −1.197329904 | −0.994665843 |
| chr5 | 45203440 | 45204460 | −0.00155074 | −0.922563322 | −1.188975948 |
| chr14 | 64413080 | 64413620 | −0.001550262 | −0.893751825 | −1.199203436 |
| chr11 | 111883820 | 111884300 | −0.001549448 | −1.000205789 | −1.116960297 |
| chr3 | 141977760 | 141978260 | −0.001549383 | −1.163726381 | −1.336305758 |
| chr9 | 121152480 | 121153160 | −0.001549272 | −0.769334793 | −1.075706504 |
| chr6 | 29052020 | 29052560 | −0.001549006 | −1.063245645 | −1.2934236 |
| chr6 | 52149140 | 52149980 | 0.001548278 | −1.220131354 | −0.970037734 |
| chr1 | 11865340 | 11866680 | −0.001548163 | −0.9307811 | −1.052281109 |
| chr6 | 26596040 | 26598260 | 0.001547451 | −1.361450155 | −1.207373165 |
| chr16 | 31438720 | 31439740 | 0.001547119 | −1.222613119 | −1.002707737 |
| chr18 | 41210920 | 41211520 | −0.001546124 | −1.002567351 | −1.296333237 |
| chr10 | 30960000 | 30960260 | 0.001545912 | −1.256186178 | −0.99648725 |
| chr10 | 46079420 | 46079780 | −0.001545424 | −0.845550128 | −1.160475667 |
| chr3 | 48442320 | 48442600 | 0.00154454 | −1.325717782 | −0.943920032 |
| chr9 | 33165980 | 33166220 | 0.001544389 | −0.926745294 | −0.617754809 |
| chr14 | 44908660 | 44909200 | −0.001544007 | −0.990389207 | −1.195928563 |
| chr5 | 159835560 | 159836100 | −0.001543652 | −0.995621206 | −1.319329846 |
| chr5 | 26811320 | 26811860 | −0.001543176 | −0.91651889 | −1.171077986 |
| chr3 | 78685840 | 78686360 | −0.001542321 | −1.176705658 | −1.4193861 |
| chr1 | 29062980 | 29064000 | 0.001541164 | −1.058867864 | −0.909758587 |
| chr19 | 37115760 | 37116220 | −0.001540786 | −0.965068329 | −1.164887391 |
| chr4 | 187572040 | 187572420 | −0.001540274 | −0.836345637 | −1.148070929 |
| chr19 | 13212500 | 13212820 | 0.001540121 | −1.104143207 | −0.959841416 |
| chr19 | 32237880 | 32238960 | 0.001540028 | −1.069893288 | −0.969319325 |
| chr5 | 51806920 | 51807360 | −0.001539954 | −0.973809344 | −1.288346269 |
| chr3 | 115526940 | 115527220 | 0.001539716 | −1.221996654 | −1.008828902 |
| chr3 | 115526940 | 115527220 | 0.001539716 | −1.221996654 | −1.008828902 |
| chr2 | 109228160 | 109228760 | −0.001539691 | −0.96379041 | −1.156842311 |
| chr2 | 109228160 | 109228720 | −0.00153968 | −1.016516158 | −1.231900377 |
| chr13 | 86190840 | 86191660 | −0.001539207 | −0.943260775 | −1.269499956 |
| chr17 | 56076600 | 56077520 | 0.001538641 | −1.136854608 | −0.971730166 |
| chr8 | 113422600 | 113423120 | −0.001538028 | −1.071580226 | −1.331870373 |
| chr16 | 23568200 | 23569520 | 0.001537183 | −1.12220963 | −0.994556664 |
| chr3 | 22320780 | 22321260 | −0.001534906 | −0.790819497 | −1.13146817 |
| chr7 | 129073660 | 129074860 | 0.001534832 | −1.14038255 | −0.886826813 |
| chr11 | 60666860 | 60667140 | 0.001533479 | −1.33976372 | −1.075166854 |
| chr17 | 77070580 | 77071620 | 0.001533398 | −1.079890843 | −0.897720019 |
| chr17 | 29248780 | 29249640 | 0.001532524 | −1.17513259 | −0.80061273 |
| chr1 | 72364280 | 72364960 | 0.001532422 | −1.241236205 | −1.099085484 |
| chr8 | 116333480 | 116333980 | −0.001532215 | −0.9525287 | −1.194714748 |
| chr13 | 97868980 | 97869580 | −0.001532132 | −1.056090706 | −1.366018339 |
| chr1 | 166161460 | 166161920 | −0.001531356 | −0.908912522 | −1.172151698 |
| chr8 | 16546560 | 16547040 | 0.001531106 | −1.299927299 | −1.019347751 |
| chr17 | 38115240 | 38115540 | 0.001531054 | −1.271909935 | −0.988397151 |
| chr5 | 130031880 | 130032280 | −0.001531042 | −1.078325572 | −1.354242678 |
| chr3 | 16579620 | 16579920 | −0.001530068 | −1.052459028 | −1.352737068 |
| chr20 | 37100660 | 37102400 | 0.001529447 | −1.214256614 | −1.025042724 |
| chr12 | 83425880 | 83426240 | −0.001529354 | −0.935940265 | −1.139002106 |
| chr12 | 22763700 | 22764220 | 0.001529008 | −1.276045813 | −1.188003895 |
| chr13 | 87800120 | 87800620 | −0.001528977 | −0.860877255 | −1.090679186 |
| chr18 | 31376700 | 31377200 | −0.001528703 | −1.012348927 | −1.315266506 |
| chr18 | 1504520 | 1504940 | −0.001528701 | −1.084187038 | −1.325312888 |
| chr18 | 1504480 | 1504980 | −0.001528701 | −1.084187038 | −1.325312888 |
| chr4 | 115953660 | 115954960 | −0.001528437 | −0.843640427 | −1.061573845 |
| chr11 | 36531140 | 36532700 | 0.001528249 | −1.242547783 | −1.010095124 |
| chr15 | 44483920 | 44484320 | −0.001527466 | −0.838333694 | −1.131542055 |
| chr15 | 44483880 | 44484360 | −0.001527466 | −0.838333694 | −1.131542055 |
| chr2 | 232574940 | 232575280 | 0.001527448 | −1.282954858 | −1.109127243 |
| chr8 | 55078780 | 55079040 | 0.001527201 | −1.177494707 | −0.980667709 |
| chr1 | 11865340 | 11866600 | −0.001526077 | −0.929024546 | −1.024324745 |
| chr3 | 22571960 | 22572600 | −0.001525951 | −1.000140393 | −1.327303122 |
| chr16 | 23264040 | 23264420 | −0.001525847 | −0.900010023 | −1.154476132 |
| chr20 | 57580580 | 57580900 | 0.001525594 | −1.230367164 | −0.967910805 |
| chr16 | 30661580 | 30662420 | 0.001524714 | −1.167269147 | −0.970979016 |
| chr1 | 163725720 | 163726080 | −0.001524591 | −0.963571632 | −1.241980873 |
| chr17 | 70631760 | 70632200 | −0.001524262 | −0.875562446 | −1.160124962 |
| chr17 | 70631720 | 70632240 | −0.001524262 | −0.875562446 | −1.160124962 |
| chr12 | 75728120 | 75728420 | 0.001523799 | −1.31206647 | −1.054496399 |
| chr19 | 54665940 | 54666340 | 0.001523018 | −1.207765948 | −1.002621694 |
| chr16 | 4741920 | 4742220 | 0.001521989 | −1.053298183 | −0.834987204 |
| chr1 | 119272320 | 119272900 | −0.001521921 | −1.001448499 | −1.303680549 |
| chr9 | 126981020 | 126981280 | 0.001521786 | −1.013256128 | −0.781131622 |
| chr9 | 126981020 | 126981280 | 0.001521786 | −1.013256128 | −0.781131622 |
| chr15 | 64930000 | 64930420 | −0.001521263 | −0.774336255 | −1.098813107 |
| chr8 | 50801120 | 50801640 | −0.001520657 | −1.093995803 | −1.388519407 |
| chr1 | 242607740 | 242608300 | −0.001520219 | −0.898893934 | −1.182689877 |
| chr4 | 158751440 | 158751700 | 0.001519781 | −1.29238797 | −1.150783063 |
| chr4 | 158751440 | 158751700 | 0.001519781 | −1.29238797 | −1.150783063 |
| chr5 | 117875120 | 117876260 | −0.001519276 | −0.972600766 | −1.191123959 |
| chr2 | 241505200 | 241506480 | 0.001519178 | −1.10326174 | −0.967896596 |
| chr7 | 125159000 | 125159940 | −0.001519114 | −1.134017536 | −1.37321394 |
| chr7 | 143077280 | 143079060 | 0.001518856 | −1.251291979 | −1.073211217 |
| chr6 | 69236800 | 69237080 | −0.001518257 | −0.941927078 | −1.159546164 |
| chr16 | 2041700 | 2043200 | 0.001517923 | −1.261841296 | −1.070655826 |
| chr11 | 33278480 | 33280160 | 0.001517147 | −1.24358559 | −0.984890349 |
| chr9 | 75491200 | 75491640 | −0.001517139 | −0.692568637 | −0.960417528 |
| chr22 | 24551060 | 24553220 | 0.001517032 | −0.987780092 | −0.80570395 |
| chr20 | 20300740 | 20301500 | −0.001516684 | −1.037561598 | −1.322200086 |
| chr15 | 65590100 | 65590360 | 0.001516258 | −1.128429446 | −0.915959659 |
| chr2 | 120516560 | 120518420 | 0.001515343 | −1.164304273 | −0.89815653 |
| chr18 | 24941620 | 24942260 | −0.001515212 | −1.030682732 | −1.16911929 |
| chr21 | 43933620 | 43934700 | 0.001515009 | −1.074990128 | −0.784008813 |
| chr6 | 75039880 | 75040400 | −0.001514259 | −0.994029795 | −1.304360336 |
| chr5 | 159835640 | 159836060 | −0.001514206 | −1.04740576 | −1.341884151 |
| chr17 | 27944980 | 27945340 | 0.001513651 | −1.219175939 | −0.980711226 |
| chr6 | 132203540 | 132203960 | 0.001513398 | −1.256635803 | −1.140306153 |
| chr7 | 29448420 | 29448700 | 0.001513293 | −1.280428399 | −1.021687562 |
| chr6 | 26031160 | 26031600 | −0.001512746 | −1.021233603 | −1.281434227 |
| chr1 | 72153760 | 72154240 | −0.001512361 | −0.960992306 | −1.201482563 |
| chr10 | 66313440 | 66313820 | −0.001511997 | −0.992707592 | −1.273813396 |
| chr7 | 88591780 | 88592300 | 0.001511443 | −1.135335282 | −1.013655449 |
| chr15 | 41694540 | 41694880 | 0.001511322 | −1.2855103 | −1.094417252 |
| chr15 | 41694540 | 41694880 | 0.001511322 | −1.2855103 | −1.094417252 |
| chr5 | 27636120 | 27636500 | 0.001511186 | −1.264368707 | −1.124118825 |
| chr5 | 27636120 | 27636500 | 0.001511186 | −1.264368707 | −1.124118825 |
| chr2 | 239228500 | 239229800 | −0.001511132 | −1.040983793 | −1.153680891 |
| chr16 | 68344380 | 68345520 | 0.00150991 | −1.109176455 | −0.896747692 |
| chr1 | 24155580 | 24156020 | −0.001509774 | −0.92991535 | −1.191102116 |
| chr6 | 41316620 | 41317180 | −0.001509475 | −0.882883441 | −1.209327928 |
| chr21 | 47555200 | 47556000 | −0.001508663 | −0.769546043 | −0.867540165 |
| chr6 | 139308280 | 139309960 | 0.001508503 | −1.268036539 | −0.960574011 |
| chr16 | 89574280 | 89574940 | 0.001507958 | −1.145164717 | −0.953243983 |
| chr11 | 128564740 | 128565060 | 0.001507675 | −1.151441955 | −0.853983207 |
| chr19 | 1146560 | 1147000 | 0.001507194 | −1.118656083 | −0.880862778 |
| chr15 | 92395940 | 92397700 | 0.001506916 | −1.258232023 | −1.020573288 |
| chr11 | 31987560 | 31988000 | −0.001506566 | −0.943449321 | −1.311376206 |
| chr8 | 145023280 | 145023540 | 0.001506266 | −1.153244889 | −0.923213947 |
| chr17 | 43024540 | 43025880 | 0.001505815 | −1.208900206 | −1.037386423 |
| chr2 | 205916940 | 205917360 | −0.001505333 | −1.167450951 | −1.302265037 |
| chr19 | 1855320 | 1855680 | 0.001504755 | −1.03005901 | −0.888606973 |
| chr8 | 121310640 | 121311040 | −0.001504388 | −1.07742818 | −1.281104267 |
| chr16 | 2551240 | 2551520 | 0.001504033 | −1.187391318 | −0.893436804 |
| chr2 | 162623380 | 162623980 | −0.001504007 | −1.07365009 | −1.335101856 |
| chr8 | 135724540 | 135725960 | 0.001503225 | −1.27893126 | −1.132817148 |
| chr2 | 127807480 | 127808360 | 0.001503165 | −1.18760904 | −0.925364834 |
| chr2 | 46682960 | 46683460 | −0.001502807 | −0.910116094 | −1.164250162 |
| chr11 | 12303140 | 12304020 | 0.001502573 | −1.308463657 | −1.085984096 |
| chr7 | 81774400 | 81775240 | −0.001502357 | −0.929904735 | −1.195951571 |
| chr7 | 81774360 | 81775280 | −0.001502357 | −0.929904735 | −1.195951571 |
| chr19 | 14428900 | 14429340 | −0.001501718 | −0.92749128 | −1.158924235 |
| chr7 | 129697060 | 129697440 | −0.0015012 | −1.097873849 | −1.335046655 |
| chr8 | 109905540 | 109905940 | −0.001500894 | −0.965796876 | −1.202000538 |
| chr12 | 1702960 | 1704500 | −0.001500792 | −1.117942899 | −1.229586746 |
| chr1 | 156115820 | 156116080 | 0.001500676 | −1.167615656 | −0.883774245 |
| chr2 | 198227000 | 198227600 | 0.001500606 | −1.255235725 | −1.179416973 |
| chr14 | 85558700 | 85559360 | −0.001500606 | −0.936544958 | −1.172460593 |
| chr14 | 48009540 | 48010000 | 0.001500076 | −1.126728693 | −0.921083094 |
| chr6 | 75039940 | 75040360 | −0.00150006 | −1.009438658 | −1.279184566 |
| chr1 | 36240340 | 36240600 | 0.001499808 | −1.242686912 | −0.967155275 |
| chr10 | 95545360 | 95546380 | 0.001499788 | −1.38669291 | −1.222485934 |
| chr17 | 5319440 | 5319680 | 0.00149935 | −0.9357823 | −0.791207127 |
| chr5 | 25257840 | 25258360 | −0.00149873 | −0.866752019 | −1.180493986 |
| chr22 | 45831600 | 45831900 | 0.001497999 | −1.063202178 | −0.886143711 |
| chr11 | 40782200 | 40782660 | −0.001497929 | −0.989516488 | −1.169373239 |
| chr1 | 115052800 | 115054680 | 0.001497683 | −1.261156534 | −1.024133395 |
| chr2 | 172260340 | 172260820 | −0.001496702 | −0.991953703 | −1.217862581 |
| chr18 | 71151620 | 71152080 | −0.001496373 | −1.176439797 | −1.365719861 |
| chr17 | 7566360 | 7566620 | −0.001494871 | −0.669385954 | −0.954574768 |
| chr18 | 19903480 | 19904080 | −0.001494779 | −0.995059167 | −1.319525312 |
| chr6 | 154132220 | 154132680 | −0.001494773 | −1.102690991 | −1.353735252 |
| chr17 | 33390580 | 33390960 | 0.001494018 | −1.152518702 | −0.868324805 |
| chr4 | 187997460 | 187997960 | −0.001493842 | −1.066179951 | −1.268682536 |
| chr19 | 33382860 | 33383200 | 0.001493281 | −1.076583589 | −0.985068107 |
| chr19 | 33382860 | 33383200 | 0.001493281 | −1.076583589 | −0.985068107 |
| chr3 | 132135600 | 132136960 | 0.001492677 | −1.223227574 | −1.016514706 |
| chr2 | 173817760 | 173818340 | −0.001492121 | −0.959513867 | −1.283204553 |
| chr2 | 173817760 | 173818340 | −0.001492121 | −0.959513867 | −1.283204553 |
| chr1 | 192777600 | 192778460 | 0.001491751 | −1.284605232 | −1.048939135 |
| chr17 | 7253500 | 7253920 | 0.001491127 | −1.010430569 | −0.86491446 |
| chr6 | 48858000 | 48858460 | −0.001490789 | −0.925397227 | −1.258514175 |
| chr6 | 48857980 | 48858480 | −0.001490789 | −0.925397227 | −1.258514175 |
| chr15 | 50971500 | 50971960 | −0.001490508 | −1.016808968 | −1.232502259 |
| chr1 | 5871200 | 5871660 | −0.001490334 | −1.033131356 | −1.177239554 |
| chr11 | 7166260 | 7166700 | −0.00149022 | −0.954867457 | −1.240492526 |
| chr2 | 173486200 | 173486500 | −0.001490046 | −0.863051591 | −1.201048699 |
| chr2 | 173486200 | 173486500 | −0.001490046 | −0.863051591 | −1.201048699 |
| chr16 | 25026060 | 25027360 | 0.001489233 | −1.204091409 | −0.990719479 |
| chr3 | 182697320 | 182699140 | 0.001488411 | −1.233196703 | −0.989689651 |
| chr3 | 141977800 | 141978200 | −0.001488118 | −1.172173076 | −1.349013807 |
| chr21 | 29028000 | 29028600 | −0.001487698 | −0.894218737 | −1.151688078 |
| chr12 | 57753600 | 57753900 | −0.001487521 | −1.031662497 | −1.191449797 |
| chr8 | 65266100 | 65266600 | −0.001487014 | −1.059620384 | −1.335748212 |
| chr10 | 70287020 | 70287480 | 0.001486673 | −1.256816394 | −1.001330288 |
| chr15 | 101457900 | 101458800 | 0.001486592 | −1.154274754 | −0.958792453 |
| chr4 | 166247760 | 166249300 | 0.001486575 | −1.377867498 | −1.174409938 |
| chr4 | 31331860 | 31332300 | −0.001486334 | −1.034436774 | −1.222650299 |
| chr17 | 61776760 | 61777240 | 0.001485967 | −1.239618482 | −0.97687258 |
| chr10 | 33692380 | 33692800 | 0.001485933 | −1.0061804 | −1.155218718 |
| chr10 | 33692380 | 33692800 | −0.001485933 | −1.0061804 | −1.155218718 |
| chr18 | 41940120 | 41940480 | −0.001485899 | −1.002658796 | −1.248161593 |
| chr2 | 203499360 | 203500900 | 0.001484981 | −1.169217672 | −0.9601797 |
| chr20 | 42272060 | 42273140 | 0.00148485 | −1.332411501 | −1.172827976 |
| chr20 | 42272060 | 42273140 | 0.00148485 | −1.332411501 | −1.172827976 |
| chr12 | 123850300 | 123850540 | 0.001484722 | −1.200657204 | −0.975864961 |
| chr14 | 102185000 | 102185740 | −0.001483931 | −0.952971462 | −1.102026296 |
| chr14 | 102184980 | 102185760 | −0.001483931 | −0.952971462 | −1.102026296 |
| chr9 | 27887800 | 27888260 | 0.0014838 | −0.938687255 | −0.858457396 |
| chr10 | 86261420 | 86261840 | −0.001483513 | −0.890954548 | −1.076236071 |
| chr20 | 25370560 | 25371820 | 0.001482714 | −1.372598886 | −1.134417862 |
| chr17 | 7738180 | 7739140 | 0.001482531 | −1.132365921 | −0.946937515 |
| chr8 | 33443680 | 33444140 | −0.001482367 | −1.044129478 | −1.281632377 |
| chr6 | 9787580 | 9788160 | 0.001482328 | −1.143932826 | −1.028654826 |
| chr6 | 9787600 | 9788140 | 0.001482328 | −1.143932826 | −1.028654826 |
| chr2 | 124159760 | 124160120 | −0.00148196 | −1.04461634 | −1.201964065 |
| chr11 | 65192740 | 65192940 | 0.001481843 | −1.232327613 | −1.019497428 |
| chr10 | 57301800 | 57302340 | 0.001481522 | −1.114454218 | −1.005654604 |
| chr5 | 148085040 | 148085240 | −0.001481377 | −0.894351035 | −1.186616515 |
| chr5 | 148085040 | 148085240 | −0.001481377 | −0.894351035 | −1.186616515 |
| chr16 | 76754340 | 76754640 | −0.001480798 | −0.676387671 | −1.042152789 |
| chr16 | 76754340 | 76754640 | −0.001480798 | −0.676387671 | −1.042152789 |
| chr17 | 76347200 | 76347580 | 0.001480392 | −1.079669266 | −0.88005833 |
| chr11 | 6703800 | 6705280 | 0.001480169 | −1.152490979 | −0.970457783 |
| chr9 | 100916480 | 100916800 | −0.001479956 | −0.72768643 | −1.049632787 |
| chr19 | 41814460 | 41814720 | 0.001479692 | −1.18742561 | −0.999889369 |
| chr1 | 215202660 | 215203180 | 0.00147921 | −1.160309782 | −1.10450622 |
| chr18 | 48657480 | 48657960 | −0.001478619 | −1.008979256 | −1.217979855 |
| chr4 | 158667240 | 158667820 | −0.001478519 | −1.084838791 | −1.392285559 |
| chr17 | 7342600 | 7342880 | 0.001477739 | −0.986259785 | −0.775387464 |
| chr3 | 118653920 | 118654420 | −0.001476673 | −0.959592504 | −1.179910323 |
| chr17 | 67565220 | 67565500 | 0.001476447 | −1.24505474 | −1.077809833 |
| chr7 | 53436740 | 53437380 | −0.001475994 | −0.722967328 | −0.997844986 |
| chr3 | 162566540 | 162566740 | 0.001475749 | −1.425553627 | −1.222765299 |
| chr14 | 72070580 | 72070880 | −0.001475431 | −0.921370385 | −1.148453247 |
| chr14 | 72070580 | 72070880 | −0.001475431 | −0.921370385 | −1.148453247 |
| chr11 | 6494220 | 6495500 | 0.001475131 | −1.203951587 | −0.918679478 |
| chr1 | 109942040 | 109942320 | 0.001474932 | −1.194986089 | −1.0100893 |
| chr1 | 109942040 | 109942320 | 0.001474932 | −1.194986089 | −1.0100893 |
| chr16 | 20991340 | 20991760 | −0.001474898 | −0.917156678 | −1.245456233 |
| chr14 | 72849720 | 72850100 | 0.001474562 | −1.184742975 | −1.028977429 |
| chr5 | 58912520 | 58912980 | −0.001474497 | −1.012138025 | −1.25547214 |
| chr5 | 58912500 | 58913000 | −0.001474497 | −1.012138025 | −1.25547214 |
| chr6 | 90077600 | 90078460 | 0.001474288 | −1.132591839 | −0.949336228 |
| chr3 | 107436840 | 107437400 | −0.001473968 | −1.019915243 | −1.215837885 |
| chr13 | 29232460 | 29234020 | 0.0014739 | −1.326175133 | −1.03053414 |
| chr17 | 63096460 | 63097380 | 0.001473891 | −1.101566576 | −0.910762776 |
| chr3 | 197236540 | 197237800 | 0.001473775 | −1.301610206 | −1.082307979 |
| chr11 | 106882080 | 106882500 | −0.001473736 | −0.921072069 | −1.25915629 |
| chr12 | 56440900 | 56441300 | 0.001473681 | −1.15746157 | −0.965505448 |
| chr11 | 63974980 | 63975220 | 0.001473632 | −1.130377952 | −0.887251879 |
| chr17 | 28258420 | 28258680 | 0.001473452 | −1.051189776 | −1.009864147 |
| chr9 | 92221620 | 92221980 | −0.001472816 | −0.757670747 | −0.862411352 |
| chr7 | 125730880 | 125731500 | −0.00147264 | −0.972561623 | −1.270415794 |
| chr20 | 30101900 | 30102400 | 0.001471765 | −1.329491898 | −1.114019872 |
| chr6 | 16070760 | 16071220 | −0.00147156 | −0.977489715 | −1.29740558 |
| chr17 | 8648940 | 8649740 | 0.001471187 | −1.122659106 | −0.934493378 |
| chr17 | 8648940 | 8649740 | 0.001471187 | −1.122659106 | −0.934493378 |
| chr2 | 123477020 | 123477400 | −0.001470845 | −1.073406361 | −1.315550387 |
| chr3 | 29511240 | 29511760 | 0.001470465 | −1.198260694 | −1.118572894 |
| chr3 | 87399720 | 87400220 | 0.001470286 | −1.305266741 | −1.071381226 |
| chr9 | 76289320 | 76290320 | −0.001470157 | −0.897014528 | −1.228504179 |
| chr13 | 45012180 | 45012440 | 0.001470064 | −1.212193837 | −1.024611683 |
| chr13 | 45012180 | 45012440 | 0.001470064 | −1.212193837 | −1.024611683 |
| chr13 | 61752120 | 61752520 | −0.00146977 | −0.930757105 | −1.188405511 |
| chr19 | 52097040 | 52098260 | 0.001469677 | −1.15506519 | −0.922807632 |
| chr21 | 17736760 | 17737260 | −0.001469578 | −0.712763283 | −0.969308837 |
| chr19 | 796640 | 798380 | 0.001469426 | −1.192750729 | −1.045742068 |
| chr14 | 77339180 | 77339440 | 0.001469188 | −1.352863634 | −1.029725588 |
| chr20 | 6787380 | 6787880 | 0.001468892 | −1.183067642 | −1.099765336 |
| chr13 | 50639180 | 50639400 | −0.001468816 | −1.0772544 | −1.267959581 |
| chr13 | 50639180 | 50639400 | −0.001468816 | −1.0772544 | −1.267959581 |
| chr2 | 150633680 | 150634260 | −0.001468461 | −0.893717583 | −1.130230342 |
| chr4 | 109025340 | 109025840 | −0.001468401 | −0.895341052 | −1.189149135 |
| chr11 | 2216180 | 2216440 | −0.00146803 | −0.853918539 | −0.983133651 |
| chr8 | 103643720 | 103644540 | −0.001468017 | −1.077520799 | −1.384889414 |
| chr12 | 126330740 | 126331220 | −0.001467969 | −0.992731775 | −1.195159116 |
| chr2 | 220142440 | 220142700 | 0.001467941 | −1.360907556 | −1.033373989 |
| chr2 | 220142440 | 220142700 | 0.001467941 | −1.360907556 | −1.033373989 |
| chr11 | 567800 | 569280 | −0.001466893 | −1.045502775 | −1.114934749 |
| chr15 | 40613140 | 40613420 | 0.001466508 | −1.231494384 | −0.983414554 |
| chr8 | 20807140 | 20807560 | −0.001466371 | −1.062630901 | −1.301326813 |
| chr8 | 84332600 | 84333080 | −0.001466359 | −1.05389805 | −1.192870493 |
| chr1 | 167503100 | 167503380 | 0.001466238 | −1.23556292 | −0.99664247 |
| chr13 | 64647960 | 64648540 | −0.001465974 | −1.117138768 | −1.348889828 |
| chr19 | 54061940 | 54063380 | 0.001465019 | −0.971926567 | −0.792135223 |
| chr19 | 2307800 | 2308720 | 0.001464643 | −1.134280639 | −0.97119594 |
| chr13 | 54228040 | 54228520 | −0.001464474 | −1.067628614 | −1.328747895 |
| chr17 | 3868400 | 3868680 | 0.001463555 | −1.114802789 | −0.910186534 |
| chr7 | 71570580 | 71571020 | −0.001463458 | −0.887765707 | −1.114819309 |
| chr7 | 71570560 | 71571040 | −0.001463458 | −0.887765707 | −1.114819309 |
| chr3 | 81746900 | 81747500 | −0.001463243 | −0.903104719 | −1.173024915 |
| chr13 | 104296060 | 104296320 | −0.001462913 | −0.801437034 | −0.944285099 |
| chr20 | 41396140 | 41396620 | −0.001462798 | −0.956024097 | −1.256907709 |
| chr10 | 125754160 | 125754440 | 0.001462783 | −1.330803748 | −1.060405745 |
| chr5 | 108024880 | 108025340 | −0.001462705 | −0.971930952 | −1.192975425 |
| chr14 | 94468180 | 94468440 | 0.001461127 | −1.211298598 | −0.991168138 |
| chr10 | 127511240 | 127512240 | 0.001461032 | −1.198921449 | −0.99374395 |
| chr8 | 68402560 | 68402840 | 0.001460833 | −1.184641244 | −1.022193621 |
| chr4 | 48780800 | 48782760 | 0.00146039 | −1.280733137 | −1.060899854 |
| chr10 | 24617760 | 24618380 | −0.001460073 | −0.95491963 | −1.320825755 |
| chr14 | 46954720 | 46955180 | −0.001459978 | −0.987432147 | −1.205233246 |
| chr9 | 22227940 | 22228480 | −0.001459633 | −0.922231497 | −1.124752463 |
| chr6 | 127850260 | 127850920 | −0.001459511 | −0.988497756 | −1.329549767 |
| chr10 | 26624040 | 26624320 | 0.001459425 | −1.191651933 | −0.975722582 |
| chr7 | 140291540 | 140291920 | −0.001458664 | −1.119363194 | −1.312999525 |
| chr19 | 45946680 | 45947840 | 0.001458249 | −1.006881991 | −0.843013892 |
| chr19 | 45946660 | 45947860 | 0.001458249 | −1.006881991 | −0.843013892 |
| chr2 | 181318140 | 181318520 | −0.001457287 | −0.940451536 | −1.187305649 |
| chr2 | 181318100 | 181318560 | −0.001457287 | −0.940451536 | −1.187305649 |
| chr7 | 128544760 | 128545020 | 0.001456791 | −1.074098476 | −0.81108403 |
| chr7 | 128544760 | 128545020 | 0.001456791 | −1.074098476 | −0.81108403 |
| chr22 | 46402260 | 46402520 | 0.001456653 | −1.178284895 | −0.995456444 |
| chr21 | 46312740 | 46313000 | 0.001456395 | −1.149343406 | −0.866281386 |
| chr19 | 10526900 | 10528120 | 0.001456097 | −1.111876888 | −0.893748837 |
| chr4 | 106835440 | 106835660 | −0.001456046 | −1.091819655 | −1.318528486 |
| chr5 | 23418200 | 23418660 | −0.001455801 | −0.94542242 | −1.24605923 |
| chr11 | 92204880 | 92205360 | −0.001454996 | −0.991982825 | −1.239841643 |
| chr3 | 197281500 | 197281980 | 0.001454681 | −1.290936593 | −1.092912012 |
| chr14 | 73490040 | 73490320 | −0.00145438 | −0.911932645 | −1.199930155 |
| chr3 | 192875500 | 192876380 | 0.001454372 | −1.408587272 | −1.250941495 |
| chr4 | 16900080 | 16900700 | −0.001453541 | −1.071163773 | −1.266419273 |
| chr1 | 28198880 | 28199240 | 0.001453373 | −1.216789058 | −1.065658013 |
| chr13 | 88557600 | 88558080 | 0.001453184 | −1.269062958 | −1.197004047 |
| chr11 | 96105640 | 96106060 | −0.001452728 | −1.08430322 | −1.365726795 |
| chr12 | 78267220 | 78267740 | −0.001452479 | −1.023335171 | −1.244128653 |
| chr17 | 78316220 | 78316660 | −0.001452152 | −0.803255643 | −1.084355219 |
| chr1 | 48667560 | 48668480 | 0.001452049 | −1.184290146 | −0.861638411 |
| chr20 | 30200580 | 30201000 | 0.001451538 | −1.288097153 | −0.995581488 |
| chr13 | 76396660 | 76397140 | −0.001451053 | −1.031777562 | −1.257354025 |
| chr17 | 4848860 | 4849120 | 0.001451049 | −1.061595294 | −0.867375196 |
| chr8 | 113422180 | 113423360 | −0.001450939 | −1.052578959 | −1.286778373 |
| chr1 | 24155640 | 24155980 | −0.001450486 | −0.947979925 | −1.179026099 |
| chr5 | 26398300 | 26398860 | −0.001450461 | −1.041689955 | −1.297952841 |
| chr12 | 20285040 | 20285740 | −0.001450398 | −0.889075099 | −1.179602588 |
| chr10 | 134525560 | 134526840 | 0.001449831 | −1.095191503 | −0.823486752 |
| chr20 | 22064020 | 22064860 | −0.00144933 | −0.941853607 | −1.233429485 |
| chr11 | 67168540 | 67169700 | −0.001449187 | −1.002380249 | −1.178515177 |
| chr8 | 71421940 | 71422420 | −0.001448553 | −1.154813275 | −1.283103378 |
| chr5 | 28358080 | 28358580 | −0.001448197 | −1.083497656 | −1.383220814 |
| chr20 | 8661420 | 8662000 | 0.001447998 | −1.254621848 | −1.11761289 |
| chr12 | 53764580 | 53764800 | 0.001447718 | −1.160800893 | −0.951709383 |
| chr6 | 129521060 | 129521960 | 0.001447011 | −1.215210198 | −1.110390849 |
| chr21 | 22894220 | 22894720 | 0.001446442 | −1.115063746 | −1.060620103 |
| chr5 | 180525640 | 180525860 | −0.001446173 | −1.107995153 | −1.261877829 |
| chr21 | 31220940 | 31221300 | −0.001445959 | −0.670730744 | −0.873282366 |
| chr8 | 18089920 | 18090440 | −0.001445796 | −1.021375422 | −1.224146089 |
| chr8 | 18089900 | 18090460 | −0.001445796 | −1.021375422 | −1.224146089 |
| chr6 | 56410920 | 56411400 | −0.001445142 | −1.065548407 | −1.236419738 |
| chr9 | 75298460 | 75298840 | −0.001445019 | −0.799324809 | −1.037004953 |
| chr14 | 72849700 | 72850100 | 0.001444724 | −1.12560354 | −0.991099741 |
| chr14 | 102783200 | 102783920 | 0.001444485 | −1.197051008 | −0.955558498 |
| chr5 | 128045820 | 128046260 | −0.001444098 | −0.934893852 | −1.123086227 |
| chr1 | 196405980 | 196406580 | −0.001443933 | −1.036160396 | −1.275033934 |
| chr1 | 9598920 | 9600340 | 0.001442292 | −1.208993616 | −1.042789413 |
| chr19 | 44258320 | 44260080 | 0.001442146 | −1.154231312 | −0.977252017 |
| chr8 | 50801120 | 50801580 | −0.00144212 | −1.111680243 | −1.410326996 |
| chr8 | 69109740 | 69110240 | −0.001442079 | −0.987559133 | −1.184285447 |
| chr5 | 110582780 | 110583300 | −0.001441858 | −0.846224046 | −1.164782254 |
| chr20 | 57210800 | 57211460 | −0.001441573 | −0.999155381 | −1.123556055 |
| chr2 | 66889860 | 66890360 | −0.001441539 | −1.024515115 | −1.175857133 |
| chr19 | 911960 | 913660 | 0.001441268 | −1.147326368 | −0.919746804 |
| chr5 | 110582720 | 110583420 | −0.001441093 | −0.887349195 | −1.180446107 |
| chr11 | 97131260 | 97132440 | 0.00144096 | −1.154024344 | −1.063311025 |
| chr7 | 124790000 | 124790520 | −0.001440616 | −0.977370407 | −1.187837391 |
| chr12 | 6978640 | 6978860 | 0.001440447 | −1.241565028 | −1.160925104 |
| chr12 | 6978640 | 6978860 | 0.001440447 | −1.241565028 | −1.160925104 |
| chr17 | 8450820 | 8451320 | −0.0014404 | −0.882830077 | −1.122160353 |
| chr9 | 14739220 | 14739800 | −0.001439649 | −0.780324743 | −1.061710966 |
| chr7 | 80221300 | 80221740 | −0.001439148 | −0.933107002 | −1.232555112 |
| chr14 | 24583600 | 24585160 | 0.001438536 | −1.243858703 | −0.978927937 |
| chr6 | 168613080 | 168613600 | 0.001438284 | −1.277384236 | −1.00190447 |
| chr2 | 71080140 | 71080640 | −0.001437913 | −0.970395423 | −1.285020682 |
| chr17 | 49197380 | 49198680 | 0.001437327 | −1.155343837 | −0.899600959 |
| chr17 | 49197440 | 49198620 | 0.001437327 | −1.155343837 | −0.899600959 |
| chr9 | 131901140 | 131902080 | 0.001437055 | −1.172346402 | −0.908594655 |
| chr20 | 4129260 | 4130100 | 0.00143691 | −1.254283936 | −0.990141228 |
| chr5 | 141348280 | 141349040 | 0.001436809 | −1.29142018 | −1.089501336 |
| chr13 | 21803220 | 21803780 | −0.001436269 | −1.137762142 | −1.266820797 |
| chr20 | 34790660 | 34791220 | 0.001435952 | −1.376240648 | −1.004264362 |
| chr17 | 57183580 | 57184540 | 0.001435757 | −1.133741475 | −0.926299035 |
| chr2 | 135487200 | 135487680 | −0.001435496 | −0.960178861 | −1.221580838 |
| chr16 | 84764060 | 84764360 | 0.001435309 | −1.184279779 | −1.023412004 |
| chr1 | 247357580 | 247358040 | −0.001435265 | −0.92656584 | −1.135862679 |
| chr17 | 80188800 | 80189720 | 0.001435023 | −1.116782899 | −0.865673204 |
| chr14 | 23797280 | 23797580 | 0.001434722 | −1.291123338 | −1.006354108 |
| chr1 | 69124780 | 69125160 | −0.001434095 | −0.953603504 | −1.249627712 |
| chr3 | 32163080 | 32163360 | −0.001433972 | −1.163899138 | −1.316414167 |
| chr3 | 32163080 | 32163360 | −0.001433972 | −1.163899138 | −1.316414167 |
| chr6 | 31514440 | 31516140 | 0.001433455 | −1.30990742 | −1.12545764 |
| chr22 | 46672460 | 46672940 | −0.001432298 | −0.967638256 | −1.204866004 |
| chr8 | 145023260 | 145023600 | 0.001432248 | −1.151761228 | −0.934194815 |
| chr11 | 24009020 | 24009420 | 0.00143149 | −1.265999979 | −1.069878982 |
| chr8 | 33443600 | 33444160 | −0.001431292 | −1.045479484 | −1.203318618 |
| chr11 | 92204940 | 92205280 | −0.001430293 | −0.996356364 | −1.248748418 |
| chr2 | 31956480 | 31957120 | −0.001429798 | −1.067426775 | −1.327695239 |
| chr19 | 41916460 | 41917180 | −0.001429537 | −0.821184499 | −0.972993066 |
| chr9 | 121152420 | 121153200 | −0.001429223 | −0.785180073 | −1.070028907 |
| chr19 | 33165160 | 33167220 | 0.001428439 | −1.2480865 | −1.058694995 |
| chr16 | 81040020 | 81041300 | 0.001428263 | −1.105410891 | −0.831945342 |
| chr4 | 122735120 | 122735500 | 0.001427907 | −1.010177589 | −0.957741006 |
| chr1 | 204600240 | 204600620 | −0.001427541 | −0.997704897 | −1.261934087 |
| chr6 | 43930780 | 43931960 | −0.001427272 | −1.060502919 | −1.342740463 |
| chr21 | 34099600 | 34100720 | 0.00142687 | −1.063303221 | −0.768099332 |
| chr17 | 53585600 | 53586000 | −0.001426044 | −0.920800909 | −1.173235562 |
| chr17 | 53585600 | 53586000 | −0.001426044 | −0.920800909 | −1.173235562 |
| chr5 | 159448880 | 159449180 | 0.001425616 | −1.268049499 | −1.069049708 |
| chr16 | 23568080 | 23569680 | 0.001425026 | −1.12011808 | −1.007228888 |
| chr8 | 113385020 | 113385440 | −0.001424976 | −0.855915281 | −1.133291201 |
| chr11 | 115271140 | 115271620 | −0.00142442 | −1.112793849 | −1.363570955 |
| chr10 | 17085860 | 17086520 | −0.001423974 | −0.971226971 | −1.292948286 |
| chr4 | 39844200 | 39844760 | −0.001423522 | −0.981667814 | −1.120456886 |
| chr18 | 62856740 | 62857420 | −0.001423295 | −0.913676561 | −1.153148869 |
| chr11 | 20937040 | 20937480 | −0.001423287 | −1.070806489 | −1.220806589 |
| chr1 | 9598760 | 9600380 | 0.001423112 | −1.319543499 | −1.124535897 |
| chr15 | 34393500 | 34394640 | 0.001423083 | −1.172589843 | −0.964421223 |
| chr2 | 179273140 | 179273740 | −0.001422873 | −1.041715874 | −1.239121085 |
| chr3 | 163004500 | 163005220 | 0.001422459 | −1.091567251 | −0.937053418 |
| chr10 | 57700180 | 57700660 | −0.001421578 | −0.982335674 | −1.219539089 |
| chr20 | 57210860 | 57211440 | −0.001421443 | −1.020576279 | −1.10921861 |
| chr20 | 23058060 | 23058460 | −0.001420908 | −0.996443138 | −1.090931829 |
| chr7 | 17026200 | 17026660 | −0.001420824 | −0.961183416 | −1.227205225 |
| chr10 | 114939320 | 114939820 | −0.00142052 | −1.015180481 | −1.157198591 |
| chr3 | 10052680 | 10053060 | 0.001420484 | −1.304906348 | −1.114127584 |
| chr4 | 158915600 | 158916000 | −0.001420065 | −0.956466198 | −1.205735274 |
| chr7 | 6565960 | 6566500 | 0.001419863 | −1.251097039 | −1.02392748 |
| chr8 | 108294440 | 108295060 | −0.001419464 | −0.922802751 | −1.188869781 |
| chr8 | 20939500 | 20939900 | 0.001419351 | −1.056603132 | −1.009865537 |
| chr4 | 14965620 | 14966040 | −0.001419152 | −0.934125722 | −1.227453006 |
| chr17 | 44018780 | 44019300 | 0.001418991 | −1.126556697 | −0.837622655 |
| chr12 | 62653660 | 62655020 | 0.001418711 | −1.291403376 | −1.05281487 |
| chr11 | 23113060 | 23113620 | 0.001418482 | −1.346379145 | −1.171493841 |
| chr9 | 139760140 | 139761300 | 0.001418002 | −1.052439998 | −0.815831972 |
| chr12 | 727140 | 727720 | 0.001417526 | −1.257843816 | −0.932831755 |
| chr1 | 234613960 | 234615140 | 0.001417281 | −1.325499724 | −1.044191673 |
| chr8 | 144922240 | 144923820 | −0.001417161 | −0.994231659 | −1.12944699 |
| chr16 | 80239520 | 80240240 | −0.001417142 | −0.805744676 | −1.171411612 |
| chr3 | 37216920 | 37218840 | 0.001416595 | −1.362492434 | −1.013081819 |
| chr8 | 120628100 | 120628480 | −0.001416478 | −0.887303837 | −1.107368745 |
| chr7 | 125857620 | 125858020 | −0.001416366 | −0.920762362 | −1.244478805 |
| chr2 | 199713260 | 199713480 | −0.001416301 | −1.083999512 | −1.26220219 |
| chr2 | 199713260 | 199713480 | −0.001416301 | −1.083999512 | −1.26220219 |
| chr7 | 90657180 | 90657420 | −0.001416088 | −0.804383784 | −1.067714775 |
| chr3 | 162976300 | 162976840 | −0.001416029 | −0.996533318 | −1.300459866 |
| chr8 | 63124060 | 63124380 | −0.001415919 | −0.957853254 | −1.2683195 |
| chr8 | 63124060 | 63124380 | −0.001415919 | −0.957853254 | −1.2683195 |
| chr6 | 75939660 | 75940240 | −0.001415915 | −1.063813977 | −1.345312033 |
| chr8 | 37978620 | 37979140 | −0.001415714 | −1.085497143 | −1.26725493 |
| chr18 | 19375000 | 19375540 | −0.001415491 | −1.118723093 | −1.35096958 |
| chr18 | 19374940 | 19375600 | −0.001415491 | −1.118723093 | −1.35096958 |
| chr17 | 48100500 | 48102840 | 0.001415215 | −1.101118635 | −0.933520726 |
| chr4 | 148076060 | 148076400 | 0.00141453 | −1.090595906 | −1.016160424 |
| chr4 | 17655320 | 17655780 | −0.001414156 | −0.977619828 | −1.103257433 |
| chr12 | 65284720 | 65285000 | 0.001413928 | −1.313716929 | −1.056675065 |
| chr2 | 53106420 | 53106920 | 0.001413818 | −1.110185559 | −1.028157633 |
| chr8 | 113385040 | 113385380 | −0.001412555 | −0.869549381 | −1.149969307 |
| chr4 | 148964560 | 148965160 | −0.001412413 | −1.030597057 | −1.288689264 |
| chr4 | 109420320 | 109420760 | −0.001412363 | −0.972208959 | −1.163528984 |
| chr15 | 98407560 | 98408000 | −0.001412162 | −0.865293509 | −1.08355857 |
| chr10 | 22650560 | 22651380 | −0.001412075 | −0.995064147 | −1.273080312 |
| chr5 | 127290240 | 127290740 | −0.001411643 | −1.033452296 | −1.249648455 |
| chr17 | 3868420 | 3868640 | 0.001411491 | −1.102256008 | −0.87101027 |
| chr18 | 41401120 | 41401520 | −0.001411382 | −1.042273342 | −1.143872934 |
| chr18 | 41401120 | 41401520 | −0.001411382 | −1.042273342 | −1.143872934 |
| chr22 | 37882300 | 37882620 | 0.001410839 | −1.085344734 | −0.84308942 |
| chr5 | 83984700 | 83985380 | 0.001410285 | −1.079939961 | −0.93037352 |
| chr17 | 48228200 | 48228700 | 0.001410191 | −1.246729449 | −1.065953415 |
| chr3 | 6169200 | 6169640 | −0.001410027 | −1.123560491 | −1.322969849 |
| chr3 | 6169180 | 6169660 | −0.001410027 | −1.123560491 | −1.322969849 |
| chr9 | 132123380 | 132123760 | −0.001409972 | −0.820399837 | −0.902447314 |
| chr11 | 64614620 | 64614900 | 0.001409908 | −1.374892427 | −1.16767682 |
| chr12 | 39315960 | 39316440 | 0.001409544 | −1.220473696 | −1.163055245 |
| chr22 | 38447860 | 38449100 | 0.001409382 | −1.102082895 | −0.87988093 |
| chr8 | 88914140 | 88914640 | −0.001409273 | −1.040023607 | −1.337792506 |
| chr1 | 68819900 | 68820420 | 0.001409153 | −1.202082916 | −1.120655864 |
| chr4 | 112963580 | 112964020 | −0.001409124 | −1.125074428 | −1.304117926 |
| chr19 | 796620 | 798320 | 0.001408806 | −1.192750729 | −1.050926439 |
| chr16 | 57054160 | 57054720 | −0.001408786 | −0.894753751 | −1.032170398 |
| chr2 | 141730320 | 141730880 | −0.001408756 | −1.040660464 | −1.240655253 |
| chr9 | 24718040 | 24718300 | 0.001408529 | −1.036652293 | −0.94552991 |
| chr9 | 24718040 | 24718300 | 0.001408529 | −1.036652293 | −0.94552991 |
| chr1 | 20129240 | 20129640 | −0.001408518 | −0.771397684 | −0.999596249 |
| chr20 | 31735000 | 31735420 | 0.001408172 | −1.129089968 | −0.995483757 |
| chr20 | 31735000 | 31735420 | 0.001408172 | −1.129089968 | −0.995483757 |
| chr2 | 40511260 | 40512000 | −0.001408059 | −0.917736495 | −1.226681044 |
| chr5 | 105658300 | 105659200 | −0.001407732 | −0.893250838 | −1.218153684 |
| chr3 | 42632040 | 42632700 | 0.001407621 | −1.318026205 | −1.128200004 |
| chr18 | 7269140 | 7269660 | −0.001407341 | −1.00272068 | −1.256239719 |
| chr1 | 28652100 | 28652360 | −0.0014073 | −0.920339857 | −1.120063721 |
| chr1 | 105627420 | 105627900 | −0.001406653 | −0.910979314 | −1.20163367 |
| chr17 | 7253520 | 7253940 | 0.001406617 | −1.029406197 | −0.878261006 |
| chr12 | 95610720 | 95611780 | 0.001406497 | −1.309990938 | −1.118393218 |
| chr12 | 92078620 | 92079320 | −0.001405895 | −1.131879565 | −1.344294041 |
| chr17 | 35849220 | 35850560 | 0.001404265 | −1.249521768 | −1.044667639 |
| chr19 | 10652160 | 10652400 | 0.001404236 | −1.073348052 | −0.88315584 |
| chr19 | 10652160 | 10652400 | 0.001404236 | −1.073348052 | −0.88315584 |
| chr14 | 24603180 | 24603380 | −0.001404214 | −0.932814267 | −1.16721229 |
| chr10 | 101988520 | 101990160 | 0.001404203 | −1.173163605 | −0.938567057 |
| chr2 | 9606140 | 9606780 | 0.001404154 | −1.28939094 | −1.039262289 |
| chr3 | 179899280 | 179899700 | −0.001403989 | −1.202277883 | −1.341157683 |
| chr9 | 131533760 | 131534440 | −0.001403669 | −0.745372125 | −0.872696377 |
| chr5 | 21886460 | 21886880 | −0.001402982 | −0.927205812 | −1.228153052 |
| chr10 | 64511020 | 64511620 | −0.001402639 | −0.997971755 | −1.286118535 |
| chr7 | 28698360 | 28698800 | 0.001401793 | −1.054345706 | −0.957470843 |
| chr7 | 150223680 | 150224060 | −0.00140142 | −1.021948126 | −1.174420708 |
| chr2 | 65282800 | 65284160 | 0.001401368 | −1.350461992 | −1.119430097 |
| chr6 | 52058840 | 52059340 | −0.001401329 | −1.06240472 | −1.372139348 |
| chr1 | 27814140 | 27814420 | −0.001401039 | −1.034303744 | −1.267867917 |
| chr6 | 108615880 | 108616260 | 0.001400701 | −1.313962923 | −1.108830504 |
| chr10 | 50213740 | 50214140 | −0.001400374 | −0.917986302 | −1.210939527 |
| chr22 | 37988520 | 37988860 | 0.001400342 | −0.979629603 | −0.782125006 |
| chr2 | 220507240 | 220507580 | 0.001399322 | −1.251010037 | −0.987816005 |
| chr20 | 31073040 | 31073300 | 0.001398961 | −1.212125518 | −1.024226352 |
| chr20 | 31073040 | 31073300 | 0.001398961 | −1.212125518 | −1.024226352 |
| chr11 | 126350640 | 126350960 | 0.001398955 | −1.316781596 | −1.0691452 |
| chr1 | 115052820 | 115054560 | 0.00139887 | −1.24302352 | −1.017085637 |
| chr5 | 1464560 | 1466120 | 0.001398359 | −1.209136152 | −0.973261816 |
| chr15 | 69168200 | 69168480 | 0.001397166 | −1.211547586 | −1.041502958 |
| chr15 | 69167760 | 69168920 | 0.001397166 | −1.211547586 | −1.041502958 |
| chr15 | 41196220 | 41196520 | 0.001397132 | −1.275110882 | −1.072756509 |
| chr4 | 33132120 | 33132980 | −0.001397098 | −1.088758379 | −1.282301703 |
| chr4 | 15651640 | 15651880 | 0.001396644 | −1.215196361 | −0.888042658 |
| chr18 | 75876940 | 75877560 | −0.001395974 | −1.12077355 | −1.330540413 |
| chr9 | 74137300 | 74137760 | 0.001395614 | −1.062534127 | −0.903283333 |
| chr7 | 117018940 | 117019460 | −0.0013956 | −0.999827986 | −1.200493479 |
| chr19 | 3500560 | 3501360 | 0.001395356 | −1.170157942 | −1.004839106 |
| chr14 | 62228360 | 62229640 | −0.001395116 | −0.988181343 | −1.101410265 |
| chr9 | 137203000 | 137203660 | −0.001395034 | −0.85287308 | −1.137485975 |
| chr2 | 210300480 | 210300900 | −0.001394775 | −0.822939099 | −1.11338811 |
| chr12 | 92812440 | 92813120 | −0.001394759 | −1.029704631 | −1.197826576 |
| chr17 | 65082620 | 65082960 | −0.0013942 | −0.898528771 | −1.111643665 |
| chr15 | 34330540 | 34331680 | 0.001393966 | −1.060040027 | −0.777810521 |
| chr21 | 20687320 | 20688240 | 0.001393525 | −1.078145418 | −0.92490548 |
| chr8 | 93719820 | 93720320 | −0.001392571 | −0.94603442 | −1.256998287 |
| chr19 | 52281160 | 52281480 | 0.001392544 | −0.946245878 | −0.759667435 |
| chr18 | 12883020 | 12884740 | −0.001392184 | −1.158029845 | −1.295429931 |
| chr4 | 64508880 | 64509280 | 0.001392046 | −1.266990232 | −1.151628124 |
| chr1 | 11301780 | 11302080 | 0.001391637 | −1.055830354 | −0.829858283 |
| chr5 | 18179240 | 18179660 | −0.001391105 | −0.880002182 | −1.183985349 |
| chr18 | 61089100 | 61090520 | 0.001390894 | −1.345779867 | −1.155386025 |
| chr7 | 118731700 | 118732100 | 0.001390853 | −1.26484642 | −1.154994433 |
| chr4 | 74987260 | 74987600 | −0.001390743 | −1.106026431 | −1.364325547 |
| chr2 | 174828280 | 174830300 | 0.001390348 | −1.37732452 | −1.149178971 |
| chr4 | 152020140 | 152021240 | 0.001389875 | −1.353849805 | −1.116192327 |
| chr9 | 115123620 | 115124340 | 0.001388858 | −1.02086417 | −0.871903262 |
| chr1 | 22378500 | 22379940 | −0.001388453 | −1.05848813 | −1.109885477 |
| chr17 | 36833340 | 36833620 | 0.001388065 | −1.118493796 | −0.933766634 |
| chr21 | 26682220 | 26682660 | 0.001387847 | −1.001041457 | −0.763779612 |
| chr18 | 58091440 | 58092100 | 0.001387661 | −1.285788722 | −1.222160364 |
| chr8 | 1949440 | 1951440 | −0.001387074 | −0.997324697 | −1.082452649 |
| chr1 | 79707920 | 79708320 | −0.001386868 | −0.956334207 | −1.170790223 |
| chr17 | 43138260 | 43139100 | 0.001386813 | −1.195896657 | −1.01174043 |
| chr4 | 88928200 | 88929780 | 0.001385695 | −1.213731867 | −0.978622408 |
| chr11 | 27947340 | 27947760 | −0.001385563 | −1.054534289 | −1.250132947 |
| chr17 | 36833320 | 36833600 | 0.001385526 | −1.165084131 | −0.93963394 |
| chr17 | 1390660 | 1390940 | 0.001385523 | −1.168140291 | −0.992314721 |
| chr3 | 50275360 | 50275640 | 0.001385145 | −1.283073904 | −1.087182745 |
| chr3 | 50275360 | 50275640 | 0.001385145 | −1.283073904 | −1.087182745 |
| chr5 | 4261280 | 4261700 | 0.00138464 | −1.087081463 | −1.007626854 |
| chr2 | 80210160 | 80210500 | −0.001384615 | −0.773960987 | −1.052492446 |
| chr8 | 4926560 | 4927220 | −0.001384477 | −0.895124945 | −1.23201143 |
| chr6 | 95667660 | 95668020 | −0.001384174 | −0.955532913 | −1.164276169 |
| chr16 | 50785720 | 50786100 | 0.001383302 | −1.03997329 | −1.021055468 |
| chr19 | 30515940 | 30516380 | −0.001383142 | −0.914385056 | −1.152705586 |
| chr17 | 74732500 | 74734340 | 0.001381748 | −1.176281586 | −1.028416461 |
| chr21 | 18222620 | 18223000 | −0.001381457 | −0.821102575 | −1.14683259 |
| chr21 | 18222580 | 18223040 | −0.001381457 | −0.821102575 | −1.14683259 |
| chr3 | 102126020 | 102126520 | −0.001381314 | −1.170943478 | −1.388357471 |
| chr18 | 69398520 | 69399120 | −0.001381107 | −1.026037327 | −1.303520372 |
| chr4 | 112174840 | 112175360 | −0.001380984 | −1.080024031 | −1.25117999 |
| chr19 | 39894380 | 39894760 | 0.00138074 | −1.135273219 | −0.915994813 |
| chr20 | 633140 | 634660 | 0.001380563 | −1.216577301 | −1.057627746 |
| chr1 | 221376840 | 221377120 | 0.001380447 | −1.254192871 | −0.999779602 |
| chr4 | 124842860 | 124843420 | −0.00138036 | −0.831265011 | −1.096510314 |
| chr16 | 1967960 | 1968680 | 0.001380161 | −1.139479932 | −0.941985333 |
| chr16 | 77709660 | 77709940 | 0.001379958 | −1.031924234 | −0.84593696 |
| chr8 | 138777400 | 138777820 | −0.001379562 | −0.917040517 | −1.11393177 |
| chr12 | 107463140 | 107463720 | −0.001379413 | −1.102007 | −1.391834578 |
| chr6 | 123258620 | 123259140 | 0.001379125 | −1.111890673 | −1.018834023 |
| chr9 | 82358160 | 82358860 | −0.001379059 | −0.947595313 | −1.215009876 |
| chr10 | 135349920 | 135350620 | 0.001379046 | −1.306997631 | −1.105880955 |
| chr5 | 122827980 | 122828380 | −0.001379043 | −1.075763098 | −1.264998149 |
| chr5 | 122827940 | 122828420 | −0.001379043 | −1.075763098 | −1.264998149 |
| chr8 | 144371480 | 144371800 | −0.001378696 | −1.044557284 | −1.07981843 |
| chr16 | 30675540 | 30675960 | −0.001378674 | −0.890872903 | −1.107056602 |
| chr14 | 64413060 | 64413600 | −0.001378657 | −0.938630477 | −1.19686456 |
| chr7 | 121180380 | 121180960 | 0.00137851 | −1.188367613 | −1.121494632 |
| chr10 | 6244620 | 6245620 | −0.001378452 | −0.974425388 | −1.056896455 |
| chr19 | 42774840 | 42775120 | 0.00137843 | −1.015677133 | −0.863981322 |
| chr11 | 64126420 | 64126820 | 0.001378393 | −1.301041481 | −1.059474665 |
| chr4 | 57844840 | 57845160 | 0.001378342 | −1.356482211 | −1.134110814 |
| chr17 | 29248620 | 29249840 | 0.001377993 | −1.17513259 | −0.833363173 |
| chr4 | 148964600 | 148965160 | −0.001377801 | −1.039555427 | −1.268851977 |
| chr2 | 205916960 | 205917400 | −0.001377785 | −1.08947245 | −1.219320488 |
| chr11 | 43665300 | 43666240 | 0.001376303 | −1.255220504 | −1.052299627 |
| chr12 | 109676040 | 109676380 | −0.001375161 | −0.998813503 | −1.249981053 |
| chr4 | 177826140 | 177826520 | −0.001374558 | −1.133858347 | −1.284246377 |
| chr20 | 34359540 | 34360500 | 0.001374538 | −1.271998413 | −1.060734276 |
| chr1 | 194368220 | 194368640 | −0.001374502 | −1.031097563 | −1.25308583 |
| chr4 | 148139140 | 148139700 | −0.001374132 | −0.955031871 | −1.24496481 |
| chr2 | 111435360 | 111436260 | 0.001374063 | −1.2637367 | −1.041268475 |
| chr6 | 147465980 | 147466360 | −0.001373216 | −0.964674523 | −1.191023597 |
| chr4 | 48768800 | 48769240 | −0.001372619 | −0.940273659 | −1.138253162 |
| chr16 | 82619660 | 82620020 | −0.001371914 | −0.873791946 | −1.176127601 |
| chr5 | 147328420 | 147328880 | 0.001371375 | −1.105696082 | −1.064672648 |
| chr5 | 147328420 | 147328880 | 0.001371375 | −1.105696082 | −1.064672648 |
| chr18 | 63867220 | 63867640 | −0.00137128 | −0.937479136 | −1.197192744 |
| chr8 | 1052060 | 1052500 | −0.001371146 | −0.880381406 | −1.185085078 |
| chr8 | 143700900 | 143701280 | 0.001370599 | −1.063327124 | −0.794127559 |
| chr14 | 21979280 | 21979820 | 0.001369894 | −1.281376685 | −1.114977926 |
| chr18 | 40519840 | 40520540 | −0.001369881 | −0.992585509 | −1.146131137 |
| chr6 | 24053980 | 24054580 | −0.001369841 | −1.049498294 | −1.243628441 |
| chr17 | 33390560 | 33390940 | 0.001369839 | −1.09615754 | −0.857897005 |
| chr19 | 53934900 | 53935380 | 0.001369638 | −1.080103462 | −0.952295239 |
| chr19 | 1132040 | 1132460 | 0.001369575 | −1.050683515 | −0.852998451 |
| chr19 | 42774860 | 42775120 | 0.001369412 | −1.015677133 | −0.871636552 |
| chr1 | 89121260 | 89121840 | 0.001369373 | −1.120048994 | −0.939202077 |
| chr13 | 20534280 | 20534600 | 0.001369218 | −1.319030802 | −1.159566119 |
| chr19 | 8643660 | 8643900 | 0.00136888 | −1.223272643 | −0.955529062 |
| chr19 | 8643660 | 8643900 | 0.00136888 | −1.223272643 | −0.955529062 |
| chr2 | 180737200 | 180737540 | 0.001368706 | −1.347163165 | −1.146246188 |
| chr6 | 134273780 | 134274880 | 0.001368152 | −1.371551308 | −1.14177632 |
| chr3 | 163306520 | 163307200 | −0.001367991 | −0.98362702 | −1.209726796 |
| chr12 | 128179520 | 128180060 | −0.001367361 | −1.022296672 | −1.333919784 |
| chr13 | 60942080 | 60942620 | −0.001367133 | −1.140120157 | −1.392979325 |
| chr5 | 83984780 | 83985380 | 0.001366703 | −1.210737106 | −1.045709022 |
| chr11 | 23652740 | 23653380 | −0.001366239 | −0.993937615 | −1.217956902 |
| chr2 | 172165500 | 172165940 | −0.001366207 | −1.068962821 | −1.303482538 |
| chr2 | 172165460 | 172165980 | −0.001366207 | −1.068962821 | −1.303482538 |
| chr17 | 79111580 | 79112920 | 0.001365946 | −1.161358212 | −0.956257004 |
| chr20 | 59202460 | 59202900 | −0.001365896 | −0.930048326 | −1.233267571 |
| chr12 | 59966300 | 59967060 | −0.001365763 | −1.113728244 | −1.356505689 |
| chr5 | 18346420 | 18346880 | 0.00136501 | −1.181038877 | −1.12151889 |
| chr5 | 18346420 | 18346880 | 0.00136501 | −1.181038877 | −1.12151889 |
| chr4 | 48768720 | 48769280 | −0.001364976 | −0.956602105 | −1.123992339 |
| chr13 | 105945880 | 105946320 | 0.001364651 | −1.057838733 | −1.044229525 |
| chr17 | 32729660 | 32730140 | 0.001364483 | −1.0372551 | −0.90864129 |
| chr19 | 3134060 | 3134480 | 0.001364346 | −1.144172402 | −1.01392409 |
| chr17 | 7259340 | 7259660 | 0.001363859 | −1.068050112 | −0.895377914 |
| chr2 | 146589040 | 146589720 | −0.001363331 | −1.082423734 | −1.317247143 |
| chr2 | 146589020 | 146589760 | −0.001363331 | −1.082423734 | −1.311670217 |
| chr20 | 22064020 | 22064880 | −0.001363143 | −0.959462091 | −1.233429485 |
| chr15 | 68737140 | 68737700 | 0.001362971 | −1.115970777 | −0.941993762 |
| chr8 | 92081840 | 92083160 | 0.001362849 | −1.288162225 | −1.122110844 |
| chr1 | 168008580 | 168009200 | −0.001362421 | −0.925099398 | −1.243894609 |
| chr6 | 34359680 | 34360800 | 0.001362381 | −1.32512074 | −1.091187799 |
| chr11 | 74112520 | 74114040 | −0.001362032 | −1.038819151 | −1.331366414 |
| chr19 | 7598860 | 7599140 | 0.001361847 | −1.060105219 | −0.846272736 |
| chr2 | 55844300 | 55845480 | 0.001361819 | −1.329658438 | −1.12453105 |
| chr2 | 55844320 | 55845460 | 0.001361819 | −1.329658438 | −1.12453105 |
| chr1 | 25869860 | 25870800 | 0.001361591 | −1.19380012 | −0.937448541 |
| chr11 | 18752520 | 18752920 | −0.001361558 | −0.920899097 | −1.173382972 |
| chr2 | 80143480 | 80143760 | −0.001361495 | −0.958338297 | −1.133049582 |
| chr9 | 37632760 | 37633100 | −0.001361492 | −0.680371256 | −0.927338301 |
| chr18 | 63487100 | 63487480 | 0.001360871 | −1.152668916 | −1.046153015 |
| chr16 | 67840100 | 67841100 | 0.001360742 | −1.128530974 | −0.932277515 |
| chr19 | 4606720 | 4607120 | 0.001360228 | −1.045286544 | −0.85738392 |
| chr17 | 27224320 | 27225160 | 0.001360014 | −1.188553677 | −1.034370768 |
| chr19 | 54663120 | 54663900 | 0.001359854 | −1.306030593 | −1.121304315 |
| chr16 | 30197260 | 30197480 | 0.00135985 | −1.151529401 | −0.919270673 |
| chr3 | 32006820 | 32008100 | −0.001359679 | −1.111055729 | −1.318816863 |
| chr15 | 75337100 | 75337320 | 0.001359641 | −1.062825833 | −0.874498008 |
| chr2 | 166809880 | 166810900 | 0.001358899 | −1.222562804 | −1.030568927 |
| chr22 | 37852940 | 37854160 | 0.001358801 | −1.096330419 | −0.85894679 |
| chr1 | 182050660 | 182051140 | 0.001358731 | −1.238100596 | −0.972486144 |
| chr2 | 113484320 | 113484620 | 0.001358646 | −1.288499472 | −1.043970142 |
| chr11 | 71935840 | 71936260 | 0.001358093 | −1.175568356 | −0.985007481 |
| chr11 | 77122860 | 77123200 | 0.001357799 | −1.215806937 | −0.923072708 |
| chr10 | 78386740 | 78387260 | −0.001357619 | −1.143229332 | −1.300892449 |
| chr20 | 8661480 | 8661960 | 0.001357599 | −1.311471423 | −1.17237025 |
| chr12 | 8261160 | 8262040 | 0.001357047 | −1.418430104 | −1.165340936 |
| chr11 | 25594200 | 25594660 | −0.001356757 | −1.090754329 | −1.310046629 |
| chr11 | 25594160 | 25594700 | −0.001356757 | −1.090754329 | −1.310046629 |
| chr2 | 155483120 | 155483620 | −0.001356548 | −0.94388862 | −1.154744009 |
| chr2 | 65282740 | 65284160 | 0.001356194 | −1.331816042 | −1.115052001 |
| chr14 | 86785360 | 86785740 | −0.001355817 | −1.094177579 | −1.238331543 |
| chr10 | 103590200 | 103591240 | 0.001355058 | −1.233078858 | −1.020990768 |
| chr16 | 50247880 | 50248180 | 0.001355021 | −1.100330818 | −0.866723894 |
| chr3 | 172050360 | 172050620 | 0.001354733 | −1.187048071 | −1.024632875 |
| chr6 | 159524900 | 159525780 | 0.001354555 | −1.34142018 | −1.090183948 |
| chr6 | 48801500 | 48801960 | −0.001354194 | −1.004621519 | −1.178752487 |
| chr4 | 188507000 | 188507300 | −0.00135417 | −0.972275547 | −1.127629037 |
| chr17 | 7119060 | 7119400 | 0.001354125 | −1.100826443 | −0.890140198 |
| chr1 | 38461660 | 38462040 | 0.00135406 | −1.030720325 | −0.830444837 |
| chr12 | 24123580 | 24123900 | −0.001353951 | −0.878301091 | −1.127834164 |
| chr3 | 90123360 | 90124180 | −0.001353838 | −0.725314423 | −1.085024883 |
| chr8 | 137838320 | 137838940 | −0.001353729 | −1.052531526 | −1.289785671 |
| chr19 | 46498120 | 46499160 | 0.001353716 | −1.118638072 | −0.927099087 |
| chr21 | 42281540 | 42282020 | −0.001353629 | −0.707547049 | −0.982208789 |
| chr3 | 187459220 | 187460120 | 0.001353451 | −1.240461069 | −0.998531483 |
| chr3 | 82514840 | 82515480 | −0.001353233 | −0.992436082 | −1.26367929 |
| chr12 | 96792900 | 96794560 | 0.001352738 | −1.259479932 | −1.020526095 |
| chr22 | 39264920 | 39265160 | 0.001352667 | −1.209576351 | −1.035369622 |
| chr2 | 193856260 | 193856660 | −0.001352336 | −0.888598031 | −1.145666253 |
| chr10 | 78784740 | 78785260 | 0.001351901 | −1.140360496 | −1.007896781 |
| chr5 | 153824800 | 153826400 | 0.001351821 | −1.369053351 | −1.093884851 |
| chr2 | 119981080 | 119982260 | 0.001351724 | −1.194302564 | −0.990532858 |
| chr21 | 32137880 | 32138360 | −0.001351471 | −0.884537286 | −1.169485625 |
| chr17 | 56608700 | 56610060 | 0.001351358 | −1.134531372 | −0.896232574 |
| chr14 | 74148080 | 74148540 | 0.001351011 | −1.185388906 | −1.075980358 |
| chr14 | 74148080 | 74148540 | 0.001351011 | −1.185388906 | −1.075980358 |
| chr4 | 141444740 | 141445840 | 0.001350972 | −1.341203802 | −1.056815677 |
| chr19 | 19143680 | 19145260 | 0.001350965 | −1.113741368 | −0.893447116 |
| chr6 | 51603620 | 51604140 | −0.001350669 | −0.962955585 | −1.264836177 |
| chr6 | 51603620 | 51604140 | −0.001350669 | −0.962955585 | −1.264836177 |
| chr13 | 87431620 | 87432060 | −0.001350202 | −0.946013701 | −1.098717063 |
| chr9 | 81430580 | 81430960 | −0.0013501 | −0.737387806 | −0.931518536 |
| chr21 | 40423900 | 40424760 | 0.001350086 | −1.122938688 | −1.01755153 |
| chr4 | 83251940 | 83252500 | −0.001349064 | −1.018057392 | −1.233911312 |
| chr2 | 146472520 | 146473020 | 0.001349029 | −1.205062661 | −1.105902508 |
| chr14 | 43283920 | 43284420 | 0.001349025 | −1.2219218 | −1.173752967 |
| chr17 | 73458880 | 73459600 | −0.001349021 | −0.927863761 | −1.149504077 |
| chr1 | 45958860 | 45959100 | 0.001348891 | −1.231839966 | −1.019652724 |
| chr14 | 41362880 | 41363520 | −0.001348609 | −1.10016125 | −1.286934046 |
| chr4 | 68253480 | 68253980 | −0.001348605 | −1.104577917 | −1.305891347 |
| chr11 | 107304400 | 107304820 | −0.001348602 | −1.035703151 | −1.243556396 |
| chr1 | 200276360 | 200277260 | 0.00134856 | −1.324381347 | −1.063146901 |
| chr14 | 96806240 | 96806720 | 0.00134851 | −1.096591265 | −1.062879113 |
| chr9 | 74895620 | 74896400 | −0.00134827 | −0.866005855 | −0.947487745 |
| chr12 | 6934480 | 6935400 | 0.0013476 | −1.315800051 | −1.108251521 |
| chr5 | 22978560 | 22978980 | −0.001347579 | −0.908231069 | −1.177997364 |
| chr15 | 41954020 | 41954240 | 0.00134748 | −0.983393877 | −0.685353631 |
| chr4 | 55903820 | 55904260 | −0.001346999 | −0.950771984 | −1.203295399 |
| chr13 | 55712860 | 55713300 | 0.001346836 | −1.160233476 | −1.028248922 |
| chr8 | 50317540 | 50318340 | −0.001346549 | −0.988790284 | −1.223773385 |
| chr4 | 177689420 | 177689860 | −0.001346535 | −1.041932744 | −1.18976041 |
| chr10 | 123617960 | 123618340 | −0.001346171 | −1.043097638 | −1.246541133 |
| chr14 | 24583660 | 24584580 | 0.001346139 | −1.078441254 | −0.914829471 |
| chr4 | 159455840 | 159456320 | −0.00134608 | −0.925345672 | −1.144045602 |
| chr4 | 91851840 | 91852560 | −0.001345485 | −0.915428309 | −1.102155348 |
| chr2 | 88990400 | 88991700 | 0.001345429 | −1.397344431 | −1.142716165 |
| chr17 | 74814220 | 74814680 | −0.001345117 | −0.972530244 | −1.216395238 |
| chr2 | 83004940 | 83005280 | −0.001345081 | −0.77965316 | −1.055227221 |
| chr19 | 5084160 | 5084900 | −0.001345071 | −1.02653112 | −1.307259418 |
| chr6 | 104085560 | 104086000 | −0.001344201 | −1.04579216 | −1.30180522 |
| chr3 | 166003280 | 166003780 | −0.001344149 | −1.013959104 | −1.233695797 |
| chr20 | 5591080 | 5591940 | 0.001344032 | −1.310445459 | −1.072561696 |
| chr13 | 83178060 | 83178480 | 0.001344023 | −1.190423111 | −1.146212696 |
| chr9 | 27061500 | 27061840 | −0.001343884 | −0.730620743 | −0.994503451 |
| chr9 | 27061460 | 27061880 | −0.001343884 | −0.730620743 | −0.994503451 |
| chr9 | 28797100 | 28797540 | 0.001343767 | −1.061885855 | −0.943308738 |
| chr17 | 38115240 | 38115560 | 0.001343642 | −1.250522442 | −1.02793554 |
| chr3 | 36342180 | 36342420 | 0.001343619 | −1.330667886 | −1.132117306 |
| chr3 | 36342180 | 36342420 | 0.001343619 | −1.330667886 | −1.132117306 |
| chr6 | 150180280 | 150180740 | −0.001343476 | −0.941673652 | −1.11759458 |
| chr6 | 150180280 | 150180740 | −0.001343476 | −0.941673652 | −1.11759458 |
| chr1 | 182689680 | 182690400 | 0.001343029 | −1.187440504 | −1.108205382 |
| chr10 | 30874100 | 30874940 | −0.001342943 | −0.993036599 | −1.254220041 |
| chr4 | 131463500 | 131463980 | −0.001342861 | −1.095239844 | −1.287403945 |
| chr4 | 121914660 | 121915160 | −0.001342684 | −1.048646538 | −1.323801607 |
| chr3 | 64016060 | 64016960 | 0.001341679 | −1.329704154 | −1.126579752 |
| chr4 | 123500820 | 123501180 | −0.001341189 | −0.814847888 | −1.08892918 |
| chr8 | 84332580 | 84333160 | −0.00134069 | −1.065920653 | −1.24613645 |
| chr18 | 41940160 | 41940540 | −0.001340536 | −1.170841394 | −1.384289587 |
| chr13 | 35337420 | 35337740 | −0.001340479 | −0.909611025 | −1.098214753 |
| chr14 | 57729040 | 57729640 | −0.00134018 | −1.059231883 | −1.239369142 |
| chr6 | 42981320 | 42982220 | 0.001339871 | −1.44867681 | −1.25590291 |
| chr7 | 81508260 | 81508680 | −0.001339814 | −1.059714248 | −1.29359973 |
| chr2 | 22551520 | 22551960 | −0.001339535 | −1.104522911 | −1.358976957 |
| chr2 | 22551460 | 22552020 | −0.001339535 | −1.104522911 | −1.358976957 |
| chr7 | 95488060 | 95488420 | −0.001339345 | −1.007565243 | −1.228295528 |
| chr2 | 203499380 | 203500840 | 0.001339203 | −1.094955791 | −0.915433647 |
| chr11 | 42516180 | 42517200 | 0.001338899 | −1.17140976 | −1.12809252 |
| chr11 | 42516100 | 42517280 | 0.001338899 | −1.17140976 | −1.12809252 |
| chr15 | 65127620 | 65128600 | 0.001338809 | −1.118308896 | −0.960963415 |
| chr15 | 89448020 | 89448300 | 0.001338732 | −1.12351811 | −0.870887538 |
| chr13 | 72127520 | 72127960 | 0.001338665 | −1.123986273 | −1.11273914 |
| chr5 | 83371540 | 83372020 | −0.001338436 | −0.956208821 | −1.084031138 |
| chr8 | 14803320 | 14803760 | −0.001338431 | −1.153032236 | −1.352478681 |
| chr8 | 14803320 | 14803760 | −0.001338431 | −1.153032236 | −1.352478681 |
| chr6 | 122719800 | 122721400 | 0.001338379 | −1.247759621 | −0.969594588 |
| chr2 | 40859180 | 40859680 | 0.001338353 | −1.11800176 | −1.088857299 |
| chr14 | 75412960 | 75413500 | 0.001338171 | −1.257612577 | −1.037755983 |
| chr1 | 246885720 | 246885920 | −0.001338119 | −0.934636258 | −1.164707597 |
| chr1 | 246885720 | 246885920 | −0.001338119 | −0.934636258 | −1.164707597 |
| chr2 | 156026840 | 156027340 | 0.001337801 | −1.101130937 | −1.041368457 |
| chr2 | 156026780 | 156027400 | 0.001337801 | −1.101130937 | −1.041368457 |
| chr8 | 37766480 | 37766780 | −0.001337404 | −0.919010263 | −1.125946261 |
| chr16 | 27165920 | 27167100 | 0.00133689 | −1.168889976 | −0.865584815 |
| chr4 | 103681520 | 103682820 | 0.001336574 | −1.320543247 | −1.13457801 |
| chr5 | 172260620 | 172262000 | 0.001336558 | −1.292717779 | −1.003359235 |
| chr8 | 79075080 | 79075760 | −0.001336359 | −0.973000909 | −1.248915475 |
| chr8 | 12873520 | 12874080 | 0.00133503 | −1.193659981 | −1.18372906 |
| chr12 | 86891460 | 86892000 | −0.001334855 | −1.039053415 | −1.273029689 |
| chr12 | 48099160 | 48100640 | 0.00133397 | −1.294913869 | −1.133889164 |
| chr17 | 73584880 | 73586200 | 0.001333901 | −1.147835135 | −0.946531125 |
| chr4 | 149879000 | 149879380 | −0.001333868 | −0.923536129 | −1.189214669 |
| chr8 | 16682360 | 16682880 | −0.001333586 | −1.025095406 | −1.206943863 |
| chr14 | 105944940 | 105945400 | 0.001333306 | −1.278204773 | −1.037494372 |
| chr20 | 48400860 | 48401180 | −0.001333217 | −0.948598807 | −1.204478906 |
| chr18 | 9471420 | 9471740 | −0.001333029 | −1.063420329 | −1.278019644 |
| chr18 | 9471420 | 9471740 | −0.001333029 | −1.063420329 | −1.278019644 |
| chr2 | 99265620 | 99266500 | 0.001333014 | −1.385612061 | −1.152527639 |
| chr1 | 68231820 | 68232120 | 0.001332952 | −1.183308391 | −0.968120791 |
| chr13 | 59313000 | 59313400 | −0.001332861 | −1.201748877 | −1.366562486 |
| chr16 | 17566420 | 17566860 | −0.001332488 | −0.954833802 | −1.112991686 |
| chr3 | 59303780 | 59304580 | −0.00133198 | −1.084644813 | −1.315623296 |
| chr3 | 42601420 | 42601940 | −0.001331913 | −0.953820146 | −1.236461889 |
| chr21 | 45138180 | 45140460 | 0.001331867 | −1.121633314 | −0.885512297 |
| chr1 | 17362980 | 17363460 | −0.001331712 | −0.948047635 | −1.204239766 |
| chr2 | 135487220 | 135487640 | −0.001331656 | −1.012930525 | −1.245758822 |
| chr20 | 40898640 | 40899180 | 0.001331267 | −1.318406454 | −1.088476591 |
| chr21 | 47705680 | 47706740 | 0.001331255 | −1.166379731 | −0.972981064 |
| chr3 | 106588000 | 106588760 | −0.001331065 | −0.971377575 | −1.289266059 |
| chr1 | 44404760 | 44405160 | 0.001330931 | −1.252277472 | −1.022005356 |
| chr1 | 44404740 | 44405180 | 0.001330931 | −1.252277472 | −1.022005356 |
| chr12 | 71122160 | 71122500 | −0.001330302 | −0.983488155 | −1.225947093 |
| chr12 | 71122160 | 71122500 | −0.001330302 | −0.983488155 | −1.225947093 |
| chr2 | 237399380 | 237400080 | −0.001330044 | −1.049482428 | −1.261147655 |
| chr8 | 144371460 | 144371840 | −0.001329313 | −1.042876842 | −1.070868259 |
| chr13 | 92031620 | 92032140 | −0.001329081 | −1.134007736 | −1.307718622 |
| chr12 | 81528840 | 81529400 | −0.001328992 | −1.076575822 | −1.331735575 |
| chr9 | 105583960 | 105584460 | 0.001328469 | −1.044515546 | −0.856661879 |
| chr2 | 194182040 | 194182460 | 0.001327922 | −1.092839989 | −0.942010532 |
| chr19 | 31839760 | 31842020 | 0.001327402 | −1.167347055 | −0.927302039 |
| chr6 | 163828960 | 163829440 | −0.001327378 | −0.925920774 | −1.125158452 |
| chr3 | 50230100 | 50230340 | 0.001327194 | −1.320041414 | −1.028085562 |
| chr5 | 21183240 | 21183660 | −0.001327112 | −0.949662807 | −1.229371255 |
| chr12 | 59966340 | 59967040 | −0.001327041 | −1.128510364 | −1.356505689 |
| chr20 | 277760 | 278760 | 0.00132697 | −1.100525544 | −0.944035741 |
| chr2 | 166377040 | 166377560 | −0.001326899 | −1.09000418 | −1.291051131 |
| chr19 | 58911720 | 58913540 | 0.001326812 | −1.072918267 | −0.914477684 |
| chr10 | 78398720 | 78399400 | −0.001326529 | −1.129511977 | −1.296077271 |
| chr20 | 30200620 | 30201000 | 0.001325682 | −1.288097153 | −1.042281593 |
| chr14 | 102605460 | 102606860 | 0.001325602 | −1.359107749 | −1.144386105 |
| chr7 | 143594020 | 143594400 | 0.00132552 | −1.224425702 | −1.072354061 |
| chr19 | 34083620 | 34084040 | −0.001325057 | −0.785604617 | −1.120769312 |
| chr19 | 34083560 | 34084100 | −0.001325057 | −0.785604617 | −1.120769312 |
| chr9 | 83608540 | 83609140 | −0.001325013 | −0.967690566 | −1.156628921 |
| chr5 | 109421820 | 109422280 | −0.001324838 | −1.12955048 | −1.308210415 |
| chr19 | 1132060 | 1132460 | 0.001324816 | −1.055310739 | −0.887830248 |
| chr4 | 35219800 | 35220160 | −0.00132467 | −1.226216225 | −1.378968304 |
| chr2 | 41437800 | 41438220 | −0.001324431 | −0.818000051 | −1.108581135 |
| chr2 | 41437760 | 41438260 | −0.001324431 | −0.818000051 | −1.108581135 |
| chr12 | 39830080 | 39830460 | −0.001324398 | −0.970122535 | −1.179886756 |
| chr12 | 39830060 | 39830480 | −0.001324398 | −0.970122535 | −1.179886756 |
| chr8 | 34540240 | 34540660 | 0.001324286 | −1.158750859 | −1.090214641 |
| chr15 | 53931460 | 53931940 | −0.001324258 | −0.835245912 | −1.100917288 |
| chr3 | 29371120 | 29371660 | −0.001323883 | −0.951372873 | −1.277541302 |
| chr19 | 1240180 | 1240680 | 0.001323881 | −1.071752044 | −0.927323105 |
| chr9 | 76289320 | 76290500 | −0.001323525 | −0.939572646 | −1.241455334 |
| chr15 | 68132320 | 68133160 | 0.001322775 | −1.159878813 | −0.926296891 |
| chr15 | 68132320 | 68133160 | 0.001322775 | −1.159878813 | −0.926296891 |
| chr9 | 81536740 | 81537060 | −0.001322593 | −0.842517377 | −1.051962394 |
| chr10 | 110674500 | 110674960 | −0.001322533 | −0.846317479 | −1.215737707 |
| chr7 | 13472900 | 13473780 | −0.001322455 | −0.890585019 | −1.241797953 |
| chr16 | 4380340 | 4380600 | 0.001322249 | −1.104163131 | −0.896018656 |
| chr8 | 135724600 | 135725940 | 0.001322218 | −1.296897905 | −1.163747465 |
| chr16 | 69165800 | 69166800 | 0.001321576 | −1.119612466 | −0.906726525 |
| chr2 | 62026640 | 62027040 | −0.001321491 | −0.952686299 | −1.213581304 |
| chr2 | 20246320 | 20246720 | −0.001321426 | −1.153647563 | −1.343929615 |
| chr21 | 35287660 | 35288620 | 0.001321241 | −1.093279805 | −0.905417067 |
| chr2 | 3605380 | 3606520 | 0.001320426 | −1.398672353 | −1.164054674 |
| chr12 | 116648140 | 116648600 | −0.001319745 | −0.956053682 | −1.165480062 |
| chr1 | 106331620 | 106332300 | −0.001319711 | −0.765343674 | −1.055787691 |
| chr17 | 3817100 | 3817500 | 0.001319593 | −1.202126886 | −0.961289506 |
| chr17 | 3817120 | 3817480 | 0.001319593 | −1.202126886 | −0.961289506 |
| chr13 | 31773380 | 31775200 | 0.001319544 | −1.428005136 | −1.147594108 |
| chr20 | 18879060 | 18879980 | −0.001319184 | −0.853223732 | −1.131172959 |
| chr8 | 79794760 | 79795420 | 0.001318949 | −1.296933798 | −1.216100716 |
| chr2 | 5482620 | 5483420 | −0.001318873 | −1.08442778 | −1.313628643 |
| chr6 | 41316680 | 41317140 | −0.001318769 | −0.833621028 | −1.141838604 |
| chr21 | 29165820 | 29166240 | 0.00131848 | −0.927249815 | −0.849905424 |
| chr9 | 25047700 | 25048200 | −0.001317898 | −0.883036954 | −1.098042263 |
| chr1 | 219107240 | 219107720 | −0.001317664 | −0.897939105 | −1.175577513 |
| chr1 | 219107220 | 219107740 | −0.001317664 | −0.897939105 | −1.175577513 |
| chr22 | 50320420 | 50320740 | 0.00131759 | −1.14220886 | −1.016834299 |
| chr22 | 50320420 | 50320740 | 0.00131759 | −1.14220886 | −1.016834299 |
| chr6 | 32939040 | 32940300 | 0.001317557 | −1.23306365 | −1.056580107 |
| chr16 | 3257080 | 3257340 | 0.00131745 | −1.018501732 | −0.864570962 |
| chr16 | 3257080 | 3257340 | 0.00131745 | −1.018501732 | −0.864570962 |
| chr4 | 156301200 | 156301740 | 0.001317315 | −1.235922928 | −1.125747031 |
| chr17 | 27090780 | 27091060 | 0.001316947 | −1.217515286 | −0.996660487 |
| chr12 | 93834900 | 93836500 | 0.001316591 | −1.204392909 | −0.926503578 |
| chr19 | 19302820 | 19303860 | 0.001316484 | −1.088525495 | −0.979091605 |
| chr2 | 77951980 | 77952460 | 0.001316219 | −1.321945478 | −1.069616564 |
| chr10 | 110069460 | 110069880 | −0.001315991 | −0.966245391 | −1.13570794 |
| chr10 | 10248260 | 10248780 | −0.00131554 | −1.027567024 | −1.305772631 |
| chr15 | 36887380 | 36887720 | −0.001315497 | −0.724012652 | −0.939766164 |
| chr15 | 36887320 | 36887780 | −0.001315497 | −0.724012652 | −0.939766164 |
| chr19 | 7598820 | 7599160 | 0.001314399 | −1.060046837 | −0.848358722 |
| chr20 | 37091800 | 37092060 | −0.001314308 | −1.152278142 | −1.379276535 |
| chr20 | 37091800 | 37092060 | −0.001314308 | −1.152278142 | −1.379276535 |
| chr10 | 6244600 | 6245680 | −0.001314085 | −0.958685818 | −1.028434024 |
| chr3 | 142359880 | 142360300 | −0.00131372 | −1.052777423 | −1.304601441 |
| chr2 | 5482700 | 5483360 | −0.001312779 | −1.105794132 | −1.338355641 |
| chr7 | 135723020 | 135723480 | −0.001312726 | −0.811213966 | −1.055492619 |
| chr1 | 160576920 | 160577240 | 0.001312401 | −1.07051367 | −0.87224719 |
| chr3 | 71704740 | 71705180 | −0.001312366 | −1.143610589 | −1.352120657 |
| chr3 | 71704680 | 71705240 | −0.001312366 | −1.143610589 | −1.352120657 |
| chr13 | 63370820 | 63371380 | −0.001312219 | −1.174511533 | −1.412890997 |
| chr8 | 143813460 | 143813800 | −0.001312081 | −1.06163822 | −1.259531539 |
| chr19 | 38826160 | 38827840 | 0.00131163 | −1.158106041 | −0.928661013 |
| chr17 | 26971700 | 26972460 | 0.001311612 | −1.151608812 | −0.917755058 |
| chr7 | 10433120 | 10433520 | −0.001311431 | −1.027492204 | −1.294246218 |
| chr20 | 23976580 | 23977100 | −0.001311324 | −1.006513639 | −1.296331723 |
| chr20 | 23976580 | 23977120 | −0.001311324 | −1.006513639 | −1.301941171 |
| chr4 | 5104520 | 5104880 | −0.001311311 | −0.984340726 | −1.059054375 |
| chr4 | 6213200 | 6213680 | −0.001311264 | −1.105072304 | −1.312401558 |
| chr15 | 49484460 | 49484940 | −0.001310936 | −0.877531769 | −1.104705062 |
| chr18 | 33552100 | 33553040 | 0.001310366 | −1.315179517 | −1.16956243 |
| chr4 | 104709400 | 104709800 | −0.00130991 | −1.124520443 | −1.286339644 |
| chr8 | 76581940 | 76582420 | −0.001309484 | −0.941065078 | −1.187757735 |
| chr19 | 14675020 | 14675540 | 0.001309479 | −1.191928147 | −1.008301132 |
| chr16 | 84629260 | 84629640 | 0.001309043 | −0.989626383 | −0.89985445 |
| chr7 | 146781500 | 146782120 | 0.001308874 | −1.234056111 | −1.192068314 |
| chr2 | 73297800 | 73299420 | 0.001308731 | −1.280682818 | −1.092192324 |
| chr11 | 45201740 | 45202820 | 0.001308639 | −1.400565937 | −1.14817463 |
| chr19 | 55752300 | 55752720 | 0.001307075 | −1.120173417 | −0.883083769 |
| chr19 | 55752300 | 55752720 | 0.001307075 | −1.120173417 | −0.883083769 |
| chr8 | 22461500 | 22462840 | −0.001306911 | −1.096908346 | −1.203883626 |
| chr17 | 2652260 | 2653380 | 0.001306788 | −1.157115654 | −0.928349038 |
| chr16 | 47467540 | 47467920 | −0.001306524 | −0.8700122 | −1.138902496 |
| chr1 | 244553000 | 244553480 | 0.001306421 | −1.182332107 | −1.104044084 |
| chr2 | 71272240 | 71272440 | 0.001306319 | −1.136117074 | −1.113953805 |
| chr2 | 71272240 | 71272440 | 0.001306319 | −1.136117074 | −1.113953805 |
| chr4 | 112736960 | 112737420 | −0.001306249 | −1.097918782 | −1.281823184 |
| chr4 | 68166780 | 68167260 | 0.001306165 | −1.163921257 | −1.054787703 |
| chr4 | 68166780 | 68167260 | 0.001306165 | −1.163921257 | −1.054787703 |
| chr2 | 71080160 | 71080600 | −0.001306128 | −0.978343857 | −1.278900978 |
| chr10 | 104670540 | 104671160 | −0.001306085 | −0.978626804 | −1.269192841 |
| chr4 | 137816460 | 137816880 | −0.001305861 | −0.869783338 | −1.083453591 |
| chr14 | 47469240 | 47469860 | −0.001305856 | −1.031601482 | −1.306997664 |
| chr8 | 90277960 | 90278500 | −0.001305774 | −1.102991723 | −1.329571023 |
| chr11 | 64889040 | 64890200 | 0.001305623 | −1.331200551 | −1.098743647 |
| chr4 | 57844860 | 57845180 | 0.001305494 | −1.356482211 | −1.145652104 |
| chr6 | 47786900 | 47787380 | −0.001305315 | −1.034722685 | −1.271108525 |
| chr21 | 28959700 | 28960480 | 0.001305266 | −0.994256901 | −0.978827622 |
| chr13 | 49421340 | 49421840 | 0.001305157 | −1.214999108 | −1.075348026 |
| chr5 | 64183960 | 64184520 | −0.001304033 | −0.978457109 | −1.345760555 |
| chr14 | 56514080 | 56514700 | −0.001303945 | −1.191612692 | −1.368542649 |
| chr11 | 47395500 | 47395760 | 0.001303904 | −1.213686575 | −1.012514385 |
| chr8 | 19519400 | 19519780 | 0.001303857 | −1.31174065 | −1.049073971 |
| chr16 | 48643260 | 48645140 | 0.00130372 | −1.221202001 | −1.084262331 |
| chr12 | 121142360 | 121142780 | −0.001303418 | −0.888986044 | −1.143324091 |
| chr19 | 14485940 | 14486200 | 0.001303165 | −1.054676599 | −0.914090585 |
| chr1 | 76539680 | 76541040 | 0.001303087 | −1.262324103 | −0.985759734 |
| chr1 | 67171800 | 67172280 | −0.001302376 | −0.979968119 | −1.248994764 |
| chr3 | 57002200 | 57002560 | −0.001302325 | −1.006503436 | −1.185209422 |
| chr1 | 241206660 | 241207220 | −0.001302162 | −0.880551227 | −1.195420178 |
| chr5 | 94694920 | 94695400 | 0.001301998 | −1.20092746 | −1.010735018 |
| chr7 | 30323560 | 30324420 | 0.001301995 | −1.307176917 | −1.086009872 |
| chr7 | 30323560 | 30324420 | 0.001301995 | −1.307176917 | −1.086009872 |
| chr3 | 57839680 | 57840000 | −0.001301787 | −1.040855825 | −1.245525854 |
| chr11 | 2909660 | 2909920 | 0.001301374 | −1.403051073 | −1.168607778 |
| chr12 | 14762320 | 14762700 | −0.001301263 | −0.953534304 | −1.241921061 |
| chr17 | 42083180 | 42083520 | 0.001300387 | −1.044454819 | −0.837307777 |
| chr8 | 4627560 | 4628020 | −0.001299589 | −0.952029771 | −1.254692408 |
| chr22 | 30782580 | 30783940 | 0.00129911 | −1.135823051 | −0.973387732 |
| chr16 | 52809520 | 52810040 | −0.001298984 | −0.861366966 | −1.148845897 |
| chr17 | 54132720 | 54133100 | −0.001298896 | −0.879729773 | −1.108348829 |
| chr17 | 54132700 | 54133120 | −0.001298896 | −0.879729773 | −1.108348829 |
| chr6 | 143885060 | 143885640 | −0.001298668 | −1.107194295 | −1.259849239 |
| chr19 | 45594740 | 45595440 | 0.001298473 | −1.083618699 | −0.953092028 |
| chr7 | 92485140 | 92485900 | −0.001298307 | −0.871192657 | −1.131029423 |
| chr17 | 48100580 | 48102840 | 0.001297979 | −1.102055457 | −0.995433424 |
| chr19 | 18223820 | 18224100 | 0.001297892 | −1.175656334 | −0.966289682 |
| chr3 | 57134160 | 57134440 | 0.001297728 | −1.230145374 | −1.07559375 |
| chr16 | 65499600 | 65500400 | 0.001297678 | −1.106701084 | −1.041442934 |
| chr6 | 38271400 | 38272040 | 0.001297662 | −1.229058217 | −1.055245122 |
| chr15 | 91072500 | 91074160 | 0.001297529 | −1.113694697 | −0.786806223 |
| chr12 | 50793680 | 50795560 | 0.001297268 | −1.222239955 | −1.060864783 |
| chr17 | 8062140 | 8062400 | 0.001297146 | −1.227811679 | −0.937886561 |
| chr10 | 6877500 | 6879820 | 0.001297053 | −1.117979152 | −1.116624246 |
| chr5 | 10284140 | 10284420 | 0.00129661 | −1.276544527 | −1.019011878 |
| chr11 | 127867900 | 127868440 | −0.001296432 | −1.062473067 | −1.253776676 |
| chr7 | 117844320 | 117844880 | −0.00129642 | −0.985750662 | −1.196186411 |
| chr19 | 3500540 | 3501360 | 0.00129635 | −1.170157942 | −1.0042955 |
| chr11 | 67416840 | 67417600 | 0.001296047 | −1.164174423 | −0.917436398 |
| chr13 | 66344920 | 66345260 | −0.001295883 | −1.040551776 | −1.25096276 |
| chr4 | 76745800 | 76746340 | −0.001295588 | −1.151691728 | −1.366314839 |
| chr22 | 24407040 | 24408380 | 0.001295119 | −1.085620769 | −0.895622566 |
| chr13 | 57279460 | 57279900 | 0.001295119 | −1.1971191 | −1.021741915 |
| chr6 | 43477460 | 43478720 | 0.00129503 | −1.236151695 | −1.126489387 |
| chr6 | 43477440 | 43478740 | 0.00129503 | −1.236151695 | −1.126489387 |
| chr20 | 60168980 | 60169440 | −0.001294192 | −1.054450479 | −1.190425091 |
| chr18 | 1515500 | 1515980 | −0.001294095 | −1.164057504 | −1.376724616 |
| chr4 | 35219560 | 35220440 | −0.001293902 | −0.964840364 | −1.166275402 |
| chr21 | 40423900 | 40424740 | 0.001293844 | −1.101936399 | −1.009798398 |
| chr8 | 78432180 | 78432580 | −0.001293082 | −0.872288505 | −1.160412742 |
| chr19 | 2274860 | 2275060 | 0.001292595 | −1.020878332 | −0.820019056 |
| chr4 | 76745800 | 76746360 | −0.00129253 | −1.111383728 | −1.352762707 |
| chr12 | 17439420 | 17439940 | −0.001291814 | −0.999596076 | −1.274256691 |
| chr1 | 230451140 | 230452180 | −0.001291787 | −0.91335764 | −1.138156476 |
| chr2 | 175632500 | 175633280 | −0.0012914 | −1.130051739 | −1.374818129 |
| chr2 | 175632440 | 175633340 | −0.0012914 | −1.130051739 | −1.374818129 |
| chr2 | 140850480 | 140850920 | −0.001291361 | −0.958269921 | −1.151242831 |
| chr4 | 89575300 | 89575960 | −0.001291305 | −0.95736932 | −1.210125675 |
| chr15 | 81768840 | 81769220 | −0.001291209 | −0.730681343 | −1.091486076 |
| chr7 | 128045080 | 128046600 | 0.001290998 | −1.164157417 | −0.970100598 |
| chr10 | 74451080 | 74452940 | 0.0012909 | −1.222194545 | −0.967573571 |
| chr11 | 66313080 | 66313960 | 0.001290715 | −1.24316175 | −1.043456304 |
| chr3 | 151478700 | 151479240 | −0.00129064 | −1.125162179 | −1.288002369 |
| chr16 | 20991320 | 20991760 | −0.001290433 | −0.959395834 | −1.253234655 |
| chr10 | 36017800 | 36018380 | −0.001289367 | −0.995362914 | −1.275556179 |
| chr22 | 33589740 | 33590180 | 0.00128922 | −1.034523341 | −1.012950301 |
| chr2 | 160118140 | 160118640 | −0.001288878 | −1.080487195 | −1.266191268 |
| chr4 | 36486360 | 36486920 | −0.001288775 | −1.151604028 | −1.358812173 |
| chr4 | 187531580 | 187532040 | −0.001288539 | −1.101431149 | −1.374132177 |
| chr17 | 78866680 | 78867520 | 0.001288437 | −1.203402246 | −1.062333336 |
| chr5 | 33521300 | 33521780 | −0.001288261 | −1.094412265 | −1.239263432 |
| chr18 | 9073560 | 9073960 | 0.001288085 | −1.349469458 | −1.179110074 |
| chr2 | 22141280 | 22141680 | −0.001288031 | −0.826821589 | −1.058211527 |
| chr3 | 102408980 | 102409480 | −0.001287926 | −1.083894983 | −1.265122814 |
| chr4 | 116904540 | 116905040 | −0.001287493 | −1.120441788 | −1.29780412 |
| chr8 | 16546560 | 16547060 | 0.001287435 | −1.314627447 | −1.074215993 |
| chr12 | 124938080 | 124938400 | 0.001286695 | −1.110642205 | −0.858991761 |
| chr4 | 173704380 | 173704820 | −0.001286129 | −0.915479126 | −1.216759685 |
| chr9 | 81430600 | 81430960 | −0.001285704 | −0.770062208 | −0.949038586 |
| chr19 | 51250960 | 51251400 | 0.001285697 | −1.151890493 | −1.068231229 |
| chr20 | 48544960 | 48545820 | 0.001285538 | −1.327836372 | −1.121111056 |
| chr19 | 59068940 | 59069140 | 0.001284232 | −1.048288591 | −0.824270571 |
| chr1 | 33721460 | 33722580 | 0.001284105 | −1.299042437 | −1.125156356 |
| chr18 | 27174640 | 27174980 | −0.001284101 | −1.158265416 | −1.362409758 |
| chr16 | 66784960 | 66786380 | 0.001284006 | −1.226350518 | −1.01523537 |
| chr11 | 133304520 | 133304960 | −0.001283843 | −0.944481226 | −1.141906787 |
| chr2 | 40859140 | 40859740 | 0.001283836 | −1.123099451 | −1.126433063 |
| chr1 | 51655160 | 51655420 | 0.001283459 | −1.153238488 | −0.991550334 |
| chr1 | 51654840 | 51655740 | 0.001283459 | −1.153238488 | −0.991550334 |
| chr20 | 40898680 | 40899180 | 0.001283275 | −1.31585831 | −1.096192554 |
| chr12 | 7000080 | 7000640 | 0.00128323 | −1.411501585 | −1.21780598 |
| chr20 | 31070540 | 31071820 | 0.001283143 | −1.305393319 | −1.068631483 |
| chr1 | 19387640 | 19388100 | 0.001282937 | −1.080744281 | −0.869917209 |
| chr7 | 64908100 | 64908540 | −0.001282705 | −0.94950974 | −1.259278372 |
| chr15 | 46005060 | 46005680 | −0.001282663 | −0.885169482 | −0.967734746 |
| chr6 | 31461580 | 31461860 | 0.00128217 | −1.336795862 | −1.160489579 |
| chr7 | 5566480 | 5566800 | −0.001282145 | −0.958179657 | −0.990987423 |
| chr3 | 1684440 | 1684780 | −0.001281931 | −1.039141821 | −1.181793812 |
| chr3 | 1684440 | 1684780 | −0.001281931 | −1.039141821 | −1.181793812 |
| chr3 | 159545740 | 159546580 | 0.001281586 | −1.232729383 | −1.098260273 |
| chr11 | 65819420 | 65820100 | 0.001281394 | −1.401058782 | −1.149252603 |
| chr12 | 40104880 | 40108320 | 0.001281263 | −1.311410791 | −1.20316178 |
| chr2 | 22912600 | 22913120 | −0.001281214 | −1.117969782 | −1.301766344 |
| chr4 | 147629260 | 147629780 | −0.001281211 | −1.02470363 | −1.210860164 |
| chr10 | 129442940 | 129443420 | −0.0012811 | −0.890262345 | −1.055939438 |
| chr4 | 112174880 | 112175340 | −0.00128092 | −1.030131209 | −1.194877522 |
| chr4 | 187469780 | 187470160 | −0.001280758 | −0.981053 | −1.185261221 |
| chr11 | 127040400 | 127040740 | 0.001280554 | −1.110806093 | −1.029387833 |
| chr11 | 127040400 | 127040740 | 0.001280554 | −1.110806093 | −1.029387833 |
| chr7 | 12100200 | 12100620 | −0.001280491 | −1.054417102 | −1.274866083 |
| chr5 | 62500540 | 62501100 | 0.001279837 | −1.029326733 | −0.848386487 |
| chr2 | 196290720 | 196291140 | −0.001279808 | −1.072610865 | −1.316131384 |
| chr13 | 60942040 | 60942640 | −0.00127929 | −1.159408007 | −1.381910577 |
| chr14 | 81453360 | 81454580 | 0.001279209 | −1.215216539 | −1.010806801 |
| chr19 | 49149520 | 49150400 | 0.001279136 | −1.107068873 | −0.929547947 |
| chr19 | 49149500 | 49150420 | 0.001279136 | −1.107068873 | −0.929547947 |
| chr8 | 140699380 | 140700380 | −0.001279007 | −1.014487817 | −1.24079654 |
| chr22 | 50294960 | 50295420 | 0.001278724 | −0.996408589 | −0.861007455 |
| chr15 | 69780880 | 69781300 | −0.001278661 | −1.041170821 | −1.250551368 |
| chr7 | 6387900 | 6388860 | 0.001278589 | −1.215546473 | −0.9836575 |
| chr9 | 25940100 | 25940840 | −0.001278564 | −0.954189014 | −1.194255559 |
| chr9 | 25940100 | 25940860 | −0.001278564 | −0.954189014 | −1.194255559 |
| chr12 | 69226900 | 69227540 | −0.00127844 | −1.034440078 | −1.277957507 |
| chr16 | 4673940 | 4675520 | 0.001277993 | −1.215373073 | −1.049611704 |
| chr16 | 14726080 | 14727340 | 0.00127792 | −1.156842618 | −0.968604281 |
| chr19 | 18042820 | 18044240 | 0.001277916 | −1.12521708 | −1.017926904 |
| chr15 | 59312140 | 59312580 | −0.001277671 | −0.829882809 | −1.143492704 |
| chr12 | 80908080 | 80908900 | −0.001277659 | −0.960296076 | −1.240996523 |
| chr19 | 42721140 | 42722980 | 0.001277107 | −1.045359678 | −0.852000391 |
| chr6 | 48668760 | 48669020 | −0.001276936 | −0.837116503 | −1.027000909 |
| chr7 | 155725060 | 155726120 | 0.001276912 | −1.225667874 | −1.081692045 |
| chr16 | 67839880 | 67841420 | 0.001276871 | −1.280805007 | −1.130034717 |
| chr19 | 4553500 | 4553940 | −0.001276684 | −0.953115313 | −1.030425014 |
| chr17 | 73963540 | 73963840 | −0.001276409 | −0.885431421 | −1.108147271 |
| chr9 | 11474100 | 11474460 | −0.00127603 | −0.619695541 | −0.847380491 |
| chr11 | 131035680 | 131036240 | 0.001275503 | −1.026717185 | −1.059415096 |
| chr8 | 19370740 | 19371200 | −0.001275483 | −0.982561343 | −1.20723539 |
| chr12 | 56211220 | 56212440 | 0.001275231 | −1.355596222 | −1.141956193 |
| chr22 | 30649240 | 30649620 | 0.001275214 | −0.861995753 | −0.702630276 |
| chr10 | 4708420 | 4710600 | 0.001275167 | −1.341761952 | −1.155741605 |
| chr10 | 4708420 | 4710600 | 0.001275167 | −1.341761952 | −1.155741605 |
| chr11 | 122932460 | 122933840 | 0.001275033 | −1.348702691 | −1.129935226 |
| chr1 | 28198820 | 28199260 | 0.001275012 | −1.253418012 | −1.099542543 |
| chr10 | 32143740 | 32144260 | 0.001274936 | −1.30243442 | −1.093214105 |
| chr15 | 75337020 | 75337380 | 0.001274742 | −1.005047082 | −0.86619503 |
| chr13 | 66286160 | 66286500 | −0.001274596 | −1.009303162 | −1.293037145 |
| chr4 | 57073700 | 57074200 | −0.001274312 | −0.979461732 | −1.178620645 |
| chr20 | 12374220 | 12375060 | −0.001273711 | −0.954279479 | −1.26283386 |
| chr19 | 50391560 | 50391900 | 0.001273627 | −1.028481343 | −0.807615732 |
| chr2 | 157292380 | 157293100 | 0.001273552 | −1.203006212 | −0.970321864 |
| chr4 | 173704260 | 173704880 | −0.001273107 | −0.937422555 | −1.238563288 |
| chr11 | 1245540 | 1246040 | 0.001272944 | −1.220443359 | −0.940293464 |
| chr13 | 64464520 | 64465140 | −0.001272415 | −1.030895687 | −1.241261125 |
| chr8 | 37978760 | 37979080 | −0.001271992 | −1.067229463 | −1.261335966 |
| chr9 | 79821940 | 79822420 | 0.001271889 | −1.148613894 | −1.070662061 |
| chr9 | 79821820 | 79822540 | 0.001271889 | −1.148613894 | −1.070662061 |
| chr9 | 91932580 | 91932880 | 0.001271663 | −1.003379663 | −0.774008558 |
| chr3 | 61236420 | 61238020 | −0.00127146 | −1.025605667 | −1.146197803 |
| chr1 | 60551460 | 60551780 | −0.001271335 | −0.95262197 | −1.17798082 |
| chr16 | 7944280 | 7945340 | −0.001271237 | −0.882901337 | −1.147992716 |
| chr2 | 161405360 | 161405680 | −0.001270947 | −0.822785843 | −1.122437279 |
| chr12 | 128179500 | 128180100 | −0.001270513 | −1.036360168 | −1.335091218 |
| chr13 | 101625560 | 101626380 | 0.001270426 | −1.151009963 | −0.986227271 |
| chr11 | 7044280 | 7045040 | −0.001270299 | −1.018223556 | −1.279936486 |
| chr7 | 75676880 | 75677820 | 0.001270238 | −1.197224217 | −1.047331758 |
| chr7 | 75676880 | 75677820 | 0.001270238 | −1.197224217 | −1.047331758 |
| chr15 | 98109300 | 98109840 | −0.001270035 | −0.769743145 | −1.033030436 |
| chr5 | 171615060 | 171616480 | 0.001270001 | −1.272395651 | −1.085752148 |
| chr6 | 67086540 | 67087120 | −0.001269749 | −0.997517463 | −1.301752124 |
| chr9 | 28470720 | 28471120 | −0.001269624 | −0.761322592 | −1.038136946 |
| chr6 | 90538880 | 90540180 | 0.001269532 | −1.394738755 | −1.152285047 |
| chr16 | 46722560 | 46723980 | 0.001269468 | −1.112462125 | −0.905036132 |
| chr5 | 117479440 | 117480120 | −0.001269418 | −1.019492606 | −1.237150864 |
| chr2 | 157291920 | 157293540 | 0.001269108 | −1.199126875 | −0.949470215 |
| chr12 | 125347760 | 125349220 | 0.001269012 | −1.13943172 | −1.053745238 |
| chr1 | 97707480 | 97707940 | −0.00126881 | −0.940127053 | −1.191639252 |
| chr19 | 51842080 | 51844060 | −0.001268728 | −0.919222715 | −1.053188579 |
| chr1 | 57830380 | 57831240 | −0.001268697 | −0.856779923 | −1.188725088 |
| chr11 | 110518640 | 110519220 | −0.001268634 | −1.056695387 | −1.265587733 |
| chr16 | 70380440 | 70380940 | 0.001268532 | −1.208754045 | −1.046545202 |
| chr1 | 60164480 | 60165720 | 0.00126851 | −1.290743745 | −1.081272302 |
| chr2 | 69613740 | 69615120 | −0.001268478 | −1.138226147 | −1.243653208 |
| chr4 | 14965580 | 14966120 | −0.001267951 | −0.919422472 | −1.208815247 |
| chr3 | 1750180 | 1750440 | −0.001267888 | −0.958717158 | −1.240067021 |
| chr11 | 123000120 | 123000640 | −0.001267506 | −0.988111775 | −1.260570358 |
| chr15 | 85523560 | 85525740 | 0.001267395 | −1.093254197 | −0.931984395 |
| chr15 | 34634480 | 34636200 | 0.001267379 | −1.012959297 | −0.825940186 |
| chr22 | 26824900 | 26826160 | 0.001265403 | −1.131892947 | −0.977327163 |
| chr1 | 153721420 | 153722700 | 0.001264638 | −1.187718191 | −1.053573406 |
| chr7 | 64411680 | 64411960 | 0.001264353 | −1.142852792 | −0.949682405 |
| chr6 | 152895120 | 152895520 | −0.001264293 | −0.951453887 | −1.143466695 |
| chr6 | 44306300 | 44306580 | 0.001264237 | −1.257950848 | −1.039484106 |
| chr13 | 26463200 | 26463580 | −0.001264029 | −1.028591147 | −1.201346805 |
| chr15 | 92395960 | 92397860 | 0.001263841 | −1.259405351 | −1.014104196 |
| chr16 | 81109900 | 81111300 | 0.00126357 | −1.087925681 | −0.822613139 |
| chr10 | 49590680 | 49591140 | −0.001263432 | −0.948836514 | −1.16454218 |
| chr8 | 50616300 | 50616780 | 0.001263417 | −1.182113453 | −0.974727771 |
| chr21 | 46408120 | 46408560 | 0.001263313 | −0.919030905 | −0.69503189 |
| chr5 | 143339020 | 143339320 | −0.001263304 | −1.041919741 | −1.234962456 |
| chr7 | 90404760 | 90405180 | −0.001262709 | −0.992343025 | −1.087412563 |
| chr7 | 149194160 | 149195280 | 0.001262682 | −1.224420023 | −0.966535154 |
| chr10 | 107562200 | 107562820 | −0.001262667 | −0.928936431 | −1.260321314 |
| chr20 | 32441280 | 32441760 | −0.001262497 | −0.755845944 | −0.962959144 |
| chr7 | 87998220 | 87998740 | 0.001262443 | −1.183334704 | −1.055361427 |
| chr19 | 854520 | 854880 | −0.001262377 | −0.951933522 | −1.168786733 |
| chr6 | 100266180 | 100266680 | 0.001262376 | −1.264846181 | −1.05234604 |
| chr6 | 100266160 | 100266700 | 0.001262376 | −1.264846181 | −1.05234604 |
| chr2 | 40511220 | 40512000 | −0.001262334 | −0.957973882 | −1.243798709 |
| chr6 | 7140920 | 7141940 | 0.001262041 | −1.371067329 | −1.153991038 |
| chr2 | 221892840 | 221893100 | 0.001261565 | −1.098176788 | −0.983711072 |
| chr2 | 221892840 | 221893100 | 0.001261565 | −1.098176788 | −0.983711072 |
| chr1 | 229323720 | 229324200 | 0.001261468 | −1.141041527 | −1.021295504 |
| chr2 | 25193980 | 25195260 | 0.001261359 | −1.321753108 | −1.162604746 |
| chr7 | 41982660 | 41982980 | 0.001261288 | −1.217749676 | −0.971127316 |
| chr3 | 22572040 | 22572480 | −0.001261266 | −1.080904277 | −1.357455367 |
| chr15 | 52121180 | 52122540 | 0.001261095 | −1.173848185 | −0.958198146 |
| chr4 | 15893700 | 15894220 | −0.001260738 | −1.042972612 | −1.224678812 |
| chr20 | 34787400 | 34788280 | 0.001260703 | −1.102360759 | −0.919195351 |
| chr19 | 13212460 | 13212820 | 0.001260587 | −1.049746447 | −0.949849156 |
| chr21 | 37441220 | 37443420 | 0.001260279 | −1.109895572 | −0.929222103 |
| chr5 | 53809980 | 53810340 | −0.001260047 | −1.140075294 | −1.334488909 |
| chr2 | 40005980 | 40007060 | 0.001260026 | −1.277736439 | −1.007502049 |
| chr12 | 102091120 | 102092100 | 0.001259893 | −1.218647169 | −1.102778029 |
| chr16 | 30197240 | 30197460 | 0.001259747 | −1.155255761 | −0.90909447 |
| chr2 | 208509920 | 208510520 | −0.001259744 | −0.907191449 | −1.217498111 |
| chr1 | 35978660 | 35979120 | −0.001259342 | −0.892797798 | −1.097099492 |
| chr2 | 174798120 | 174798620 | 0.001259161 | −1.32870782 | −1.196069691 |
| chr21 | 25755700 | 25756140 | −0.001258903 | −0.849179425 | −1.039812249 |
| chr7 | 74071680 | 74073420 | −0.001258339 | −1.00372973 | −1.18247468 |
| chr3 | 102724760 | 102725180 | 0.001258053 | −1.332355637 | −1.174765951 |
| chr14 | 102605480 | 102606860 | 0.001257864 | −1.359107749 | −1.150584129 |
| chr2 | 180128520 | 180129820 | 0.001257731 | −1.375667277 | −1.118448759 |
| chr3 | 99836480 | 99836920 | −0.001257586 | −1.091024888 | −1.275865249 |
| chr8 | 69109780 | 69110180 | −0.00125757 | −1.003339847 | −1.176544175 |
| chr6 | 83407460 | 83408020 | −0.001257531 | −1.039780013 | −1.215964639 |
| chr17 | 78316300 | 78316660 | −0.001257289 | −0.915199094 | −1.165191148 |
| chr1 | 92788340 | 92788860 | −0.001256874 | −0.978907932 | −1.223094092 |
| chr11 | 34764800 | 34765340 | −0.001256773 | −1.05743841 | −1.218534281 |
| chr6 | 141693280 | 141693600 | −0.001256655 | −0.945008613 | −1.166205984 |
| chr17 | 1162480 | 1162720 | 0.001256594 | −1.175680671 | −0.937962348 |
| chr11 | 41989820 | 41990300 | −0.001256586 | −1.084176232 | −1.363620527 |
| chr1 | 16297660 | 16298240 | −0.001256462 | −1.015828091 | −1.213931435 |
| chr15 | 44167400 | 44167680 | −0.001256433 | −0.918232107 | −1.02471468 |
| chr12 | 21653800 | 21655400 | 0.001256344 | −1.273161171 | −1.125178544 |
| chr10 | 107852840 | 107853320 | −0.001256286 | −0.931580618 | −1.174204514 |
| chr22 | 32149340 | 32150700 | 0.001256204 | −1.168937531 | −1.005177608 |
| chr1 | 16623220 | 16623620 | −0.001256195 | −1.031504034 | −1.175628303 |
| chr12 | 1702920 | 1704480 | −0.001256146 | −1.138590663 | −1.226642757 |
| chr3 | 195635680 | 195636860 | 0.001255999 | −1.302663151 | −1.061105759 |
| chr3 | 195635580 | 195636960 | 0.001255999 | −1.302663151 | −1.061105759 |
| chr8 | 137634240 | 137634740 | −0.001255727 | −0.898083775 | −1.160119598 |
| chr11 | 45167560 | 45169320 | −0.001255341 | −1.077253368 | −1.213619618 |
| chr1 | 66562000 | 66562480 | −0.001255208 | −0.941787698 | −1.182589994 |
| chr6 | 159555800 | 159556080 | 0.001255067 | −1.278036076 | −1.021909792 |
| chr4 | 156301220 | 156301700 | 0.001254973 | −1.201696977 | −1.151969991 |
| chr9 | 8776600 | 8777120 | −0.001254733 | −0.864464147 | −1.049981038 |
| chr2 | 62026660 | 62027040 | −0.001254354 | −0.969145724 | −1.211937997 |
| chr7 | 148663880 | 148664200 | 0.001254341 | −1.268479191 | −1.045635513 |
| chr1 | 107379020 | 107379540 | −0.001254241 | −1.046167107 | −1.265583221 |
| chr1 | 231493600 | 231493920 | −0.001254072 | −0.949679023 | −1.063418942 |
| chr15 | 93321020 | 93321300 | −0.001253908 | −1.029505644 | −1.303317593 |
| chr15 | 93321020 | 93321300 | −0.001253908 | −1.029505644 | −1.303317593 |
| chr12 | 81528880 | 81529400 | −0.001253905 | −1.110080476 | −1.356630811 |
| chr4 | 109168300 | 109168800 | −0.00125389 | −1.00226486 | −1.209027417 |
| chr13 | 22051180 | 22051480 | 0.001253887 | −1.316408478 | −1.066431207 |
| chr7 | 4680940 | 4682260 | 0.001253864 | −1.2554475 | −1.063257352 |
| chr5 | 10353400 | 10354600 | 0.001253727 | −1.099229169 | −0.909736886 |
| chr16 | 2469100 | 2469500 | −0.001253587 | −0.803466434 | −0.986193322 |
| chr9 | 105684400 | 105685020 | −0.001253095 | −0.973145395 | −1.175999836 |
| chr3 | 78303100 | 78303480 | −0.00125286 | −1.0630862 | −1.273146439 |
| chr1 | 23740320 | 23740680 | −0.001252568 | −0.896737506 | −1.106377961 |
| chr1 | 11322160 | 11323000 | 0.001252562 | −1.110564748 | −0.967297769 |
| chr15 | 48801340 | 48802020 | −0.001252438 | −1.022796063 | −1.249728777 |
| chr12 | 14782740 | 14783680 | 0.001251682 | −1.166413887 | −0.950282534 |
| chr22 | 43010480 | 43011460 | 0.00125164 | −1.260097442 | −1.130959223 |
| chr22 | 43010460 | 43011480 | 0.00125164 | −1.260097442 | −1.130959223 |
| chr10 | 110069440 | 110069880 | −0.001251375 | −0.966245391 | −1.116068674 |
| chr2 | 133873160 | 133873580 | 0.001251169 | −1.171924537 | −1.047197722 |
| chr3 | 129345560 | 129345980 | 0.001250817 | −1.156642857 | −0.949521749 |
| chr3 | 153169080 | 153169560 | −0.001250797 | −1.069423185 | −1.260633395 |
| chr4 | 21104760 | 21105160 | −0.001250533 | −1.105370683 | −1.296763172 |
| chr4 | 21104720 | 21105200 | −0.001250533 | −1.105370683 | −1.296763172 |
| chr8 | 142094440 | 142094740 | 0.001250467 | −1.343389493 | −1.070923982 |
| chr8 | 142094140 | 142095040 | 0.001250467 | −1.343389493 | −1.070923982 |
| chr4 | 15003500 | 15004200 | 0.001250387 | −1.222529577 | −1.010706727 |
| chr3 | 1426160 | 1426680 | −0.001250326 | −1.016666177 | −1.275658101 |
| chr4 | 62872900 | 62873320 | −0.001250275 | −1.188830951 | −1.332761465 |
| chr16 | 68297940 | 68299020 | 0.001250057 | −1.182644914 | −0.975841076 |
| chr8 | 24231720 | 24232180 | −0.001250021 | −0.903834781 | −1.140436692 |
| chr8 | 11417460 | 11417960 | −0.001249893 | −1.115809841 | −1.327248465 |
| chr1 | 58071720 | 58072900 | −0.001249832 | −1.126231302 | −1.354325079 |
| chr12 | 78982180 | 78983040 | 0.001249649 | −1.313587161 | −1.175770332 |
| chr1 | 27021920 | 27024460 | −0.001249089 | −1.057033327 | −1.110300003 |
| chr1 | 27021920 | 27024460 | −0.001249089 | −1.057033327 | −1.110300003 |
| chr19 | 36207880 | 36209540 | 0.001248519 | −1.079482477 | −0.921833216 |
| chr17 | 617640 | 618600 | 0.00124827 | −1.29091295 | −1.135504947 |
| chr2 | 119802420 | 119802720 | −0.001248003 | −0.989748267 | −1.200735227 |
| chr2 | 119802420 | 119802720 | −0.001248003 | −0.989748267 | −1.200735227 |
| chr8 | 93886900 | 93887520 | −0.001247946 | −0.906968498 | −1.182888868 |
| chr3 | 194463320 | 194463700 | −0.001247795 | −1.087573112 | −1.28846085 |
| chr3 | 194463300 | 194463720 | −0.001247795 | −1.087573112 | −1.28846085 |
| chr7 | 48653220 | 48653940 | −0.00124748 | −0.852315824 | −1.090918517 |
| chr5 | 11128660 | 11129060 | −0.001246776 | −1.098840228 | −1.288923551 |
| chr21 | 28959780 | 28960420 | 0.001246473 | −0.988834273 | −0.959150289 |
| chr6 | 52058840 | 52059300 | −0.001246403 | −1.094995972 | −1.375163778 |
| chr13 | 67564540 | 67564940 | 0.001246297 | −1.138608747 | −1.002708751 |
| chr2 | 185337780 | 185338700 | −0.001246217 | −0.989636691 | −1.251767133 |
| chr1 | 145575040 | 145576740 | 0.001246158 | −1.254004338 | −1.006946631 |
| chr15 | 90728000 | 90728580 | 0.001246102 | −1.093648608 | −0.816125856 |
| chr10 | 11502460 | 11502860 | 0.001246002 | −1.06078671 | −1.026488239 |
| chr1 | 60762080 | 60762480 | −0.001245879 | −0.986237259 | −1.1746743 |
| chr11 | 2448940 | 2450240 | 0.001245768 | −1.209561951 | −0.961474641 |
| chr4 | 61896040 | 61896420 | −0.001245766 | −0.954014421 | −1.21818172 |
| chr4 | 61896000 | 61896460 | −0.001245766 | −0.954014421 | −1.21818172 |
| chr8 | 35445340 | 35445700 | −0.001245031 | −1.002157233 | −1.215926647 |
| chr6 | 15244840 | 15246400 | −0.001244917 | −1.16982519 | −1.306963627 |
| chr15 | 89877640 | 89878740 | 0.001244698 | −1.129370054 | −0.970191471 |
| chr17 | 37850220 | 37850760 | −0.001244306 | −0.912654416 | −1.143334405 |
| chr14 | 51296720 | 51298460 | 0.001244214 | −1.276310025 | −1.039828922 |
| chr20 | 59202440 | 59202900 | −0.001243841 | −0.910169107 | −1.221768068 |
| chr7 | 33605200 | 33605660 | −0.001243559 | −1.05681874 | −1.199811941 |
| chr14 | 100841880 | 100843200 | 0.00124308 | −1.236332114 | −1.024558532 |
| chr14 | 77519200 | 77520180 | −0.001243075 | −0.998868234 | −1.031431912 |
| chr2 | 7567140 | 7568040 | 0.001242865 | −1.097489207 | −0.951694185 |
| chr16 | 24107820 | 24108180 | −0.001242833 | −0.875289689 | −1.074563384 |
| chr4 | 141025300 | 141025760 | −0.001242831 | −1.126277324 | −1.267357655 |
| chr3 | 85650060 | 85650540 | −0.001242596 | −0.946942885 | −1.189357438 |
| chr2 | 73089360 | 73090320 | 0.00124136 | −1.162181994 | −0.919272962 |
| chr2 | 73089400 | 73090280 | 0.00124136 | −1.162181994 | −0.919272962 |
| chr11 | 3012780 | 3014640 | 0.001241229 | −1.109006348 | −0.98522046 |
| chr4 | 980020 | 981540 | 0.00124071 | −1.041603146 | −0.861819793 |
| chr4 | 32816800 | 32817340 | −0.001240354 | −0.947637919 | −1.157929021 |
| chr6 | 142956980 | 142957440 | −0.001240167 | −1.208949276 | −1.393944317 |
| chr4 | 184546260 | 184546640 | 0.001240138 | −1.191641078 | −1.065730227 |
| chr10 | 17085800 | 17086500 | −0.001239926 | −1.007521171 | −1.253741894 |
| chr8 | 21595480 | 21595880 | −0.001239343 | −1.14675345 | −1.267226754 |
| chr1 | 243856380 | 243856780 | −0.001239033 | −0.993725558 | −1.23402378 |
| chr18 | 21491500 | 21492180 | −0.001238922 | −1.057852953 | −1.336954546 |
| chr2 | 194182040 | 194182480 | 0.001238578 | −1.056416023 | −0.91568798 |
| chr5 | 12046400 | 12046900 | 0.00123841 | −1.139783196 | −1.074692854 |
| chr15 | 72492000 | 72492400 | 0.001238261 | −1.110061804 | −0.847157557 |
| chr16 | 80745680 | 80746280 | 0.001237728 | −1.021650871 | −0.962768704 |
| chr2 | 43202120 | 43202680 | 0.001237318 | −1.260057619 | −0.998881346 |
| chr17 | 29662060 | 29662560 | −0.001237275 | −0.818863155 | −1.054095977 |
| chr16 | 65499600 | 65500340 | 0.001236964 | −1.123443894 | −1.058110514 |
| chr8 | 130951400 | 130952700 | −0.00123689 | −0.971161961 | −1.089405497 |
| chr11 | 37617920 | 37618480 | 0.001236856 | −1.107037678 | −1.131819582 |
| chr10 | 135085720 | 135086040 | −0.001236594 | −0.869140538 | −0.996978108 |
| chr12 | 108154120 | 108155360 | 0.00123649 | −1.265642902 | −1.081869747 |
| chr10 | 15204520 | 15205060 | −0.001236422 | −0.941553393 | −1.17259693 |
| chr4 | 148814440 | 148814960 | −0.001236402 | −1.004877845 | −1.222856736 |
| chr20 | 30467280 | 30468340 | 0.001236202 | −1.192025969 | −1.056028773 |
| chr5 | 67558000 | 67558540 | −0.001235826 | −1.053267462 | −1.189893452 |
| chr8 | 112037040 | 112037440 | −0.001235797 | −1.014877828 | −1.282090136 |
| chr12 | 29913480 | 29914300 | −0.001235735 | −0.855470712 | −1.21303912 |
| chr21 | 23422380 | 23422640 | −0.001235692 | −0.858926608 | −0.973477078 |
| chr21 | 23422380 | 23422640 | −0.001235692 | −0.858926608 | −0.973477078 |
| chr19 | 48825040 | 48825560 | 0.001234844 | −1.227374635 | −1.06608124 |
| chr19 | 54665920 | 54666280 | 0.001234614 | −1.18750323 | −1.032118493 |
| chr19 | 36504260 | 36506340 | 0.001234525 | −1.233700526 | −1.091664129 |
| chr7 | 113316740 | 113317220 | −0.001234442 | −0.733222502 | −1.137536278 |
| chr21 | 24493080 | 24493680 | −0.001234306 | −1.012786161 | −1.212455539 |
| chr5 | 117876460 | 117876700 | 0.001234057 | −1.199512715 | −1.118093825 |
| chr15 | 101459300 | 101460160 | 0.00123392 | −1.147649092 | −0.969089683 |
| chr14 | 82753700 | 82754100 | 0.00123242 | −1.145338798 | −1.126889401 |
| chr19 | 34345720 | 34346140 | −0.001232133 | −0.895611935 | −1.12147347 |
| chr5 | 5778240 | 5778760 | −0.001232058 | −0.984095411 | −1.297446159 |
| chr13 | 49421320 | 49421840 | 0.001231882 | −1.193771131 | −1.050781794 |
| chr8 | 125027600 | 125028040 | −0.001231794 | −1.010603182 | −1.166037773 |
| chr5 | 83337160 | 83337680 | 0.001231516 | −1.147937019 | −1.096044682 |
| chr5 | 83337160 | 83337680 | 0.001231516 | −1.147937019 | −1.096044682 |
| chr8 | 113355020 | 113355460 | −0.001231449 | −1.113804954 | −1.335170136 |
| chr11 | 94963720 | 94964840 | 0.001230729 | −1.336027486 | −1.051012506 |
| chr4 | 115654620 | 115655300 | −0.001230712 | −0.990318859 | −1.164966167 |
| chr14 | 60557960 | 60559140 | 0.001230483 | −1.248378928 | −1.096419433 |
| chr10 | 60528900 | 60529400 | 0.001230146 | −1.130007101 | −0.996562046 |
| chr5 | 88164860 | 88165440 | −0.00122985 | −0.86815934 | −1.159297628 |
| chr20 | 30467400 | 30468160 | 0.001229831 | −1.192025969 | −1.015004785 |
| chr12 | 78462220 | 78462580 | −0.001229667 | −1.047998575 | −1.217034314 |
| chr11 | 85779360 | 85781160 | 0.001229409 | −1.397330828 | −1.245990445 |
| chr3 | 195148540 | 195149420 | −0.00122904 | −0.960093668 | −1.154889502 |
| chr1 | 175288500 | 175289420 | 0.001228768 | −1.27238938 | −1.008634273 |
| chr4 | 184546280 | 184546640 | 0.001228739 | −1.191641078 | −1.068763582 |
| chr6 | 82632540 | 82633100 | −0.001228667 | −1.137258197 | −1.30184378 |
| chr14 | 24299120 | 24299540 | 0.001228434 | −1.171629468 | −1.014297172 |
| chr3 | 166089780 | 166090140 | −0.001228355 | −0.916302111 | −1.171123213 |
| chr2 | 233724900 | 233725380 | −0.001228331 | −0.851359762 | −1.083915606 |
| chr18 | 58091520 | 58092060 | 0.001227852 | −1.1973431 | −1.178113063 |
| chr8 | 120628040 | 120628560 | −0.001227599 | −0.904582861 | −1.114870594 |
| chr5 | 53809980 | 53810360 | −0.001227268 | −1.122140036 | −1.315318418 |
| chr17 | 56076620 | 56077480 | 0.001227264 | −1.174905274 | −1.019833426 |
| chr2 | 198317780 | 198318420 | 0.001226987 | −1.374657418 | −1.249508593 |
| chr2 | 52748740 | 52748980 | −0.001226837 | −0.809806039 | −1.058244098 |
| chr2 | 180302340 | 180303040 | −0.001226787 | −1.123577967 | −1.35103458 |
| chr18 | 69631700 | 69632080 | −0.001226515 | −0.883503955 | −1.144481284 |
| chr17 | 70324220 | 70324600 | −0.001226261 | −0.899221231 | −1.099423777 |
| chr17 | 70324200 | 70324620 | −0.001226261 | −0.899221231 | −1.099423777 |
| chr8 | 83130600 | 83131140 | −0.001226261 | −1.002574428 | −1.282177505 |
| chr15 | 52068920 | 52069820 | 0.001226101 | −1.098532203 | −0.861044064 |
| chr15 | 52068920 | 52069820 | 0.001226101 | −1.098532203 | −0.861044064 |
| chr5 | 2623180 | 2623700 | 0.001225608 | −1.123881742 | −1.023094117 |
| chr11 | 77408360 | 77408780 | −0.00122549 | −1.17935792 | −1.392809348 |
| chr11 | 77408320 | 77408820 | −0.00122549 | −1.17935792 | −1.392809348 |
| chr2 | 137693240 | 137693780 | −0.00122503 | −0.944149241 | −1.232332636 |
| chr16 | 88803140 | 88803420 | 0.001224872 | −0.998621445 | −0.836302108 |
| chr4 | 135053820 | 135054760 | −0.001224843 | −1.148528371 | −1.379962881 |
| chr1 | 197962480 | 197963400 | −0.001224771 | −1.019547002 | −1.294557981 |
| chr1 | 225522460 | 225523100 | −0.001224296 | −0.988452591 | −1.158805255 |
| chr6 | 129577660 | 129578140 | −0.001224277 | −1.092813138 | −1.296752235 |
| chr6 | 46109840 | 46110420 | −0.001224198 | −1.040949366 | −1.316641926 |
| chr15 | 34330540 | 34331580 | 0.001224138 | −1.011604216 | −0.794166643 |
| chr5 | 20356740 | 20357260 | 0.001223696 | −1.182940293 | −1.054085126 |
| chr3 | 168428880 | 168429300 | 0.001223459 | −1.290588961 | −1.10371017 |
| chr5 | 1545280 | 1546180 | 0.001223098 | −1.136727061 | −0.947375722 |
| chr14 | 103584400 | 103584660 | −0.001222904 | −1.098359687 | −1.197198416 |
| chr10 | 74451640 | 74452540 | 0.001222337 | −1.226686018 | −1.036633226 |
| chr1 | 228296260 | 228298060 | 0.001222216 | −1.2728646 | −1.116766101 |
| chr10 | 8304620 | 8305220 | −0.001222085 | −1.045000204 | −1.193784338 |
| chr11 | 118798800 | 118799060 | 0.00122196 | −1.30953162 | −1.137461569 |
| chr2 | 193856200 | 193856680 | −0.001221934 | −0.964767422 | −1.19699076 |
| chr4 | 164960940 | 164961840 | −0.001221764 | −0.995189241 | −1.285572417 |
| chr4 | 164960940 | 164961840 | −0.001221764 | −0.995189241 | −1.285572417 |
| chr3 | 182510640 | 182511960 | 0.001221681 | −1.416713496 | −1.257156022 |
| chr10 | 103235500 | 103235900 | −0.001221654 | −1.044282655 | −1.277372865 |
| chr7 | 1619160 | 1619460 | 0.001221492 | −1.244081246 | −0.995194527 |
| chr2 | 135962040 | 135962520 | −0.001221398 | −1.153836075 | −1.327232836 |
| chr2 | 135962000 | 135962560 | −0.001221398 | −1.153836075 | −1.327232836 |
| chr17 | 1370700 | 1371280 | −0.001221326 | −0.996014428 | −1.166462994 |
| chr2 | 170683780 | 170685000 | 0.001221137 | −1.162772163 | −0.92420656 |
| chr9 | 77338560 | 77338940 | −0.001221009 | −0.837797991 | −1.083740661 |
| chr5 | 142812780 | 142813300 | −0.001220843 | −0.962802376 | −1.219526606 |
| chr11 | 115990080 | 115990820 | −0.001220779 | −0.909597968 | −1.171677551 |
| chr1 | 210500100 | 210500440 | −0.001220333 | −0.954956627 | −1.143554959 |
| chr14 | 102785900 | 102786660 | −0.001220263 | −1.136836769 | −1.286157085 |
| chr2 | 203102740 | 203103900 | −0.00122006 | −0.969674624 | −1.075670569 |
| chr3 | 90161160 | 90161640 | 0.001219663 | −1.172918979 | −1.127332233 |
| chr11 | 34182000 | 34182480 | 0.00121956 | −1.332214618 | −1.04941521 |
| chr19 | 24228940 | 24229400 | 0.001219218 | −1.04198085 | −0.940405427 |
| chr8 | 31905640 | 31906140 | −0.001219192 | −1.017189678 | −1.26694241 |
| chr8 | 31905560 | 31906220 | −0.001219192 | −1.017189678 | −1.26694241 |
| chr17 | 2206100 | 2207580 | 0.001219076 | −1.16527235 | −0.989417252 |
| chr11 | 33795260 | 33796820 | 0.00121902 | −1.248344184 | −1.015507341 |
| chr3 | 80452940 | 80453460 | 0.001218823 | −1.084714145 | −1.025031274 |
| chr8 | 11417440 | 11418000 | −0.001218372 | −1.096218728 | −1.319116011 |
| chr11 | 65479220 | 65480820 | −0.001218302 | −0.982650276 | −1.155879345 |
| chr17 | 8092540 | 8092860 | 0.001217821 | −1.036922216 | −0.848273617 |
| chr2 | 219261400 | 219261740 | 0.001217759 | −1.141817801 | −0.872902401 |
| chr1 | 64970800 | 64971780 | −0.001217377 | −1.068582627 | −1.177948532 |
| chr12 | 53614680 | 53615060 | 0.001217339 | −1.125121196 | −1.038935329 |
| chr10 | 84955020 | 84955480 | −0.001216471 | −1.005887896 | −1.143556285 |
| chr14 | 64280920 | 64281380 | −0.00121638 | −1.012408808 | −1.212852085 |
| chr1 | 113181480 | 113181800 | 0.001216107 | −1.11396219 | −1.076532212 |
| chr9 | 21366180 | 21366640 | −0.001215638 | −0.799671797 | −1.025649014 |
| chr2 | 154599260 | 154599760 | 0.001215634 | −1.314598594 | −1.108797972 |
| chr20 | 50029340 | 50029740 | −0.001215527 | −1.072859135 | −1.167005198 |
| chr7 | 113316640 | 113317280 | −0.001215409 | −0.751799974 | −1.121363055 |
| chr21 | 21068780 | 21069260 | 0.001215213 | −0.923413956 | −0.865773906 |
| chr20 | 47257220 | 47257660 | 0.001214853 | −1.111964146 | −0.942221049 |
| chr20 | 37590360 | 37591560 | 0.001214737 | −1.282843604 | −1.157496246 |
| chr5 | 55330060 | 55330600 | −0.001214727 | −0.834417876 | −1.133766913 |
| chr6 | 51142620 | 51143040 | −0.001214461 | −1.034405041 | −1.275945136 |
| chr6 | 51142560 | 51143100 | −0.001214461 | −1.034405041 | −1.275945136 |
| chr19 | 58330900 | 58331220 | 0.001214301 | −1.080997095 | −0.943600857 |
| chr2 | 118571640 | 118572840 | 0.001214091 | −1.220769573 | −0.991634321 |
| chr14 | 27232520 | 27232840 | −0.001213784 | −1.021162682 | −1.237688853 |
| chr11 | 59973760 | 59974220 | 0.00121368 | −1.146104595 | −1.014154631 |
| chr16 | 70464820 | 70465200 | 0.00121346 | −1.097188906 | −0.912412559 |
| chr6 | 2840880 | 2842840 | 0.001213386 | −1.187003626 | −1.020839382 |
| chr12 | 97832100 | 97832480 | −0.001213302 | −0.953375581 | −1.224810179 |
| chr12 | 54697880 | 54698360 | 0.001213066 | −1.119906636 | −0.91483072 |
| chr1 | 115003760 | 115004100 | −0.001212992 | −0.994538849 | −1.157797212 |
| chr13 | 47353680 | 47354100 | −0.001212944 | −1.045499244 | −1.279028225 |
| chr10 | 27015080 | 27015360 | 0.001212873 | −1.228250032 | −1.032648259 |
| chr21 | 47731460 | 47732160 | −0.001212617 | −0.980322935 | −1.055045786 |
| chr12 | 89094180 | 89094420 | −0.00121244 | −1.027028168 | −1.247536423 |
| chr4 | 11164760 | 11165240 | −0.001211829 | −1.038335685 | −1.299214695 |
| chr4 | 29265820 | 29266600 | −0.001211683 | −1.13257152 | −1.329896231 |
| chr9 | 13710900 | 13711520 | −0.001211636 | −0.82031097 | −1.058491404 |
| chr11 | 16806780 | 16807700 | 0.001211606 | −1.221352575 | −0.880283256 |
| chr1 | 198125320 | 198126940 | 0.001211498 | −1.294595579 | −1.089822367 |
| chr15 | 49154080 | 49154360 | 0.001211221 | −1.093363453 | −0.82782685 |
| chr2 | 185337860 | 185338660 | −0.001211173 | −0.97143687 | −1.235280258 |
| chr10 | 73533740 | 73534060 | 0.001210786 | −1.170480968 | −0.915533243 |
| chr8 | 120844620 | 120845560 | 0.001210655 | −1.341988445 | −1.010202938 |
| chr21 | 24576560 | 24577000 | −0.001210605 | −0.891622268 | −1.080253565 |
| chr15 | 74428680 | 74429080 | 0.001210396 | −1.164200732 | −0.908676589 |
| chr2 | 6459560 | 6460100 | −0.001210352 | −0.998491663 | −1.159085174 |
| chr9 | 136703360 | 136704340 | 0.001209591 | −1.017861564 | −0.759138484 |
| chr6 | 13892980 | 13893380 | −0.001209436 | −0.939170542 | −1.277893058 |
| chr10 | 111682600 | 111683780 | 0.001209054 | −1.284999496 | −1.137368099 |
| chr1 | 8419980 | 8421300 | −0.001208927 | −0.871658824 | −1.062075454 |
| chr1 | 243856300 | 243856800 | −0.001208579 | −1.026389742 | −1.248032019 |
| chr16 | 88372600 | 88373580 | 0.001208482 | −1.165362821 | −1.025697893 |
| chr6 | 126092800 | 126093220 | −0.001208404 | −0.94575872 | −1.194077996 |
| chr19 | 19051600 | 19052380 | 0.001207969 | −1.239019085 | −0.964019199 |
| chr19 | 19051440 | 19052540 | 0.001207969 | −1.239019085 | −0.964019199 |
| chr17 | 80681160 | 80681800 | 0.001207769 | −1.141852759 | −0.938696361 |
| chr19 | 14228120 | 14229320 | 0.001207731 | −1.305613471 | −1.152804905 |
| chr5 | 176000600 | 176001800 | 0.001207673 | −1.257058591 | −1.106470668 |
| chr7 | 38368000 | 38368620 | −0.001207579 | −1.041605026 | −1.288042924 |
| chr17 | 7751800 | 7752060 | −0.001207574 | −0.901914986 | −1.029214119 |
| chr19 | 30335220 | 30336480 | 0.001207473 | −1.069259459 | −0.877502835 |
| chr8 | 93508880 | 93509320 | −0.001207464 | −0.911554656 | −1.196768897 |
| chr17 | 21191080 | 21191760 | 0.001207319 | −1.101621059 | −0.868294246 |
| chr22 | 43601800 | 43602600 | 0.001207169 | −1.140369367 | −0.87652587 |
| chr6 | 117452920 | 117453500 | −0.001206976 | −0.963118978 | −1.216508145 |
| chr22 | 23714340 | 23715240 | 0.001206806 | −1.116828183 | −1.121903181 |
| chr17 | 27053880 | 27054180 | 0.001206799 | −1.265634737 | −1.06124339 |
| chr14 | 74959620 | 74961000 | 0.001206574 | −1.429330183 | −1.268459656 |
| chr1 | 173760280 | 173760700 | −0.001206171 | −0.926896196 | −1.148514365 |
| chr1 | 173760240 | 173760740 | −0.001206171 | −0.926896196 | −1.148514365 |
| chr11 | 114614360 | 114614840 | −0.001206128 | −0.833584385 | −1.060573345 |
| chr1 | 105627380 | 105627920 | −0.001205945 | −0.896334398 | −1.14153036 |
| chr13 | 34116440 | 34117260 | 0.001205758 | −1.342450095 | −1.167756458 |
| chr13 | 34116400 | 34117300 | 0.001205758 | −1.342450095 | −1.167756458 |
| chr9 | 28425040 | 28425560 | −0.001205599 | −0.742354182 | −1.005239558 |
| chr13 | 54771360 | 54771720 | −0.001205278 | −1.003191182 | −1.23093907 |
| chr4 | 187997460 | 187997940 | −0.001205087 | −1.118643855 | −1.294389273 |
| chr7 | 115162700 | 115163160 | 0.001204999 | −1.339030026 | −1.232172328 |
| chr21 | 22519740 | 22520360 | −0.001204929 | −0.820860419 | −1.102227749 |
| chr10 | 110075840 | 110076060 | −0.001204207 | −0.863514893 | −1.18311166 |
| chr10 | 110075840 | 110076060 | −0.001204207 | −0.863514893 | −1.18311166 |
| chr8 | 24077320 | 24077880 | −0.001204033 | −1.12952115 | −1.314799374 |
| chr18 | 62856700 | 62857480 | −0.00120401 | −0.935258714 | −1.153148869 |
| chr13 | 39030440 | 39030940 | −0.001203971 | −0.940403428 | −1.083594628 |
| chr21 | 17736760 | 17737220 | −0.00120394 | −0.806744724 | −0.970807482 |
| chr17 | 7745980 | 7746200 | −0.001203913 | −0.940500011 | −1.052260744 |
| chr19 | 29703600 | 29704360 | 0.001203835 | −1.086074906 | −1.0045817 |
| chr6 | 16183400 | 16183640 | 0.00120328 | −1.101015072 | −0.88839328 |
| chr10 | 67542120 | 67542560 | −0.001203212 | −1.013385985 | −1.22649943 |
| chr7 | 99005940 | 99006820 | 0.001203185 | −1.207817485 | −0.921019727 |
| chr13 | 67564460 | 67564960 | 0.001202977 | −1.171501003 | −1.042427981 |
| chr19 | 18528480 | 18528680 | 0.001202764 | −1.032990905 | −0.856041634 |
| chr9 | 33024660 | 33025900 | 0.001202604 | −1.184381131 | −1.022054537 |
| chr19 | 54919680 | 54920780 | 0.001202591 | −1.270170808 | −1.096662319 |
| chr3 | 185006020 | 185006420 | −0.001202328 | −0.852331539 | −1.107270292 |
| chr3 | 185005980 | 185006460 | −0.001202328 | −0.852331539 | −1.107270292 |
| chr16 | 1661880 | 1662780 | 0.001202308 | −1.273612253 | −1.08503488 |
| chr13 | 95362880 | 95364540 | 0.001202284 | −1.191224466 | −1.018070997 |
| chr2 | 67681080 | 67681540 | −0.001202144 | −0.88942552 | −1.139414448 |
| chr3 | 152881180 | 152881420 | 0.001201766 | −1.115171139 | −0.873132894 |
| chr3 | 44902840 | 44904000 | 0.001201578 | −1.248934501 | −1.113724575 |
| chr2 | 181291460 | 181291920 | 0.001201571 | −1.120400795 | −1.070809981 |
| chr16 | 54647040 | 54647680 | −0.001201321 | −0.75250432 | −1.087478963 |
| chr3 | 9978040 | 9978420 | 0.001201259 | −1.16744819 | −1.082596736 |
| chr5 | 17812460 | 17812840 | −0.001201093 | −0.91135017 | −1.065935311 |
| chr20 | 633120 | 634660 | 0.001201093 | −1.218610529 | −1.075063977 |
| chr2 | 117962340 | 117963180 | −0.001201005 | −1.11518608 | −1.237301669 |
| chr2 | 81297460 | 81297920 | −0.001200998 | −0.845786056 | −1.149326115 |
| chr17 | 37097660 | 37098100 | −0.001200876 | −0.88152282 | −1.144677796 |
| chr17 | 37097660 | 37098100 | −0.001200876 | −0.88152282 | −1.144677796 |
| chr6 | 70630480 | 70631160 | −0.001200845 | −0.98913684 | −1.20579959 |
| chr17 | 72489520 | 72489820 | 0.0012007 | −1.239543611 | −1.032240078 |
| chr17 | 72489520 | 72489820 | 0.0012007 | −1.239543611 | −1.032240078 |
| chr1 | 110182880 | 110183160 | 0.001200531 | −1.300899059 | −1.105708691 |
| chr3 | 154851260 | 154851720 | −0.001200142 | −1.079695337 | −1.221571731 |
| chr16 | 46864220 | 46865920 | 0.001200072 | −1.182265437 | −0.968443028 |
| chr12 | 5345160 | 5345540 | −0.001200027 | −0.952917171 | −1.207606887 |
| chr19 | 1094840 | 1095680 | 0.001199939 | −1.054474012 | −0.871313224 |
| chr11 | 41989760 | 41990340 | −0.00119985 | −1.084176232 | −1.356422789 |
| chr8 | 57255680 | 57256240 | −0.001199308 | −1.140671651 | −1.383641872 |
| chr11 | 90968720 | 90969240 | −0.00119922 | −1.025254497 | −1.318031987 |
| chr12 | 40769480 | 40769900 | 0.001198906 | −1.132401584 | −1.072273022 |
| chr16 | 66901500 | 66902340 | 0.00119872 | −1.146082674 | −1.007084748 |
| chr16 | 66901500 | 66902340 | 0.00119872 | −1.146082674 | −1.007084748 |
| chr14 | 96829220 | 96830360 | 0.001198625 | −1.413652008 | −1.209057677 |
| chr11 | 46402720 | 46403520 | 0.001198254 | −1.368472191 | −1.173418473 |
| chr4 | 69416900 | 69417160 | −0.001197804 | −1.206826006 | −1.317193331 |
| chr1 | 17655300 | 17655720 | −0.001197803 | −0.778013258 | −1.034152628 |
| chr9 | 10403020 | 10403460 | 0.001197693 | −0.98988162 | −0.911057544 |
| chr8 | 38914660 | 38915060 | 0.001197641 | −1.125497783 | −1.076287175 |
| chr8 | 38914640 | 38915080 | 0.001197641 | −1.125497783 | −1.076287175 |
| chr2 | 7923820 | 7924340 | −0.001197635 | −0.98185277 | −1.176862082 |
| chr12 | 22763680 | 22764260 | 0.001197537 | −1.216524757 | −1.194092265 |
| chr13 | 79056940 | 79057380 | −0.001197525 | −1.115228205 | −1.21357472 |
| chr2 | 179273140 | 179273640 | −0.001197222 | −1.110933584 | −1.319363818 |
| chr7 | 101791760 | 101792160 | −0.001196774 | −0.971198905 | −1.136602889 |
| chr7 | 101791760 | 101792160 | −0.001196774 | −0.971198905 | −1.136602889 |
| chr7 | 124130820 | 124131300 | −0.001196579 | −1.125197427 | −1.292664642 |
| chr12 | 53765920 | 53766180 | 0.001196474 | −1.171356778 | −0.900945301 |
| chr15 | 98109380 | 98109780 | −0.001196383 | −0.757055245 | −1.002288551 |
| chr17 | 73092300 | 73092640 | 0.001196189 | −1.159572842 | −0.880572792 |
| chr2 | 102146000 | 102148080 | −0.001196133 | −1.105352017 | −1.306713786 |
| chr21 | 19876980 | 19877240 | −0.001196011 | −0.825730433 | −1.017447062 |
| chr17 | 29814420 | 29815580 | 0.001196 | −1.303400487 | −1.123467166 |
| chr2 | 212182400 | 212183040 | −0.001195893 | −1.002307536 | −1.269967787 |
| chr2 | 235018480 | 235018880 | 0.00119561 | −0.985994924 | −0.839009989 |
| chr2 | 180357840 | 180358820 | −0.001195075 | −0.932599651 | −1.078881452 |
| chr2 | 180357800 | 180358860 | −0.001195075 | −0.932599651 | −1.078881452 |
| chr21 | 47705660 | 47706740 | 0.001194989 | −1.169336392 | −0.977406546 |
| chr5 | 94132100 | 94132580 | −0.001194534 | −1.150123791 | −1.337144713 |
| chr6 | 42930780 | 42931240 | 0.0011945 | −1.203549464 | −0.987484698 |
| chr10 | 19229780 | 19230460 | −0.001194467 | −0.960447923 | −1.226065319 |
| chr4 | 35200700 | 35201140 | −0.001194133 | −1.127312099 | −1.290346334 |
| chr4 | 35200680 | 35201160 | −0.001194133 | −1.127312099 | −1.290346334 |
| chr6 | 137520320 | 137521180 | −0.001194037 | −1.044209324 | −1.280740993 |
| chr1 | 60551140 | 60552040 | −0.001194013 | −1.105087343 | −1.293150848 |
| chr19 | 2270480 | 2270780 | 0.001193947 | −1.127831248 | −0.935318657 |
| chr8 | 65170660 | 65171160 | −0.001193798 | −0.960047134 | −1.129175353 |
| chr8 | 113839200 | 113839600 | 0.001193051 | −1.346925355 | −1.270103544 |
| chr8 | 113839180 | 113839620 | 0.001193051 | −1.346925355 | −1.270103544 |
| chr9 | 132125080 | 132125660 | −0.001193041 | −1.085880252 | −1.239888573 |
| chr13 | 65715260 | 65715540 | −0.001192966 | −0.937563155 | −1.077979258 |
| chr2 | 198370360 | 198370620 | −0.001192925 | −0.926624004 | −1.065313884 |
| chr11 | 118964840 | 118966580 | 0.001192457 | −1.269699199 | −1.102087793 |
| chr13 | 98628060 | 98629520 | 0.00119221 | −1.324717678 | −1.092043924 |
| chr4 | 31331860 | 31332340 | −0.001192183 | −1.037872441 | −1.209479521 |
| chr7 | 26903200 | 26904600 | 0.001191973 | −1.21156736 | −1.091918802 |
| chr17 | 30813400 | 30814620 | −0.00119189 | −1.05674441 | −1.152605544 |
| chr1 | 179779020 | 179779300 | 0.001191865 | −1.227873789 | −0.976292507 |
| chr2 | 128615440 | 128616160 | 0.00119171 | −1.273671637 | −1.041881857 |
| chr6 | 56500800 | 56501420 | 0.001191551 | −1.019671547 | −0.991105821 |
| chr17 | 8092540 | 8092800 | 0.00119152 | −1.018218671 | −0.818128033 |
| chr7 | 84969140 | 84969560 | 0.00119143 | −1.177848808 | −1.131689787 |
| chr10 | 35624640 | 35626480 | 0.001190985 | −1.186243438 | −0.990493343 |
| chr6 | 82177320 | 82177820 | −0.001190881 | −0.94629633 | −1.267910115 |
| chr16 | 10592100 | 10592560 | 0.001190852 | −1.070248133 | −0.995314852 |
| chr2 | 215376400 | 215376800 | 0.001190843 | −0.984899844 | −0.955759673 |
| chr3 | 196359180 | 196359880 | 0.001190682 | −1.321825176 | −1.165470865 |
| chr10 | 121355740 | 121357700 | 0.001190582 | −1.308272527 | −1.187382596 |
| chr7 | 114855400 | 114855920 | −0.001190155 | −1.119083556 | −1.226173303 |
| chr2 | 88957460 | 88957960 | −0.001190123 | −0.810593811 | −1.023112753 |
| chr5 | 13265300 | 13266700 | −0.001189511 | −1.013771797 | −1.243218452 |
| chr5 | 32710900 | 32711420 | 0.001189177 | −1.085512423 | −0.943509072 |
| chr2 | 55065740 | 55066040 | 0.001189177 | −1.144090399 | −0.956174777 |
| chr2 | 145877960 | 145878320 | −0.001188693 | −0.964885586 | −1.186844406 |
| chr19 | 36239100 | 36239880 | −0.00118852 | −0.750848322 | −0.906264486 |
| chr11 | 67416860 | 67417500 | 0.001188492 | −1.123810936 | −0.911067839 |
| chr1 | 38147640 | 38148840 | 0.001188408 | −1.154823 | −0.931904496 |
| chr10 | 49513860 | 49515380 | 0.001188371 | −1.345423874 | −1.167232208 |
| chr9 | 22882040 | 22882560 | −0.001188298 | −0.855901938 | −1.049158829 |
| chr20 | 33291560 | 33293020 | 0.001187674 | −1.193665395 | −1.048082766 |
| chr3 | 10289100 | 10290960 | 0.00118763 | −1.300058251 | −1.108756523 |
| chr17 | 8198260 | 8199240 | 0.001187142 | −1.225581699 | −1.057200993 |
| chr2 | 230156580 | 230157100 | 0.00118705 | −1.139634485 | −1.056813439 |
| chr17 | 48243920 | 48244580 | 0.00118673 | −1.30282605 | −1.112496479 |
| chr13 | 38609760 | 38610640 | 0.001186525 | −1.15770934 | −1.17570584 |
| chr1 | 35978720 | 35979080 | −0.001186242 | −0.893375416 | −1.072877969 |
| chr7 | 68740580 | 68741100 | −0.001186138 | −1.050919334 | −1.17729582 |
| chr4 | 159689680 | 159690700 | 0.001185984 | −1.427776411 | −1.229291691 |
| chr12 | 3913080 | 3913380 | 0.001185616 | −1.151591676 | −0.961157022 |
| chr17 | 73092280 | 73092640 | 0.001185511 | −1.181173326 | −0.906397821 |
| chr17 | 76127420 | 76128580 | 0.00118549 | −1.168058075 | −0.991229665 |
| chr22 | 51015960 | 51017040 | 0.001185438 | −0.940388765 | −0.80023369 |
| chr14 | 92587780 | 92588800 | 0.001185334 | −1.328621012 | −1.124079096 |
| chr8 | 76221760 | 76222180 | −0.001185251 | −0.949271317 | −1.143164195 |
| chr12 | 121668140 | 121668380 | 0.001185145 | −1.14871018 | −0.97486945 |
| chr13 | 35584660 | 35585120 | −0.001185098 | −1.067656615 | −1.267223017 |
| chr16 | 29801680 | 29802500 | 0.001185092 | −1.118391281 | −1.0247297 |
| chr13 | 84125380 | 84126060 | 0.001184924 | −1.271718315 | −1.168909752 |
| chr6 | 83854580 | 83855360 | −0.001184551 | −1.004254501 | −1.255365219 |
| chr16 | 84705960 | 84706280 | 0.001184505 | −1.107343315 | −0.993945478 |
| chr17 | 8198280 | 8199240 | 0.001184501 | −1.225581699 | −1.051078725 |
| chr3 | 12045260 | 12047200 | −0.001184283 | −1.134864826 | −1.218046833 |
| chr16 | 3442940 | 3443540 | −0.001184056 | −1.050657965 | −1.216459836 |
| chr6 | 66269940 | 66270440 | −0.001183899 | −1.11823642 | −1.377301441 |
| chr9 | 22411680 | 22412100 | 0.001183823 | −1.212639766 | −1.10737586 |
| chr1 | 219102100 | 219102940 | −0.00118363 | −1.052471849 | −1.305445638 |
| chr9 | 138986500 | 138987600 | 0.00118339 | −1.019662725 | −0.828391443 |
| chr2 | 123293280 | 123293660 | −0.001183136 | −1.087150118 | −1.238855108 |
| chr5 | 86708600 | 86709100 | 0.001182369 | −1.1145563 | −0.982813194 |
| chr7 | 44597920 | 44598220 | −0.001182254 | −1.068588387 | −1.168155356 |
| chr9 | 8776580 | 8777160 | −0.00118224 | −0.8664225 | −1.079186428 |
| chr20 | 20438880 | 20439680 | −0.001182217 | −0.955598227 | −1.162519406 |
| chr10 | 83106120 | 83106600 | −0.001182027 | −1.000927804 | −1.261313409 |
| chr2 | 211366960 | 211367400 | 0.001181917 | −1.155586841 | −1.089851525 |
| chr9 | 138739620 | 138739960 | −0.00118134 | −1.007159495 | −1.129314591 |
| chr14 | 96829240 | 96830400 | 0.001181308 | −1.413652008 | −1.191436619 |
| chr3 | 6219940 | 6220380 | 0.001181015 | −1.203103708 | −1.045859112 |
| chr3 | 6219900 | 6220420 | 0.001181015 | −1.203103708 | −1.045859112 |
| chr2 | 95916420 | 95916960 | −0.001180617 | −1.002016816 | −1.159933362 |
| chr15 | 89448020 | 89448280 | 0.001180543 | −1.111245866 | −0.88909863 |
| chr17 | 65821300 | 65822300 | 0.001180356 | −1.062103798 | −0.934075718 |
| chr8 | 106884820 | 106885220 | −0.001180096 | −0.94776627 | −1.195876122 |
| chr17 | 44344060 | 44344580 | −0.00118008 | −0.973379838 | −1.181841893 |
| chr17 | 44344040 | 44344600 | −0.00118008 | −0.973379838 | −1.181841893 |
| chr21 | 30445680 | 30446360 | 0.001179805 | −1.009936497 | −0.869164799 |
| chr1 | 150045100 | 150045380 | 0.001179613 | −0.979298964 | −0.912281131 |
| chr4 | 109168300 | 109168740 | −0.001179512 | −1.048098194 | −1.204064846 |
| chr9 | 31663720 | 31664220 | −0.001179192 | −0.765452265 | −0.994050551 |
| chr21 | 24576520 | 24577060 | −0.001179017 | −0.915820206 | −1.139263789 |
| chr17 | 61816920 | 61817460 | 0.001178977 | −1.096899985 | −1.040641534 |
| chr17 | 74518920 | 74519820 | 0.001178958 | −1.249779958 | −1.059153482 |
| chr20 | 34787340 | 34788320 | 0.001178755 | −1.131120795 | −0.925539546 |
| chr5 | 149828500 | 149830100 | −0.001178399 | −1.058882817 | −1.154800097 |
| chr19 | 56135380 | 56136180 | 0.001178339 | −1.130879139 | −0.983293139 |
| chr19 | 14007040 | 14007660 | 0.001178207 | −1.056615763 | −0.991755151 |
| chr2 | 175206260 | 175206560 | 0.00117772 | −1.26946639 | −1.156902665 |
| chr2 | 175206240 | 175206580 | 0.00117772 | −1.26946639 | −1.156902665 |
| chr1 | 99468440 | 99468800 | −0.001177617 | −1.031380716 | −1.240685848 |
| chr10 | 104670540 | 104671120 | −0.001177603 | −1.014532474 | −1.244310296 |
| chr19 | 45375880 | 45376420 | −0.001177051 | −0.853545333 | −1.126838819 |
| chr4 | 32297480 | 32298160 | −0.001176987 | −1.211156079 | −1.388273995 |
| chr9 | 30080960 | 30081560 | −0.001176736 | −0.87850423 | −1.088554918 |
| chr11 | 1962720 | 1963840 | −0.001176719 | −0.926171512 | −1.084404958 |
| chr8 | 93719860 | 93720300 | −0.001176601 | −0.982082889 | −1.249336564 |
| chr4 | 74044940 | 74045440 | −0.001176308 | −1.108883837 | −1.337501815 |
| chr13 | 63316520 | 63316980 | −0.001176206 | −1.160712677 | −1.37895351 |
| chr9 | 127702700 | 127703900 | 0.001176172 | −1.110539522 | −0.871517021 |
| chr9 | 105798700 | 105799140 | −0.001175789 | −0.963889346 | −1.139473449 |
| chr11 | 72492240 | 72493740 | 0.001175766 | −1.185450973 | −1.005833425 |
| chr22 | 49016580 | 49017420 | 0.001175661 | −1.096217079 | −0.946364598 |
| chr19 | 14485920 | 14486240 | 0.001175621 | −1.03793287 | −0.913128625 |
| chr15 | 48801460 | 48801980 | −0.001175427 | −0.985259147 | −1.225699074 |
| chr16 | 29823220 | 29823640 | −0.001175392 | −0.88944917 | −0.918133563 |
| chr21 | 39079800 | 39080180 | 0.001175386 | −0.88779233 | −0.740492862 |
| chr12 | 6480500 | 6480900 | 0.001175231 | −1.373911298 | −1.199836555 |
| chr12 | 118214700 | 118215200 | 0.001174956 | −1.107007671 | −1.039492493 |
| chr15 | 54018220 | 54018660 | −0.001174727 | −0.858123091 | −0.990219866 |
| chr11 | 30246300 | 30246620 | −0.001174675 | −0.99797602 | −1.227530576 |
| chr22 | 47781840 | 47782420 | 0.001174501 | −1.067599388 | −0.968005739 |
| chr22 | 47148720 | 47149040 | 0.001174439 | −0.955882806 | −0.749115328 |
| chr19 | 14062560 | 14063940 | 0.001174382 | −1.263817125 | −1.124700433 |
| chr6 | 141364480 | 141364960 | −0.001174221 | −1.171533632 | −1.382523352 |
| chr2 | 21649140 | 21649600 | −0.001174084 | −1.152986586 | −1.371829475 |
| chr1 | 72364300 | 72364880 | 0.00117384 | −1.147426044 | −1.117446301 |
| chr21 | 21068680 | 21069240 | 0.001173833 | −0.949034352 | −0.937288214 |
| chr6 | 141364480 | 141365000 | −0.001173806 | −1.165858059 | −1.387570656 |
| chr10 | 45454240 | 45455980 | 0.001173602 | −1.400620717 | −1.228709675 |
| chr19 | 2720760 | 2721780 | 0.001173521 | −1.198458328 | −1.052677645 |
| chr10 | 29191080 | 29191320 | −0.001173456 | −0.880129584 | −1.100141935 |
| chr10 | 29191080 | 29191320 | −0.001173456 | −0.880129584 | −1.100141935 |
| chr19 | 5719700 | 5721000 | 0.001173299 | −1.142295381 | −1.041133364 |
| chr18 | 70891100 | 70891580 | −0.001173241 | −0.958163141 | −1.144912761 |
| chr4 | 15005180 | 15005600 | 0.001172937 | −1.387401143 | −1.245306738 |
| chr8 | 67836860 | 67838660 | 0.001172846 | −1.242751365 | −1.051933224 |
| chr19 | 1028100 | 1028340 | 0.001172764 | −1.032655836 | −0.897287648 |
| chr18 | 31376760 | 31377160 | −0.0011725 | −1.06696631 | −1.294729173 |
| chr2 | 208030520 | 208031900 | 0.001172497 | −1.234194465 | −1.003836505 |
| chr12 | 76477660 | 76479280 | 0.001172232 | −1.249042033 | −1.02166796 |
| chr2 | 125830280 | 125831020 | −0.001172096 | −0.967915207 | −1.24802433 |
| chr6 | 85145360 | 85145840 | −0.001171955 | −1.047857857 | −1.211797042 |
| chr13 | 53902380 | 53903140 | −0.001171859 | −1.115445994 | −1.309269516 |
| chr11 | 118888960 | 118889900 | 0.001171352 | −1.401177853 | −1.201084692 |
| chr12 | 6797800 | 6799740 | 0.001171047 | −1.44515329 | −1.271487415 |
| chr1 | 194368160 | 194368680 | −0.001170539 | −1.054621545 | −1.232415104 |
| chr9 | 92136540 | 92136760 | −0.001170022 | −0.816691854 | −0.980908363 |
| chr9 | 92136540 | 92136760 | −0.001170022 | −0.816691854 | −0.980908363 |
| chr12 | 73863900 | 73864140 | 0.001169856 | −1.243013346 | −1.120962903 |
| chr12 | 73863900 | 73864140 | 0.001169856 | −1.243013346 | −1.120962903 |
| chr8 | 109905500 | 109906000 | −0.001169787 | −1.021813515 | −1.232369686 |
| chr19 | 56014720 | 56015880 | 0.001169768 | −1.105090997 | −0.944169086 |
| chr4 | 104401820 | 104402340 | −0.001169747 | −1.129591013 | −1.268712083 |
| chr22 | 36839200 | 36839640 | 0.001169686 | −0.993637237 | −0.862907234 |
| chr2 | 198138460 | 198138980 | −0.001169684 | −1.030770031 | −1.161274136 |
| chr11 | 106890580 | 106891080 | −0.001169633 | −0.876028252 | −1.171019014 |
| chr5 | 170480700 | 170481140 | −0.001169136 | −1.116438317 | −1.315049953 |
| chr15 | 93211060 | 93211540 | −0.001168735 | −0.930711249 | −1.026667942 |
| chr15 | 74045100 | 74045820 | −0.001168725 | −0.946514025 | −1.017555228 |
| chr1 | 156023020 | 156023840 | 0.001168639 | −1.284439158 | −0.987676655 |
| chr19 | 35632000 | 35632540 | 0.001168548 | −1.271783081 | −1.117086311 |
| chr1 | 237245640 | 237246020 | −0.001168144 | −0.963173557 | −1.11635275 |
| chr6 | 77489120 | 77489560 | −0.00116813 | −1.048340518 | −1.247434226 |
| chr9 | 29868280 | 29868780 | −0.00116796 | −0.949601447 | −1.180924832 |
| chr2 | 184790480 | 184791120 | −0.001167716 | −0.965104782 | −1.24209538 |
| chr6 | 110028360 | 110028840 | −0.001167693 | −1.108680684 | −1.328584161 |
| chr4 | 93398660 | 93399180 | −0.00116759 | −0.940853293 | −1.147305183 |
| chr17 | 80681260 | 80681740 | 0.001167535 | −1.166693585 | −0.937403333 |
| chr14 | 63266280 | 63266800 | −0.001167299 | −1.115922387 | −1.329189877 |
| chr17 | 7120400 | 7121060 | −0.001167287 | −0.993549598 | −1.133765649 |
| chr1 | 32965740 | 32967020 | 0.001167168 | −1.087923051 | −0.972463632 |
| chr13 | 50589000 | 50589500 | −0.001167071 | −1.111814278 | −1.370030881 |
| chr15 | 81927640 | 81927920 | −0.001167014 | −0.961879365 | −1.15764957 |
| chr15 | 81927640 | 81927920 | −0.001167014 | −0.961879365 | −1.15764957 |
| chr2 | 32390600 | 32391080 | 0.001166975 | −1.228726322 | −1.089712264 |
| chr2 | 142311060 | 142311400 | −0.001166919 | −0.983710379 | −1.138446928 |
| chr9 | 20565940 | 20566160 | −0.001166843 | −0.983615313 | −1.152319932 |
| chr10 | 8304680 | 8305140 | −0.001166815 | −1.067553258 | −1.232843793 |
| chr1 | 228565960 | 228566400 | 0.001166762 | −1.244583255 | −0.981060238 |
| chr3 | 180184200 | 180184580 | 0.00116668 | −1.029031279 | −1.066635475 |
| chr1 | 29426940 | 29427480 | −0.001166627 | −1.030436308 | −1.211792473 |
| chr1 | 203940420 | 203940860 | 0.001166603 | −1.125773431 | −1.047784907 |
| chr17 | 40915500 | 40915780 | 0.001166522 | −1.087886311 | −0.917283623 |
| chr17 | 79934900 | 79936140 | 0.001166501 | −1.061797578 | −0.918900031 |
| chr19 | 48248580 | 48249500 | 0.001166229 | −1.090131986 | −0.951030474 |
| chr6 | 97857400 | 97858300 | 0.001166216 | −1.151393244 | −1.168385663 |
| chr10 | 80062400 | 80063760 | −0.00116609 | −1.024552077 | −1.127485587 |
| chr16 | 34949840 | 34950160 | −0.001166089 | −0.908601327 | −1.111679598 |
| chr2 | 6439480 | 6440020 | −0.001165853 | −1.084320171 | −1.276866457 |
| chr19 | 31474400 | 31475140 | −0.001165673 | −0.996676593 | −1.259615179 |
| chr1 | 198837680 | 198838140 | 0.001165654 | −0.815249712 | −0.853931852 |
| chr8 | 16785020 | 16785600 | −0.001165335 | −1.001383833 | −1.280293615 |
| chr17 | 65712940 | 65714660 | 0.001165275 | −1.175244563 | −0.968458987 |
| chr2 | 48712860 | 48713400 | −0.001165115 | −1.029933038 | −1.204973494 |
| chr13 | 54080640 | 54081080 | −0.001164734 | −1.123848131 | −1.261936703 |
| chr2 | 188678580 | 188679020 | −0.001164732 | −0.913091148 | −1.085884119 |
| chr1 | 983400 | 983740 | 0.001164599 | −1.151636674 | −0.859750098 |
| chr8 | 21936700 | 21936920 | −0.001164542 | −0.992154137 | −1.151507399 |
| chr1 | 199528540 | 199528980 | −0.001164538 | −0.905148786 | −1.263387241 |
| chr13 | 84125340 | 84126200 | 0.001164507 | −1.161321722 | −1.121527041 |
| chr6 | 41034140 | 41035360 | 0.001163946 | −1.206898496 | −1.122832649 |
| chr2 | 52870800 | 52871160 | −0.001163885 | −0.946863609 | −1.170752029 |
| chr1 | 27560560 | 27562180 | −0.001163657 | −0.979015718 | −1.026154428 |
| chr12 | 54718460 | 54719040 | 0.001163581 | −1.440163337 | −1.254659087 |
| chr8 | 17103540 | 17105180 | 0.001163383 | −1.29817102 | −1.175196882 |
| chr21 | 24615520 | 24616020 | −0.001163347 | −0.855560278 | −1.135781434 |
| chr7 | 105773260 | 105773680 | −0.001163208 | −0.907652348 | −1.058015652 |
| chr2 | 26256360 | 26257380 | 0.001162843 | −1.358953895 | −1.156420462 |
| chr9 | 113913200 | 113914100 | −0.001162535 | −0.80978964 | −1.119922917 |
| chr15 | 63481240 | 63482500 | 0.001162527 | −1.046714998 | −0.803686236 |
| chr5 | 149815240 | 149816440 | 0.001162327 | −1.298756123 | −1.098150517 |
| chr11 | 89938080 | 89938340 | 0.001162299 | −1.137671916 | −0.947502677 |
| chr7 | 7456480 | 7457800 | −0.001162279 | −1.058988008 | −1.22145308 |
| chr1 | 76539540 | 76541080 | 0.001162183 | −1.182002367 | −0.949140447 |
| chr8 | 126444320 | 126444600 | 0.001161908 | −1.273138499 | −1.064536255 |
| chr14 | 54192720 | 54193180 | −0.001161794 | −1.00364779 | −1.235312918 |
| chr15 | 66094540 | 66094980 | −0.001161549 | −0.849157162 | −1.06827742 |
| chr7 | 5594340 | 5595200 | 0.001161531 | −1.198372624 | −0.939365144 |
| chr7 | 121842300 | 121842780 | −0.001161503 | −1.102154205 | −1.280262089 |
| chr5 | 36294700 | 36295280 | −0.001161436 | −1.024847482 | −1.248431292 |
| chr2 | 97001160 | 97002400 | 0.00116141 | −1.340151167 | −1.106800577 |
| chr1 | 103219440 | 103219940 | 0.001161372 | −1.156310293 | −1.055402096 |
| chr7 | 4680960 | 4682280 | 0.001161065 | −1.235371705 | −1.05767703 |
| chr16 | 8921580 | 8922080 | −0.001160979 | −0.856285373 | −1.096220349 |
| chr1 | 173400380 | 173400640 | 0.001160814 | −1.188946315 | −1.048218443 |
| chr1 | 173400060 | 173400960 | 0.001160814 | −1.188946315 | −1.048218443 |
| chr20 | 32580520 | 32582480 | −0.001160756 | −1.06085479 | −1.169362548 |
| chr6 | 30512200 | 30513080 | 0.001160755 | −1.263321596 | −1.024929143 |
| chr16 | 9184300 | 9184580 | 0.001160667 | −1.12465124 | −0.95394327 |
| chr11 | 60372580 | 60372840 | −0.001160386 | −0.894082614 | −1.1217568 |
| chr21 | 34960160 | 34961500 | 0.001160258 | −1.016462904 | −0.755562528 |
| chr3 | 181683100 | 181683700 | 0.001159893 | −1.196836381 | −1.104979824 |
| chr21 | 35770040 | 35770560 | −0.001159836 | −0.908009057 | −1.012960625 |
| chr7 | 100340440 | 100340680 | 0.001159455 | −1.221055851 | −1.027761987 |
| chr7 | 100340440 | 100340680 | 0.001159455 | −1.221055851 | −1.027761987 |
| chr10 | 104263420 | 104264160 | 0.001159322 | −1.23639184 | −1.036876378 |
| chr9 | 28479660 | 28480140 | −0.001159284 | −0.825999734 | −0.993378597 |
| chr20 | 23058000 | 23058460 | −0.001159198 | −1.083616082 | −1.14630394 |
| chr20 | 60757200 | 60758740 | 0.001159164 | −1.25712566 | −1.052461836 |
| chr21 | 45552920 | 45554200 | 0.001158813 | −1.101865625 | −0.865858677 |
| chr3 | 54769820 | 54770380 | −0.001158157 | −1.112168161 | −1.296372658 |
| chr8 | 98709300 | 98709580 | 0.001157994 | −1.146682618 | −1.0673386 |
| chr2 | 47669060 | 47669620 | 0.00115795 | −1.306146494 | −1.154832926 |
| chr2 | 47669060 | 47669620 | 0.00115795 | −1.306146494 | −1.154832926 |
| chr14 | 74550400 | 74552440 | 0.001157927 | −1.319432116 | −1.080244258 |
| chr3 | 14885500 | 14886000 | −0.001157844 | −1.252930822 | −1.349997114 |
| chr2 | 46843560 | 46844760 | 0.001157825 | −1.267578254 | −1.07105819 |
| chr5 | 85677020 | 85677560 | −0.001157778 | −1.147164692 | −1.414761937 |
| chr1 | 17362960 | 17363500 | −0.001157667 | −0.962752449 | −1.189710439 |
| chr1 | 200772060 | 200772340 | 0.001157657 | −1.12216908 | −0.924801941 |
| chr8 | 100922580 | 100923180 | −0.00115764 | −1.069832622 | −1.3113692 |
| chr2 | 122512300 | 122513820 | 0.001157548 | −1.285328164 | −1.083833534 |
| chr5 | 81147200 | 81148500 | 0.001157311 | −1.248715161 | −1.071687249 |
| chr15 | 61639940 | 61640720 | −0.001157195 | −0.973152379 | −1.195932383 |
| chr17 | 8065160 | 8065460 | 0.001157072 | −1.076417684 | −0.957658533 |
| chr7 | 26903240 | 26904620 | 0.001157061 | −1.17099827 | −1.065759221 |
| chr9 | 101896860 | 101897320 | −0.001156841 | −0.880464615 | −1.064110436 |
| chr9 | 96755820 | 96756260 | −0.001156547 | −0.884622736 | −1.0819933 |
| chr17 | 29814280 | 29815580 | 0.001156523 | −1.223729984 | −1.04668171 |
| chr20 | 43050340 | 43050680 | −0.00115618 | −1.072718887 | −1.248186933 |
| chr6 | 143949860 | 143950340 | −0.001155744 | −1.028930335 | −1.171581305 |
| chr19 | 3985100 | 3985960 | 0.00115532 | −1.254780252 | −1.109181652 |
| chr5 | 158639560 | 158639860 | −0.001155023 | −1.045786839 | −1.229242632 |
| chr6 | 57533000 | 57533280 | −0.001155004 | −0.946264886 | −1.179919455 |
| chr19 | 56165040 | 56167340 | 0.001154836 | −1.227349805 | −1.009225469 |
| chr3 | 103912720 | 103913100 | 0.001154548 | −1.087411468 | −1.097066674 |
| chr7 | 148830580 | 148830900 | −0.00115441 | −1.030407896 | −1.175483978 |
| chr1 | 30952040 | 30952740 | 0.001154314 | −1.339983695 | −1.11565532 |
| chr8 | 1377960 | 1378440 | −0.001154052 | −1.054691318 | −1.205925002 |
| chr7 | 149019860 | 149020220 | −0.001153828 | −1.017918886 | −1.270510171 |
| chr6 | 33395520 | 33396500 | −0.001153802 | −1.122606036 | −1.280788396 |
| chr2 | 9695760 | 9696440 | 0.001153765 | −1.377184761 | −1.187619018 |
| chr8 | 135358440 | 135358980 | −0.001153622 | −0.94290058 | −1.203517823 |
| chr2 | 74681800 | 74682320 | 0.001153463 | −1.277226257 | −1.057394607 |
| chr19 | 46366120 | 46366760 | 0.001153368 | −1.190477347 | −1.006143671 |
| chr19 | 53238160 | 53238560 | 0.00115328 | −1.157818905 | −0.994029112 |
| chr17 | 62213780 | 62214080 | 0.001153076 | −1.180513158 | −1.109902111 |
| chr17 | 62213780 | 62214080 | 0.001153076 | −1.180513158 | −1.109902111 |
| chr2 | 58121100 | 58121580 | −0.001152924 | −1.140086665 | −1.296845704 |
| chr6 | 26740200 | 26740620 | 0.001152898 | −1.272166092 | −1.103000734 |
| chr8 | 52717620 | 52718200 | −0.001152865 | −0.984340563 | −1.107936789 |
| chr1 | 36839420 | 36839700 | 0.001152862 | −1.086002107 | −0.923740519 |
| chr19 | 38261320 | 38261720 | −0.001152826 | −0.845101719 | −1.039047155 |
| chr1 | 63909940 | 63910700 | 0.001152806 | −1.113959747 | −1.081563155 |
| chr2 | 212182440 | 212182960 | −0.001152764 | −1.016185258 | −1.258278331 |
| chr19 | 8578380 | 8579280 | 0.001152763 | −1.217373753 | −1.040826891 |
| chr15 | 69109820 | 69111320 | 0.00115243 | −1.235872118 | −1.03122407 |
| chr19 | 47759600 | 47760900 | 0.001152229 | −1.282941031 | −1.162570256 |
| chr2 | 33823880 | 33824980 | 0.001152214 | −1.258830005 | −0.963859515 |
| chr22 | 36924800 | 36925760 | 0.001151941 | −1.16641726 | −1.003102165 |
| chr22 | 36924440 | 36926120 | 0.001151941 | −1.16641726 | −1.003102165 |
| chr12 | 103276140 | 103276700 | −0.001151901 | −0.949671173 | −1.203309099 |
| chr12 | 20303040 | 20303700 | −0.001151738 | −0.979336196 | −1.214415519 |
| chr3 | 31613280 | 31613920 | −0.001151206 | −1.12570122 | −1.400253337 |
| chr15 | 59396620 | 59397940 | 0.001151175 | −1.115879175 | −0.933063899 |
| chr5 | 54267800 | 54268380 | 0.001151074 | −1.12201684 | −1.120087074 |
| chr5 | 62767960 | 62768540 | −0.001151073 | −0.994029719 | −1.244690302 |
| chr3 | 132135820 | 132136940 | 0.001150902 | −1.304900497 | −1.116165873 |
| chr2 | 45877720 | 45879060 | −0.001150889 | −1.147398116 | −1.155134052 |
| chr17 | 73774980 | 73776260 | 0.001150875 | −1.205246975 | −0.988614655 |
| chr2 | 54013520 | 54014800 | 0.001150683 | −1.269216102 | −1.050751903 |
| chr2 | 215039780 | 215040180 | −0.0011505 | −0.957461695 | −1.191715987 |
| chr2 | 215039760 | 215040200 | −0.0011505 | −0.957461695 | −1.191715987 |
| chr7 | 98740060 | 98742100 | −0.001150243 | −1.090907952 | −1.203917474 |
| chr7 | 156399120 | 156400560 | 0.001150183 | −1.26632252 | −1.048618147 |
| chr5 | 163421000 | 163421400 | −0.001149988 | −0.813497125 | −1.0338553 |
| chr12 | 83032920 | 83033260 | −0.001149777 | −1.204069112 | −1.403848943 |
| chr17 | 27920040 | 27921100 | −0.00114974 | −0.967462682 | −1.13416305 |
| chr14 | 71786800 | 71788360 | 0.001149145 | −1.276591654 | −1.151489642 |
| chr7 | 117251840 | 117252340 | −0.001149022 | −1.061645882 | −1.268057562 |
| chr19 | 14227980 | 14229360 | 0.001148937 | −1.288324785 | −1.151636523 |
| chr8 | 71421960 | 71422420 | −0.001148854 | −1.192403294 | −1.28360466 |
| chr10 | 25830700 | 25831080 | −0.001148789 | −0.960559903 | −1.198284386 |
| chr10 | 25830680 | 25831100 | −0.001148789 | −0.960559903 | −1.198284386 |
| chr3 | 61000440 | 61000720 | −0.001148758 | −0.970930753 | −1.12355145 |
| chr1 | 154909020 | 154909940 | −0.001148692 | −1.115134139 | −1.251517765 |
| chr19 | 3135860 | 3136160 | 0.001148523 | −1.119468043 | −0.893894558 |
| chr12 | 121084780 | 121085040 | 0.001147863 | −1.228663943 | −1.056367317 |
| chr2 | 55276120 | 55276440 | 0.001147825 | −1.196413275 | −0.970594386 |
| chr19 | 50094280 | 50094860 | 0.001147814 | −1.067569621 | −0.836827468 |
| chr20 | 24937320 | 24937980 | 0.001147724 | −1.349635068 | −1.190116029 |
| chr10 | 32545340 | 32546220 | 0.001147685 | −1.184052692 | −0.968772217 |
| chr17 | 79790680 | 79791800 | −0.001147604 | −0.86371908 | −1.131252054 |
| chr14 | 27910060 | 27910500 | 0.00114721 | −1.187098455 | −1.142793431 |
| chr10 | 87746980 | 87747500 | 0.001147081 | −1.107161464 | −0.887471861 |
| chr3 | 115435340 | 115435820 | −0.001146881 | −1.075597564 | −1.330965788 |
| chr9 | 14938120 | 14938560 | −0.001146683 | −0.771741988 | −1.001898368 |
| chr3 | 157230860 | 157232240 | −0.001146144 | −1.101415368 | −1.247849503 |
| chr3 | 109698340 | 109699060 | −0.001146139 | −1.044159269 | −1.347189651 |
| chr4 | 135510240 | 135510720 | −0.00114606 | −1.095701736 | −1.294169756 |
| chr19 | 13262440 | 13262900 | 0.001145751 | −1.215582025 | −1.093765608 |
| chr10 | 32855620 | 32856220 | −0.001145677 | −1.032092992 | −1.251288351 |
| chr10 | 24617740 | 24618500 | −0.001145499 | −0.896685776 | −1.270081323 |
| chr17 | 48228100 | 48228780 | 0.00114539 | −1.18878053 | −1.039673439 |
| chr1 | 247553120 | 247554040 | 0.00114519 | −1.234670118 | −0.946174744 |
| chr12 | 9246780 | 9247180 | −0.001145179 | −1.008227867 | −1.213174878 |
| chr9 | 109304640 | 109305220 | −0.001145089 | −0.752187211 | −1.017954024 |
| chr1 | 9188460 | 9189980 | 0.001144939 | −1.280231811 | −1.110605116 |
| chr3 | 98904340 | 98904680 | −0.001144794 | −0.911784238 | −1.080512948 |
| chr5 | 157952120 | 157952640 | −0.001144639 | −0.952520494 | −1.219433847 |
| chr13 | 63226660 | 63227020 | −0.001144586 | −1.04516006 | −1.22921002 |
| chr17 | 80967440 | 80968220 | 0.001144541 | −1.075414937 | −0.850356935 |
| chr2 | 26204660 | 26206200 | 0.001144314 | −1.283640139 | −1.050053693 |
| chr11 | 70116060 | 70117620 | 0.00114388 | −1.43554101 | −1.258910385 |
| chr20 | 4065940 | 4066220 | 0.00114381 | −1.286775169 | −1.165351583 |
| chr20 | 4065620 | 4066540 | 0.00114381 | −1.286775169 | −1.165351583 |
| chr11 | 105947560 | 105948920 | 0.001143779 | −1.367300636 | −1.137511933 |
| chr21 | 45206580 | 45207340 | 0.001143374 | −1.132753201 | −0.956571469 |
| chr2 | 237399300 | 237400120 | −0.001142881 | −1.032320441 | −1.2470809 |
| chr7 | 122372460 | 122373180 | −0.001142651 | −1.019160159 | −1.23577733 |
| chr2 | 170681140 | 170681680 | 0.001142615 | −1.235611074 | −0.983610349 |
| chr4 | 140221940 | 140223280 | 0.001142559 | −1.221916145 | −1.061781908 |
| chr1 | 32644460 | 32646120 | 0.001142229 | −1.237492556 | −1.087496615 |
| chr12 | 61485420 | 61485960 | 0.001141987 | −1.170675557 | −1.107306593 |
| chr14 | 54790200 | 54790700 | −0.001141833 | −1.036590322 | −1.320808643 |
| chr3 | 157305860 | 157306280 | −0.00114176 | −0.831238432 | −1.204942446 |
| chr1 | 101004620 | 101005900 | −0.001141669 | −0.993153381 | −1.044981587 |
| chr11 | 18752500 | 18753000 | −0.001141517 | −0.961537336 | −1.192921976 |
| chr20 | 33543400 | 33543900 | 0.001141391 | −1.219379966 | −0.99748424 |
| chr16 | 72042120 | 72043120 | 0.001141379 | −1.178841917 | −0.999855075 |
| chr15 | 67238960 | 67239480 | −0.001141216 | −0.91957505 | −1.03915644 |
| chr17 | 73105380 | 73106640 | 0.001141133 | −1.274023953 | −1.121380508 |
| chr11 | 64889020 | 64890200 | 0.001141118 | −1.29617486 | −1.08761099 |
| chr5 | 156209020 | 156209400 | −0.001141076 | −1.036171464 | −1.291133091 |
| chr11 | 37766520 | 37766980 | −0.001141055 | −0.974494899 | −1.233706905 |
| chr4 | 106422400 | 106422880 | −0.001140858 | −0.966056867 | −1.26074684 |
| chr19 | 32237920 | 32239020 | 0.001140848 | −1.040760554 | −0.983505996 |
| chr4 | 88565600 | 88566500 | −0.001140812 | −0.971844364 | −1.214576379 |
| chr19 | 31474520 | 31475100 | −0.001140791 | −1.035429283 | −1.282255786 |
| chr3 | 164783040 | 164783500 | −0.001140521 | −1.078169131 | −1.247971878 |
| chr8 | 103062680 | 103063340 | −0.00114035 | −1.098909839 | −1.351601297 |
| chr1 | 231493620 | 231493880 | −0.001140334 | −0.98517739 | −1.068557478 |
| chr9 | 24781380 | 24781880 | −0.001140227 | −0.776320105 | −0.900631848 |
| chr19 | 18716720 | 18718260 | −0.001140097 | −0.906232641 | −1.11638837 |
| chr19 | 18716720 | 18718260 | −0.001140097 | −0.906232641 | −1.11638837 |
| chr17 | 40830260 | 40830560 | 0.001139697 | −1.160604825 | −0.964857947 |
| chr17 | 17175080 | 17175420 | 0.001139209 | −1.051180074 | −0.936696343 |
| chr17 | 17175080 | 17175420 | 0.001139209 | −1.051180074 | −0.936696343 |
| chr10 | 5562940 | 5563420 | −0.001139207 | −0.949123632 | −1.154628063 |
| chr11 | 7044320 | 7044980 | −0.001138811 | −1.039280559 | −1.279936486 |
| chr7 | 124130820 | 124131280 | −0.001138746 | −1.108262752 | −1.277732753 |
| chr8 | 95565260 | 95566100 | 0.001138682 | −1.219106395 | −1.022943145 |
| chr1 | 97955680 | 97956200 | −0.001138465 | −0.93590336 | −1.194983389 |
| chr12 | 123937660 | 123937860 | −0.00113838 | −0.957883178 | −1.095001399 |
| chr2 | 78224000 | 78224500 | −0.001138374 | −0.975245582 | −1.24014951 |
| chr16 | 23607180 | 23608360 | 0.001138291 | −0.965954945 | −0.845272721 |
| chr2 | 161405320 | 161405760 | −0.001138166 | −0.950419834 | −1.156582055 |
| chr17 | 80407720 | 80408920 | 0.001138067 | −1.134415215 | −0.975359496 |
| chr5 | 39246960 | 39247340 | 0.001138024 | −1.247408866 | −1.118613665 |
| chr6 | 126216960 | 126217380 | −0.001137896 | −0.944552842 | −1.142391215 |
| chr2 | 70815480 | 70815960 | −0.001137791 | −0.971748768 | −1.255843372 |
| chr7 | 69469200 | 69469580 | −0.001137726 | −0.996554093 | −1.216783366 |
| chr9 | 140116360 | 140116940 | 0.001137629 | −1.178997363 | −1.014464114 |
| chr4 | 19502880 | 19503220 | −0.001137579 | −0.972754329 | −1.15144365 |
| chr2 | 108687880 | 108688340 | −0.001137503 | −0.886677846 | −1.116739902 |
| chr6 | 24719580 | 24721740 | 0.001137373 | −1.329523014 | −1.122641954 |
| chr4 | 115654640 | 115655300 | −0.001137332 | −1.023237852 | −1.181445731 |
| chr7 | 15200980 | 15201500 | 0.00113726 | −1.020399351 | −1.030941367 |
| chr8 | 88144720 | 88145180 | −0.001136728 | −0.860043047 | −1.164175012 |
| chr8 | 35445320 | 35445740 | −0.001136429 | −0.966708666 | −1.181195106 |
| chr4 | 180599960 | 180600820 | 0.001136428 | −1.264461892 | −1.123760265 |
| chr3 | 83965500 | 83966400 | −0.001136405 | −0.975170772 | −1.256957258 |
| chr5 | 15096620 | 15096820 | 0.001136293 | −1.311295098 | −1.135702128 |
| chr5 | 15096620 | 15096820 | 0.001136293 | −1.311295098 | −1.135702128 |
| chr4 | 86396380 | 86397380 | 0.001136018 | −1.306353373 | −1.119241079 |
| chr4 | 92920440 | 92920840 | −0.001135704 | −1.016483318 | −1.223449845 |
| chr3 | 191641820 | 191642300 | −0.001135698 | −0.959005572 | −1.120434716 |
| chr3 | 156855280 | 156855560 | 0.001135634 | −1.112213549 | −0.970979607 |
| chr8 | 100509160 | 100509840 | −0.001135509 | −1.13363627 | −1.353619757 |
| chr3 | 144344520 | 144345000 | −0.001135415 | −0.926451006 | −1.106674744 |
| chr5 | 1651440 | 1652160 | −0.001135342 | −1.172678182 | −1.226548804 |
| chr3 | 13133360 | 13134260 | 0.001135294 | −1.283927143 | −1.129549081 |
| chr3 | 13133340 | 13134280 | 0.001135294 | −1.283927143 | −1.129549081 |
| chr9 | 77392040 | 77392280 | 0.001135176 | −0.979112407 | −0.742126813 |
| chr1 | 90541440 | 90541800 | −0.001134799 | −0.873516057 | −1.076155547 |
| chr15 | 50646380 | 50647820 | 0.001134656 | −1.020069345 | −0.825723727 |
| chr6 | 114180080 | 114180400 | 0.001134613 | −1.399820903 | −1.234756282 |
| chr12 | 109535160 | 109536580 | 0.001134564 | −1.35404279 | −1.185623256 |
| chr19 | 56631580 | 56633420 | 0.001134498 | −1.203354235 | −1.101927637 |
| chr1 | 201099940 | 201100500 | −0.001134496 | −0.961620831 | −1.224764301 |
| chr11 | 29481440 | 29482020 | 0.001134462 | −1.256926898 | −1.136622933 |
| chr11 | 29481400 | 29482060 | 0.001134462 | −1.256926898 | −1.136622933 |
| chr13 | 65637600 | 65638380 | 0.001134392 | −1.427887725 | −1.245241542 |
| chr14 | 85558780 | 85559220 | −0.001134371 | −1.040248671 | −1.235539049 |
| chr6 | 16128900 | 16130160 | 0.001134291 | −1.235075535 | −0.998159502 |
| chr13 | 63316480 | 63317060 | −0.001133969 | −1.14213951 | −1.357935551 |
| chr16 | 29972980 | 29974020 | −0.001133963 | −0.88812365 | −0.969309645 |
| chr1 | 229763720 | 229764040 | −0.001133925 | −1.047537926 | −1.25463225 |
| chr19 | 49121880 | 49123220 | 0.001133818 | −1.308444168 | −1.118023919 |
| chr1 | 245206000 | 245206300 | 0.001133696 | −1.277422257 | −1.09044818 |
| chr12 | 54718420 | 54719040 | 0.001133561 | −1.419043888 | −1.264571542 |
| chr8 | 77401640 | 77402100 | −0.001133247 | −0.933537813 | −1.189754947 |
| chr9 | 91650660 | 91650940 | −0.001132976 | −0.789505842 | −0.941251888 |
| chr15 | 85196640 | 85198720 | 0.001132793 | −1.114346531 | −0.887929971 |
| chr1 | 97956480 | 97956960 | 0.001132628 | −1.194546859 | −1.158963043 |
| chr7 | 100381380 | 100381640 | 0.001132552 | −1.10992712 | −0.921840432 |
| chr19 | 49588200 | 49589120 | 0.001132524 | −1.128891559 | −1.038462198 |
| chr21 | 19605080 | 19605480 | −0.001132358 | −0.926850289 | −1.102055958 |
| chr2 | 187102220 | 187102780 | −0.001132213 | −1.110573007 | −1.320226397 |
| chr2 | 187102200 | 187102800 | −0.001132213 | −1.110573007 | −1.320226397 |
| chr5 | 174477600 | 174478300 | −0.001132129 | −1.041642898 | −1.290815934 |
| chr16 | 26911120 | 26912160 | −0.001131767 | −0.839262886 | −0.973684583 |
| chr1 | 69124700 | 69125220 | −0.001131727 | −0.951958155 | −1.218081054 |
| chr20 | 32580780 | 32582460 | −0.001131685 | −1.043499776 | −1.100010994 |
| chr19 | 4400080 | 4400860 | 0.001131635 | −1.203576894 | −1.039568734 |
| chr11 | 33294160 | 33294600 | 0.001131487 | −1.357886136 | −1.16699939 |
| chr16 | 65746500 | 65746900 | −0.001131469 | −0.918331872 | −1.107158579 |
| chr12 | 106129120 | 106129500 | −0.001131124 | −1.084334693 | −1.207842955 |
| chr12 | 106129100 | 106129520 | −0.001131124 | −1.084334693 | −1.207842955 |
| chr10 | 36017820 | 36018380 | −0.001131054 | −1.022276408 | −1.289904546 |
| chr2 | 26256440 | 26257360 | 0.001130928 | −1.375107758 | −1.195286368 |
| chr2 | 162493360 | 162493780 | 0.001130799 | −1.2055101 | −1.079878731 |
| chr21 | 45826640 | 45827320 | 0.001130798 | −0.982912739 | −0.842889837 |
| chr6 | 64755580 | 64756220 | −0.001130536 | −1.064343084 | −1.33129667 |
| chr12 | 84653940 | 84654420 | −0.001130487 | −1.041948056 | −1.299603064 |
| chr18 | 52603120 | 52603780 | −0.001130431 | −1.116600604 | −1.415878175 |
| chr19 | 2189780 | 2190060 | 0.001130251 | −1.048554456 | −0.799100618 |
| chr12 | 20285140 | 20285600 | −0.00113018 | −1.004129911 | −1.235126711 |
| chr19 | 3990200 | 3990520 | 0.001130116 | −1.148484323 | −0.951790286 |
| chr19 | 3990200 | 3990520 | 0.001130116 | −1.148484323 | −0.951790286 |
| chr12 | 56709540 | 56710800 | 0.001130108 | −1.153849535 | −1.012015065 |
| chr20 | 5595660 | 5596060 | 0.001129569 | −1.152239028 | −1.034978512 |
| chr1 | 66597060 | 66597460 | −0.001129219 | −0.917447877 | −1.166034845 |
| chr5 | 472240 | 473960 | −0.001129087 | −1.075290863 | −1.141967456 |
| chr2 | 204501160 | 204501540 | −0.001128942 | −0.975684563 | −1.164734583 |
| chr11 | 6675940 | 6677460 | 0.001128689 | −1.326973437 | −1.148867195 |
| chr2 | 23123160 | 23123880 | −0.001128497 | −0.934205448 | −1.174256504 |
| chr18 | 42994520 | 42994920 | −0.00112845 | −1.101097498 | −1.287193263 |
| chr1 | 243911740 | 243912660 | 0.001128436 | −1.124392772 | −0.997174799 |
| chr3 | 100309180 | 100310060 | 0.001128352 | −1.294734666 | −1.010365893 |
| chr13 | 62236540 | 62237260 | −0.001128226 | −1.043470071 | −1.217184028 |
| chr5 | 18929820 | 18930260 | −0.001127978 | −0.839520793 | −1.055777967 |
| chr21 | 34960360 | 34961500 | 0.001127974 | −1.085764994 | −0.848697908 |
| chr2 | 64506640 | 64507000 | −0.001127947 | −1.081725564 | −1.207333137 |
| chr2 | 64506560 | 64507080 | −0.001127947 | −1.081725564 | −1.207333137 |
| chr4 | 71381800 | 71382360 | −0.001127866 | −1.183401538 | −1.372196705 |
| chr19 | 29687380 | 29688000 | −0.001127725 | −0.931260045 | −1.203890773 |
| chr11 | 30340300 | 30341060 | −0.001127677 | −1.103901593 | −1.337669185 |
| chr1 | 239567840 | 239568260 | −0.001127371 | −1.064845959 | −1.169035577 |
| chr8 | 42126800 | 42127300 | −0.001127365 | −1.152372883 | −1.284789458 |
| chr5 | 173596520 | 173597100 | −0.00112701 | −1.005413784 | −1.279390155 |
| chr1 | 16623160 | 16623620 | −0.001126853 | −0.965628285 | −1.105348608 |
| chr15 | 41522520 | 41524080 | 0.001126646 | −1.223560492 | −1.008101958 |
| chr20 | 8497160 | 8497500 | −0.00112664 | −1.094770177 | −1.296144999 |
| chr20 | 42274140 | 42274340 | 0.001126413 | −1.315524646 | −1.162828637 |
| chr20 | 42274140 | 42274340 | 0.001126413 | −1.315524646 | −1.162828637 |
| chr1 | 217657740 | 217658260 | 0.001125802 | −1.173666385 | −1.104115239 |
| chr3 | 153051400 | 153052160 | −0.001125438 | −0.99451193 | −1.208137389 |
| chr3 | 83965460 | 83966380 | −0.001125365 | −1.002177203 | −1.273529113 |
| chr6 | 92065560 | 92066200 | −0.00112525 | −1.032058478 | −1.253813343 |
| chr1 | 36869640 | 36869920 | −0.001124906 | −0.866356642 | −1.106734375 |
| chr5 | 67558020 | 67558540 | −0.00112471 | −1.072569993 | −1.198287286 |
| chr4 | 180599980 | 180600820 | 0.001124549 | −1.228493608 | −1.095170229 |
| chr3 | 196438660 | 196439660 | 0.001124377 | −1.410483211 | −1.22348077 |
| chr11 | 535020 | 536200 | 0.001124199 | −1.394803128 | −1.229342641 |
| chr7 | 107055900 | 107056960 | 0.00112409 | −1.088646505 | −0.938961414 |
| chr2 | 117801200 | 117802720 | 0.001123844 | −1.233730088 | −1.185205102 |
| chr20 | 8141580 | 8142060 | 0.001123814 | −0.970030219 | −0.977849271 |
| chr19 | 48770300 | 48770600 | 0.001123463 | −1.135730546 | −1.006824031 |
| chr4 | 129432820 | 129433300 | −0.001123462 | −1.161003896 | −1.29064776 |
| chr1 | 102559220 | 102559800 | −0.001123384 | −1.11370035 | −1.177607611 |
| chr4 | 21347300 | 21348140 | −0.001122937 | −1.02068659 | −1.192800604 |
| chr8 | 145687440 | 145688840 | −0.00112291 | −1.175756771 | −1.228881145 |
| chr2 | 4158380 | 4158900 | 0.001122898 | −1.332918825 | −1.128579419 |
| chr2 | 4158380 | 4158900 | 0.001122898 | −1.332918825 | −1.128579419 |
| chr10 | 3901900 | 3902260 | −0.001122748 | −1.042569049 | −1.250764133 |
| chr9 | 130632220 | 130632500 | 0.001122689 | −1.094774663 | −0.873974171 |
| chr2 | 10219600 | 10220920 | 0.001122665 | −1.179083573 | −1.035567809 |
| chr7 | 122637900 | 122638440 | −0.001122655 | −0.886989278 | −1.196361455 |
| chr6 | 148764420 | 148764760 | −0.001122607 | −1.038831046 | −1.224342262 |
| chr6 | 22481600 | 22482120 | −0.001122238 | −0.885240307 | −1.209091425 |
| chr4 | 172376420 | 172376620 | 0.001122237 | −1.426327096 | −1.268411574 |
| chr9 | 33264020 | 33265920 | 0.0011222 | −1.173668256 | −1.037777493 |
| chr19 | 31839740 | 31842060 | 0.001121394 | −1.172644063 | −0.969488876 |
| chr19 | 58919740 | 58920140 | 0.001121206 | −1.268373501 | −1.102997442 |
| chr11 | 45168040 | 45169320 | 0.001121116 | −1.350107732 | −1.165279993 |
| chr22 | 38239380 | 38240680 | 0.001121012 | −1.044212692 | −0.851669495 |
| chr8 | 88914080 | 88914680 | −0.00112101 | −1.117759011 | −1.328287316 |
| chr19 | 40696760 | 40698420 | 0.001120971 | −1.194777212 | −0.970914781 |
| chr21 | 35445100 | 35446600 | −0.001120914 | −1.08052349 | −1.180491364 |
| chr19 | 46520040 | 46520380 | 0.001120854 | −1.289323129 | −1.137254443 |
| chr3 | 50337740 | 50337940 | −0.001120559 | −0.828507476 | −0.945247469 |
| chr19 | 2950540 | 2951280 | 0.001120494 | −1.075599092 | −0.946001874 |
| chr14 | 99247220 | 99247920 | −0.001120115 | −1.033460546 | −1.285129205 |
| chr1 | 231015140 | 231015960 | 0.001120044 | −1.201718553 | −0.885925716 |
| chr3 | 89059260 | 89059740 | −0.001119815 | −1.100841778 | −1.202583128 |
| chr7 | 36797240 | 36797900 | −0.001119331 | −1.065118594 | −1.305357555 |
| chr7 | 5405700 | 5405960 | −0.00111932 | −0.960656086 | −1.058456147 |
| chr17 | 43393900 | 43395480 | 0.001119296 | −1.177521408 | −1.036676354 |
| chr4 | 155651900 | 155652280 | −0.001119259 | −0.775520003 | −1.067081554 |
| chr4 | 177240500 | 177241880 | 0.001119171 | −1.342779907 | −1.149136899 |
| chr19 | 33571920 | 33572240 | 0.001119029 | −1.241294754 | −1.067876961 |
| chr10 | 34955580 | 34955820 | −0.001118756 | −0.831937311 | −0.987734967 |
| chr8 | 145132760 | 145134020 | 0.001118737 | −1.277098199 | −1.110055454 |
| chr19 | 42103640 | 42104560 | 0.001118399 | −1.151407518 | −0.971630291 |
| chr14 | 92320580 | 92321020 | 0.001118308 | −1.184014416 | −0.956207613 |
| chr19 | 5084160 | 5084860 | −0.001118278 | −1.059710319 | −1.298218786 |
| chr16 | 67142980 | 67144460 | 0.001118267 | −1.274435382 | −1.031651884 |
| chr15 | 76109940 | 76110320 | 0.001118142 | −0.912242945 | −0.846722625 |
| chr6 | 33289820 | 33291160 | 0.001118023 | −1.056676032 | −0.909268063 |
| chr19 | 56110320 | 56112400 | 0.001117962 | −1.023340817 | −0.988172521 |
| chr8 | 76301960 | 76302440 | −0.001117917 | −1.007777762 | −1.242820893 |
| chr1 | 228369840 | 228370680 | 0.001117752 | −1.225032704 | −1.106815887 |
| chr8 | 137838340 | 137838900 | −0.001117541 | −1.092193816 | −1.29642051 |
| chr10 | 111767280 | 111768520 | −0.001117293 | −0.786626739 | −0.877342738 |
| chr20 | 60941620 | 60943020 | −0.001117245 | −0.913677156 | −0.987785148 |
| chr16 | 84481040 | 84481700 | −0.001117133 | −0.958864366 | −1.197615923 |
| chr15 | 96103540 | 96104020 | 0.001117001 | −1.042403779 | −1.016709208 |
| chr6 | 79263300 | 79264120 | −0.001116893 | −1.084551423 | −1.282262551 |
| chr19 | 16738280 | 16739880 | 0.001116883 | −1.283490539 | −1.161702176 |
| chr13 | 83752300 | 83752800 | −0.001116802 | −0.982574501 | −1.150677658 |
| chr12 | 17439480 | 17439900 | −0.001116519 | −1.004948823 | −1.267973689 |
| chr14 | 24610640 | 24611120 | 0.001116455 | −1.299304629 | −1.17497304 |
| chr14 | 24610620 | 24611140 | 0.001116455 | −1.299304629 | −1.17497304 |
| chr19 | 41076060 | 41077360 | 0.00111641 | −1.180481949 | −1.060701466 |
| chr1 | 152452400 | 152452860 | −0.001116347 | −1.075847208 | −1.196734636 |
| chr1 | 152452380 | 152452880 | −0.001116347 | −1.075847208 | −1.196734636 |
| chr2 | 124074060 | 124074660 | −0.001116339 | −1.067357276 | −1.271746145 |
| chr11 | 69191700 | 69192420 | 0.001115972 | −1.131786822 | −1.007487428 |
| chr21 | 35770040 | 35770500 | −0.001115937 | −0.964110681 | −1.064844399 |
| chr14 | 27162300 | 27162980 | −0.001115893 | −1.070561085 | −1.176495445 |
| chr2 | 226618300 | 226618740 | 0.00111587 | −0.931024566 | −1.189451748 |
| chr2 | 226618280 | 226618760 | −0.00111587 | −0.931024566 | −1.189451748 |
| chr14 | 85364120 | 85364580 | −0.001115834 | −1.175280367 | −1.326095968 |
| chr16 | 27199700 | 27200340 | 0.001115761 | −1.260707376 | −1.116895942 |
| chr18 | 29443820 | 29444200 | −0.001115705 | −0.926894587 | −1.184438149 |
| chr3 | 167443340 | 167443820 | −0.001115384 | −1.121556921 | −1.23329988 |
| chr14 | 76415500 | 76415960 | 0.001115382 | −1.133198006 | −1.090222245 |
| chr1 | 60762080 | 60762500 | −0.001115358 | −0.982789554 | −1.116177628 |
| chr2 | 2113500 | 2113920 | −0.001114995 | −1.019835229 | −1.134846458 |
| chr16 | 89783220 | 89783700 | −0.001114951 | −0.999204351 | −1.118888071 |
| chr21 | 25248920 | 25249240 | −0.001114911 | −0.802651704 | −0.956548276 |
| chr10 | 102672140 | 102673320 | 0.001114825 | −1.19200308 | −1.006318755 |
| chr3 | 183068780 | 183069240 | 0.001114713 | −1.221137217 | −1.140934269 |
| chr8 | 3380840 | 3382380 | 0.001114553 | −1.184901933 | −1.014544373 |
| chr12 | 15397620 | 15398140 | 0.00111455 | −1.30934827 | −1.156424928 |
| chr9 | 29913660 | 29914220 | −0.001114521 | −0.816202868 | −1.085463902 |
| chr21 | 46256260 | 46256540 | −0.001114516 | −0.917816936 | −0.925514626 |
| chr5 | 28966980 | 28967660 | −0.00111415 | −1.048729 | −1.295236978 |
| chr19 | 41283340 | 41284980 | 0.001113898 | −1.10285478 | −0.952502089 |
| chr14 | 55030800 | 55031020 | 0.0011136 | −1.156533503 | −1.049368055 |
| chr14 | 55737260 | 55738740 | 0.001113468 | −1.33420851 | −1.040059857 |
| chr2 | 46847720 | 46847980 | −0.00111319 | −0.863555477 | −1.056419343 |
| chr2 | 46847660 | 46848040 | −0.00111319 | −0.863555477 | −1.056419343 |
| chr3 | 13983660 | 13984040 | −0.001113138 | −0.830867367 | −1.185744431 |
| chr2 | 193765260 | 193765900 | −0.001113119 | −1.078239583 | −1.327371432 |
| chr4 | 27050000 | 27050300 | 0.00111294 | −1.209494807 | −1.05380676 |
| chr1 | 32014200 | 32015120 | 0.001112905 | −1.252102869 | −1.014673637 |
| chr20 | 57617280 | 57618520 | 0.001112624 | −1.303587265 | −1.144255648 |
| chr5 | 161111580 | 161112100 | −0.001112329 | −1.100409392 | −1.319142569 |
| chr2 | 99224460 | 99225660 | 0.001112213 | −1.369638572 | −1.157429396 |
| chr4 | 127533920 | 127534460 | −0.001112 | −1.129557742 | −1.303446464 |
| chr4 | 108910520 | 108911480 | 0.001111941 | −1.174085499 | −0.941813424 |
| chr6 | 48552000 | 48552380 | −0.001111881 | −0.853231275 | −1.203657782 |
| chr1 | 90800040 | 90800980 | −0.00111188 | −1.067844192 | −1.307120189 |
| chr11 | 90458960 | 90459360 | −0.001111741 | −0.914072172 | −1.12725033 |
| chr5 | 167218120 | 167218760 | −0.001111722 | −1.127266981 | −1.329005372 |
| chr17 | 80967640 | 80968180 | 0.001111385 | −1.198388215 | −1.020425933 |
| chr21 | 20526120 | 20526520 | −0.00111119 | −0.846278415 | −0.999212391 |
| chr21 | 20526080 | 20526560 | −0.00111119 | −0.846278415 | −0.999212391 |
| chr2 | 137188660 | 137189220 | −0.001111118 | −1.011205108 | −1.210302551 |
| chr13 | 55876980 | 55877340 | −0.001111035 | −1.028383806 | −1.213789354 |
| chr7 | 30174080 | 30175340 | 0.001111001 | −1.169601408 | −1.032121283 |
| chr11 | 65097440 | 65097700 | 0.00111079 | −1.278904405 | −1.13729655 |
| chr3 | 38203780 | 38204040 | −0.001110736 | −1.043187181 | −1.267779153 |
| chr3 | 38203780 | 38204040 | −0.001110736 | −1.043187181 | −1.267779153 |
| chr8 | 126009800 | 126011200 | 0.001110577 | −1.225607156 | −1.029513898 |
| chr17 | 65713100 | 65714480 | 0.001110433 | −1.060762879 | −0.849793157 |
| chr7 | 148292140 | 148293020 | −0.001110241 | −1.110868635 | −1.38000018 |
| chr9 | 19432380 | 19432940 | −0.001109838 | −0.906777604 | −1.047021544 |
| chr1 | 204463320 | 204464320 | 0.001109662 | −1.160275821 | −0.962974817 |
| chr12 | 60278380 | 60279160 | −0.001109632 | −1.145182368 | −1.371858177 |
| chr3 | 131519980 | 131520600 | −0.001109233 | −1.065702335 | −1.253109212 |
| chr4 | 171895040 | 171895320 | −0.001109221 | −1.188100726 | −1.329545183 |
| chr4 | 171895040 | 171895320 | −0.001109221 | −1.188100726 | −1.329545183 |
| chr7 | 150948700 | 150949240 | 0.00110916 | −1.176275801 | −1.000721177 |
| chr3 | 187471920 | 187472320 | −0.001109075 | −1.010081127 | −1.201979465 |
| chr9 | 107255100 | 107255360 | −0.001108693 | −0.788941911 | −0.996220795 |
| chr19 | 45251560 | 45252340 | 0.001108604 | −1.125192241 | −0.957077582 |
| chr10 | 102858380 | 102858880 | 0.001108592 | −1.326574452 | −1.231909776 |
| chr20 | 32617120 | 32617480 | −0.001108515 | −0.96447458 | −1.099950838 |
| chr3 | 175940780 | 175941100 | −0.001108397 | −1.021356737 | −1.153948644 |
| chr13 | 101732600 | 101733320 | −0.001108267 | −1.065862241 | −1.371189059 |
| chr13 | 101732580 | 101733340 | −0.001108267 | −1.065862241 | −1.371189059 |
| chr8 | 113795400 | 113795920 | 0.001108178 | −1.287508235 | −1.041498426 |
| chr14 | 32545760 | 32546960 | 0.001108123 | −1.331727282 | −1.104769089 |
| chr14 | 32545580 | 32547140 | 0.001108123 | −1.331727282 | −1.104769089 |
| chr7 | 2749360 | 2749660 | 0.00110797 | −1.099791669 | −0.963744313 |
| chr2 | 180128620 | 180129840 | 0.001107824 | −1.359182984 | −1.11439275 |
| chr10 | 103744760 | 103745380 | −0.001107812 | −1.085420594 | −1.271328849 |
| chr5 | 146776200 | 146776900 | −0.001107667 | −1.012483053 | −1.236119827 |
| chr5 | 146776200 | 146776900 | −0.001107667 | −1.012483053 | −1.236119827 |
| chr4 | 88574320 | 88574820 | −0.001107463 | −1.155274433 | −1.287118148 |
| chr13 | 100612600 | 100613100 | −0.001107232 | −1.030237645 | −1.245534107 |
| chr19 | 55999420 | 55999960 | 0.00110723 | −1.164295262 | −1.032694178 |
| chr13 | 98223520 | 98224420 | 0.001107111 | −1.218091689 | −1.132991922 |
| chr8 | 28930380 | 28930820 | 0.001107104 | −1.163634311 | −1.002917542 |
| chr17 | 43091540 | 43091880 | −0.001107086 | −1.066531017 | −1.268892329 |
| chr21 | 18884680 | 18885820 | 0.001107086 | −0.911982507 | −0.642185424 |
| chr4 | 71570200 | 71571620 | 0.001106177 | −1.179700166 | −0.961242605 |
| chr15 | 75164820 | 75166040 | 0.001105956 | −1.197027278 | −1.043164783 |
| chr15 | 42499580 | 42501460 | 0.001105943 | −1.119390287 | −1.023250186 |
| chr4 | 172377520 | 172379560 | 0.00110585 | −1.413560809 | −1.289344738 |
| chr9 | 77995640 | 77995980 | −0.001105782 | −0.859771582 | −1.058143293 |
| chr17 | 47646760 | 47647080 | 0.001105742 | −1.179704185 | −1.020616566 |
| chr5 | 149339380 | 149341000 | 0.001105696 | −1.329290316 | −1.1052653 |
| chr19 | 918620 | 919120 | 0.001105665 | −1.193126015 | −0.988711704 |
| chr12 | 80946180 | 80946680 | 0.001105542 | −1.248979071 | −1.118391957 |
| chr16 | 4012880 | 4013320 | 0.001105481 | −0.986897932 | −0.913261925 |
| chr6 | 104834880 | 104835600 | −0.00110538 | −1.148770254 | −1.361949852 |
| chr16 | 76341200 | 76341640 | −0.001105313 | −0.860072583 | −1.03761654 |
| chr14 | 54790180 | 54790700 | −0.001105228 | −1.014925905 | −1.273216775 |
| chr12 | 58005400 | 58005920 | 0.001105225 | −1.274916687 | −1.083672963 |
| chr17 | 28543080 | 28543500 | 0.00110518 | −1.08692176 | −0.856566125 |
| chr5 | 180162560 | 180163140 | −0.001105065 | −0.930764908 | −1.192195198 |
| chr6 | 155054220 | 155055540 | 0.001104816 | −1.194462809 | −1.008763291 |
| chr17 | 80477100 | 80478540 | 0.001104658 | −1.282374857 | −1.148826953 |
| chr11 | 66623620 | 66625040 | −0.001104626 | −0.997960155 | −1.049134801 |
| chr4 | 1080160 | 1081500 | 0.001104473 | −1.322799963 | −1.093214382 |
| chr12 | 106695600 | 106697480 | 0.001104437 | −1.285150932 | −1.048520032 |
| chr5 | 67558880 | 67559280 | −0.001104418 | −0.974748799 | −1.08187751 |
| chr5 | 67558880 | 67559280 | −0.001104418 | −0.974748799 | −1.08187751 |
| chr3 | 80713280 | 80713840 | −0.001103741 | −1.113039043 | −1.368171391 |
| chr2 | 3605340 | 3606680 | 0.001103586 | −1.370873561 | −1.122037621 |
| chr3 | 140949980 | 140951400 | −0.001103438 | −1.159002703 | −1.208638239 |
| chr11 | 62554000 | 62555160 | 0.001103415 | −1.191797991 | −1.075183772 |
| chr11 | 46382920 | 46383180 | 0.001103177 | −1.1893118 | −0.991511254 |
| chr2 | 85197640 | 85199320 | 0.001102977 | −1.273278124 | −1.112673488 |
| chr1 | 150336020 | 150337300 | 0.001102852 | −1.085425464 | −0.947321156 |
| chr6 | 126216900 | 126217400 | −0.001102692 | −0.949887483 | −1.136352635 |
| chr20 | 43948200 | 43948540 | 0.001102689 | −1.239445031 | −1.005015613 |
| chr11 | 2400340 | 2400800 | 0.001102652 | −1.399573295 | −1.177097548 |
| chr16 | 28099980 | 28101420 | 0.001102207 | −1.072212805 | −0.881094079 |
| chr22 | 37623540 | 37624000 | 0.001102157 | −1.0421623 | −0.867519228 |
| chr5 | 105900100 | 105900780 | −0.001102124 | −1.083621017 | −1.260232218 |
| chr3 | 154851260 | 154851780 | −0.001102118 | −1.054997965 | −1.193860898 |
| chr1 | 40254040 | 40255180 | 0.001101727 | −1.253137358 | −1.08225816 |
| chr3 | 54769880 | 54770360 | −0.001101714 | −1.095202633 | −1.290807407 |
| chr3 | 23335900 | 23336500 | −0.001101337 | −0.979964724 | −1.289308281 |
| chr1 | 169323640 | 169324200 | −0.001101321 | −0.947386528 | −1.091804501 |
| chr9 | 28470640 | 28471160 | −0.001101059 | −0.726924312 | −1.009727056 |
| chr20 | 23342280 | 23343500 | 0.001100957 | −1.356818021 | −1.172992329 |
| chr2 | 198575320 | 198575820 | −0.001100952 | −0.718785184 | −1.050527239 |
| chr17 | 74235520 | 74237280 | 0.001100693 | −1.230262266 | −1.019993099 |
| chr21 | 45410820 | 45411060 | −0.001100684 | −0.792276369 | −0.965143807 |
| chr21 | 45410820 | 45411060 | −0.001100684 | −0.792276369 | −0.965143807 |
| chr15 | 84343200 | 84343780 | 0.001100151 | −1.094850715 | −1.084557239 |
| chr22 | 27131840 | 27132880 | −0.001100052 | −0.797451886 | −0.972393274 |
| chr22 | 27131820 | 27132900 | −0.001100052 | −0.797451886 | −0.972393274 |
| chr5 | 158639520 | 158639920 | −0.001099874 | −1.010582153 | −1.216650835 |
| chr16 | 54180400 | 54180760 | −0.001099667 | −0.807984262 | −1.128075658 |
| chr4 | 163470660 | 163471100 | −0.001099603 | −0.993675793 | −1.060225907 |
| chr4 | 163470620 | 163471140 | −0.001099603 | −0.993675793 | −1.060225907 |
| chr8 | 97742940 | 97743320 | 0.001099399 | −1.162384109 | −0.956382864 |
| chr7 | 89697620 | 89698040 | 0.001099327 | −1.097587384 | −1.071577078 |
| chr7 | 89697580 | 89698080 | 0.001099327 | −1.097587384 | −1.071577078 |
| chr6 | 147523520 | 147523900 | 0.001099054 | −1.357609421 | −1.153512978 |
| chr6 | 125465720 | 125466180 | −0.001098988 | −1.165933018 | −1.366378828 |
| chr22 | 24407240 | 24408240 | 0.001098737 | −1.047785904 | −0.893603964 |
| chr17 | 57183580 | 57184880 | 0.001098567 | −1.202976747 | −1.04662245 |
| chr8 | 86868800 | 86869500 | −0.001098248 | −0.999644117 | −1.207265331 |
| chr19 | 56594960 | 56595380 | 0.001098019 | −1.165147244 | −1.103724531 |
| chr19 | 56594940 | 56595400 | 0.001098019 | −1.165147244 | −1.103724531 |
| chr7 | 111738420 | 111738920 | −0.001097921 | −0.826923421 | −1.103707589 |
| chr1 | 95699180 | 95700400 | 0.001097621 | −1.149401607 | −0.944441514 |
| chr7 | 148755200 | 148755580 | −0.001097488 | −0.880013089 | −1.096136148 |
| chr7 | 148755160 | 148755620 | −0.001097488 | −0.880013089 | −1.096136148 |
| chr4 | 33693140 | 33693540 | −0.001097471 | −0.865137637 | −1.044955392 |
| chr19 | 3134060 | 3134420 | 0.001097417 | −1.144341277 | −1.037608229 |
| chr9 | 117493200 | 117493680 | 0.001097371 | −1.151888295 | −0.934357278 |
| chr9 | 33264260 | 33265600 | 0.001097052 | −1.189286187 | −1.036962144 |
| chr4 | 177005700 | 177006180 | −0.001097042 | −0.9330834 | −1.197246707 |
| chr13 | 104688320 | 104688920 | −0.001097009 | −0.895616496 | −1.077176686 |
| chr10 | 30702980 | 30704440 | −0.001096979 | −0.918247112 | −1.215735334 |
| chr19 | 57791180 | 57792280 | 0.001096317 | −1.116616158 | −0.982731292 |
| chr1 | 156906060 | 156906500 | −0.001096258 | −1.107613253 | −1.233349572 |
| chr8 | 79385240 | 79385780 | −0.001096241 | −0.78238081 | −1.038704805 |
| chr7 | 50571980 | 50572500 | 0.001096197 | −1.102394373 | −1.040023417 |
| chr19 | 10023860 | 10025700 | 0.001096082 | −1.017057898 | −0.851433151 |
| chr11 | 38971200 | 38971740 | −0.001096043 | −1.193209348 | −1.315427705 |
| chr21 | 48081540 | 48082200 | 0.001096021 | −1.188069531 | −1.060079015 |
| chr10 | 74123880 | 74124520 | 0.001095907 | −1.196371136 | −1.004279083 |
| chr5 | 62838940 | 62839380 | −0.001095898 | −0.996690402 | −1.20226593 |
| chr12 | 122471900 | 122472300 | −0.001095878 | −1.095755515 | −1.180200784 |
| chr2 | 111435360 | 111436240 | 0.001095734 | −1.306648796 | −1.089836851 |
| chr14 | 64280940 | 64281380 | −0.001095723 | −1.052770315 | −1.259899842 |
| chr10 | 93169340 | 93170900 | −0.001095696 | −1.183676112 | −1.257490586 |
| chr8 | 106226580 | 106227200 | −0.001095579 | −0.998208783 | −1.258040604 |
| chr22 | 48503440 | 48504300 | −0.001095499 | −0.842729841 | −1.088430367 |
| chr7 | 3386180 | 3386680 | 0.001095364 | −1.117001369 | −1.033633415 |
| chr2 | 77683860 | 77684400 | −0.001095151 | −0.980148032 | −1.219285231 |
| chr20 | 55921500 | 55921780 | 0.001094637 | −1.199103232 | −0.982504957 |
| chr6 | 30512500 | 30512760 | 0.001094599 | −1.277684009 | −1.017752192 |
| chr10 | 62060680 | 62061180 | −0.001094445 | −0.921909654 | −1.121344318 |
| chr18 | 9474780 | 9476080 | 0.00109429 | −1.393981088 | −1.202955433 |
| chr5 | 131028660 | 131029220 | 0.001094068 | −1.272436106 | −1.126649192 |
| chr9 | 130533240 | 130534160 | −0.00109376 | −0.943225458 | −1.026449294 |
| chr20 | 10282300 | 10282740 | −0.001093575 | −1.050034265 | −1.323189808 |
| chr5 | 67045300 | 67045980 | −0.001093574 | −0.89430094 | −1.092175864 |
| chr3 | 166425180 | 166425580 | −0.001093367 | −0.964526762 | −1.089029838 |
| chr3 | 166425160 | 166425600 | −0.001093367 | −0.964526762 | −1.089029838 |
| chr2 | 41307060 | 41307560 | −0.001093192 | −0.909671379 | −1.186745892 |
| chr11 | 97716180 | 97716680 | 0.001093177 | −1.144286241 | −1.143598928 |
| chr6 | 111579900 | 111581120 | 0.001093171 | −1.382949313 | −1.187148683 |
| chr2 | 180737200 | 180737580 | 0.001093025 | −1.244250984 | −1.094186565 |
| chr16 | 11679900 | 11681240 | 0.001092484 | −1.160749397 | −0.892544027 |
| chr16 | 4321800 | 4322300 | 0.001092378 | −1.075430045 | −0.880071276 |
| chr19 | 13213460 | 13213880 | 0.001092025 | −1.147840876 | −0.932274767 |
| chr2 | 47482920 | 47483460 | −0.001092018 | −1.009927199 | −1.243062235 |
| chr11 | 57143540 | 57144180 | 0.00109195 | −1.263768564 | −0.998729933 |
| chr5 | 108265880 | 108266500 | −0.001091944 | −1.178237488 | −1.259500336 |
| chr15 | 40649620 | 40651400 | 0.001091915 | −1.246592501 | −1.024580214 |
| chr6 | 25206440 | 25207300 | 0.001091743 | −1.347147337 | −1.241971103 |
| chr20 | 48785940 | 48786220 | 0.001091691 | −1.331968932 | −1.165253538 |
| chr5 | 26793020 | 26794260 | −0.001091617 | −1.172252717 | −1.371655031 |
| chr10 | 78386740 | 78387280 | −0.001091606 | −1.143229332 | −1.260482582 |
| chr11 | 65097420 | 65097700 | 0.001091543 | −1.261145394 | −1.123884619 |
| chr13 | 101625560 | 101626360 | 0.001091447 | −1.185539583 | −1.017533128 |
| chr7 | 6565820 | 6566700 | 0.001091413 | −1.174796013 | −0.938375745 |
| chr16 | 50699300 | 50699580 | 0.001091292 | −1.108813454 | −0.898498286 |
| chr2 | 52382440 | 52382820 | −0.001091135 | −0.871756956 | −1.111138895 |
| chr6 | 152345560 | 152346160 | −0.00109113 | −1.061589304 | −1.219961692 |
| chr9 | 110877680 | 110878320 | −0.001091126 | −0.955019969 | −1.201101392 |
| chr20 | 35374080 | 35374780 | 0.001091076 | −1.24962973 | −1.094375442 |
| chr9 | 11474040 | 11474500 | −0.001091071 | −0.619300316 | −0.840528515 |
| chr21 | 30160360 | 30160680 | −0.001091053 | −0.832952112 | −1.083452822 |
| chr21 | 30160360 | 30160680 | −0.001091053 | −0.832952112 | −1.083452822 |
| chr18 | 65661240 | 65661640 | −0.001090935 | −1.111679364 | −1.339489902 |
| chr16 | 57713380 | 57714500 | −0.001090639 | −0.876149696 | −0.990918627 |
| chr17 | 4099080 | 4099680 | −0.001090536 | −1.001857562 | −1.114591404 |
| chr11 | 71791120 | 71792520 | 0.001090346 | −1.362064533 | −1.191380275 |
| chr2 | 77683900 | 77684320 | −0.001090293 | −0.937454227 | −1.179498658 |
| chr9 | 103593940 | 103594260 | 0.001090214 | −0.974978529 | −0.957714593 |
| chr4 | 12795580 | 12796660 | −0.001090206 | −1.019977757 | −1.180906224 |
| chr17 | 74931680 | 74932240 | −0.001090016 | −1.049052857 | −1.280155807 |
| chr8 | 70173320 | 70173740 | −0.001089965 | −1.082218406 | −1.25877137 |
| chr6 | 159429680 | 159429920 | 0.001089775 | −1.117883278 | −0.942911914 |
| chr2 | 21370140 | 21370520 | −0.001089559 | −0.946711317 | −1.161338823 |
| chr2 | 21370080 | 21370580 | −0.001089559 | −0.946711317 | −1.161338823 |
| chr12 | 21122900 | 21123460 | −0.001089421 | −0.983488598 | −1.26567191 |
| chr7 | 35879540 | 35879980 | −0.001089057 | −0.97710005 | −1.10700535 |
| chr2 | 186867040 | 186867540 | −0.001088855 | −0.854030232 | −1.054433747 |
| chr2 | 61900300 | 61900760 | −0.001088855 | −1.028951469 | −1.209383461 |
| chr2 | 61900300 | 61900760 | −0.001088855 | −1.028951469 | −1.209383461 |
| chr6 | 9777940 | 9778660 | 0.001088589 | −1.049101968 | −1.052998008 |
| chr14 | 86290520 | 86290920 | −0.001088568 | −0.951366952 | −1.210529378 |
| chr19 | 48672780 | 48674520 | 0.001088556 | −1.291425723 | −1.129446179 |
| chr1 | 203008500 | 203008760 | 0.001088448 | −1.193390262 | −1.009547415 |
| chr21 | 40751580 | 40752840 | 0.001088434 | −1.104389244 | −0.943329724 |
| chr16 | 9358600 | 9359020 | 0.001088145 | −1.239415843 | −1.136710051 |
| chr9 | 130747820 | 130748180 | −0.001088099 | −0.932356577 | −1.069537787 |
| chr9 | 6930300 | 6930920 | −0.001088089 | −0.842753247 | −1.133149393 |
| chr5 | 26882960 | 26883420 | −0.001088071 | −1.081484022 | −1.249026938 |
| chr16 | 54646980 | 54647700 | −0.001087959 | −0.755494408 | −1.073637836 |
| chr8 | 76163520 | 76164180 | 0.001087914 | −1.316302402 | −1.246208936 |
| chr7 | 4721560 | 4722840 | 0.001087844 | −1.311288871 | −1.054803664 |
| chr11 | 69177440 | 69178640 | 0.001087801 | −1.329029567 | −1.252189201 |
| chr10 | 30702980 | 30704220 | −0.001087709 | −0.920711138 | −1.155284202 |
| chr11 | 59962720 | 59963280 | −0.001087708 | −1.084098737 | −1.333081606 |
| chr4 | 151096220 | 151096580 | −0.00108765 | −0.973051645 | −1.145733493 |
| chr17 | 54990900 | 54991900 | 0.001087472 | −1.077709902 | −0.933148836 |
| chr2 | 195033040 | 195033560 | −0.001087231 | −1.052065488 | −1.300784911 |
| chr1 | 200456720 | 200457140 | 0.001087135 | −1.151795933 | −0.985817069 |
| chr11 | 13428180 | 13428460 | −0.001087083 | −0.695226343 | −0.914646217 |
| chr17 | 75301820 | 75302340 | −0.00108699 | −0.924115612 | −1.051372544 |
| chr17 | 617500 | 618620 | 0.001086786 | −1.130163325 | −0.924921129 |
| chr4 | 31338280 | 31338880 | 0.001086682 | −1.36449743 | −1.264994252 |
| chr19 | 39902480 | 39903660 | 0.001086566 | −1.195637078 | −1.022827429 |
| chr19 | 39902480 | 39903660 | 0.001086566 | −1.195637078 | −1.022827429 |
| chr6 | 52149340 | 52150000 | 0.001086476 | −1.26766284 | −1.004076996 |
| chr4 | 110132180 | 110132700 | −0.001086464 | −1.138841769 | −1.331830486 |
| chr7 | 107365640 | 107365900 | 0.00108646 | −1.012598736 | −0.871277019 |
| chr4 | 66440040 | 66440680 | −0.001086375 | −1.045819546 | −1.308331721 |
| chr6 | 30687820 | 30689700 | 0.001086324 | −1.201706216 | −1.014122001 |
| chr2 | 97559940 | 97560820 | −0.001086285 | −1.177050644 | −1.223466985 |
| chr15 | 59396620 | 59397920 | 0.001086248 | −1.115879175 | −0.938755101 |
| chr11 | 47950860 | 47951560 | −0.001086148 | −1.19312658 | −1.378975663 |
| chr13 | 91960820 | 91961420 | −0.001086113 | −1.175733858 | −1.333323847 |
| chr4 | 177240460 | 177241900 | 0.001085891 | −1.358104108 | −1.191522454 |
| chr16 | 68056960 | 68057380 | 0.001085785 | −1.277916694 | −1.090985884 |
| chr21 | 18457280 | 18457780 | 0.001085706 | −1.145080666 | −1.025117018 |
| chr21 | 18457280 | 18457780 | 0.001085706 | −1.145080666 | −1.025117018 |
| chr20 | 48428740 | 48430060 | 0.001085699 | −1.239621471 | −0.991289923 |
| chr5 | 25257920 | 25258320 | −0.001085404 | −0.941933463 | −1.191757354 |
| chr14 | 71702040 | 71702500 | −0.00108529 | −1.06837264 | −1.283937185 |
| chr14 | 71702040 | 71702500 | −0.00108529 | −1.06837264 | −1.283937185 |
| chr15 | 61332120 | 61332580 | −0.001085275 | −0.94707246 | −1.198116328 |
| chr8 | 117496400 | 117496800 | −0.001085113 | −1.06069849 | −1.263252543 |
| chr9 | 29868280 | 29868800 | −0.001084945 | −0.912245924 | −1.154374064 |
| chr16 | 21036480 | 21037020 | 0.001084684 | −1.240437358 | −1.171655265 |
| chr1 | 239567800 | 239568260 | −0.001084609 | −1.015917406 | −1.180758439 |
| chr14 | 95979960 | 95980320 | −0.001084594 | −1.015591952 | −1.130026049 |
| chr13 | 106018760 | 106019360 | −0.001084511 | −1.045106176 | −1.316070496 |
| chr13 | 106018740 | 106019380 | −0.001084511 | −1.045106176 | −1.316070496 |
| chr1 | 36590460 | 36591160 | 0.001084453 | −1.138734614 | −0.926862975 |
| chr3 | 12933540 | 12934700 | 0.001084349 | −1.424673647 | −1.229486585 |
| chr3 | 12933520 | 12934720 | 0.001084349 | −1.424673647 | −1.229486585 |
| chr13 | 40479660 | 40480040 | −0.001084332 | −1.000230845 | −1.158038231 |
| chr2 | 148517700 | 148518200 | −0.001084279 | −1.158928725 | −1.308702313 |
| chr20 | 1630460 | 1630920 | −0.001084267 | −0.972196519 | −1.157315592 |
| chr12 | 9246740 | 9247180 | −0.001084082 | −1.010508681 | −1.204558992 |
| chr7 | 16684280 | 16686780 | 0.001083934 | −1.261899363 | −1.048029884 |
| chr17 | 56640740 | 56641220 | −0.001083676 | −0.990746174 | −1.103155557 |
| chr8 | 42248640 | 42250180 | 0.00108362 | −1.194967705 | −1.075724318 |
| chr1 | 215799320 | 215799960 | 0.001083569 | −1.13347652 | −0.963342446 |
| chr19 | 5719700 | 5720900 | 0.001083034 | −1.22493442 | −1.112173442 |
| chr6 | 99917160 | 99917540 | −0.001082943 | −0.991931711 | −1.193051155 |
| chr1 | 72209700 | 72210280 | −0.001082904 | −0.90094982 | −1.173500667 |
| chr4 | 31403380 | 31404040 | −0.001082883 | −1.10086198 | −1.280549441 |
| chr14 | 50697520 | 50699160 | 0.001082812 | −1.201542342 | −1.035111146 |
| chr3 | 144344520 | 144344960 | −0.001082675 | −1.040066072 | −1.214584111 |
| chr7 | 38368080 | 38368580 | −0.001082317 | −1.057372202 | −1.294187446 |
| chr15 | 69754900 | 69755460 | 0.001082204 | −1.244763015 | −1.046679509 |
| chr4 | 165676540 | 165676900 | −0.001082168 | −1.246323043 | −1.35151528 |
| chr9 | 74886840 | 74887140 | 0.001081757 | −1.154021774 | −0.922963744 |
| chr6 | 79263420 | 79264120 | −0.001081571 | −1.073521395 | −1.267507841 |
| chr5 | 23597340 | 23597780 | −0.001081564 | −0.959265119 | −1.189786331 |
| chr8 | 23549880 | 23550780 | 0.001081403 | −1.202319047 | −1.033046182 |
| chr4 | 93047220 | 93047640 | −0.001081343 | −0.871557241 | −1.104076297 |
| chr20 | 59928500 | 59928960 | 0.001080798 | −1.18315954 | −0.993317187 |
| chr1 | 33721440 | 33722700 | 0.001080763 | −1.242691513 | −1.093058437 |
| chr1 | 197144360 | 197144780 | −0.001080757 | −0.909456484 | −1.090633947 |
| chr2 | 102968660 | 102969120 | −0.001080716 | −1.218820791 | −1.379238452 |
| chr15 | 74753180 | 74754080 | 0.00108058 | −1.245652291 | −1.0899561 |
| chr15 | 84347280 | 84347880 | −0.001080538 | −0.907233661 | −1.226336505 |
| chr19 | 40969280 | 40969660 | −0.001080498 | −0.955821473 | −1.015675027 |
| chr3 | 151478780 | 151479200 | −0.001080426 | −1.165490309 | −1.320855829 |
| chr1 | 147830900 | 147831220 | −0.001080351 | −1.061224256 | −1.280212429 |
| chr1 | 147830900 | 147831220 | −0.001080351 | −1.061224256 | −1.280212429 |
| chr4 | 186145480 | 186146020 | −0.001080326 | −1.198639956 | −1.358394434 |
| chr4 | 81122900 | 81125140 | 0.001080144 | −1.293817655 | −1.079769653 |
| chr16 | 50099220 | 50101100 | 0.001080075 | −1.010754521 | −0.850087949 |
| chr13 | 58506380 | 58506900 | 0.001080037 | −1.351863529 | −1.283092785 |
| chr8 | 35646400 | 35646920 | −0.001079917 | −1.131315312 | −1.282473847 |
| chr3 | 158509140 | 158509420 | 0.001079803 | −1.195370738 | −0.949044602 |
| chr11 | 2035840 | 2036240 | −0.001079244 | −1.066866339 | −1.177756373 |
| chr11 | 2035820 | 2036260 | −0.001079244 | −1.066866339 | −1.177756373 |
| chr17 | 29885720 | 29887080 | 0.001078951 | −1.094353005 | −0.893687438 |
| chr3 | 4073140 | 4073360 | −0.00107891 | −1.116432483 | −1.285169114 |
| chr14 | 91867420 | 91867760 | 0.001078678 | −1.288606733 | −1.186163427 |
| chr9 | 94185600 | 94187160 | 0.00107862 | −1.169725633 | −0.971245736 |
| chr9 | 106171140 | 106171580 | −0.00107857 | −0.723609441 | −1.005076883 |
| chr6 | 40604380 | 40605000 | −0.001078501 | −1.238874938 | −1.423049398 |
| chr10 | 78398720 | 78399340 | −0.001078198 | −1.186595766 | −1.334389869 |
| chr2 | 195612000 | 195612400 | −0.001078125 | −0.806389814 | −1.028394919 |
| chr1 | 117959960 | 117960380 | −0.001078115 | −0.91729762 | −1.163141634 |
| chr1 | 40960180 | 40960680 | 0.001078026 | −1.152912469 | −1.106243345 |
| chr2 | 84459120 | 84459700 | −0.001078 | −1.099961143 | −1.298223503 |
| chr16 | 82149060 | 82149540 | −0.001077948 | −0.732934058 | −1.041386856 |
| chr16 | 82149060 | 82149540 | −0.001077948 | −0.732934058 | −1.041386856 |
| chr10 | 101573660 | 101574600 | 0.001077648 | −1.1916788 | −1.028861027 |
| chr19 | 14007040 | 14007680 | 0.001077495 | −1.032599654 | −0.977924358 |
| chr12 | 81334760 | 81335300 | −0.001077476 | −0.978616934 | −1.247633058 |
| chr16 | 88819300 | 88819940 | −0.001077439 | −1.107692769 | −1.225744556 |
| chr2 | 163197560 | 163198020 | −0.001077184 | −1.007479553 | −1.199894016 |
| chr16 | 11690160 | 11690760 | −0.001077164 | −0.916069173 | −0.988061817 |
| chr11 | 3012980 | 3014180 | 0.001077146 | −1.221445826 | −1.067656126 |
| chr7 | 29163020 | 29163460 | −0.001077136 | −0.931198914 | −1.174397974 |
| chr19 | 50528020 | 50529540 | 0.001077005 | −1.242319446 | −1.088896485 |
| chr8 | 6350040 | 6350480 | −0.001076898 | −0.957041394 | −1.15872325 |
| chr2 | 102593960 | 102594460 | −0.001076402 | −1.141313388 | −1.267769129 |
| chr6 | 31734660 | 31735040 | 0.001076257 | −1.286360503 | −1.210203247 |
| chr6 | 31734600 | 31735100 | 0.001076257 | −1.286360503 | −1.210203247 |
| chr17 | 17942060 | 17943240 | 0.001075921 | −1.299273369 | −1.12763084 |
| chr6 | 135989480 | 135990200 | −0.001075774 | −1.16586908 | −1.355166773 |
| chr5 | 126582280 | 126582860 | −0.001075322 | −1.094803334 | −1.356181351 |
| chr3 | 119641080 | 119641500 | −0.001075297 | −1.032733709 | −1.279359395 |
| chr14 | 102248240 | 102248540 | −0.00107522 | −1.161393245 | −1.256975047 |
| chr19 | 50676240 | 50676560 | 0.001075141 | −1.061388134 | −0.816153937 |
| chr19 | 50676240 | 50676560 | 0.001075141 | −1.061388134 | −0.816153937 |
| chr1 | 110025960 | 110027540 | 0.001074968 | −1.049686735 | −0.918270279 |
| chr4 | 152690260 | 152690860 | −0.001074942 | −0.985186366 | −1.180098605 |
| chr2 | 219134740 | 219135620 | 0.001074744 | −1.401773131 | −1.172962585 |
| chr3 | 55748440 | 55748980 | −0.001074418 | −1.087554017 | −1.228678293 |
| chr3 | 95740600 | 95741140 | −0.001074171 | −1.252988882 | −1.398877526 |
| chr11 | 125462260 | 125463180 | 0.001074055 | −1.325602908 | −1.213919689 |
| chr16 | 30758880 | 30760660 | 0.001073868 | −1.180113818 | −0.931229794 |
| chr15 | 84470160 | 84470660 | −0.001073684 | −0.996136368 | −1.178800006 |
| chr19 | 18043340 | 18044240 | 0.001073222 | −1.249012506 | −1.077929733 |
| chr19 | 41304580 | 41305860 | 0.001072977 | −1.122493246 | −0.942796719 |
| chr19 | 41304560 | 41305900 | 0.001072977 | −1.122493246 | −0.942796719 |
| chr1 | 179956320 | 179956900 | −0.001072826 | −0.91859188 | −1.197742184 |
| chr4 | 85892560 | 85892820 | 0.001072801 | −1.242412642 | −0.981761846 |
| chr10 | 123617920 | 123618340 | −0.001072633 | −1.000239366 | −1.148501706 |
| chr11 | 10955300 | 10955620 | 0.001072609 | −1.26339542 | −1.131005529 |
| chr11 | 10955300 | 10955620 | 0.001072609 | −1.26339542 | −1.131005529 |
| chr6 | 27763680 | 27763960 | −0.001072333 | −1.046884755 | −1.150170129 |
| chr2 | 222246580 | 222246920 | −0.001071874 | −1.057221584 | −1.255963393 |
| chr9 | 37485060 | 37486700 | 0.001071609 | −1.100013475 | −0.901000507 |
| chr17 | 78866800 | 78867500 | 0.001071499 | −1.161495437 | −0.988537634 |
| chr9 | 137299160 | 137300040 | −0.001071479 | −0.886572606 | −0.992375213 |
| chr22 | 48377280 | 48377740 | −0.001071361 | −0.884940862 | −1.159891969 |
| chr13 | 25874880 | 25876600 | 0.001071334 | −1.320467827 | −1.116968099 |
| chr8 | 77477240 | 77477660 | −0.001071148 | −1.052401443 | −1.235159457 |
| chr10 | 43950820 | 43951440 | 0.001071118 | −1.22334767 | −1.072625184 |
| chr11 | 127538480 | 127539040 | −0.001071029 | −1.141499427 | −1.345315146 |
| chr17 | 40828760 | 40829420 | 0.001070921 | −1.11756298 | −0.913943178 |
| chr11 | 65651680 | 65652900 | 0.00107059 | −1.332162327 | −1.072456699 |
| chr17 | 61689640 | 61689920 | 0.001070581 | −1.189480905 | −1.079107101 |
| chr16 | 29973040 | 29973940 | −0.001070569 | −0.913083159 | −0.985061516 |
| chr18 | 29443800 | 29444240 | −0.001070559 | −0.925180144 | −1.18684329 |
| chr4 | 127572440 | 127572920 | −0.001070403 | −1.022117379 | −1.20466167 |
| chr8 | 78598560 | 78599020 | −0.001070395 | −1.160520366 | −1.27915887 |
| chr13 | 46731200 | 46731500 | 0.001070004 | −1.163551199 | −0.991231116 |
| chr12 | 7348440 | 7349080 | −0.001069869 | −1.109491932 | −1.334518168 |
| chr1 | 10270200 | 10271460 | 0.001069739 | −1.30706331 | −1.110155677 |
| chr9 | 140116340 | 140116940 | 0.001069703 | −1.16128793 | −1.014464114 |
| chr6 | 145676380 | 145676980 | −0.001069494 | −1.032671415 | −1.205769579 |
| chr9 | 91550660 | 91551040 | −0.001069343 | −0.740921933 | −0.965256916 |
| chr12 | 131384320 | 131384780 | −0.001069058 | −1.038058239 | −1.234661768 |
| chr8 | 124002000 | 124002820 | −0.001069016 | −0.989419183 | −1.249941196 |
| chr20 | 16390420 | 16391140 | −0.001068989 | −0.998401428 | −1.227355101 |
| chr17 | 7513040 | 7513540 | −0.001068899 | −0.896293203 | −1.131054629 |
| chr22 | 42228740 | 42230040 | 0.001068519 | −1.010291621 | −0.822129629 |
| chr5 | 24151400 | 24151920 | −0.001068472 | −1.090300811 | −1.268814755 |
| chr7 | 92485300 | 92485840 | −0.001068407 | −1.028620201 | −1.214796373 |
| chr19 | 12958120 | 12959020 | 0.001068094 | −1.161456091 | −1.00350827 |
| chr5 | 19219880 | 19220520 | −0.001068094 | −1.063706416 | −1.32330015 |
| chr13 | 56317400 | 56318000 | −0.001068081 | −1.19610983 | −1.39716488 |
| chr5 | 141071200 | 141072440 | 0.001068081 | −1.314697074 | −1.069061508 |
| chr12 | 8113300 | 8113620 | 0.001068025 | −1.137035588 | −1.014357338 |
| chr21 | 47411140 | 47411540 | 0.001067421 | −0.92463981 | −0.758667893 |
| chr15 | 75166780 | 75167220 | 0.001067315 | −1.129674989 | −1.006773293 |
| chr14 | 44022060 | 44022480 | 0.001067255 | −1.413207475 | −1.295845023 |
| chr1 | 166476500 | 166477040 | −0.001067119 | −0.905208659 | −1.16300159 |
| chr13 | 92031580 | 92032200 | −0.001067109 | −1.170640376 | −1.314348922 |
| chr4 | 26858780 | 26859820 | 0.001067069 | −1.341157125 | −1.159014698 |
| chr9 | 115982920 | 115984240 | 0.001066943 | −1.049368032 | −0.937549885 |
| chr17 | 3641020 | 3641320 | 0.001066931 | −1.217200109 | −1.010630052 |
| chr5 | 92645780 | 92646220 | −0.001066815 | −0.915512364 | −1.168415949 |
| chr5 | 92645740 | 92646260 | −0.001066815 | −0.915512364 | −1.168415949 |
| chr17 | 27919760 | 27921120 | −0.001066697 | −1.064903666 | −1.180061922 |
| chr12 | 27295100 | 27295360 | 0.001066646 | −1.200450851 | −1.052592689 |
| chr11 | 38195860 | 38196400 | 0.001066617 | −1.158351559 | −1.132538695 |
| chr13 | 89925960 | 89926520 | −0.001066543 | −1.168112382 | −1.336081471 |
| chr11 | 28039860 | 28040220 | −0.001066397 | −0.992700451 | −1.238555256 |
| chr10 | 17313520 | 17313860 | −0.001065984 | −0.983547071 | −1.172097644 |
| chr19 | 10683080 | 10683320 | 0.001065894 | −1.027905854 | −0.889412828 |
| chr19 | 10683060 | 10683340 | 0.001065894 | −1.027905854 | −0.889412828 |
| chr13 | 73301140 | 73302520 | 0.001065801 | −1.372077535 | −1.113430359 |
| chr20 | 48428480 | 48430340 | 0.001065726 | −1.237099156 | −1.00116725 |
| chr3 | 192883060 | 192885520 | 0.001065555 | −1.428786053 | −1.322844775 |
| chr19 | 39894460 | 39894780 | 0.001065426 | −1.187715458 | −0.996313451 |
| chr6 | 120401720 | 120402180 | −0.001065284 | −1.009549245 | −1.233510065 |
| chr6 | 120401700 | 120402200 | −0.001065284 | −1.009549245 | −1.233510065 |
| chr4 | 40619520 | 40619900 | −0.001065136 | −0.847431075 | −0.990053967 |
| chr8 | 15633020 | 15633420 | −0.001065105 | −0.915016761 | −1.171364044 |
| chr1 | 210000840 | 210001760 | 0.001065077 | −1.132260504 | −1.006573082 |
| chr11 | 123000120 | 123000580 | −0.00106498 | −1.155015901 | −1.343874883 |
| chr7 | 121842240 | 121842780 | −0.001063834 | −0.991937899 | −1.172896997 |
| chr17 | 74502140 | 74503080 | 0.0010638 | −1.175635356 | −0.995409738 |
| chr11 | 34460040 | 34460640 | 0.001063626 | −1.244403653 | −1.06108186 |
| chr11 | 64014880 | 64015260 | 0.001063601 | −1.055952472 | −0.976471255 |
| chr17 | 7782040 | 7782460 | −0.001063588 | −0.771238911 | −0.916943133 |
| chr17 | 7781960 | 7782540 | −0.001063588 | −0.771238911 | −0.916943133 |
| chr2 | 4914080 | 4914760 | −0.001063554 | −1.115782401 | −1.362849942 |
| chr7 | 1014620 | 1015760 | 0.001063502 | −1.332682407 | −1.139674366 |
| chr2 | 224869500 | 224870020 | −0.001063485 | −1.121608458 | −1.345308212 |
| chr1 | 91924480 | 91925140 | −0.001062913 | −0.988434207 | −1.247800755 |
| chr10 | 119289420 | 119289880 | −0.001062847 | −1.041094563 | −1.205617396 |
| chr3 | 30550760 | 30551360 | −0.001062722 | −1.074033384 | −1.222912042 |
| chr17 | 11282280 | 11282740 | −0.001062695 | −0.843223284 | −1.102557041 |
| chr16 | 12605980 | 12606500 | −0.001062683 | −0.955491967 | −1.139242583 |
| chr9 | 81450580 | 81450980 | 0.001062223 | −1.178520377 | −0.997277182 |
| chr3 | 29371160 | 29371600 | −0.001062193 | −0.969882253 | −1.251612963 |
| chr1 | 172501180 | 172503040 | 0.001062019 | −1.378691639 | −1.202672334 |
| chr6 | 31125400 | 31126740 | 0.0010616 | −1.275532421 | −1.133590191 |
| chr3 | 78278820 | 78279320 | −0.00106157 | −1.20665777 | −1.325978123 |
| chr9 | 98746200 | 98746760 | −0.001061455 | −0.85577376 | −1.055729949 |
| chr1 | 155107540 | 155108940 | 0.001061352 | −1.236326257 | −1.065068866 |
| chr1 | 32538080 | 32539360 | −0.001061303 | −0.97766906 | −1.045095951 |
| chr1 | 169706360 | 169706680 | −0.001061262 | −0.961621347 | −1.243096952 |
| chr13 | 76396700 | 76397160 | −0.001061199 | −1.173925455 | −1.320046209 |
| chr1 | 72766240 | 72767200 | 0.001061046 | −1.346491081 | −1.257503207 |
| chr1 | 72766240 | 72767200 | 0.001061046 | −1.346491081 | −1.257503207 |
| chr1 | 53661880 | 53662980 | 0.001060978 | −1.206076294 | −1.037833011 |
| chr7 | 26207980 | 26208280 | −0.001060572 | −0.997341977 | −1.158020464 |
| chr7 | 26207980 | 26208280 | −0.001060572 | −0.997341977 | −1.158020464 |
| chr9 | 133587700 | 133589100 | 0.001060417 | −0.94101629 | −0.827412466 |
| chr19 | 58816180 | 58817100 | 0.001060272 | −1.173942315 | −1.009236523 |
| chr18 | 40774940 | 40775360 | −0.00106021 | −1.026941616 | −1.261038438 |
| chr2 | 238506540 | 238507100 | 0.001060157 | −1.320035887 | −1.114541906 |
| chr2 | 105200540 | 105201000 | 0.00106015 | −1.078135131 | −0.928193763 |
| chr2 | 32594760 | 32595300 | −0.001060123 | −1.069905578 | −1.212855621 |
| chr12 | 110869660 | 110870120 | −0.001059809 | −0.986023797 | −1.193076598 |
| chr17 | 14685820 | 14686380 | −0.001059752 | −1.042592464 | −1.185601123 |
| chr9 | 35071820 | 35073400 | 0.001059672 | −1.168023078 | −1.003122186 |
| chr8 | 126009920 | 126011160 | 0.001059389 | −1.2835234 | −1.091255914 |
| chr1 | 210110680 | 210112220 | −0.001059239 | −0.962066236 | −0.989257339 |
| chr17 | 21156100 | 21156980 | 0.001059148 | −1.18253498 | −0.965354581 |
| chr6 | 26031260 | 26031560 | −0.001059033 | −1.081290913 | −1.314958039 |
| chr4 | 118660820 | 118661240 | −0.001058997 | −1.169830156 | −1.357892553 |
| chr4 | 118660820 | 118661240 | −0.001058997 | −1.169830156 | −1.357892553 |
| chr10 | 9427700 | 9428240 | −0.00105898 | −0.905293485 | −1.203393664 |
| chr22 | 37418060 | 37418300 | 0.001058928 | −1.030127693 | −0.827593202 |
| chr15 | 68569580 | 68571240 | 0.001058791 | −1.075288729 | −0.934970205 |
| chr13 | 96328620 | 96330060 | 0.001058774 | −1.446473859 | −1.266739818 |
| chr3 | 95740640 | 95741080 | −0.001058529 | −1.252988882 | −1.399572594 |
| chr6 | 26216520 | 26217560 | 0.001058465 | −1.191862664 | −1.092257409 |
| chr17 | 46774020 | 46774560 | −0.001058378 | −0.941862056 | −1.158447808 |
| chr15 | 69754920 | 69755500 | 0.001058136 | −1.207111847 | −0.992770563 |
| chr5 | 154412720 | 154413080 | −0.001058102 | −1.121563868 | −1.291053254 |
| chr5 | 154412720 | 154413080 | −0.001058102 | −1.121563868 | −1.291053254 |
| chr10 | 75531880 | 75533060 | 0.001057866 | −1.170109273 | −0.894167031 |
| chr6 | 11616900 | 11618120 | −0.001057435 | −1.135866592 | −1.369043879 |
| chr8 | 78598540 | 78599060 | −0.001057194 | −1.107000595 | −1.243146023 |
| chr12 | 128411460 | 128411920 | 0.001057176 | −1.283965561 | −1.200746793 |
| chr5 | 36294740 | 36295260 | −0.001057089 | −1.044447333 | −1.237221841 |
| chr6 | 42981220 | 42982560 | 0.001056974 | −1.407680512 | −1.257385674 |
| chr8 | 51278960 | 51279660 | −0.001056695 | −1.039352915 | −1.304280031 |
| chr16 | 11680340 | 11681200 | 0.001056632 | −1.122679556 | −0.996384727 |
| chr3 | 44902720 | 44904060 | 0.001056525 | −1.24377157 | −1.070364239 |
| chr5 | 89295020 | 89295520 | −0.001055942 | −1.068407178 | −1.280313917 |
| chr16 | 60808940 | 60809460 | −0.001055615 | −0.960905219 | −1.040569046 |
| chr16 | 60808940 | 60809460 | −0.001055615 | −0.960905219 | −1.040569046 |
| chr1 | 240085100 | 240085620 | −0.001055522 | −1.100107209 | −1.243136578 |
| chr4 | 132511940 | 132512460 | −0.001055503 | −1.174290979 | −1.329169268 |
| chr3 | 193972460 | 193973140 | −0.001055213 | −1.1040549 | −1.271111533 |
| chr4 | 84561100 | 84561400 | 0.00105502 | −1.359227955 | −1.122811932 |
| chr4 | 84560620 | 84561880 | 0.00105502 | −1.359227955 | −1.122811932 |
| chr5 | 180162520 | 180163200 | −0.001054928 | −0.930764908 | −1.190277427 |
| chr14 | 65462220 | 65462620 | −0.001054812 | −1.029955404 | −1.213119916 |
| chr17 | 2415040 | 2415460 | 0.001054785 | −1.073421307 | −0.878431761 |
| chr17 | 70083280 | 70084160 | −0.001054762 | −0.840422439 | −1.136458952 |
| chr17 | 70083280 | 70084160 | −0.001054762 | −0.840422439 | −1.136458952 |
| chr5 | 180475540 | 180476080 | −0.001054739 | −1.176059266 | −1.29224788 |
| chr22 | 45199300 | 45199880 | 0.001054419 | −1.044819971 | −0.898708091 |
| chr6 | 24581380 | 24581640 | 0.001054385 | −1.156837084 | −0.985477973 |
| chr12 | 32816300 | 32816540 | 0.001054202 | −1.157069946 | −1.098421716 |
| chr19 | 41945220 | 41946520 | 0.001054171 | −1.142778686 | −1.017073445 |
| chr6 | 13820680 | 13821200 | −0.00105412 | −1.0183836 | −1.237296366 |
| chr7 | 138795320 | 138795540 | −0.00105407 | −0.95259166 | −1.095802873 |
| chr7 | 138795200 | 138795660 | −0.00105407 | −0.95259166 | −1.095802873 |
| chr8 | 74205740 | 74207700 | 0.001054062 | −1.266437646 | −1.059859639 |
| chr2 | 220110020 | 220110340 | 0.001053938 | −1.294935042 | −1.093058622 |
| chr10 | 15138920 | 15139760 | 0.001053861 | −1.364591561 | −1.187598941 |
| chr15 | 95783860 | 95784600 | −0.001053785 | −1.097385483 | −1.282715393 |
| chr17 | 73963480 | 73963840 | −0.001053728 | −0.840794181 | −0.990590009 |
| chr5 | 142812820 | 142813200 | −0.001053667 | −1.044501112 | −1.284213529 |
| chr16 | 2569700 | 2571060 | 0.001053614 | −1.272552672 | −1.099963268 |
| chr7 | 89080940 | 89081460 | −0.001053606 | −1.084725687 | −1.28482253 |
| chr21 | 46970660 | 46971100 | 0.001053316 | −1.205210653 | −1.054990352 |
| chr2 | 64502180 | 64502640 | −0.001053238 | −0.870377643 | −1.092364105 |
| chr1 | 20349000 | 20349360 | 0.001053204 | −0.923967606 | −0.969043516 |
| chr2 | 86796220 | 86796680 | −0.00105314 | −0.916241203 | −1.124275766 |
| chr20 | 62169300 | 62170240 | 0.0010531 | −1.239357951 | −1.0917641 |
| chr22 | 46672420 | 46673080 | −0.001052902 | −0.827307748 | −1.041129131 |
| chr16 | 30640120 | 30640400 | 0.00105268 | −1.116391515 | −1.008873486 |
| chr13 | 50509800 | 50511420 | 0.001052663 | −1.459747054 | −1.250420055 |
| chr4 | 176557240 | 176559200 | −0.001052534 | −1.164248369 | −1.379842265 |
| chr15 | 26521920 | 26522420 | −0.00105236 | −0.966202001 | −1.217235476 |
| chr18 | 72920540 | 72921680 | 0.001052166 | −1.271782813 | −1.076891793 |
| chr18 | 72920540 | 72921680 | 0.001052166 | −1.271782813 | −1.076891793 |
| chr4 | 1080160 | 1081380 | 0.001052116 | −1.226323512 | −1.091732508 |
| chr16 | 88636100 | 88637400 | 0.001052098 | −1.330903406 | −1.161781357 |
| chr9 | 113913280 | 113914100 | −0.001051937 | −0.870674027 | −1.139839818 |
| chr20 | 62710540 | 62711620 | 0.001051919 | −1.131035301 | −1.035979026 |
| chr6 | 167411640 | 167413520 | 0.00105173 | −1.314382345 | −1.099150256 |
| chr1 | 59969040 | 59969540 | −0.001051639 | −1.007436307 | −1.153061663 |
| chr16 | 34949800 | 34950160 | −0.001051578 | −0.792529345 | −1.0044325 |
| chr5 | 81329440 | 81330060 | −0.001051555 | −1.078813213 | −1.329758415 |
| chr19 | 7935220 | 7935820 | 0.001051412 | −1.167761077 | −0.998484007 |
| chr4 | 117793120 | 117793560 | −0.001051298 | −1.077207454 | −1.287824449 |
| chr13 | 20534300 | 20534620 | 0.001051294 | −1.319030802 | −1.207032867 |
| chr4 | 190273440 | 190274160 | 0.001050982 | −1.037432748 | −1.114125884 |
| chr21 | 24615440 | 24616040 | −0.001050944 | −0.884373533 | −1.110456905 |
| chr4 | 94834420 | 94834820 | −0.001050638 | −1.024403454 | −1.233405774 |
| chr4 | 94834420 | 94834820 | −0.001050638 | −1.024403454 | −1.233405774 |
| chr9 | 131102340 | 131103480 | 0.001050457 | −1.078808257 | −0.878672257 |
| chr15 | 26521880 | 26522480 | −0.00105045 | −0.981726058 | −1.210502704 |
| chr4 | 170139120 | 170140020 | −0.001050445 | −1.098048845 | −1.317733743 |
| chr12 | 33388380 | 33388580 | 0.001050242 | −1.358689159 | −1.178523009 |
| chr5 | 142391860 | 142392240 | −0.001050122 | −1.010366161 | −1.126460115 |
| chr5 | 4641340 | 4641820 | −0.001050048 | −1.049357907 | −1.183513707 |
| chr1 | 43996240 | 43997540 | −0.001050011 | −0.993795294 | −1.051836111 |
| chr3 | 121078640 | 121079100 | −0.001049671 | −1.10855977 | −1.324536722 |
| chr5 | 157952120 | 157952600 | −0.001049561 | −0.897121959 | −1.174393449 |
| chr9 | 83372340 | 83373420 | −0.001049483 | −0.834580183 | −1.050214646 |
| chr17 | 40915480 | 40915780 | 0.001049477 | −1.087886311 | −0.932373982 |
| chr15 | 65596600 | 65596880 | 0.001049316 | −1.191586944 | −0.986076481 |
| chr2 | 161349100 | 161351020 | 0.00104924 | −1.334989767 | −1.181955237 |
| chr2 | 161349080 | 161351040 | 0.00104924 | −1.334989767 | −1.181955237 |
| chr10 | 5854680 | 5856060 | 0.001049027 | −1.27734593 | −1.134878048 |
| chr2 | 29308280 | 29308840 | −0.001048929 | −1.142272031 | −1.364632436 |
| chr2 | 29308280 | 29308840 | −0.001048929 | −1.142272031 | −1.364632436 |
| chr22 | 39333340 | 39334040 | 0.001048896 | −0.970638658 | −0.782262958 |
| chr11 | 518440 | 519220 | 0.001048872 | −1.387567932 | −1.178343219 |
| chr2 | 126648800 | 126649060 | −0.001048773 | −1.142601142 | −1.303093218 |
| chr1 | 115655460 | 115655740 | 0.001048711 | −1.227564012 | −1.077490343 |
| chr2 | 75103460 | 75103720 | −0.001048701 | −1.039942112 | −1.129865086 |
| chr10 | 97123120 | 97123400 | 0.001048658 | −1.2218975 | −1.081500806 |
| chr19 | 33403900 | 33404260 | 0.001048601 | −1.039054655 | −1.03105683 |
| chr12 | 113772760 | 113773160 | 0.001048499 | −1.290131494 | −1.095278831 |
| chr1 | 210500080 | 210500500 | −0.001048306 | −0.996227495 | −1.160705944 |
| chr13 | 114238460 | 114239880 | 0.001048278 | −1.353233352 | −1.160457646 |
| chr1 | 115631360 | 115632840 | 0.001048228 | −1.215295542 | −1.073467119 |
| chr4 | 176756560 | 176757140 | 0.001048038 | −1.267741661 | −1.151414268 |
| chr14 | 34953700 | 34954120 | 0.001047922 | −1.057368905 | −1.09191628 |
| chr2 | 195611940 | 195612440 | −0.001047885 | −0.778437718 | −1.027926196 |
| chr1 | 240085100 | 240085660 | −0.001047754 | −1.095250316 | −1.240452182 |
| chr8 | 112037040 | 112037480 | −0.00104759 | −1.038691409 | −1.293912507 |
| chr15 | 34385760 | 34386260 | −0.001047464 | −0.984287736 | −1.146529764 |
| chr10 | 11149660 | 11150280 | −0.001047451 | −1.080606915 | −1.231481909 |
| chr15 | 69109920 | 69111280 | 0.001047261 | −1.084201115 | −0.965159757 |
| chr3 | 42814060 | 42815460 | 0.001047243 | −1.256536608 | −1.077111924 |
| chr12 | 27932420 | 27933820 | 0.001047096 | −1.189247386 | −1.052587982 |
| chr9 | 33288240 | 33288460 | 0.001047029 | −0.953171773 | −0.753821117 |
| chr9 | 33288240 | 33288460 | 0.001047029 | −0.953171773 | −0.753821117 |
| chr22 | 42486620 | 42487260 | 0.001046271 | −1.221148335 | −1.088720805 |
| chr22 | 42486640 | 42487240 | 0.001046271 | −1.221148335 | −1.088720805 |
| chr13 | 87088980 | 87090120 | −0.00104611 | −1.256993779 | −1.407430352 |
| chr6 | 155635020 | 155636380 | 0.001046089 | −1.325558338 | −1.113042836 |
| chr9 | 73901620 | 73902280 | −0.001045726 | −1.02085973 | −1.233777496 |
| chr9 | 19432460 | 19432920 | −0.001045646 | −0.884064194 | −1.099833131 |
| chr1 | 9551160 | 9551440 | 0.001045488 | −1.239031512 | −0.991781812 |
| chr3 | 183903000 | 183904660 | 0.00104544 | −1.422200466 | −1.237315145 |
| chr9 | 139886300 | 139887160 | 0.001045367 | −1.152617441 | −0.968207988 |
| chr3 | 47091120 | 47091620 | −0.001045299 | −1.223009732 | −1.404493284 |
| chr21 | 34142900 | 34144700 | 0.001045213 | −1.045825156 | −0.93741751 |
| chr12 | 91288920 | 91289460 | −0.001045192 | −1.165412054 | −1.324801926 |
| chr15 | 45906480 | 45906740 | 0.001045113 | −0.989524142 | −0.828891063 |
| chr4 | 25792020 | 25792600 | −0.001045068 | −1.222344757 | −1.305864602 |
| chr4 | 168037060 | 168038280 | −0.001044929 | −1.110658799 | −1.275995386 |
| chr4 | 126503820 | 126504300 | −0.001044837 | −1.103033332 | −1.259425048 |
| chr11 | 65343220 | 65343900 | 0.001044791 | −1.380304195 | −1.141174136 |
| chr1 | 66562020 | 66562440 | −0.001044539 | −0.920312868 | −1.160888226 |
| chr13 | 38609800 | 38610660 | 0.00104434 | −1.153969885 | −1.187525489 |
| chr21 | 24493120 | 24493600 | −0.001044044 | −1.041730186 | −1.243697347 |
| chr16 | 82163980 | 82164860 | 0.001044 | −1.140134382 | −1.047528024 |
| chr8 | 109527560 | 109528100 | −0.001043833 | −0.960430969 | −1.203817408 |
| chr5 | 60627400 | 60629780 | −0.001043822 | −1.245959246 | −1.373666286 |
| chr18 | 50219520 | 50220020 | −0.001043788 | −1.076246504 | −1.309957788 |
| chr12 | 75545460 | 75545960 | −0.001043539 | −0.887021945 | −1.228922708 |
| chr20 | 40904940 | 40905360 | 0.001043497 | −1.171588811 | −1.052317453 |
| chr20 | 40904940 | 40905360 | 0.001043497 | −1.171588811 | −1.052317453 |
| chr15 | 51422960 | 51423860 | 0.001043497 | −1.063197713 | −1.013243248 |
| chr3 | 105701360 | 105702080 | −0.001043388 | −1.081581672 | −1.311459979 |
| chr6 | 139094420 | 139095340 | 0.001043374 | −1.392269637 | −1.26048435 |
| chr19 | 13262460 | 13262900 | 0.001043243 | −1.215582025 | −1.092441132 |
| chr9 | 113578380 | 113578840 | −0.001043239 | −0.713926174 | −0.854864478 |
| chr9 | 98746260 | 98746720 | −0.001043141 | −0.887780016 | −1.068501807 |
| chr6 | 35551640 | 35551920 | 0.001043061 | −1.239825798 | −0.994828173 |
| chr3 | 178248200 | 178248600 | −0.001042791 | −0.982464942 | −1.193383167 |
| chr15 | 37903880 | 37904300 | −0.001042654 | −0.837021284 | −1.126239515 |
| chr21 | 40738580 | 40739120 | −0.001042586 | −1.037304534 | −1.233053819 |
| chr5 | 22283120 | 22283700 | 0.001042419 | −1.20705177 | −1.140087031 |
| chr8 | 68130240 | 68130660 | −0.00104238 | −1.028420036 | −1.282934807 |
| chr19 | 35631060 | 35631280 | 0.001042325 | −0.852750804 | −0.672366654 |
| chr12 | 110887520 | 110888860 | −0.001042168 | −1.164581556 | −1.24214092 |
| chr5 | 112287580 | 112288060 | −0.001042146 | −1.000453775 | −1.223758202 |
| chr19 | 3698760 | 3699080 | 0.001042076 | −1.021923994 | −0.877124593 |
| chr4 | 110132200 | 110132580 | −0.001041994 | −1.055748279 | −1.202631411 |
| chr4 | 14979100 | 14979620 | −0.001041941 | −0.93824057 | −1.204743542 |
| chr15 | 49447560 | 49448140 | 0.001041906 | −1.173472357 | −1.065755413 |
| chr7 | 77427300 | 77428880 | 0.00104176 | −1.295102022 | −1.130363395 |
| chr3 | 3330800 | 3331220 | −0.001041758 | −1.149013268 | −1.233159737 |
| chr13 | 31111540 | 31112020 | −0.001041546 | −1.101809387 | −1.237286658 |
| chr2 | 40479740 | 40480640 | −0.001041513 | −1.155353365 | −1.318833792 |
| chr2 | 40479720 | 40480660 | −0.001041513 | −1.155353365 | −1.318833792 |
| chr13 | 45914600 | 45915780 | 0.001041439 | −1.265821901 | −1.124897095 |
| chr16 | 58300120 | 58300500 | −0.001041335 | −0.902171065 | −1.069516835 |
| chr1 | 202857500 | 202859000 | 0.0010413 | −1.200470531 | −0.980105005 |
| chr22 | 35834040 | 35834440 | 0.001041208 | −0.990790247 | −0.873137011 |
| chr1 | 19854460 | 19854880 | −0.001041116 | −0.910025256 | −1.150156266 |
| chr12 | 61485460 | 61485960 | 0.001040977 | −1.18985743 | −1.138951983 |
| chr19 | 45393600 | 45395080 | 0.00104093 | −1.267322877 | −1.134961244 |
| chr2 | 235860020 | 235861280 | 0.001040901 | −1.34480983 | −1.223342203 |
| chr12 | 111834500 | 111835420 | 0.001040707 | −1.191265495 | −1.038576313 |
| chr2 | 37319280 | 37320020 | −0.001040638 | −1.031684579 | −1.206889664 |
| chr22 | 50607940 | 50609320 | 0.001040482 | −1.098601491 | −0.938871734 |
| chr13 | 20915700 | 20916380 | −0.001040321 | −1.129477498 | −1.364759637 |
| chr2 | 171688060 | 171688560 | −0.001040286 | −1.062519176 | −1.18404087 |
| chr18 | 60506760 | 60507280 | −0.001040249 | −1.110013464 | −1.250915742 |
| chr13 | 34102180 | 34102580 | −0.001040243 | −1.125029479 | −1.27314347 |
| chr19 | 41480500 | 41480780 | 0.001040238 | −1.121953431 | −0.894125399 |
| chr19 | 41480440 | 41480840 | 0.001040238 | −1.121953431 | −0.894125399 |
| chr2 | 6817920 | 6818320 | −0.001040202 | −0.940891332 | −1.207169307 |
| chr14 | 99905720 | 99906420 | −0.001040178 | −1.079547448 | −1.183402158 |
| chr12 | 88793720 | 88794180 | −0.001039953 | −1.123103471 | −1.3238514 |
| chr6 | 137314060 | 137314300 | 0.001039629 | −1.319164151 | −1.197857271 |
| chr11 | 44578240 | 44578520 | 0.001039615 | −1.326220835 | −1.10452668 |
| chr2 | 100105740 | 100107180 | 0.001039382 | −1.338979681 | −1.189711002 |
| chr8 | 98747340 | 98747700 | −0.001039351 | −1.029302725 | −1.206063553 |
| chr3 | 110726900 | 110727420 | −0.001039299 | −1.059749298 | −1.287405257 |
| chr7 | 42852840 | 42853320 | −0.00103919 | −0.979981225 | −1.169585842 |
| chr10 | 33245960 | 33247860 | 0.001039184 | −1.32719337 | −1.100869871 |
| chr8 | 56792160 | 56793320 | 0.001039011 | −1.309293693 | −1.070084357 |
| chr7 | 71895040 | 71895520 | 0.001038687 | −1.041928243 | −1.021469528 |
| chr5 | 126582240 | 126583000 | −0.001038677 | −1.082170843 | −1.325355207 |
| chr19 | 16738360 | 16739780 | 0.001038453 | −1.283490539 | −1.176186075 |
| chr7 | 20854120 | 20854640 | −0.001038266 | −1.041103444 | −1.224792498 |
| chr6 | 32938580 | 32938960 | −0.00103815 | −0.951695526 | −1.134149147 |
| chr11 | 118868100 | 118869480 | 0.001038093 | −1.208780602 | −1.08224328 |
| chr11 | 47416300 | 47416600 | 0.001038091 | −1.291571565 | −1.102034512 |
| chr3 | 13983700 | 13984040 | −0.001038034 | −0.923041884 | −1.22183192 |
| chr13 | 69417840 | 69418420 | −0.001038007 | −1.052411101 | −1.219981931 |
| chr4 | 74044400 | 74045920 | −0.001037831 | −1.111614682 | −1.291495981 |
| chr16 | 15685700 | 15686140 | −0.001037819 | −0.887957959 | −1.014206782 |
| chr6 | 40402540 | 40403060 | −0.001037813 | −1.081796217 | −1.252929869 |
| chr11 | 90617340 | 90617760 | 0.001037698 | −1.19782644 | −1.178396128 |
| chr1 | 207970720 | 207971000 | 0.001037264 | −1.18573116 | −0.982429157 |
| chr8 | 24077280 | 24077900 | −0.001037029 | −1.112651916 | −1.296607825 |
| chr8 | 34540220 | 34540700 | 0.001036986 | −1.150515745 | −1.132518307 |
| chr2 | 60901720 | 60902020 | 0.001036728 | −1.32922863 | −1.046251321 |
| chr8 | 87647860 | 87648240 | −0.001036632 | −1.101584589 | −1.312759061 |
| chr20 | 6308000 | 6308200 | −0.001036567 | −0.976286756 | −1.164954849 |
| chr20 | 6308000 | 6308200 | −0.001036567 | −0.976286756 | −1.164954849 |
| chr16 | 451400 | 452040 | 0.001036543 | −1.12034564 | −1.002070108 |
| chr16 | 451400 | 452040 | 0.001036543 | −1.12034564 | −1.002070108 |
| chr3 | 112027620 | 112027880 | −0.001036332 | −0.888787589 | −1.139268946 |
| chr3 | 112027620 | 112027880 | −0.001036332 | −0.888787589 | −1.139268946 |
| chr11 | 37766520 | 37766940 | −0.001036094 | −1.016119349 | −1.274964931 |
| chr12 | 110561620 | 110562620 | 0.001036082 | −1.440940338 | −1.303532812 |
| chr2 | 195514940 | 195515540 | −0.001036005 | −1.151204714 | −1.359104481 |
| chr6 | 20214060 | 20214540 | −0.001035912 | −0.987433481 | −1.232278991 |
| chr19 | 41196020 | 41197320 | 0.001035906 | −1.242392499 | −1.115518264 |
| chr11 | 70116200 | 70117420 | 0.001035744 | −1.43554101 | −1.292622758 |
| chr12 | 133107160 | 133107580 | 0.001035648 | −1.383504114 | −1.233437737 |
| chr2 | 48131620 | 48134260 | 0.001035356 | −1.187996852 | −0.959575841 |
| chr9 | 99179600 | 99181720 | 0.001035356 | −1.092968833 | −0.930089506 |
| chr2 | 112942120 | 112942540 | −0.001035206 | −0.948227607 | −1.15140312 |
| chr7 | 148292160 | 148293020 | −0.001035105 | −1.130457921 | −1.38000018 |
| chr1 | 161456500 | 161457400 | −0.001035036 | −1.001964241 | −1.036876004 |
| chr11 | 2554040 | 2554320 | −0.001035014 | −1.193281415 | −1.225968969 |
| chr22 | 32725960 | 32726740 | 0.001034908 | −1.019181714 | −0.83650659 |
| chr22 | 32725960 | 32726740 | 0.001034908 | −1.019181714 | −0.83650659 |
| chr4 | 58937700 | 58938140 | −0.001034834 | −0.983927942 | −1.195835967 |
| chr16 | 25035060 | 25035580 | 0.001034659 | −1.159405753 | −1.072046336 |
| chr12 | 2049220 | 2049500 | 0.001034595 | −1.331737592 | −1.194250229 |
| chr11 | 122932400 | 122933840 | 0.00103456 | −1.348702691 | −1.160777434 |
| chr2 | 194259260 | 194259760 | −0.001034383 | −1.074462076 | −1.275914347 |
| chr3 | 81677740 | 81678140 | 0.001034162 | −1.253881897 | −1.239495453 |
| chr22 | 33589740 | 33590120 | 0.001034157 | −1.050762477 | −1.035386728 |
| chr2 | 42552540 | 42553000 | −0.001034017 | −1.05168832 | −1.239253003 |
| chr17 | 57970020 | 57971120 | 0.001033949 | −1.091429145 | −0.966564914 |
| chr2 | 194203240 | 194203740 | 0.001033807 | −1.096273995 | −1.029663758 |
| chr4 | 163469180 | 163469620 | −0.001033723 | −0.957154606 | −1.214545702 |
| chr22 | 39189580 | 39190860 | 0.001033622 | −1.047710713 | −0.894548937 |
| chr3 | 106229840 | 106230140 | −0.001033365 | −0.967647274 | −1.238896988 |
| chr5 | 178157160 | 178158460 | 0.001033248 | −1.281474656 | −1.097339286 |
| chr2 | 12812100 | 12812580 | −0.001033086 | −1.182115198 | −1.248527541 |
| chr19 | 56577680 | 56578200 | 0.001032968 | −1.039189521 | −0.863250323 |
| chr5 | 147416380 | 147416920 | 0.001032816 | −1.232594579 | −1.152383676 |
| chr20 | 33146120 | 33147400 | 0.001032689 | −1.234567853 | −1.078360152 |
| chr4 | 69479340 | 69479620 | 0.001032608 | −1.389476181 | −1.161690336 |
| chr1 | 35578020 | 35578380 | −0.001032597 | −0.983034952 | −1.189838033 |
| chr3 | 48507040 | 48508060 | 0.001032501 | −1.188477899 | −1.034494566 |
| chr19 | 42103660 | 42104560 | 0.001032413 | −1.151407518 | −0.974207826 |
| chr22 | 20118540 | 20119940 | 0.001032413 | −1.196362526 | −1.064698973 |
| chr10 | 103588960 | 103590000 | 0.001032343 | −1.345878961 | −1.183846032 |
| chr17 | 16120340 | 16120640 | 0.001032166 | −1.112941546 | −1.001057351 |
| chr17 | 16120340 | 16120640 | 0.001032166 | −1.112941546 | −1.001057351 |
| chr1 | 21835200 | 21836680 | 0.001032158 | −1.105907004 | −0.923563436 |
| chr19 | 1478480 | 1479780 | 0.00103215 | −1.134522824 | −0.932762276 |
| chr5 | 7395680 | 7396900 | −0.001031985 | −1.187239075 | −1.304303221 |
| chr11 | 14771220 | 14771600 | −0.001031847 | −1.077002299 | −1.24753692 |
| chr19 | 4400060 | 4400900 | 0.00103182 | −1.203576894 | −1.066076469 |
| chr7 | 123630980 | 123631340 | −0.001031537 | −0.775144349 | −1.05470345 |
| chr19 | 44258580 | 44259600 | 0.001031414 | −1.106293118 | −0.982902709 |
| chr2 | 229128100 | 229128500 | −0.001031312 | −0.916710877 | −1.072561928 |
| chr2 | 148517700 | 148518140 | −0.001031295 | −1.088661656 | −1.207613907 |
| chr2 | 82475320 | 82475760 | 0.00103126 | −1.142342052 | −1.064927753 |
| chr7 | 143594020 | 143594440 | 0.001031184 | −1.224425702 | −1.117740071 |
| chr20 | 17996720 | 17997100 | 0.001031123 | −1.17668744 | −1.14564244 |
| chr1 | 89989780 | 89991160 | 0.001031115 | −1.185691673 | −0.96800612 |
| chr17 | 56415380 | 56415940 | 0.001030957 | −1.008509474 | −0.825503978 |
| chr17 | 56415280 | 56416040 | 0.001030957 | −1.008509474 | −0.825503978 |
| chr12 | 99038200 | 99039600 | 0.001030954 | −1.399710594 | −1.254802698 |
| chr17 | 57830840 | 57831140 | 0.001030918 | −1.102409121 | −1.015419231 |
| chr17 | 57830840 | 57831140 | 0.001030918 | −1.102409121 | −1.015419231 |
| chr17 | 74931700 | 74932240 | −0.001030887 | −1.084806329 | −1.273153773 |
| chr12 | 113857220 | 113857720 | −0.001030785 | −1.032787104 | −1.278383652 |
| chr5 | 32612920 | 32613860 | 0.001030719 | −1.310395077 | −1.071083311 |
| chr5 | 105900100 | 105900740 | −0.001030473 | −1.098013661 | −1.274695798 |
| chr17 | 44269880 | 44271700 | 0.001030411 | −1.23484413 | −1.091094114 |
| chr12 | 133107140 | 133107680 | 0.001030388 | −1.283570655 | −1.176590817 |
| chr1 | 64971020 | 64971860 | 0.001030267 | −1.15372054 | −0.90707651 |
| chr1 | 243389120 | 243389700 | −0.001030237 | −1.022703847 | −1.207192668 |
| chr2 | 57096460 | 57096920 | 0.001029942 | −1.199710091 | −1.140910457 |
| chr11 | 728900 | 729200 | 0.001029933 | −1.167755472 | −0.929969125 |
| chr6 | 126902860 | 126903300 | −0.001029852 | −1.103744433 | −1.257122752 |
| chr19 | 442800 | 443340 | 0.001029454 | −1.066200999 | −0.874960838 |
| chr19 | 442800 | 443340 | 0.001029454 | −1.066200999 | −0.874960838 |
| chr2 | 115933680 | 115934040 | −0.001029372 | −0.854210626 | −1.181471853 |
| chr2 | 115933600 | 115934120 | −0.001029372 | −0.854210626 | −1.181471853 |
| chr1 | 44382900 | 44383840 | −0.001029336 | −0.994577154 | −1.123504451 |
| chr7 | 155725060 | 155726240 | 0.001029272 | −1.099637069 | −1.013534241 |
| chr4 | 55588800 | 55589200 | −0.001029054 | −1.010383027 | −1.250259292 |
| chr1 | 26535180 | 26535640 | −0.001028883 | −0.999654482 | −1.14337468 |
| chr5 | 133702440 | 133704000 | 0.001028589 | −1.194858041 | −0.932415791 |
| chr10 | 128460120 | 128460540 | 0.001028522 | −1.269754236 | −1.165125838 |
| chr4 | 158667240 | 158667760 | −0.001028516 | −1.123099199 | −1.342229646 |
| chr16 | 2801340 | 2803360 | 0.001028246 | −1.103195751 | −0.944174733 |
| chr10 | 63590960 | 63592100 | −0.001028075 | −1.000925287 | −1.302506495 |
| chr1 | 205718380 | 205719640 | 0.001028024 | −1.187211456 | −1.011390017 |
| chr14 | 41442740 | 41443120 | −0.001027999 | −0.834424532 | −1.151072491 |
| chr2 | 115329180 | 115329760 | −0.001027977 | −1.016553976 | −1.261079621 |
| chr19 | 46532040 | 46532580 | 0.001027648 | −1.166634719 | −1.017660851 |
| chr12 | 130122860 | 130123380 | −0.001027627 | −1.098319466 | −1.300844747 |
| chr1 | 72153860 | 72154240 | −0.001027345 | −1.043991741 | −1.287563684 |
| chr5 | 176856260 | 176856500 | 0.001027325 | −1.218106147 | −0.985866379 |
| chr18 | 558800 | 559580 | 0.001027217 | −1.265865125 | −1.134717252 |
| chr18 | 558800 | 559580 | 0.001027217 | −1.265865125 | −1.134717252 |
| chr2 | 122512620 | 122513660 | 0.001027179 | −1.262774997 | −1.101386422 |
| chr16 | 8837080 | 8837780 | −0.001027066 | −0.958782174 | −1.222570549 |
| chr17 | 53655860 | 53656200 | 0.001026913 | −0.940613069 | −0.941534123 |
| chr3 | 141456280 | 141457940 | 0.001026709 | −1.261672944 | −1.163874163 |
| chr18 | 65551880 | 65552340 | −0.001026662 | −1.090279209 | −1.237656756 |
| chr18 | 65551860 | 65552360 | −0.001026662 | −1.090279209 | −1.237656756 |
| chr8 | 113911520 | 113912200 | −0.001026639 | −1.035516716 | −1.108868283 |
| chr4 | 175364180 | 175364620 | −0.001026606 | −1.038747485 | −1.236018402 |
| chr2 | 196290480 | 196291280 | −0.001026577 | −1.144756361 | −1.323924441 |
| chr4 | 118564540 | 118565000 | −0.001026557 | −1.032385384 | −1.228991905 |
| chr22 | 19466420 | 19468000 | 0.00102654 | −1.11321642 | −0.931133504 |
| chr8 | 67691780 | 67692080 | −0.001026468 | −1.048689298 | −1.217225507 |
| chr4 | 126094340 | 126095320 | 0.001026454 | −1.331856469 | −1.286911075 |
| chr7 | 33227520 | 33227960 | 0.001026366 | −1.229993732 | −1.085175185 |
| chr18 | 64538620 | 64539100 | −0.001026358 | −1.220387336 | −1.363822694 |
| chr7 | 141654840 | 141655220 | −0.001026259 | −0.889678906 | −1.098059566 |
| chr11 | 32558760 | 32559180 | −0.001026173 | −1.130214121 | −1.229414349 |
| chr16 | 83935600 | 83935920 | −0.00102608 | −0.824706684 | −1.027714009 |
| chr15 | 67134720 | 67135420 | −0.001026016 | −0.98192262 | −1.028246363 |
| chr16 | 4303680 | 4304480 | 0.001025944 | −1.290512611 | −1.155569003 |
| chr16 | 4303660 | 4304480 | 0.001025944 | −1.290512611 | −1.151451581 |
| chr19 | 41859440 | 41860180 | 0.001025822 | −1.249359992 | −1.112027877 |
| chr17 | 1618800 | 1620680 | 0.001025676 | −1.299128418 | −1.207645436 |
| chr8 | 125126920 | 125127340 | −0.001025662 | −1.006175005 | −1.237873637 |
| chr12 | 109197020 | 109197960 | 0.001025294 | −1.328119341 | −1.15990534 |
| chr2 | 150633720 | 150634180 | −0.001025231 | −0.990424918 | −1.164779955 |
| chr3 | 48481100 | 48481940 | −0.001025161 | −1.057974952 | −1.12703377 |
| chr2 | 141060420 | 141060820 | −0.00102509 | −0.845065315 | −1.055739593 |
| chr2 | 141060420 | 141060820 | −0.00102509 | −0.845065315 | −1.055739593 |
| chr10 | 83106120 | 83106640 | −0.001024914 | −1.021719329 | −1.254470515 |
| chr11 | 68038660 | 68040180 | 0.001024702 | −1.41734785 | −1.228679402 |
| chr22 | 38054000 | 38055860 | −0.00102461 | −0.928621306 | −1.026628225 |
| chr9 | 9550240 | 9550700 | −0.00102448 | −0.754560584 | −0.962847463 |
| chr4 | 155223780 | 155224160 | 0.001024248 | −1.170344463 | −1.033833169 |
| chr1 | 1231980 | 1232500 | 0.001024212 | −1.220714262 | −1.063522523 |
| chr6 | 22432680 | 22433120 | −0.001023972 | −1.029123544 | −1.247679758 |
| chr20 | 25227680 | 25229500 | 0.001023886 | −1.096190688 | −0.934927635 |
| chr14 | 81678080 | 81678480 | −0.001023822 | −0.839128079 | −1.055570223 |
| chr11 | 67454620 | 67455180 | −0.001023763 | −1.092980776 | −1.203664623 |
| chr1 | 167708280 | 167708760 | −0.001023684 | −0.911899226 | −1.102536445 |
| chr2 | 74691420 | 74692700 | 0.001023519 | −1.095949405 | −0.860196555 |
| chr1 | 89823920 | 89824180 | 0.001023487 | −1.049022903 | −0.917053793 |
| chr16 | 47494700 | 47495680 | 0.00102336 | −1.199216958 | −0.932257389 |
| chr9 | 134406000 | 134407240 | 0.00102325 | −1.20889948 | −0.991442093 |
| chr5 | 96142920 | 96144280 | 0.001023213 | −1.274098331 | −1.096493074 |
| chr5 | 169170700 | 169171100 | 0.001023193 | −1.100807745 | −1.033387657 |
| chr6 | 98099480 | 98099880 | −0.001023178 | −1.055482443 | −1.197524783 |
| chr9 | 102863240 | 102863560 | 0.001022946 | −0.890814569 | −0.843650022 |
| chr7 | 128734340 | 128734780 | −0.001022854 | −1.10320279 | −1.238110342 |
| chr3 | 120461020 | 120462000 | 0.001022846 | −1.249691561 | −1.169671394 |
| chr14 | 104602540 | 104602900 | −0.001022713 | −1.054085585 | −1.15497804 |
| chr15 | 43476880 | 43478200 | 0.001022667 | −1.196792092 | −1.028497681 |
| chr11 | 65626820 | 65627120 | 0.001022664 | −1.326652303 | −1.201267222 |
| chr4 | 14379060 | 14379520 | −0.001022517 | −1.01552109 | −1.202764697 |
| chr4 | 14379040 | 14379540 | −0.001022517 | −1.01552109 | −1.202764697 |
| chr12 | 2049200 | 2049500 | 0.001022365 | −1.358512602 | −1.205921856 |
| chr7 | 105516520 | 105517800 | 0.001022307 | −1.347632287 | −1.186694098 |
| chr1 | 27935180 | 27935900 | −0.00102199 | −0.86459284 | −0.973894861 |
| chr1 | 107379020 | 107379600 | −0.001021922 | −1.005389283 | −1.228177914 |
| chr2 | 210288180 | 210289000 | 0.001021796 | −1.106099403 | −0.946424793 |
| chr1 | 14840800 | 14841340 | −0.001021785 | −1.208605665 | −1.343408096 |
| chr20 | 62710260 | 62711860 | 0.001021777 | −1.131035301 | −1.039988432 |
| chr10 | 24809340 | 24809840 | 0.001021737 | −1.162491528 | −1.075068847 |
| chr12 | 14518220 | 14519420 | 0.001021685 | −1.183875176 | −1.094126643 |
| chr17 | 61816880 | 61817460 | 0.001021613 | −1.149009357 | −1.099336128 |
| chr22 | 46645720 | 46646920 | 0.001021552 | −0.980070435 | −0.788707327 |
| chr9 | 35646740 | 35647700 | −0.001021479 | −0.96281032 | −0.935692683 |
| chr2 | 58655200 | 58656220 | 0.001021343 | −1.16715441 | −0.95208301 |
| chr1 | 89989840 | 89991120 | 0.001021329 | −1.185691673 | −0.963985905 |
| chr15 | 64125500 | 64126780 | 0.001021251 | −1.136823878 | −0.927147206 |
| chr1 | 40008380 | 40008700 | −0.001021233 | −0.971823031 | −1.10984453 |
| chr11 | 25671780 | 25673580 | −0.001021119 | −1.015051533 | −1.134510231 |
| chr1 | 96776640 | 96777160 | −0.001020886 | −0.953040012 | −1.148267504 |
| chr6 | 102152680 | 102153040 | −0.001020874 | −0.981697375 | −1.24456729 |
| chr6 | 102152640 | 102153080 | −0.001020874 | −0.981697375 | −1.24456729 |
| chr19 | 14543900 | 14544780 | 0.001020805 | −1.110089518 | −0.990607081 |
| chr10 | 102480700 | 102481080 | −0.001020764 | −0.940136749 | −1.183348158 |
| chr5 | 2377760 | 2378300 | 0.001020618 | −1.336078594 | −1.166885814 |
| chr19 | 14674960 | 14675540 | 0.001020588 | −1.149769137 | −1.016194888 |
| chr8 | 138959600 | 138960040 | −0.00102056 | −1.037467089 | −1.26642139 |
| chr8 | 138959580 | 138960060 | −0.00102056 | −1.037467089 | −1.26642139 |
| chr2 | 175819740 | 175820080 | −0.001020511 | −0.901979661 | −1.190676982 |
| chr2 | 175819740 | 175820080 | −0.001020511 | −0.901979661 | −1.190676982 |
| chr5 | 167218260 | 167218700 | −0.00102047 | −1.178437129 | −1.357443957 |
| chr5 | 78833880 | 78834340 | −0.001020343 | −1.067296953 | −1.290186498 |
| chr2 | 67874600 | 67875100 | −0.001019968 | −1.048975807 | −1.248735705 |
| chr6 | 84427000 | 84427860 | 0.001019918 | −1.244157619 | −1.055887387 |
| chr7 | 138326360 | 138326800 | −0.001019861 | −1.016714782 | −1.192906916 |
| chr6 | 11575780 | 11576100 | 0.001019815 | −1.201267545 | −1.018782664 |
| chr11 | 87356620 | 87357480 | −0.001019771 | −1.157587052 | −1.239433255 |
| chr21 | 35573780 | 35574920 | −0.001019686 | −0.792544464 | −0.87933119 |
| chr8 | 10609500 | 10610100 | −0.001019648 | −1.004819958 | −1.236399658 |
| chr4 | 135668400 | 135668700 | −0.001019611 | −0.948179449 | −1.121191348 |
| chr1 | 102931500 | 102932280 | −0.001019278 | −1.166775135 | −1.347543815 |
| chr1 | 194273000 | 194273300 | −0.001019251 | −0.958853209 | −1.080023133 |
| chr1 | 219102200 | 219102900 | −0.00101922 | −1.088883108 | −1.312016085 |
| chr3 | 175898800 | 175899060 | −0.001019098 | −1.052190731 | −1.22821285 |
| chr3 | 175898800 | 175899060 | −0.001019098 | −1.052190731 | −1.22821285 |
| chr2 | 129623960 | 129624640 | −0.001018974 | −0.93634957 | −1.169199021 |
| chr16 | 4233040 | 4233260 | 0.001018665 | −0.962961674 | −0.829253672 |
| chr13 | 54076100 | 54076600 | −0.001018645 | −1.118501791 | −1.213665688 |
| chr16 | 57118200 | 57118480 | 0.001018591 | −1.164007036 | −0.954553419 |
| chr15 | 102191500 | 102193500 | 0.001018485 | −1.114023095 | −0.9245272 |
| chr2 | 216973440 | 216974680 | 0.001018482 | −1.313716023 | −1.22282747 |
| chr4 | 75353900 | 75354280 | −0.001018083 | −0.927711417 | −1.142971729 |
| chr12 | 10656720 | 10657280 | −0.001017911 | −1.096839235 | −1.263721042 |
| chr2 | 64401260 | 64401720 | −0.001017756 | −0.922663582 | −1.19567174 |
| chr9 | 18287220 | 18287860 | −0.001017716 | −0.701714733 | −1.007670719 |
| chr11 | 32913680 | 32915120 | 0.001017485 | −1.212505213 | −1.036128368 |
| chr15 | 74731120 | 74731360 | 0.001017412 | −1.098242884 | −0.92098896 |
| chr19 | 19174220 | 19175380 | 0.001017399 | −1.173187964 | −0.97638886 |
| chr6 | 8819380 | 8819780 | 0.001017144 | −1.341609093 | −1.211854501 |
| chr4 | 181936880 | 181937440 | −0.001017136 | −1.212959697 | −1.371983397 |
| chr15 | 34658600 | 34660080 | 0.001017117 | −1.041992986 | −0.852053098 |
| chr15 | 67327580 | 67328380 | −0.001017067 | −1.043835059 | −1.161520109 |
| chr4 | 178191440 | 178191860 | 0.001017066 | −1.160753242 | −1.170190138 |
| chr6 | 28023200 | 28023880 | −0.001017065 | −1.133323283 | −1.323551044 |
| chr6 | 119194140 | 119194560 | −0.001016799 | −1.05863682 | −1.166103372 |
| chr1 | 193028040 | 193028880 | 0.001016692 | −1.174941757 | −0.991913303 |
| chr17 | 32073080 | 32073680 | −0.001016576 | −0.86061877 | −1.091467541 |
| chr6 | 66078980 | 66079440 | −0.001016494 | −1.135243792 | −1.327667374 |
| chr6 | 132624680 | 132625260 | −0.001016459 | −1.22152905 | −1.323860142 |
| chr2 | 231917180 | 231918380 | 0.001016439 | −1.246300467 | −1.035100581 |
| chr1 | 92788340 | 92788820 | −0.001016229 | −1.033600356 | −1.231264495 |
| chr8 | 98655840 | 98657040 | 0.001016178 | −1.274194252 | −1.162889925 |
| chr15 | 45926820 | 45927360 | 0.001016149 | −1.022263356 | −0.869739091 |
| chr3 | 161408400 | 161408760 | 0.00101586 | −1.29574725 | −1.203958632 |
| chr6 | 117291220 | 117291700 | −0.00101584 | −1.111357564 | −1.350420543 |
| chr5 | 3789420 | 3790120 | −0.001015737 | −1.14385195 | −1.319712924 |
| chr1 | 200042180 | 200042700 | −0.001015691 | −0.9655526 | −1.155153817 |
| chr11 | 20384800 | 20385800 | 0.001015663 | −1.076643285 | −0.909751866 |
| chr17 | 80019720 | 80020080 | −0.001015657 | −0.879488388 | −0.973151812 |
| chr5 | 162671200 | 162671660 | 0.001015289 | −1.221427999 | −1.137906818 |
| chr6 | 19406720 | 19407020 | −0.001015234 | −1.129023432 | −1.230412833 |
| chr6 | 19406720 | 19407020 | −0.001015234 | −1.129023432 | −1.230412833 |
| chr16 | 28935540 | 28937000 | 0.001015138 | −1.205677908 | −1.041256966 |
| chr8 | 90423060 | 90423520 | −0.001014966 | −1.272793494 | −1.421836796 |
| chr1 | 206864640 | 206865040 | −0.001014867 | −0.863026962 | −1.109267795 |
| chr20 | 21283280 | 21284660 | 0.001014755 | −1.346610617 | −1.215653689 |
| chr2 | 175009420 | 175009900 | −0.001014719 | −0.97493065 | −1.129866201 |
| chr17 | 2302900 | 2303640 | 0.001014697 | −1.054256336 | −0.914218233 |
| chr10 | 43904240 | 43904980 | 0.001014509 | −1.228754051 | −1.046849396 |
| chr2 | 117801200 | 117802760 | 0.001014488 | −1.20338275 | −1.19667025 |
| chr11 | 14771200 | 14771640 | −0.001014482 | −1.05748685 | −1.242515985 |
| chr20 | 57276020 | 57276640 | −0.001014467 | −1.079612821 | −1.200580563 |
| chr17 | 38136140 | 38137760 | 0.001014349 | −1.101004058 | −1.005079851 |
| chr3 | 183902960 | 183904460 | 0.001014304 | −1.263726965 | −1.116253901 |
| chr9 | 104000040 | 104000780 | 0.001013962 | −1.089871245 | −1.015343003 |
| chr9 | 104000040 | 104000780 | 0.001013962 | −1.089871245 | −1.015343003 |
| chr1 | 119742840 | 119743360 | −0.001013651 | −0.866124866 | −1.059701924 |
| chr16 | 67970640 | 67970840 | 0.001013606 | −0.992272669 | −0.877880738 |
| chr7 | 128065880 | 128066160 | 0.001013571 | −1.218630168 | −1.022511232 |
| chr7 | 128065880 | 128066160 | 0.001013571 | −1.218630168 | −1.022511232 |
| chr19 | 42755420 | 42755740 | 0.001013567 | −1.108454493 | −0.892627416 |
| chr6 | 162442160 | 162442440 | −0.001013526 | −1.034031618 | −1.198551186 |
| chr1 | 72767480 | 72768440 | 0.001013416 | −1.380122607 | −1.25925047 |
| chr5 | 180475540 | 180476140 | −0.001013371 | −1.193629215 | −1.352503753 |
| chr2 | 198227020 | 198227620 | 0.001013322 | −1.220711209 | −1.196552973 |
| chr2 | 157189140 | 157189440 | −0.001013166 | −1.079540428 | −1.136234403 |
| chr6 | 91775400 | 91775940 | −0.001013149 | −1.215955463 | −1.378522342 |
| chr15 | 59729960 | 59730720 | −0.001013123 | −0.967470823 | −1.111478954 |
| chr5 | 21418020 | 21418700 | −0.001013106 | −1.1056096 | −1.308333347 |
| chr20 | 48802800 | 48803080 | 0.001012923 | −1.338209917 | −1.179401773 |
| chr14 | 104387740 | 104388140 | 0.001012896 | −1.301831253 | −1.125976228 |
| chr17 | 66461840 | 66463060 | 0.001012635 | −1.102238386 | −0.985360949 |
| chr17 | 66461840 | 66463060 | 0.001012635 | −1.102238386 | −0.985360949 |
| chr12 | 56693680 | 56694760 | 0.001012583 | −1.303877842 | −1.181489037 |
| chr3 | 75211280 | 75211800 | −0.001012506 | −1.136755266 | −1.345321722 |
| chr3 | 75211260 | 75211820 | −0.001012506 | −1.136755266 | −1.345321722 |
| chr1 | 113161620 | 113163060 | 0.001012342 | −1.185945793 | −0.992810382 |
| chr1 | 12079160 | 12080020 | 0.00101221 | −1.159681637 | −1.059362816 |
| chr7 | 85827800 | 85828200 | −0.001012184 | −1.056903045 | −1.261888921 |
| chr2 | 12634540 | 12635500 | −0.001011998 | −0.969924395 | −1.23132418 |
| chr18 | 76828620 | 76829840 | 0.001011876 | −1.406630739 | −1.241781564 |
| chr4 | 93109400 | 93109820 | −0.001011848 | −0.987773145 | −1.151817442 |
| chr2 | 113506980 | 113507900 | 0.001011798 | −1.320502199 | −1.094966569 |
| chr10 | 121484780 | 121486420 | 0.001011751 | −1.322097292 | −1.178694009 |
| chr3 | 78731340 | 78732120 | −0.001011655 | −0.958296145 | −1.268480246 |
| chr4 | 33393100 | 33393600 | −0.001011546 | −1.005022371 | −1.237464653 |
| chr2 | 12636780 | 12637240 | −0.001011263 | −0.920971208 | −1.142954662 |
| chr17 | 45743660 | 45744020 | −0.001011199 | −0.979137851 | −1.152829987 |
| chr17 | 45743660 | 45744020 | −0.001011199 | −0.979137851 | −1.152829987 |
| chr9 | 105798660 | 105799240 | −0.001010893 | −0.975435893 | −1.127261442 |
| chr4 | 30349780 | 30350160 | −0.001010453 | −1.133815415 | −1.316728757 |
| chr3 | 187863740 | 187864220 | −0.001010363 | −1.0982954 | −1.330412527 |
| chr8 | 21595460 | 21595940 | −0.001010362 | −1.18615074 | −1.279957653 |
| chr4 | 63326960 | 63327360 | −0.00101025 | −1.06193471 | −1.212761529 |
| chr1 | 154932100 | 154932360 | 0.001010173 | −1.001658423 | −0.830862969 |
| chr3 | 29336560 | 29337140 | −0.001010154 | −1.004780457 | −1.276238019 |
| chr12 | 23990820 | 23991340 | −0.001010102 | −1.151804848 | −1.343662044 |
| chr1 | 196405900 | 196406580 | −0.001010018 | −1.06705074 | −1.253707223 |
| chr22 | 18303380 | 18304020 | −0.001010009 | −0.838842472 | −1.141830555 |
| chr9 | 125112880 | 125113180 | 0.001009951 | −1.140259229 | −0.961214066 |
| chr4 | 84614340 | 84615260 | −0.001009927 | −1.049482284 | −1.308179291 |
| chr10 | 129150260 | 129150760 | 0.00100991 | −1.311218418 | −1.14706214 |
| chr13 | 47696840 | 47697600 | −0.001009883 | −1.079096602 | −1.261650169 |
| chr19 | 1069220 | 1069420 | 0.001009793 | −1.136707435 | −0.967583896 |
| chr5 | 71615500 | 71616640 | 0.001009767 | −1.25511121 | −1.154674386 |
| chr5 | 71615500 | 71616640 | 0.001009767 | −1.25511121 | −1.154674386 |
| chr3 | 171356800 | 171357720 | −0.00100976 | −1.239107755 | −1.428719752 |
| chr1 | 10475140 | 10476120 | 0.001009727 | −1.037826033 | −0.950913662 |
| chr9 | 130533260 | 130534080 | −0.001009265 | −0.992851999 | −1.053859192 |
| chr17 | 7513060 | 7513500 | −0.001009104 | −0.926304366 | −1.136086571 |
| chr21 | 29028060 | 29028580 | −0.001009028 | −0.957779747 | −1.131237833 |
| chr17 | 61836880 | 61837120 | 0.001009007 | −1.071984522 | −0.955191516 |
| chr13 | 49070720 | 49071180 | −0.001008956 | −1.213520767 | −1.279422973 |
| chr13 | 49070700 | 49071200 | −0.001008956 | −1.213520767 | −1.279422973 |
| chr6 | 92202740 | 92203280 | −0.001008941 | −1.028920515 | −1.124676326 |
| chr13 | 88557580 | 88558180 | 0.001008919 | −1.200697738 | −1.195004659 |
| chr9 | 24829320 | 24829720 | −0.001008843 | −0.847873111 | −1.050566463 |
| chr1 | 240061460 | 240061760 | 0.001008688 | −1.19495552 | −1.028672627 |
| chr15 | 54845420 | 54845920 | −0.001008669 | −1.035279639 | −1.287599904 |
| chr22 | 19577000 | 19577500 | −0.001008668 | −0.928456663 | −1.063350698 |
| chr6 | 163368120 | 163368840 | −0.001008651 | −1.169777332 | −1.405738799 |
| chr9 | 75018580 | 75019120 | 0.001008612 | −0.854194818 | −0.78049023 |
| chr5 | 1008580 | 1009880 | 0.001008417 | −1.280792516 | −1.03968813 |
| chr15 | 24957340 | 24957960 | −0.001008294 | −0.774594736 | −1.067734406 |
| chr11 | 47663760 | 47664460 | 0.00100814 | −1.329506984 | −1.204809685 |
| chr11 | 47663740 | 47664480 | 0.00100814 | −1.329506984 | −1.204809685 |
| chr12 | 48172800 | 48173700 | 0.001008121 | −1.273961001 | −1.104097671 |
| chr13 | 48702060 | 48702500 | −0.0010081 | −1.004683606 | −1.124167011 |
| chr6 | 48987420 | 48988300 | −0.001008082 | −1.025967069 | −1.143308576 |
| chr3 | 34536640 | 34537140 | −0.00100788 | −1.027346004 | −1.255639467 |
| chr6 | 12168200 | 12168820 | −0.001007861 | −1.23263709 | −1.39083466 |
| chr7 | 149019780 | 149020280 | −0.00100779 | −1.033784385 | −1.253547421 |
| chr13 | 64464480 | 64465100 | −0.001007676 | −1.181077346 | −1.30774886 |
| chr2 | 45208760 | 45209100 | 0.001007585 | −1.220906561 | −1.017234054 |
| chr2 | 145168400 | 145168920 | −0.001007411 | −1.169680092 | −1.286762256 |
| chr4 | 8862500 | 8862900 | 0.001007038 | −1.209169838 | −0.981010434 |
| chr7 | 43972300 | 43972900 | −0.001006717 | −0.989962168 | −1.187321166 |
| chr12 | 89095440 | 89096480 | −0.0010065 | −1.217710385 | −1.415272939 |
| chr12 | 89095440 | 89096480 | −0.0010065 | −1.217710385 | −1.415272939 |
| chr15 | 64647820 | 64648920 | 0.001006379 | −1.09242789 | −0.948708046 |
| chr15 | 89973640 | 89974580 | 0.001006341 | −1.270409552 | −1.06288028 |
| chr1 | 16173900 | 16174980 | 0.001006259 | −1.127256665 | −0.90392289 |
| chr10 | 13814040 | 13814380 | −0.001006116 | −1.0373272 | −1.240421244 |
| chr9 | 140386720 | 140387520 | 0.001005968 | −0.994303639 | −0.858371755 |
| chr4 | 177728260 | 177729300 | −0.001005889 | −1.031818819 | −1.217851673 |
| chr8 | 8183440 | 8183920 | −0.001005881 | −1.041440323 | −1.260667888 |
| chr11 | 112096640 | 112098140 | 0.001005871 | −1.196978581 | −1.022234572 |
| chr21 | 38494520 | 38494960 | 0.00100587 | −0.977480167 | −0.900200518 |
| chr21 | 38494520 | 38494960 | 0.00100587 | −0.977480167 | −0.900200518 |
| chr14 | 95987060 | 95987580 | −0.0010056 | −0.987358711 | −1.133439845 |
| chr20 | 34580680 | 34580960 | 0.00100559 | −1.256999422 | −1.169635497 |
| chr8 | 87442800 | 87443800 | −0.001005392 | −1.135346445 | −1.356350131 |
| chr16 | 67875720 | 67876780 | 0.001005264 | −1.090281104 | −0.963365235 |
| chr13 | 86732900 | 86733440 | −0.001005214 | −1.079694044 | −1.309331558 |
| chr5 | 90976520 | 90977100 | −0.00100512 | −1.005793249 | −1.243888282 |
| chr5 | 33272940 | 33273440 | −0.001005115 | −1.209722679 | −1.391984423 |
| chr16 | 67595800 | 67597480 | −0.001005113 | −0.971644616 | −1.079401489 |
| chr22 | 37623540 | 37623920 | 0.001005078 | −1.061439863 | −0.89549802 |
| chr21 | 35565360 | 35565820 | −0.001004833 | −0.774469838 | −0.937483305 |
| chr10 | 101891180 | 101891620 | −0.001004752 | −0.91044846 | −1.129118981 |
| chr19 | 47742680 | 47743180 | 0.001004649 | −1.086706347 | −0.934225392 |
| chr2 | 157247120 | 157247660 | −0.001004553 | −0.935213989 | −1.154603676 |
| chr2 | 185889180 | 185889700 | −0.001004481 | −1.053135379 | −1.185339088 |
| chr14 | 43955800 | 43956360 | −0.001004456 | −1.007575707 | −1.237819625 |
| chr5 | 77655460 | 77657200 | 0.001004307 | −1.242532213 | −1.068457956 |
| chr3 | 196438700 | 196439640 | 0.001004256 | −1.410483211 | −1.23411157 |
| chr11 | 25541420 | 25541860 | −0.001004216 | −1.027059754 | −1.210399672 |
| chr2 | 103369760 | 103370640 | 0.001004157 | −1.321167537 | −1.073911315 |
| chr13 | 90246960 | 90247500 | 0.001004101 | −1.156487579 | −1.179515792 |
| chr14 | 74216120 | 74216440 | −0.001004003 | −1.02027969 | −1.207744134 |
| chr14 | 74216080 | 74216480 | −0.001004003 | −1.02027969 | −1.207744134 |
| chr3 | 115716320 | 115717180 | −0.001003965 | −1.072347288 | −1.305694955 |
| chr21 | 43298640 | 43299780 | −0.001003836 | −1.017412577 | −1.140110725 |
| chr12 | 125398540 | 125399940 | 0.001003825 | −1.414576153 | −1.277410922 |
| chr10 | 131934000 | 131935300 | 0.001003665 | −1.316522535 | −1.19432308 |
| chr10 | 63590980 | 63592040 | −0.001003566 | −1.016792547 | −1.29348878 |
| chr2 | 149087440 | 149087900 | 0.001003408 | −1.217109733 | −1.08930448 |
| chr11 | 92144060 | 92144480 | −0.00100339 | −1.062400463 | −1.332528499 |
| chr6 | 141809960 | 141810500 | −0.001003338 | −0.875293236 | −1.096101194 |
| chr6 | 33086100 | 33086940 | 0.001003326 | −1.229502575 | −1.031989926 |
| chr5 | 145029900 | 145030340 | −0.001003292 | −1.109252643 | −1.330173993 |
| chr7 | 98910580 | 98910840 | 0.001003095 | −1.220827754 | −1.034872113 |
| chr12 | 104234100 | 104235920 | −0.001003092 | −1.192533134 | −1.297126725 |
| chr13 | 87149880 | 87150580 | −0.001003081 | −1.264797194 | −1.443592916 |
| chr13 | 87149880 | 87150580 | −0.001003081 | −1.264797194 | −1.443592916 |
| chr17 | 36584380 | 36585180 | 0.001003001 | −0.959239556 | −0.808465772 |
| chr2 | 54197460 | 54198680 | 0.001002833 | −1.326492659 | −1.111621836 |
| chr2 | 58121140 | 58121520 | −0.001002671 | −1.177527383 | −1.292765373 |
| chr17 | 80106020 | 80107200 | 0.001002509 | −1.357373055 | −1.232140268 |
| chr5 | 157936700 | 157937220 | −0.00100246 | −1.01361207 | −1.256064636 |
| chr19 | 55615500 | 55615800 | 0.001002398 | −0.951972522 | −0.960227153 |
| chr19 | 55615500 | 55615800 | 0.001002398 | −0.951972522 | −0.960227153 |
| chr20 | 61680020 | 61681040 | 0.001002369 | −1.29490833 | −1.174780136 |
| chr4 | 26514440 | 26514880 | 0.001002288 | −1.240209995 | −1.136545969 |
| chr20 | 42273680 | 42273940 | 0.001002236 | −1.356318662 | −1.154622253 |
| chr20 | 42273680 | 42273940 | 0.001002236 | −1.356318662 | −1.154622253 |
| chr8 | 917520 | 918040 | 0.001002188 | −1.365560046 | −1.256085493 |
| chr21 | 47488260 | 47489600 | 0.001002108 | −1.139641014 | −0.959148742 |
| chr17 | 74068280 | 74068840 | 0.001002076 | −1.173151042 | −1.029670449 |
| chr4 | 38677200 | 38677480 | 0.001002062 | −1.294617492 | −1.139306021 |
| chr5 | 11623680 | 11624180 | −0.001001915 | −0.97687628 | −1.164851555 |
| chr19 | 19806280 | 19806480 | −0.001001908 | −0.672733565 | −0.792319746 |
| chr19 | 19806280 | 19806480 | −0.001001908 | −0.672733565 | −0.792319746 |
| chr6 | 103549460 | 103549720 | −0.00100188 | −1.172536528 | −1.379599301 |
| chr11 | 127932140 | 127932700 | 0.001001794 | −1.325074865 | −1.218978895 |
| chr12 | 56497600 | 56498940 | 0.001001637 | −1.373916704 | −1.225679595 |
| chr15 | 78251760 | 78251980 | −0.001001003 | −0.887390899 | −0.953858136 |
| chr15 | 78251760 | 78251980 | −0.001001003 | −0.887390899 | −0.953858136 |
| chr4 | 113065780 | 113067420 | 0.001000957 | −1.303689311 | −1.113843912 |
| chr2 | 220143540 | 220144780 | 0.001000908 | −1.290872203 | −1.090618809 |
| chr5 | 139813860 | 139814320 | −0.001000652 | −0.956373417 | −1.181754521 |
| chr10 | 19594520 | 19595640 | −0.001000638 | −1.050449724 | −1.265838906 |
| chr15 | 48581440 | 48581760 | −0.001000483 | −0.922670407 | −1.050925694 |
| chr14 | 24802840 | 24803160 | 0.001000439 | −1.24820447 | −1.047162907 |
| chr15 | 81397260 | 81397780 | 0.001000056 | −1.079324438 | −0.933330338 |

TABLE S5
Table S5: GO enrichment for class I hotspots (HCC vs. Healthy)

| # Ontology | ID | Desc | BinomRank | BinomP | BinomBonfP | BinomFdrQ | Genes |
|---|---|---|---|---|---|---|---|
| GO | GO: | neutrophil | 6 | 8.32E−09 | 1.07E−04 | 1.79E−05 | ADAM17, APAF1, ATG7, ATP11B, |
| Biological | 0002446 | mediated | | | | | ATP6V0C, B4GALT1, CAT, CCT8, |
| Process | | immunity | | | | | CD300A, CD58, CD97, CKAP4, |
| | | | | | | | CNN2, COTL1, CPNE3, CREG1, |
| | | | | | | | CRISPLD2, CTSZ, DNAJC13, |
| | | | | | | | DNAJC3, DOCK2, EEF2, EFCAB4B, |
| | | | | | | | ERP44, FPR2, GDI2, HMHA1, |
| | | | | | | | HSP90AA1, HSPA8, IMPDH1, |
| | | | | | | | ITGB2, KCMF1, LAIR1, LAMP1, |
| | | | | | | | LAMTOR2, LPCAT1, MAN2B1, |
| | | | | | | | MANBA, MLEC, NPC2, P2RX1, PA2G4, |
| | | | | | | | PDAP1, PDXK, PGLYRP1, PLAC8, |
| | | | | | | | PLEKHO2, PRCP, PRDX6, PRG3, |
| | | | | | | | PSAP, PSMD1, PSMD3, PSMD6, |
| | | | | | | | PTGES2, PTPN6,PTPRN2, PYGB, |
| | | | | | | | QPCT, RAB10, RAB4B, RAB7A, |
| | | | | | | | RAP2B, SCAMP1, SERPINB1, SIGLEC5, |
| | | | | | | | SLC2A3, STK10, SVIP, TIMP2, |
| | | | | | | | TMC6, TMEM179B, TOLLIP, TRPM2, |
| | | | | | | | TUBB, UBR4, VCP, XRCC5 |
| GO | GO: | myeloid | 7 | 1.07E−08 | 1.37E−04 | 1.96E−05 | ADAM9, APAF1, ATG7, ATP11B, |
| Biological | 0002274 | leukocyte | | | | | ATP6V0C, B4GALT1, CAT, CCT8, |
| Process | | activation | | | | | CD300A, CD58, CD97, CKAP4, |
| | | | | | | | CNN2, COTL1, CPNE3, CREG1, |
| | | | | | | | CRISPLD2, CRTC3, CTSZ, DHRS2, |
| | | | | | | | DNAJC13, DNAJC3, DOCK2, EEF2, |
| | | | | | | | EFCAB4B, ERP44, FPR2, GDI2, HMHA1, |
| | | | | | | | HSP90AA1, HSPA8, IMPDH1, ITGB2, |
| | | | | | | | KCMF1, LAIR1, LAMP1, LAMTOR2, |
| | | | | | | | LPCAT1, LYN, MAN2B1, MANBA, MAPT, |
| | | | | | | | MLEC, NDRG1, NPC2, P2RX1, PA2G4, |
| | | | | | | | PDAP1, PDXK, PGLYRP1, PLAC8, |
| | | | | | | | PLEKHO2, PRCP, PRDX6, PREX1, PRG3, |
| | | | | | | | PSAP, PSMD1, PSMD3, PSMD6, PTGES2, |
| | | | | | | | PTPN6, PTPRN2, PYGB, QPCT, RAB10, |
| | | | | | | | RAB4B, RAB7A, RAP2B, SBNO2, SCAMP1, |
| | | | | | | | SERPINB1, SIGLEC5, SLAMF1, SLC2A3, |
| | | | | | | | SPI1, STK10, SVIP, TGFB1, TIMP2, |
| | | | | | | | TMC6, TMEM179B, TOLLIP, TRAF6, |
| | | | | | | | TRPM2, TUBB, UBR4, VAMP2, VCP, |
| | | | | | | | XRCC5 |
| GO | GO: | myeloid cell | 4 | 6.09E−09 | 7.86E−05 | 1.97E−05 | APAF1, ATG7, ATP11B, ATP6V0C, |
| Biological | 0002275 | activation | | | | | B4GALT1, CAT, CCT8, CD300A, |
| Process | | involved in | | | | | CD58, CD97, CKAP4, CNN2, COTL1, |
| | | immune | | | | | CPNE3, CREG1, CRISPLD2, CTSZ, |
| | | response | | | | | DNAJC13, DNAJC3, DOCK2, EEF2, |
| | | | | | | | EFCAB4B, ERP44, FPR2, GDI2, |
| | | | | | | | HMHA1, HSP90AA1, HSPA8, IMPDH1, |
| | | | | | | | ITGB2, KCMF1, LAIR1, LAMP1, |
| | | | | | | | LAMTOR2, LPCAT1, LYN, MAN2B1, |
| | | | | | | | MANBA, MLEC, NPC2, P2RX1, PA2G4, |
| | | | | | | | PDAP1, PDXK, PGLYRP1, PLAC8, |
| | | | | | | | PLEKHO2, PRCP, PRDX6, PRG3, |
| | | | | | | | PSAP, PSMD1, PSMD3, PSMD6, |
| | | | | | | | PTGES2, PTPN6, PTPRN2, PYGB, |
| | | | | | | | QPCT, RAB10, RAB4B, RAB7A, |
| | | | | | | | RAP2B, SBNO2, SCAMP1, SERPINB1, |
| | | | | | | | SIGLEC5, SLAMF1, SLC2A3, STK10, |
| | | | | | | | SVIP, TIMP2, TMC6, TMEM179B, |
| | | | | | | | TOLLIP, TRPM2, TUBB, UBR4, |
| | | | | | | | VAMP2, VCP, XRCC5 |
| GO | GO: | granulocyte | 5 | 7.80E−09 | 1.01E−04 | 2.01E−05 | APAF1, ATG7, ATP11B, ATP6V0C, |
| Biological | 0036230 | activation | | | | | B4GALT1, CAT, CCT8, CD300A, |
| Process | | | | | | | CD58, CD97, CKAP4, CNN2, |
| | | | | | | | COTL1, CPNE3, CREG1, CRISPLD2, |
| | | | | | | | CTSZ, DNAJC13, DNAJC3, DOCK2, |
| | | | | | | | EEF2, EFCAB4B, ERP44, FPR2, |
| | | | | | | | GDI2, HMHA1, HSP90AA1, HSPA8, |
| | | | | | | | MPDH1, ITGB2, KCMF1, LAIR1, |
| | | | | | | | LAMP1, LAMTOR2, LPCAT1, MAN2B1, |
| | | | | | | | MANBA, MLEC, NPC2, P2RX1, |
| | | | | | | | PA2G4, PDAP1, PDXK, PGLYRP1, |
| | | | | | | | PLAC8, PLEKHO2, PRCP, PRDX6, |
| | | | | | | | PREX1, PRG3, PSAP, PSMD1, |
| | | | | | | | PSMD3, PSMD6, PTGES2, PTPN6, |
| | | | | | | | PTPRN2, PYGB, QPCT, RAB10, |
| | | | | | | | RAB4B, RAB7A, RAP2B, SCAMP1, |
| | | | | | | | SERPINB1, SIGLEC5, SLC2A3, |
| | | | | | | | STK10, SVIP, TIMP2, TMC6, |
| | | | | | | | TMEM179B, TOLLIP, TRPM2, |
| | | | | | | | TUBB, UBR4, VAMP2, VCP, XRCC5 |
| GO | GO: | neutrophil | 8 | 1.42E−08 | 1.83E−04 | 2.29E−05 | APAF1, ATG7, ATP11B, ATP6V0C, |
| Biological | 0042119 | activation | | | | | B4GALT1, CAT, CCT8, CD300A, |
| Process | | | | | | | CD58, CD97, CKAP4, CNN2, COTL1, |
| | | | | | | | CPNE3, CREG1, CRISPLD2, CTSZ, |
| | | | | | | | DNAJC13, DNAJC3, DOCK2, EEF2, |
| | | | | | | | EFCAB4B, ERP44, FPR2, GDI2, |
| | | | | | | | HMHA1, HSP90AA1, HSPA8, IMPDH1, |
| | | | | | | | ITGB2, KCMF1, LAIR1, LAMP1, |
| | | | | | | | LAMTOR2, LPCAT1, MAN2B1, MANBA, |
| | | | | | | | MLEC, NPC2, P2RX1, PA2G4, PDAP1, |
| | | | | | | | PDXK, PGLYRP1, PLAC8, PLEKHO2, |
| | | | | | | | PRCP, PRDX6, PREX1, PRG3, PSAP, |
| | | | | | | | PSMD1, PSMD3, PSMD6, PTGES2, |
| | | | | | | | PTPN6, PTPRN2, PYGB, QPCT, |
| | | | | | | | RAB10, RAB4B, RAB7A, RAP2B, |
| | | | | | | | SCAMP1, SERPINB1, SIGLEC5, |
| | | | | | | | SLC2A3, STK10, SVIP, TIMP2, |
| | | | | | | | TMC6, TMEM179B, TOLLIP, TRPM2, |
| | | | | | | | TUBB, UBR4, VCP, XRCC5 |
| GO | GO: | intraciliary | 9 | 1.77E−08 | 2.28E−04 | 2.54E−05 | IFT52 |
| Biological | 0035720 | anterograde | | | | | |
| Process | | transport | | | | | |
| GO | GO: | neutrophil | 3 | 6.08E−09 | 7.84E−05 | 2.61E−05 | APAF1, ATG7, ATP11B, ATP6V0C, |
| Biological | 0002283 | activation | | | | | B4GALT1, CAT, CCT8, CD300A, |
| Process | | involved in | | | | | CD58, CD97, CKAP4, CNN2, COTL1, |
| | | immune | | | | | CPNE3, CREG1, CRISPLD2, CTSZ, |
| | | response | | | | | DNAJC13, DNAJC3, DOCK2, EEF2, |
| | | | | | | | EFCAB4B, ERP44, FPR2, GDI2, |
| | | | | | | | HMHA1, HSP90AA1, HSPA8, |
| | | | | | | | IMPDH1, ITGB2, KCMF1, LAIR1, |
| | | | | | | | LAMP1, LAMTOR2, LPCAT1, MAN2B1, |
| | | | | | | | MANBA, MLEC, NPC2, P2RX1, PA2G4, |
| | | | | | | | PDAP1, PDXK, PGLYRP1, PLAC8, |
| | | | | | | | PLEKHO2, PRCP, PRDX6, PRG3, |
| | | | | | | | PSAP, PSMD1, PSMD3, PSMD6, |
| | | | | | | | PTGES2, PTPN6, PTPRN2, PYGB, |
| | | | | | | | QPCT, RAB10, RAB4B, RAB7A, |
| | | | | | | | RAP2B, SCAMP1, SERPINB1, SIGLEC5, |
| | | | | | | | SLC2A3, STK10, SVIP, TIMP2, TMC6, |
| | | | | | | | TMEM179B, TOLLIP, TRPM2, TUBB, |
| | | | | | | | UBR4, VCP, XRCC5 |
| GO | GO: | leukocyte | 2 | 5.63E−09 | 7.26E−05 | 3.63E−05 | APAF1, ATG7, ATP11B, ATP6V0C, |
| Biological | 0043299 | degranulation | | | | | B4GALT1, CAT, CCT8, CD300A, |
| Process | | | | | | | CD58, CD97, CKAP4, CNN2, |
| | | | | | | | CORO1A, COTL1, CPNE3, |
| | | | | | | | CREG1, CRISPLD2, CTSZ, DNAJC13, |
| | | | | | | | DNAJC3, DOCK2, EEF2, EFCAB4B, |
| | | | | | | | ERP44, FPR2, GDI2, HMHA1, HSP90AA1, |
| | | | | | | | HSPA8, IMPDH1, ITGB2, KCMF1, LAIR1, |
| | | | | | | | LAMP1, LAMTOR2, LPCAT1, LYN, |
| | | | | | | | MAN2B1, MANBA, MLEC, NPC2, P2RX1, |
| | | | | | | | PA2G4, PDAP1, PDXK, PGLYRP1, |
| | | | | | | | PLAC8, PLEKHO2, PRCP, PRDX6, |
| | | | | | | | PRG3, PSAP, PSMD1, PSMD3, PSMD6, |
| | | | | | | | PTGES2, PTPN6, PTPRN2, PYGB, QPCT, |
| | | | | | | | RAB10, RAB4B, RAB7A, RAP2B, SCAMP1, |
| | | | | | | | SERPINB1, SIGLEC5, SLC2A3, STK10, |
| | | | | | | | SVIP, TIMP2, TMC6, TMEM179B, |
| | | | | | | | TOLLIP, TRPM2, TUBB, UBR4, |
| | | | | | | | VAMP2, VCP, XRCC5 |
| GO | GO: | myeloid | 10 | 3.76E−08 | 4.85E−04 | 4.85E−05 | ADAM17, APAF1, ATG7, ATP11B, |
| Biological | 0002444 | leukocyte | | | | | ATP6V0C, B4GALT1, CAT, CCT8, |
| Process | | mediated | | | | | CD300A, CD58, CD97, CKAP4, |
| | | immunity | | | | | CNN2, COTL1, CPNE3, CREG1, |
| | | | | | | | CRISPLD2, CTSZ, DNAJC13, |
| | | | | | | | DNAJC3, DOCK2, EEF2, EFCAB4B, |
| | | | | | | | ERP44, FPR2, GDI2, HMHA1, |
| | | | | | | | HSP90AA1, HSPA8, IMPDH1, ITGB2, |
| | | | | | | | KCMF1, LAIR1, LAMP1, LAMTOR2, |
| | | | | | | | LPCAT1, LYN, MAN2B1, MANBA, |
| | | | | | | | MLEC, NPC2, P2RX1, PA2G4, PDAP1, |
| | | | | | | | PDXK, PGLYRP1, PLAC8, PLEKHO2, |
| | | | | | | | PRCP, PRDX6, PRG3, PSAP, PSMD1, |
| | | | | | | | PSMD3, PSMD6, PTGES2, PTPN6, |
| | | | | | | | PTPRN2, PYGB, QPCT, RAB10, RAB4B, |
| | | | | | | | RAB7A, RAP2B, SCAMP1, SERPINB1, |
| | | | | | | | SIGLEC5, SLC2A3, STK10, SVIP, |
| | | | | | | | TIMP2, TMC6, TMEM179B, TOLLIP, |
| | | | | | | | TRPM2, TUBB, UBR4, VAMP2, |
| | | | | | | | VCP, XRCC5 |
| GO | GO: | neutrophil | 1 | 3.95E−09 | 5.10E−05 | 5.10E−05 | APAF1, ATG7, ATP11B, ATP6V0C, |
| Biological | 0043312 | degranulation | | | | | B4GALT1, CAT, CCT8, CD300A, |
| Process | | | | | | | CD58, CD97, CKAP4, CNN2, COTL1, |
| | | | | | | | CPNE3, CREG1, CRISPLD2, CTSZ, |
| | | | | | | | DNAJC13, DNAJC3, DOCK2, EEF2, |
| | | | | | | | EFCAB4B, ERP44, FPR2, GDI2, |
| | | | | | | | HMHA1, HSP90AA1, HSPA8, IMPDH1, |
| | | | | | | | ITGB2, KCMF1, LAIR1, LAMP1, |
| | | | | | | | LAMTOR2, LPCAT1, MAN2B1, MANBA, |
| | | | | | | | MLEC, NPC2, P2RX1, PA2G4, PDAP1, |
| | | | | | | | PDXK, PGLYRP1, PLAC8, PLEKHO2, |
| | | | | | | | PRCP, PRDX6, PRG3, PSAP, PSMD1, |
| | | | | | | | PSMD3, PSMD6, PTGES2, PTPN6, |
| | | | | | | | PTPRN2, PYGB, QPCT, RAB10, |
| | | | | | | | RAB4B, RAB7A, RAP2B, SCAMP1, |
| | | | | | | | SERPINB1, SIGLEC5, SLC2A3, |
| | | | | | | | STK10, SVIP, TIMP2, TMC6, |
| | | | | | | | TMEM179B, TOLLIP, TRPM2, |
| | | | | | | | TUBB, UBR4, VCP, XRCC5 |
| GO | GO: | leukocyte | 11 | 6.56E−08 | 8.47E−04 | 7.70E−05 | ABL1, APAF1, APBB1IP, ATG7, ATP11B, |
| Biological | 0002366 | activation | | | | | ATP6V0C, B4GALT1, BCL3, CAT, CCT8, |
| Process | | involved in | | | | | CD300A, CD58, CD97, CKAP4, CNN2, |
| | | immune | | | | | CORO1A, COTL1, CPNE3, CREG1, |
| | | response | | | | | CRISPLD2, CTSZ, DNAJC13, DNAJC3, |
| | | | | | | | DOCK2, EEF2, EFCAB4B, ERCC1, ERP44, |
| | | | | | | | FPR2, GDI2, HMHA1, HSP90AA1, HSPA8, |
| | | | | | | | IMPDH1, ITGB2, KCMF1, LAIR1, LAMP1, |
| | | | | | | | LAMTOR2, LCP1, LFNG, LPCAT1, LYN, |
| | | | | | | | MAN2B1, MANBA, MFNG, MLEC, MSH2, |
| | | | | | | | NPC2, P2RX1, PA2G4, PDAP1, PDXK, |
| | | | | | | | PGLYRP1, PLAC8, PLCG2, PLEKHO2, |
| | | | | | | | PRCP, PRDX6, PRG3, PSAP, PSMD1, |
| | | | | | | | PSMD3, PSMD6, PTGES2, PTPN6, PTPRN2, |
| | | | | | | | PYGB, QPCT, RAB10, RAB4B, RAB7A, |
| | | | | | | | RAP2B, SBNO2, SCAMP1, SEMA4A, |
| | | | | | | | SERPINB1, SIGLEC5, SLAMF1, SLC2A3, |
| | | | | | | | STK10, SVIP, TIMP2, TMC6, TMEM179B, |
| | | | | | | | TOLLIP, TRPM2, TUBB, UBR4, VAMP2, |
| | | | | | | | VCP, XRCC5 |
| GO | GO: | cell | 12 | 1.56E−07 | 2.01E−03 | 1.68E−04 | ABL1, APAF1, APBB1IP, ATG7, ATP11B, |
| Biological | 0002263 | activation | | | | | ATP6V0C, B4GALT1, BCL3, CAT, CCT8, |
| Process | | involved in | | | | | CD300A, CD58, CD97, CKAP4, CNN2, |
| | | immune | | | | | CORO1A, COTL1, CPNE3, CREG1, |
| | | response | | | | | CRISPLD2, CTSZ, DNAJC13, DNAJC3, |
| | | | | | | | DOCK2, EEF2, EFCAB4B, ERCC1, ERP44, |
| | | | | | | | FPR2, GDI2, HMHA1, HSP90AA1, HSPA8, |
| | | | | | | | IMPDH1, ITGB2, KCMF1, LAIR1, LAMP1, |
| | | | | | | | LAMTOR2, LCP1, LFNG, LPCAT1, LYN, |
| | | | | | | | MAN2B1, MANBA, MFNG, MLEC, MSH2, |
| | | | | | | | NPC2, P2RX1, PA2G4, PDAP1, PDXK, |
| | | | | | | | PGLYRP1, PLAC8, PLCG2, PLEKHO2, |
| | | | | | | | PRCP, PRDX6, PRG3, PSAP, PSMD1, |
| | | | | | | | PSMD3, PSMD6, PTGES2, PTPN6, PTPRN2, |
| | | | | | | | PYGB, QPCT, RAB10, RAB4B, RAB7A, |
| | | | | | | | RAP2B, SBNO2, SCAMP1, SEMA4A, |
| | | | | | | | SERPINB1, SIGLEC5, SLAMF1, SLC2A3, |
| | | | | | | | STK10, SVIP, TIMP2, TMC6, TMEM179B, |
| | | | | | | | TOLLIP, TRPM2, TUBB, UBR4, VAMP2, |
| | | | | | | | VCP, XRCC5 |
| GO | GO: | protein | 14 | 1.96E−07 | 2.53E−03 | 1.81E−04 | BAG1, DNAJB12, DNAJB2, |
| Biological | 0042026 | refolding | | | | | HSP90AA1, HSPA8 |
| Process | | | | | | | |
| GO | GO: | immune | 13 | 1.95E−07 | 2.52E−03 | 1.94E−04 | ABCF3, ABI1, ABL1, ACTB, ADAM17, |
| Biological | 0002252 | effector | | | | | ADARB1, APAF1, APBB1IP, APOBEC3A, |
| Process | | process | | | | | ARPC1A, ATG7, ATP11B, ATP6V0C, |
| | | | | | | | B4GALT1, BAIAP2, BCL3, BRK1, |
| | | | | | | | C19orf66, CAT, CCT8, CD247, |
| | | | | | | | CD300A, CD46, CD58, |
| | | | | | | | CD74, CD97, CKAP4, CNN2, CNOT7, |
| | | | | | | | CORO1A, COTL1, CPNE3, CREG1, |
| | | | | | | | CRISPLD2, CRK, CTSZ, DLG1, |
| | | | | | | | DNAJC13, DNAJC3, DOCK1, DOCK2, |
| | | | | | | | EEF2, EFCAB4B, ELMOD2, EMP2, |
| | | | | | | | ERCC1, ERP44, EXOSC4, FADD, |
| | | | | | | | FPR2, GCNT3, GDI2, HMHA1, |
| | | | | | | | HSP90AA1, HSPA8, IMPDH1, IRF5, |
| | | | | | | | ITGB2, KCMF1, LAIR1, LAMP1, |
| | | | | | | | LAMTOR2, LCP1, LFNG, LPCAT1, |
| | | | | | | | LYN, MAN2B1, MANBA, MFNG, |
| | | | | | | | MICB, MLEC, MSH2, MYO1C, |
| | | | | | | | NLRC5, NLRP3, NPC2, P2RX1, |
| | | | | | | | PA2G4, PAK1, PDAP1, PDXK, |
| | | | | | | | PGLYRP1, PIK3R2, PLAC8, |
| | | | | | | | PLCG2, PLEKHO2, POLR3B, |
| | | | | | | | PRCP, PRDX6, PRG3, PSAP, |
| | | | | | | | PSMD1, PSMD3, PSMD6, PTGES2, |
| | | | | | | | PTK2, PTPN6, PTPRN2, PYGB, |
| | | | | | | | QPCT, RAB10, RAB4B, RAB7A, |
| | | | | | | | RAP2B, SBNO2, SCAMP1, SEMA4A, |
| | | | | | | | SERPINB1, SIGLEC5, SLAMF1, |
| | | | | | | | SLC2A3, STK10, SVIP, TIMP2, |
| | | | | | | | TMC6, TMEM179B, TOLLIP, |
| | | | | | | | TRIM11, TRIM25, TRPM2, TUBB, |
| | | | | | | | UBR4, VAMP2, VAV2, VAV3, |
| | | | | | | | VCP, WIPF1, XRCC5, ZNF175 |
| GO | GO: | leukocyte | 15 | 2.33E−07 | 3.01E−03 | 2.01E−04 | ADAM17, APAF1, ATG7, ATP11B, |
| Biological | 0002443 | mediated | | | | | ATP6V0C, B4GALT1, BCL3, CAT, |
| Process | | immunity | | | | | CCT8, CD300A, CD46, CD58, |
| | | | | | | | CD74, CD97, CKAP4, CNN2, |
| | | | | | | | CORO1A, COTL1, CPNE3, CREG1, |
| | | | | | | | CRISPLD2, CTSZ, DLG1, DNAJC13, |
| | | | | | | | DNAJC3, DOCK2, EEF2, EFCAB4B, |
| | | | | | | | EMP2, ERCC1, ERP44, FPR2, |
| | | | | | | | GCNT3, GDI2, HMHA1, HSP90AA1, |
| | | | | | | | HSPA8, IMPDH1, ITGB2, KCMF1, |
| | | | | | | | LAIR1, LAMP1, LAMTOR2, |
| | | | | | | | LPCAT1, LYN, MAN2B1, MANBA, |
| | | | | | | | MICB, MLEC, MSH2, NPC2, |
| | | | | | | | P2RX1, PA2G4, PDAP1, PDXK, |
| | | | | | | | PGLYRP1, PLAC8, PLEKHO2, PRCP, |
| | | | | | | | PRDX6, PRG3, PSAP, PSMD1, |
| | | | | | | | PSMD3, PSMD6, PTGES2, PTPN6, |
| | | | | | | | PTPRN2, PYGB, QPCT, RAB10, RAB4B, |
| | | | | | | | RAB7A, RAP2B, SCAMP1, SERPINB1, |
| | | | | | | | SIGLEC5, SLAMF1, SLC2A3, STK10, |
| | | | | | | | SVIP, TIMP2, TMC6, TMEM179B, |
| | | | | | | | TOLLIP, TRPM2, TUBB, UBR4, |
| | | | | | | | VAMP2, VCP, XRCC5 |
| GO | GO: | viral | 16 | 8.15E−07 | 1.05E−02 | 6.57E−04 | ABI1, ACY3, ATF7IP, ATG7, |
| Biological | 0016032 | process | | | | | ATP6V0C, ATP6V1G2-DDX39B, |
| Process | | | | | | | BIN1, BUB1, C9orf69, CCL1, |
| | | | | | | | CCNA2, CD46, CD81, CHD6, |
| | | | | | | | CHMP2A, CHMP5, CHMP6, CREBBP, |
| | | | | | | | CRTC2, CRTC3, CXADR, DAXX, |
| | | | | | | | DLG1, DPP4, DYRK1A, EIF3B, |
| | | | | | | | EIF3D, EIF3L, FADD, FBLN1, |
| | | | | | | | GTF2E1, H2AFX, HLA-B, |
| | | | | | | | HSPA8, HTATIP2, IL16, IPO5, |
| | | | | | | | ITGB1, ITGB7, KPNA2, KPNA4, |
| | | | | | | | KPNA7, LAMP1, LYN, MDFIC, |
| | | | | | | | MFGE8, MICB, MVB12A, NFX1, |
| | | | | | | | NUCKS1, NUP210, NUP88, NUPL1, |
| | | | | | | | PABPN1, PCBP1, POLR2E, POM121, |
| | | | | | | | PSMB10, PTBP1, RAB11FIP4, RAB7A, |
| | | | | | | | RAE1, RPL18, RPL32, RPL7, RPLP1, |
| | | | | | | | RPS14, RPS25, RPS26, RPS29, |
| | | | | | | | RPS3A, SCARB1, SF3B2, SLAMF1, |
| | | | | | | | SP1, SPEN, TAF4, TBKBP1, TFRC, |
| | | | | | | | TGFB1, TNFRSF1A, TRIM25, UBC, |
| | | | | | | | UBR4, UNG, USP6NL, VCP, VPS18, |
| | | | | | | | VPS37A, VPS37C, VPS4B, WAPAL, |
| | | | | | | | XRCC5, ZYX |
| GO | GO: | multi- | 17 | 1.94E−06 | 2.50E−02 | 1.47E−03 | ABI1, ACY3, ATF7IP, ATG7, |
| Biological | 0044764 | organism | | | | | ATP6V0C, ATP6V1G2-DDX39B, |
| Process | | cellular | | | | | BIN1, BUB1, C9orf69, CCL1, CCNA2, |
| | | process | | | | | CD46, CD81, CHD6, CHMP2A, CHMP5, |
| | | | | | | | CHMP6, CREBBP, CRTC2, CRTC3, |
| | | | | | | | CXADR, DAXX, DLG1, DPP4, DYRK1A, |
| | | | | | | | EIF3B, EIF3D, EIF3L, FADD, FBLN1, |
| | | | | | | | GTF2E1, H2AFX, HLA-B, HSPA8, |
| | | | | | | | HTATIP2, IL16, IPO5, ITGB1, ITGB7, |
| | | | | | | | KPNA2, KPNA4, KPNA7, LAMP1, |
| | | | | | | | LYN, MDFIC, MFGE8, MICB, MVB12A, |
| | | | | | | | NFX1, NUCKS1, NUP210, NUP88, NUPL1, |
| | | | | | | | PABPN1, PCBP1, POLR2E, POM121, |
| | | | | | | | PSMB10, PTBP1, RAB11FIP4, RAB7A, |
| | | | | | | | RAE1, RPL18, RPL32, RPL7, RPLP1, |
| | | | | | | | RPS14, RPS25, RPS26, RPS29, RPS3A, |
| | | | | | | | SCARB1, SF3B2, SLAMF1, SP1, SPEN, |
| | | | | | | | TAF4, TBKBP1, TFRC, TGFB1, TNFRSF1A, |
| | | | | | | | TRIM25, UBC, UBR4, UNG, USP6NL, |
| | | | | | | | VCP, VPS18, VPS37A, VPS37C, VPS4B, |
| | | | | | | | WAPAL, XRCC5, ZYX |
| GO | GO: | regulation of | 18 | 2.86E−06 | 3.69E−02 | 2.05E−03 | ERCC1, SLX1A, XRCC5 |
| Biological | 1904429 | t-circle | | | | | |
| Process | | formation | | | | | |
| GO | GO: | leukocyte | 20 | 3.93E−06 | 5.08E−02 | 2.54E−03 | ABL1, ADAM17, ADAM9, APAF1, |
| Biological | 0045321 | activation | | | | | APBB1IP, ATG7, ATP11B, ATP6V0C, |
| Process | | | | | | | B4GALT1, BCL3, BCL6, CAT, CCT8, |
| | | | | | | | CD300A, CD58, CD74, CD97, CHD7, |
| | | | | | | | CKAP4, CNN2, CORO1A, COTL1, |
| | | | | | | | CPNE3, CREG1, CRISPLD2, CRTC3, |
| | | | | | | | CTSZ, CXADR, CYLD, DHRS2, DLG1, |
| | | | | | | | DLL4, DNAJC13, DNAJC3, DOCK2, |
| | | | | | | | DPP4, EEF2, EFCAB4B, ERCC1, |
| | | | | | | | ERP44, FADD, FPR2, GDI2, GLI3, |
| | | | | | | | GON4L, HMHA1, HSP90AA1, HSPA8, |
| | | | | | | | ICOSLG, IL7, IMPDH1, ITGB1, |
| | | | | | | | ITGB2, KCMF1, KIF13B, KLF6, |
| | | | | | | | LAIR1, LAMP1, LAMTOR2, LCP1, |
| | | | | | | | LFNG, LPCAT1, LYL1, LYN, MAN2B1, |
| | | | | | | | MANBA, MAPT, MFNG, MICB, MLEC, |
| | | | | | | | MSH2, MYH9, NDRG1, NPC2, ONECUT1, |
| | | | | | | | P2RX1, PA2G4, PDAP1, PDXK, |
| | | | | | | | PGLYRP1, PLAC8, PLCG2, PLEKHO2, |
| | | | | | | | POU1F1, PRCP, PRDX6, PREX1, |
| | | | | | | | PRG3, PSAP, PSMD1, PSMD3, |
| | | | | | | | PSMD6, PTGES2, PTPN6, PTPRN2, |
| | | | | | | | PYGB, QPCT, RAB10, RAB4B, |
| | | | | | | | RAB7A, RAP2B, SBNO2, SCAMP1, |
| | | | | | | | SEMA4A, SERPINB1, SHH, SIGLEC5, |
| | | | | | | | SKAP2, SLAMF1, SLC2A3, SP3, |
| | | | | | | | SPI1, STK10, SVIP, TBX21, |
| | | | | | | | TGFB1, TIMP2, TMC6, TMEM179B, |
| | | | | | | | TNFSF4, TOLLIP, TRAF6, TRPM2, |
| | | | | | | | TUBB, TXLNA, UBR4, VAMP2, |
| | | | | | | | VCP, XRCC5, ZFP36L2 |
| GO | GO: | symbiosis, | 19 | 3.92E−06 | 5.05E−02 | 2.66E−03 | ABI1, ACY3, ATF7IP, ATG7, ATP6V0C, |
| Biological | 0044403 | encompassing | | | | | ATP6V1G2-DDX39B, |
| Process | | mutualism | | | | | BIN1, BUB1, C9orf69, CBLL1, |
| | | through | | | | | CCL1, CCNA2, CD46, CD81, CFL1, CHD6, |
| | | parasitism | | | | | CHMP2A, CHMP5, CHMP6, CREBBP, |
| | | | | | | | CRTC2, CRTC3, CXADR, DAXX, DLG1, |
| | | | | | | | DPP4, DYRK1A, EIF3B, EIF3D, EIF3L, |
| | | | | | | | FADD, FBLN1, GTF2E1, H2AFX, HLA-B, |
| | | | | | | | HSPA8, HTATIP2, IL16, IPO5, ITGB1, |
| | | | | | | | ITGB7, KPNA2, KPNA4, KPNA7, LAMP1, |
| | | | | | | | LYN, MDFIC, MFGE8, MICB, MVB12A, |
| | | | | | | | NFX1, NUCKS1, NUP210, NUP88, NUPL1, |
| | | | | | | | PABPN1, PCBP1, PGLYRP1, POLR2E, |
| | | | | | | | POM121, PSMB10, PTBP1, RAB11FIP4, |
| | | | | | | | RAB7A, RAE1, RPL18, RPL32, RPL7, |
| | | | | | | | RPLP1, RPS14, RPS25, RPS26, RPS29, |
| | | | | | | | RPS3A, SCARB1, SF3B2, SLAMF1, SP1, |
| | | | | | | | SPEN, TAF4, TBKBP1, TFAP4, TFRC, |
| | | | | | | | TGFB1, TNFRSF1A, TRIM25, UBC, UBR4, |
| | | | | | | | UNG, USP6NL, VCP, VPS18, VPS37A, |
| | | | | | | | VPS37C, VPS4B, WAPAL, XRCC5, ZYX |
| GO | GO: | interspecies | 21 | 4.38E−06 | 5.65E−02 | 2.69E−03 | ABI1, ACY3, ATF7IP, ATG7, |
| Biological | 0044419 | interaction | | | | | ATP6V0C, ATP6V1G2-DDX39B, |
| Process | | between | | | | | BIN1, BUB1, C9orf69, CBLL1, |
| | | organisms | | | | | CCL1, CCNA2, CD46, CD81, CFL1, CHD6, |
| | | | | | | | CHMP2A, CHMP5, CHMP6, CREBBP, |
| | | | | | | | CRTC2, CRTC3, CXADR, DAXX, DLG1, |
| | | | | | | | DPP4, DYRK1A, EIF3B, EIF3D, EIF3L, |
| | | | | | | | FADD, FBLN1, GTF2E1, H2AFX, HLA-B, |
| | | | | | | | HSPA8, HTATIP2, IL16, IPO5, ITGB1, |
| | | | | | | | ITGB7, KPNA2, KPNA4, KPNA7, LAMP1, |
| | | | | | | | LYN, MDFIC, MFGE8, MICB, MVB12A, |
| | | | | | | | NFX1, NUCKS1, NUP210, NUP88, NUPL1, |
| | | | | | | | PABPN1, PCBP1, PGLYRP1, POLR2E, |
| | | | | | | | POM121, PSMB10, PTBP1, RAB11FIP4, |
| | | | | | | | RAB7A, RAE1, RPL18, RPL32, RPL7, |
| | | | | | | | RPLP1, RPS14, RPS25, RPS26, RPS29, |
| | | | | | | | RPS3A, SCARB1, SF3B2, SLAMF1, SP1, |
| | | | | | | | SPEN, TAF4, TBKBP1, TFAP4, TFRC, |
| | | | | | | | TGFB1, TNFRSF1A, TRIM25, UBC, UBR4, |
| | | | | | | | UNG, USP6NL, VCP, VPS18, VPS37A, |
| | | | | | | | VPS37C, VPS4B, WAPAL, XRCC5, ZYX |
| GO | GO: | positive | 23 | 5.80E−06 | 7.48E−02 | 3.25E−03 | EEF2, METTL3, YTHDF2 |
| Biological | 2000767 | regulation of | | | | | |
| Process | | cytoplasmic | | | | | |
| | | translation | | | | | |
| GO | GO: | positive | 22 | 5.80E−06 | 7.48E−02 | 3.40E−03 | ADARB1, C9orf69, CD74, CFL1, |
| Biological | 0048524 | regulation | | | | | CHMP2A, CNOT7, HSPA8, MDFIC, |
| Process | | of viral | | | | | MVB12A, NELFCD, NR5A2, NUCKS1, |
| | | process | | | | | PFN1, PKN2, POLR2D, POLR2E, |
| | | | | | | | RAB7A, SP1, STAU1, SUPT4H1, |
| | | | | | | | TFAP4, TMPRSS2, TRIM11, VPS4B |
| GO | GO: | regulation of | 24 | 6.80E−06 | 8.78E−02 | 3.66E−03 | PABPN1, ZFP36 |
| Biological | 1904245 | polynucleotide | | | | | |
| Process | | adenylyltransferase | | | | | |
| | | activity | | | | | |
| GO | GO: | multivesicular | 25 | 9.06E−06 | 1.17E−01 | 4.67E−03 | CHMP2A, CHMP5, CHMP6, MVB12A, |
| Biological | 0036258 | body | | | | | VPS25, VPS37A, VPS37C, VPS4B |
| Process | | assembly | | | | | |
| GO | GO: | viral | 26 | 9.62E−06 | 1.24E−01 | 4.77E−03 | EIF3B, EIF3D, EIF3L, PTBP1 |
| Biological | 0019081 | translation | | | | | |
| Process | | | | | | | |
| GO | GO: | multivesicular | 27 | 1.17E−05 | 1.50E−01 | 5.57E−03 | CHMP2A, CHMP5, CHMP6, MVB12A, |
| Biological | 0036257 | body | | | | | VPS25, VPS37A, VPS37C, VPS4B |
| Process | | organization | | | | | |
| GO | GO: | hypermethylation | 28 | 1.66E−05 | 2.14E−01 | 7.64E−03 | GSK3A, SPI1 |
| Biological | 0044027 | of CpG | | | | | |
| Process | | island | | | | | |
| GO | GO: | viral | 31 | 2.46E−05 | 3.17E−01 | 1.02E−02 | CHMP2A, CHMP5, CHMP6, MVB12A, |
| Biological | 0046755 | budding | | | | | VPS37A, VPS37C, VPS4B |
| Process | | | | | | | |
| GO | GO: | positive | 29 | 2.32E−05 | 2.99E−01 | 1.03E−02 | ERCC1, SLX1A |
| Biological | 1904431 | regulation of | | | | | |
| Process | | t-circle | | | | | |
| | | formation | | | | | |
| GO | GO: | IRES- | 30 | 2.45E−05 | 3.16E−01 | 1.05E−02 | EIF3B, EIF3D, PTBP1 |
| Biological | 0075522 | dependent | | | | | |
| Process | | viral | | | | | |
| | | translational | | | | | |
| | | initiation | | | | | |
| GO | GO: | positive | 32 | 2.63E−05 | 3.39E−01 | 1.06E−02 | ZFP36 |
| Biological | 1901835 | regulation of | | | | | |
| Process | | deadenylation- | | | | | |
| | | independent | | | | | |
| | | decapping of | | | | | |
| | | nuclear- | | | | | |
| | | transcribed | | | | | |
| | | mRNA | | | | | |
| GO | GO: | negative | 32 | 2.63E−05 | 3.39E−01 | 1.06E−02 | ZFP36 |
| Biological | 1904246 | regulation of | | | | | |
| Process | | polynucleotide | | | | | |
| | | adenylyltransferase | | | | | |
| | | activity | | | | | |
| GO | GO: | viral | 34 | 2.82E−05 | 3.64E−01 | 1.07E−02 | EIF3B, EIF3D, EIF3L |
| Biological | 0075525 | translational | | | | | |
| Process | | termination- | | | | | |
| | | reinitiation | | | | | |
| GO | GO: | positive | 35 | 2.93E−05 | 3.78E−01 | 1.08E−02 | NRL |
| Biological | 0045872 | regulation of | | | | | |
| Process | | rhodopsin | | | | | |
| | | gene | | | | | |
| | | expression | | | | | |
| GO | GO: | cellular | 36 | 3.43E−05 | 4.43E−01 | 1.23E−02 | SBNO2 |
| Biological | 0071348 | response to | | | | | |
| Process | | interleukin-11 | | | | | |
| GO | GO: | cellular | 37 | 4.09E−05 | 5.28E−01 | 1.43E−02 | CCNA2, CRK, GHSR, SLC25A33, |
| Biological | 1990314 | response to | | | | | TGFB1 |
| Process | | insulin-like | | | | | |
| | | growth factor | | | | | |
| | | stimulus | | | | | |
| GO | GO: | viral life | 41 | 5.60E−05 | 7.22E−01 | 1.76E−02 | ATP6V1G2-DDX39B, |
| Biological | 0019058 | cycle | | | | | CD46, CD81, CHMP2A, CHMP5, |
| Process | | | | | | | CHMP6, CXADR, DPP4, EIF3B, |
| | | | | | | | EIF3D, EIF3L, ITGB1, ITGB7, |
| | | | | | | | KPNA2, KPNA4, KPNA7, LAMP1, |
| | | | | | | | MVB12A, NUP210, NUP88, NUPL1, |
| | | | | | | | PCBP1, POM121, PTBP1, RAB7A, |
| | | | | | | | RAE1, RPL18, RPL32, RPL7, |
| | | | | | | | RPLP1, RPS14, RPS25, RPS26, |
| | | | | | | | RPS29, RPS3A, SCARB1, SLAMF1, |
| | | | | | | | TFRC, UBC, USP6NL, VCP, VPS18, |
| | | | | | | | VPS37A, VPS37C, VPS4B |
| GO | GO: | virion | 40 | 5.52E−05 | 7.12E−01 | 1.78E−02 | CHMP2A, CHMP5, CHMP6, MVB12A, UBC, |
| Biological | 0019068 | assembly | | | | | USP6NL, VPS37A, VPS37C, VPS4B |
| Process | | | | | | | |
| GO | GO: | regulation of | 38 | 5.25E−05 | 6.77E−01 | 1.78E−02 | NCOR1, PPARA, SREBF2 |
| Biological | 0072367 | lipid | | | | | |
| Process | | transport by | | | | | |
| | | regulation of | | | | | |
| | | transcription | | | | | |
| | | from RNA | | | | | |
| | | polymerase | | | | | |
| | | II promoter | | | | | |
| GO | GO: | chaperone- | 39 | 5.42E−05 | 6.99E−01 | 1.79E−02 | BAG1, CALR, CCT5, CCT8, CD74, |
| Biological | 0061077 | mediated | | | | | CHORDC1, DFFA, DNAJB12, DNAJB2, |
| Process | | protein | | | | | FKBP5, HSPA8, PDIA4 |
| | | folding | | | | | |
| GO | GO: | N- | 42 | 5.98E−05 | 7.71E−01 | 1.84E−02 | AMDHD2, NAGK, NPL |
| Biological | 0019262 | acetylneuraminate | | | | | |
| Process | | catabolic | | | | | |
| | | process | | | | | |
| GO | GO: | negative | 43 | 6.58E−05 | 8.48E−01 | 1.97E−02 | EPO |
| Biological | 1902251 | regulation of | | | | | |
| Process | | erythrocyte | | | | | |
| | | apoptotic | | | | | |
| | | process | | | | | |
| GO | GO: | IRES- | 44 | 7.04E−05 | 9.09E−01 | 2.07E−02 | EIF3B, EIF3D, PTBP1 |
| Biological | 0002192 | dependent | | | | | |
| Process | | translational | | | | | |
| | | initiation | | | | | |
| GO | GO: | transcription- | 45 | 8.01E−05 | 1.00E+00 | 2.30E−02 | AQR, CCNH, COPS2, COPS3, COPS4, |
| Biological | 0006283 | coupled | | | | | ERCC1, LIG1, LIG3, POLR2D, POLR2E, |
| Process | | nucleotide- | | | | | PRPF19, RFC3, UBC, XAB2 |
| | | excision | | | | | |
| | | repair | | | | | |
| GO | GO: | cellular | 46 | 8.20E−05 | 1.00E+00 | 2.30E−02 | LARS, MTOR, RPTOR, RRAGD |
| Biological | 0071233 | response to | | | | | |
| Process | | leucine | | | | | |
| GO | GO: | exocytosis | 47 | 8.50E−05 | 1.00E+00 | 2.33E−02 | ACTN4, APAF1, ARFGEF1, ATG7, ATP11B, |
| Biological | 0006887 | | | | | | ATP6V0C, B4GALT1, CAT, CCT8, CD109, |
| Process | | | | | | | CD300A, CD58, CD97, CHP1, CKAP4, |
| | | | | | | | CNN2, CORO1A, COTL1, CPNE3, |
| | | | | | | | CREG1, CRISPLD2, CTSW, CTSZ, |
| | | | | | | | DNAJC13, DNAJC3, DOCK2, EEF2, |
| | | | | | | | EFCAB4B, ENSG00000263620, ERP44, |
| | | | | | | | EXOC6B, EXOC7, FAM3C, FERMT3, FPR2, |
| | | | | | | | GDI2, HMHA1, HSP90AA1, HSPA8, |
| | | | | | | | IMPDH1, ISLR, ITGB2, KCMF1, LAIR1, |
| | | | | | | | LAMP1, LAMTOR2, LLGL2, LPCAT1, LYN, |
| | | | | | | | MAN2B1, MANBA, MLEC, NKD2, NPC2, |
| | | | | | | | P2RX1, PA2G4, PAK1, PCDH7, PDAP1, |
| | | | | | | | PDXK, PGLYRP1, PIP5K1C, PLAC8, |
| | | | | | | | PLEKHO2, PRCP, PRDX6, PRG3, PSAP, |
| | | | | | | | PSMD1, PSMD3, PSMD6, PTGES2, PTPN6, |
| | | | | | | | PTPRN2, PYGB, QPCT, RAB10, RAB4B, |
| | | | | | | | RAB7A, RAB8B, RAP2B, SCAMP1, |
| | | | | | | | SERPINB1, SIGLEC5, SLC2A3, SPP2, |
| | | | | | | | STK10, STXBP5, SVIP, SYNGR3, SYNJ1, |
| | | | | | | | SYT1, SYT10, SYT11, SYT13, TGFB1, |
| | | | | | | | TGFB3, TIMP2, TMC6, TMEM179B, |
| | | | | | | | TOLLIP, TRPM2, TUBB, TXLNA, UBR4, |
| | | | | | | | VAMP2, VCP, VMP1, VPS18, VPS45, |
| | | | | | | | XRCC5 |
| GO | GO: | defecation | 48 | 1.02E−04 | 1.00E+00 | 2.75E−02 | MDK |
| Biological | 0030421 | | | | | | |
| Process | | | | | | | |
| GO | GO: | positive | 49 | 1.07E−04 | 1.00E+00 | 2.82E−02 | MDFIC, NELFCD, NUCKS1, PFN1, |
| Biological | 0050434 | regulation | | | | | POLR2D, POLR2E, SP1, SUPT4H1, |
| Process | | of viral | | | | | TFAP4 |
| | | transcription | | | | | |
| GO | GO: | negative | 50 | 1.11E−04 | 1.00E+00 | 2.87E−02 | CD46, FOXJ1, PTPN6, SPINK5 |
| Biological | 0002921 | regulation | | | | | |
| Process | | of humoral | | | | | |
| | | immune | | | | | |
| | | response | | | | | |
| GO | GO: | endosomal | 51 | 1.24E−04 | 1.00E+00 | 3.14E−02 | ANKRD27, ANKRD50, ARL4C, C9orf72, |
| Biological | 0016197 | transport | | | | | CHMP2A, CHMP5, CHMP6, CORO1A, |
| Process | | | | | | | DENND3, DNM1L, EMP2, FAM109A, |
| | | | | | | | HOOK1, HOOK2, LAPTM4B, MGRN1, |
| | | | | | | | MVB12A, PICALM, RAB10, RAB17, |
| | | | | | | | RAB7A, SGSM2, SNX18, SNX5, |
| | | | | | | | SNX8, SORT1, SPAG9, STX10, |
| | | | | | | | TINAGL1, UBC, USP6NL, VCP, |
| | | | | | | | VPS13A, VPS18, VPS25, VPS35, |
| | | | | | | | VPS37A, VPS37C, VPS39, VPS4B, |
| | | | | | | | VPS53, WDR81, WIPI1 |
| GO | GO: | endosome | 52 | 1.27E−04 | 1.00E+00 | 3.16E−02 | LAPTM4B, SORT1, VCP, VPS25, |
| Biological | 0032509 | transport via | | | | | VPS4B |
| Process | | multivesicular | | | | | |
| | | body | | | | | |
| | | sorting | | | | | |
| | | pathway | | | | | |
| GO | GO: | negative | 53 | 1.39E−04 | 1.00E+00 | 3.37E−02 | CD300A, SYT11 |
| Biological | 1905154 | regulation of | | | | | |
| Process | | membrane | | | | | |
| | | invagination | | | | | |
| GO | GO: | positive | 54 | 1.57E−04 | 1.00E+00 | 3.76E−02 | ATF4 |
| Biological | 0061395 | regulation of | | | | | |
| Process | | transcription | | | | | |
| | | from RNA | | | | | |
| | | polymerase | | | | | |
| | | II promoter | | | | | |
| | | in response | | | | | |
| | | to arsenic- | | | | | |
| | | containing | | | | | |
| | | substance | | | | | |
| GO | GO: | fructoselysine | 56 | 1.67E−04 | 1.00E+00 | 3.85E−02 | FN3K |
| Biological | 0030393 | metabolic | | | | | |
| Process | | process | | | | | |
| GO | GO: | RNA | 57 | 1.71E−04 | 1.00E+00 | 3.86E−02 | AAR2, AARS2, ABT1, ADARB1, |
| Biological | 0006396 | processing | | | | | APOBEC2, AQR, ATP6V1G2-DDX39B, |
| Process | | | | | | | BUD31, CACTIN, CCNH, CCNL1, |
| | | | | | | | CDC5L, CELF2, CHD7, CIR1, CIRH1A, |
| | | | | | | | CPSF2, CPSF3, CPSF6, CSNK1E, CTU2, |
| | | | | | | | CWC22, DDX19A, DDX39A, DEDD2, |
| | | | | | | | DHX35, DIEXF, DUS2, ERN1, ESRP2, |
| | | | | | | | EXOSC4, EXOSC9, GPATCH1, HNRNPF, |
| | | | | | | | HSPA8, INTS10, INTS3, KIAA1429, |
| | | | | | | | KIAA1456, KRI1, LSM7, MAPKBP1, |
| | | | | | | | MBNL1, MDN1, METTL16, METTL3, |
| | | | | | | | MPHOSPH6, NAT10, NOL11, NOP10, |
| | | | | | | | NOVA1, NSRP1, NWD1, PA2G4, |
| | | | | | | | PABPN1, PAPD5, PAPOLG,PCBP1, |
| | | | | | | | POLDIP3, POLR2D, POLR2E, PPP1R9B, |
| | | | | | | | PRKACA, PRMT7, PRPF19, PTBP1, |
| | | | | | | | PTBP2, PTBP3, PTCD2, RAVER2, |
| | | | | | | | RBMS1, RPL18, RPL32, RPL7, |
| | | | | | | | RPLP1, RPP21, RPP38, RPS14, |
| | | | | | | | RPS25, RPS26, RPS29, RPS3A, |
| | | | | | | | RRP1, SARNP, SCAF8, SF3A2, |
| | | | | | | | SF3A3, SF3B1, SF3B2, SLBP, |
| | | | | | | | SMU1, SNRNP70, SNRPE, SPEN, |
| | | | | | | | SRRM2, SRSF1, SRSF2, SUGP2, |
| | | | | | | | SUPT4H1, SYMPK, TARBP1, TFB1M, |
| | | | | | | | THUMPD2, TSN, U2AF2, UTP3, |
| | | | | | | | WDR4, XAB2, XRN2, YBEY, |
| | | | | | | | ZNF473, ZRANB2 |
| GO | GO: | rRNA | 55 | 1.67E−04 | 1.00E+00 | 3.91E−02 | ABT1, CHD7, CIRH1A, CSNK1E, DEDD2, |
| Biological | 0016072 | metabolic | | | | | DIEXF, EXOSC4, EXOSC9, KRI1, MDN1, |
| Process | | process | | | | | METTL16, MPHOSPH6, NAT10, NOL11, |
| | | | | | | | NOP10, PA2G4, PAPD5, RPL18, RPL32, |
| | | | | | | | RPL7, RPLP1, RPP21, RPP38, RPS14, |
| | | | | | | | RPS25, RPS26, RPS29, RPS3A, RRP1, |
| | | | | | | | TARBP1, TFB1M, UTP3, XRN2, YBEY |
| GO | GO: | regulated | 58 | 1.86E−04 | 1.00E+00 | 4.14E−02 | ACTN4, APAF1, ATG7, ATP11B, ATP6V0C, |
| Biological | 0045055 | exocytosis | | | | | B4GALT1, CAT, CCT8, CD109, CD300A, |
| Process | | | | | | | CD58, CD97, CHP1, CKAP4, CNN2, |
| | | | | | | | CORO1A, COTL1, CPNE3, CREG1, |
| | | | | | | | CRISPLD2, CTSW, CTSZ, DNAJC13, |
| | | | | | | | DNAJC3, DOCK2, EEF2, EFCAB4B, |
| | | | | | | | ERP44, FAM3C, FERMT3, FPR2, |
| | | | | | | | GDI2, HMHA1, HSP90AA1, |
| | | | | | | | HSPA8, IMPDH1, ISLR, ITGB2, |
| | | | | | | | KCMF1, LAIR1, LAMP1, LAMTOR2, |
| | | | | | | | LPCAT1, LYN, MAN2B1, MANBA, MLEC, |
| | | | | | | | NPC2, P2RX1, PA2G4, PCDH7, PDAP1, |
| | | | | | | | PDXK, PGLYRP1, PIP5K1C, PLAC8, |
| | | | | | | | PLEKHO2, PRCP, PRDX6, PRG3, PSAP, |
| | | | | | | | PSMD1, PSMD3, PSMD6, PTGES2, PTPN6, |
| | | | | | | | PTPRN2, PYGB, QPCT, RAB10, RAB4B, |
| | | | | | | | RAB7A, RAP2B, SCAMP1, SERPINB1, |
| | | | | | | | SIGLEC5, SLC2A3, SPP2, STK10, SVIP, |
| | | | | | | | SYNGR3, SYNJ1, SYT1, SYT10, SYT11, |
| | | | | | | | SYT13, TGFB1, TGFB3, TIMP2, TMC6, |
| | | | | | | | TMEM179B, TOLLIP, TRPM2, TUBB, |
| | | | | | | | UBR4, VAMP2, VCP, XRCC5 |
| GO | GO: | mRNA 3′- | 59 | 1.96E−04 | 1.00E+00 | 4.28E−02 | ATP6V1G2-DDX39B, |
| Biological | 0031124 | end | | | | | CPSF2, CPSF3, CPSF6, DDX39A, |
| Process | | processing | | | | | PABPN1, PAPOLG, POLDIP3, POLR2D, |
| | | | | | | | SARNP, SLBP, SRSF1, SRSF2, SYMPK, |
| | | | | | | | U2AF2, ZNF473 |
| GO | GO: | mitochondria | 60 | 2.30E−04 | 1.00E+00 | 4.94E−02 | AARS2, ATP5SL, C11orf83, C6orf57, |
| Biological | 0033108 | I respiratory | | | | | COA5, COX19, NDUFA6, NDUFAB1, |
| Process | | chain | | | | | NDUFAF1, NDUFB1, NDUFB7, |
| | | complex | | | | | NDUFC1, NDUFV2, OXA1L, |
| | | assembly | | | | | PET100, SLC25A33 |

Overall, in early-stage cancer patients, we found the increases in fragmentation levels at hotspots near microsatellites (Class II hotspots) and the decreases in fragmentation levels at hotspots near CTCF and promoters (Class I hotspots), which are enriched in the immune-related GO terms from the peripheral immune cells.

### Cell-Free DNA Fragmentation Hotspots can Mitigate the Overdiagnosis Concern.

Overdiagnosis is one of the major concerns for the diagnosis of early-stage cancer. We next explored whether or not the IFS signals from cfDNA fragmentation hotspots could also characterize the differences between early-stage HCC and non-malignant liver diseases. We identified the hotspots on the additional cfDNA WGS datasets from 67 patients with chronic HBV infection and 36 patients with HBV-associated liver cirrhosis in the same study[25]. PCA analysis of IFS signals across all the hotspots suggested a clear separation between early-stage HCC and non-malignant liver diseases, as well as healthy controls (FIG. S12a). To test if the separation of samples is due to the possible batch effect, we performed PCA on IFS from matched random genomic regions in the same sample and did not observe a clear separation between groups of samples (FIG. S12b). Another possible technical artifact for the clear separation between HCC and other conditions could be due to our pooling strategy for the hotspot calling in low-coverage WGS data. The hotspot calling on the pooled group may enrich the regions with similar depletions in the genome without any meaningful biological indications. To test if the separation of samples is due to this artifact, we randomly grouped the samples and called the hotspots from these random groups with the matched group sizes. The PCA results did not show any separations as expected (FIG. S12c). We further selected the top 30,000 most variable hotspots, performed the unsupervised hierarchical clustering, and observed the clear dynamics of the fragmentation patterns among early-stage HCC, HBV, Cirrhosis, and healthy controls (FIGS. S13-14). Finally, by 10-fold cross-validation, the linear SVM model showed a higher classification performance (83% sensitivity at 100% specificity) than other methods from the same dataset (FIG. S15, Table S6-7).

TABLE S6
Performance comparison in HCC vs. HBV + Cirrhosis (before GC bias correction)

| | | Sensitivity | | | |
|---|---|---|---|---|---|
| | | @100% | Sensitivity @98% | Sensitivity @95% | Sensitivity @85% |
| | AUC | specificity | specificity | specificity | specificity |
| IFS | 0.9770 (0.95 CI: | 0.8333 (0.95 | 0.8333 (0.95 | 0.8333 (0.95 | 0.9556 (0.95 |
| hotspots | 0.9588-0.9951) | CI: 0.6670- | CI: 0.6670- | CI: 0.6670- | CI: 0.9074- |
| | | 0.9997) | 0.9997) | 0.9997) | 1.0000) |
| CNV | 0.7400 (0.95 CI: | 0.5333 (0.95 | 0.6222 (0.95 | 0.6222 (0.95 | 0.7611 (0.95 |
| | 0.7025-0.7775) | CI: 0.4127- | CI: 0.5173- | CI: 0.5173- | CI: 0.6474- |
| | | 0.6539) | 0.7272) | 0.7272) | 0.8748) |
| mtDNA | 0.9414 | 0.1556 | 0.6556 | 0.7556 | 0.8444 |

TABLE S7
Performance in HCC vs. HBV + Cirrhosis (after GC bias correction)

| | | Sensitivity | | | |
|---|---|---|---|---|---|
| | | @100% | Sensitivity @98% | Sensitivity @95% | Sensitivity @85% |
| | AUC | specificity | specificity | specificity | specificity |
| IFS | 0.9772 (0.95 CI: | 0.9111 (0.95 | 0.9111 (0.95 | 0.9111 (0.95 | 0.9333 (0.95 |
| hotspots | 0.9603-0.9940) | CI: 0.8676- | CI: 0.8676- | CI: 0.8676- | CI: 0.8852- |
| | | 0.9547) | 0.9547) | 0.9547) | 0.9815) |

### Cell-Free DNA Fragmentation Hotspots Boost the Power for the Detection and Localization of Multiple Early-Stage Cancers.

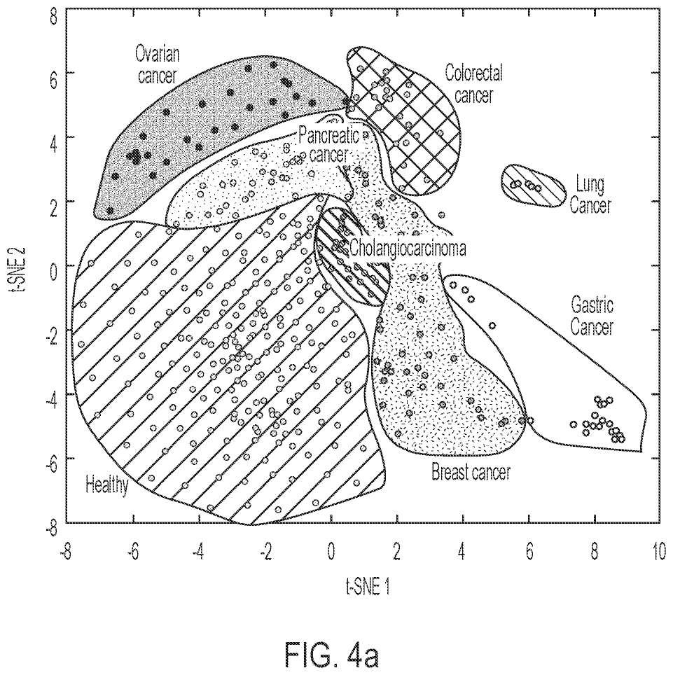

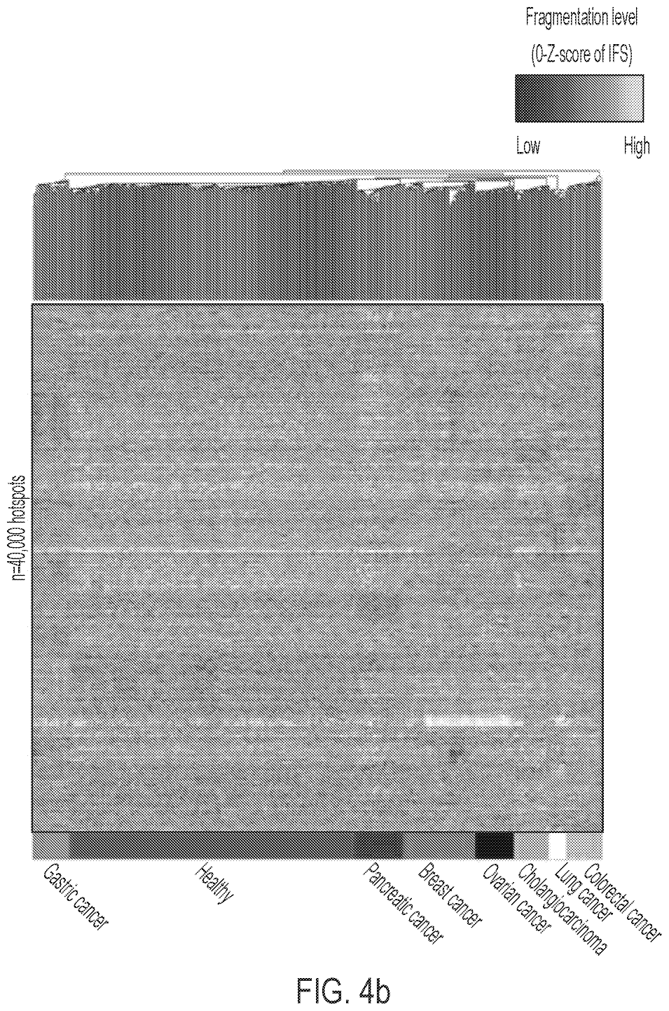

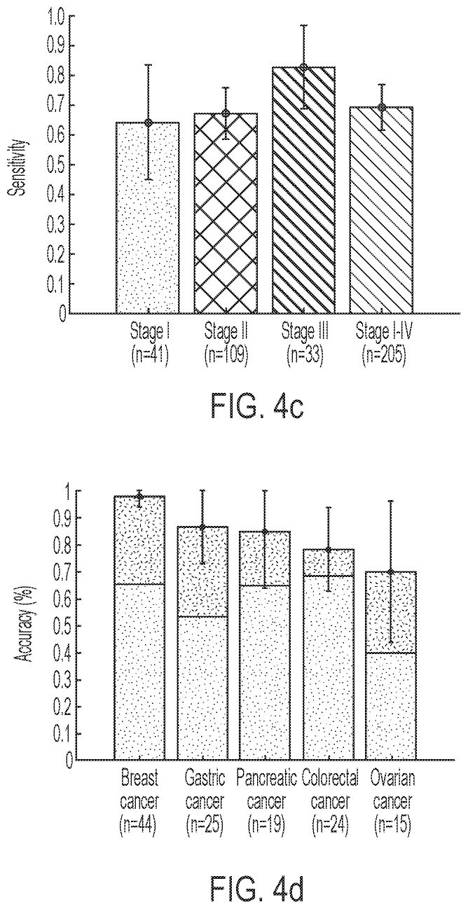

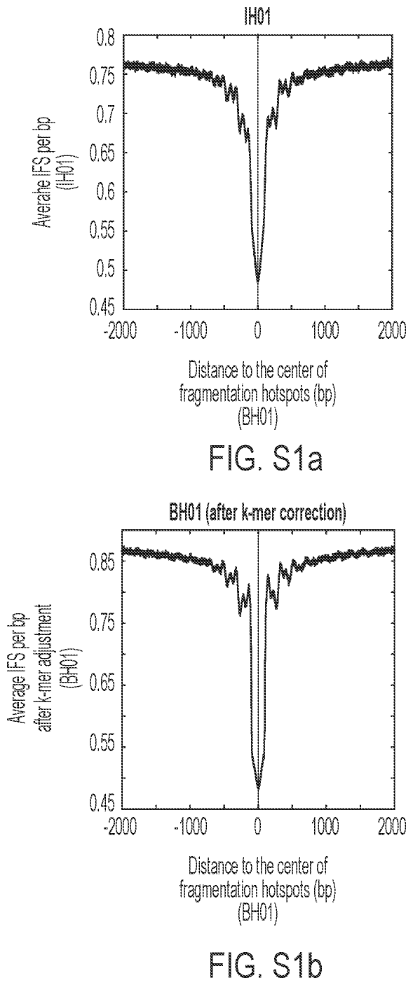

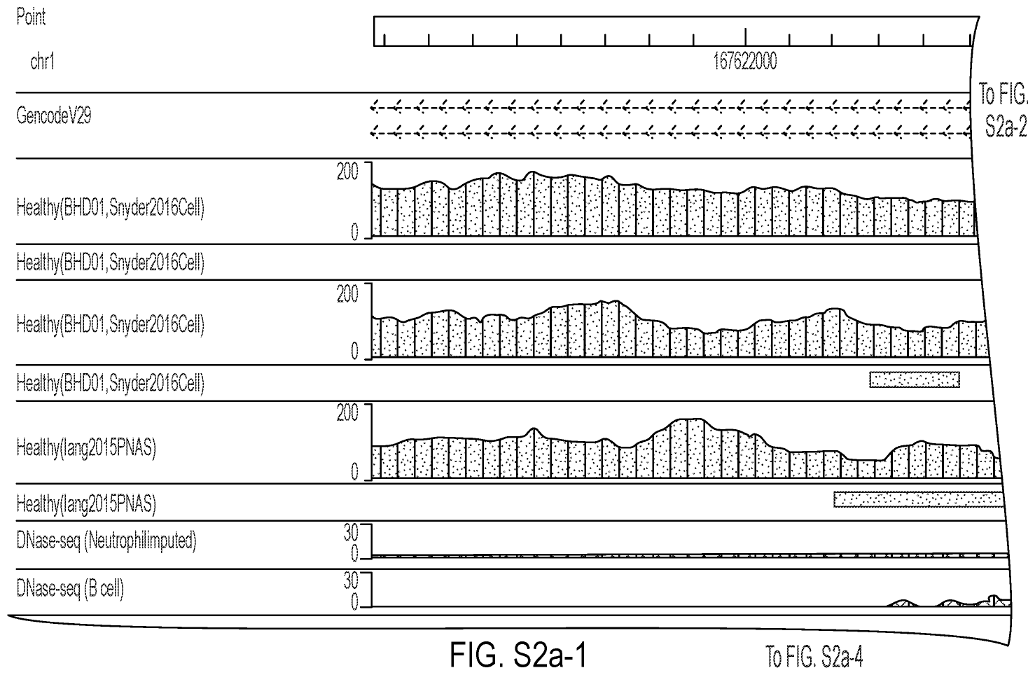

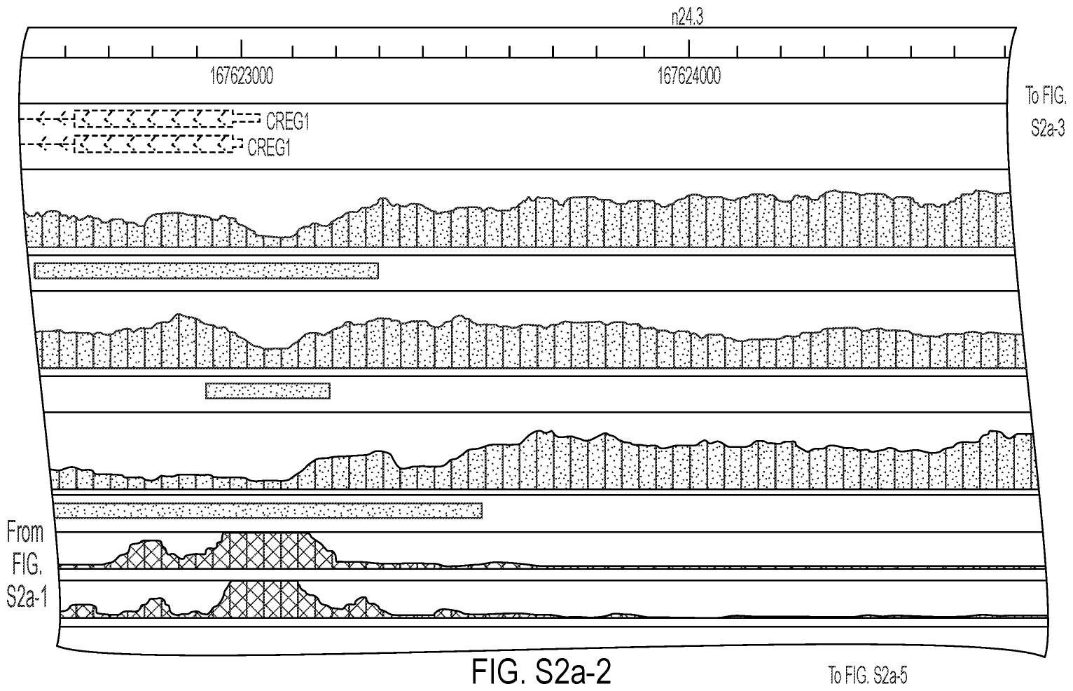

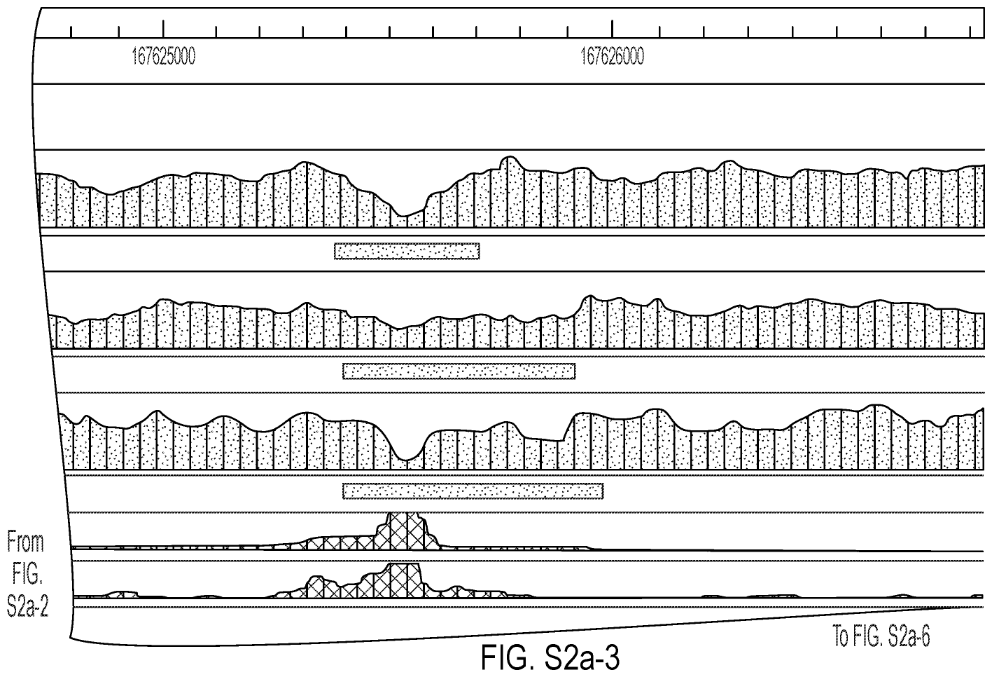

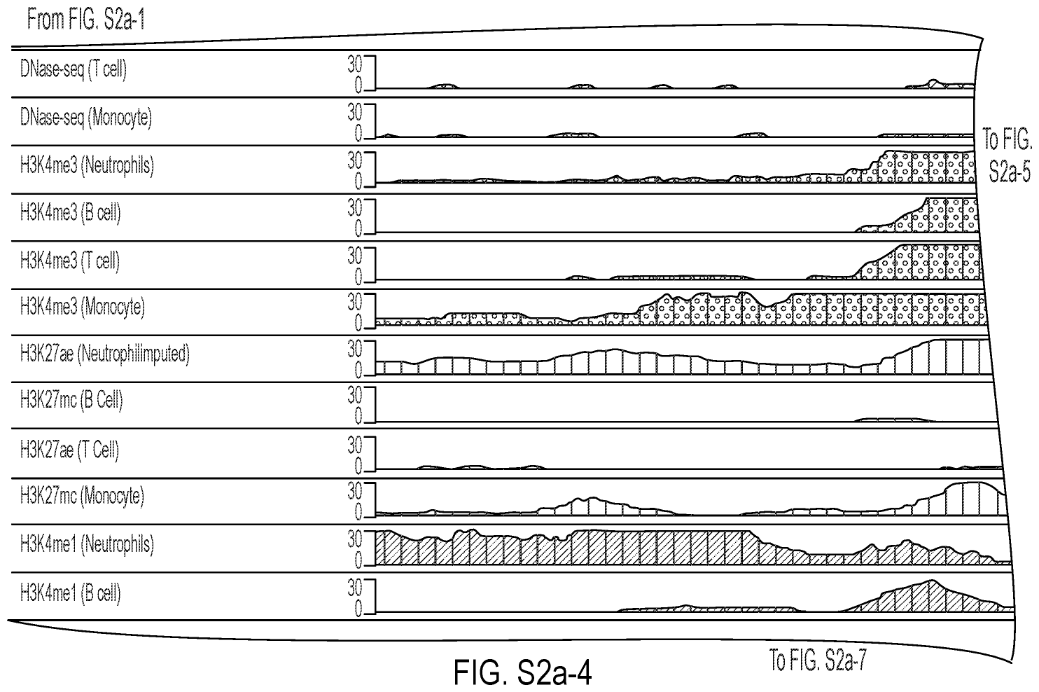

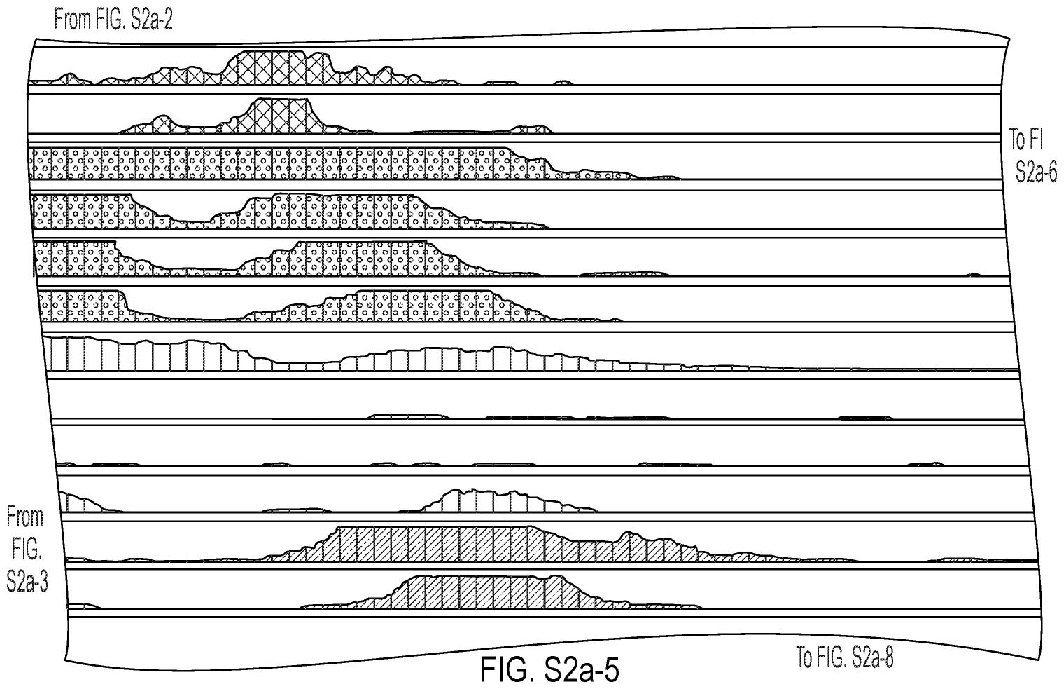

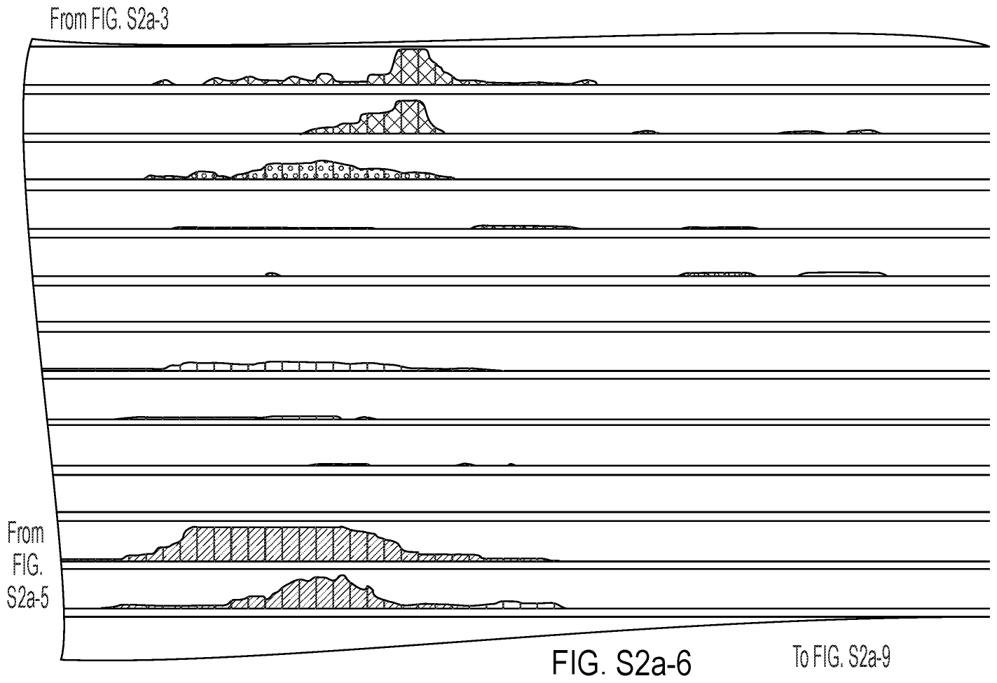

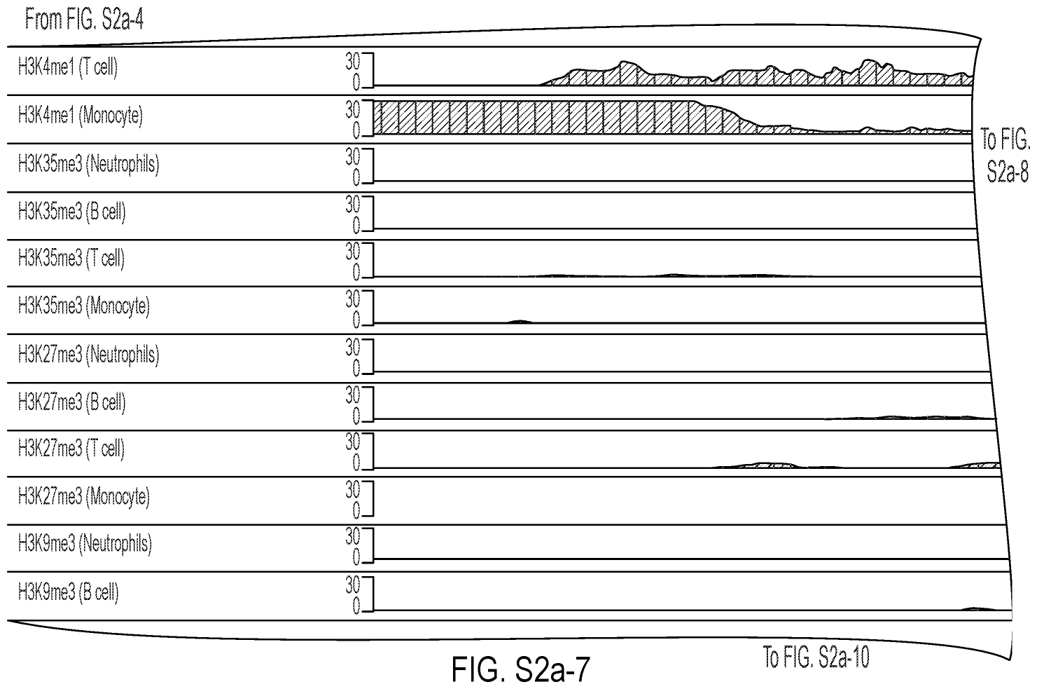

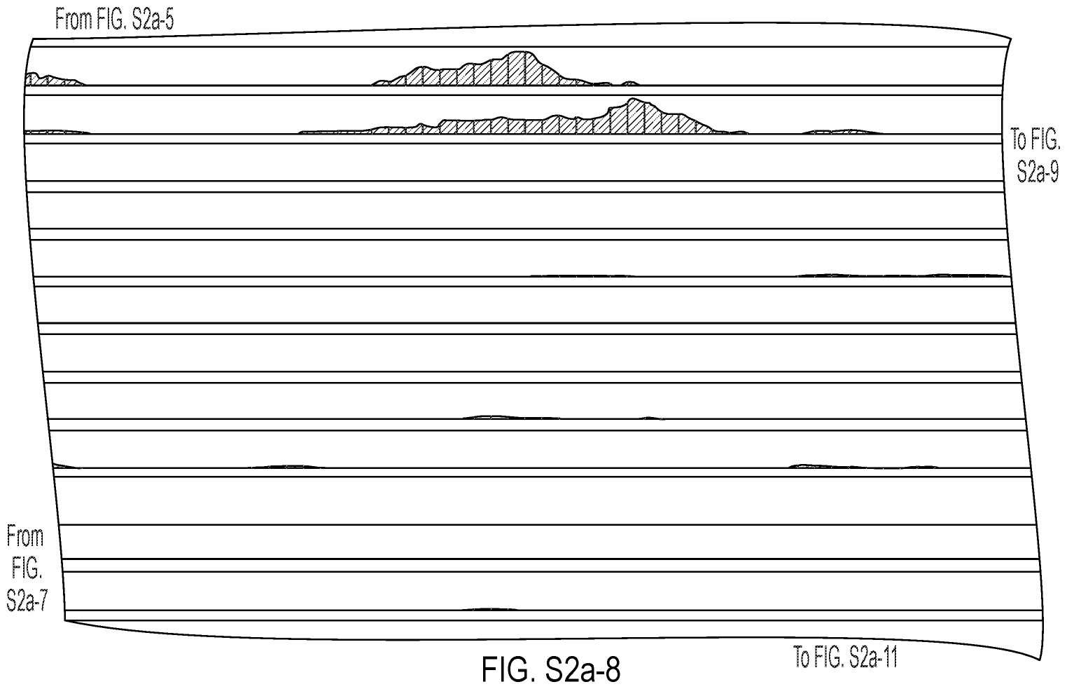

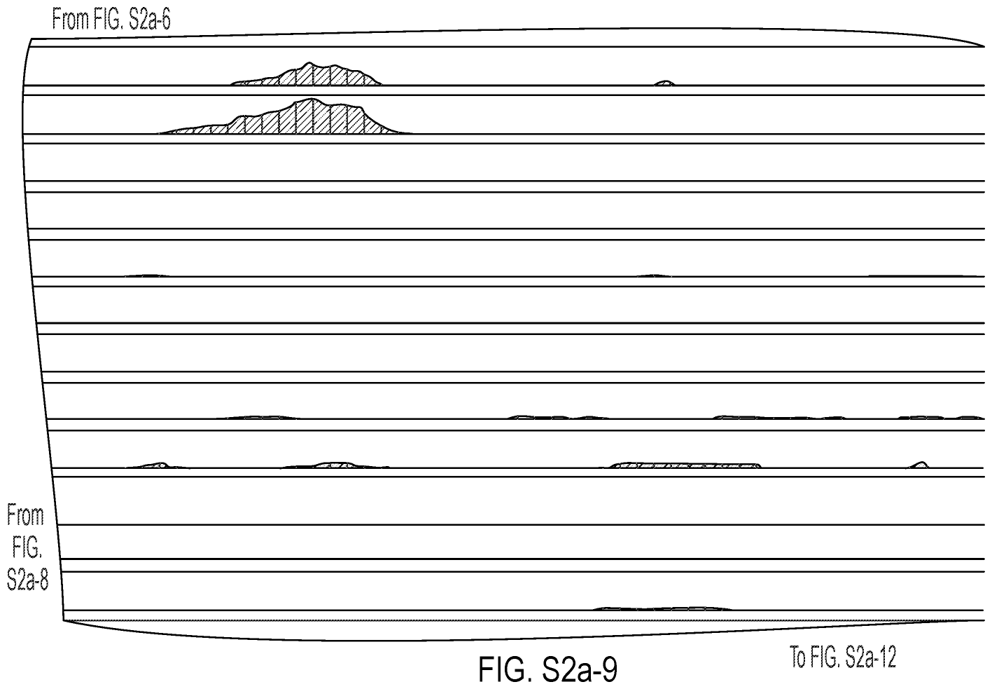

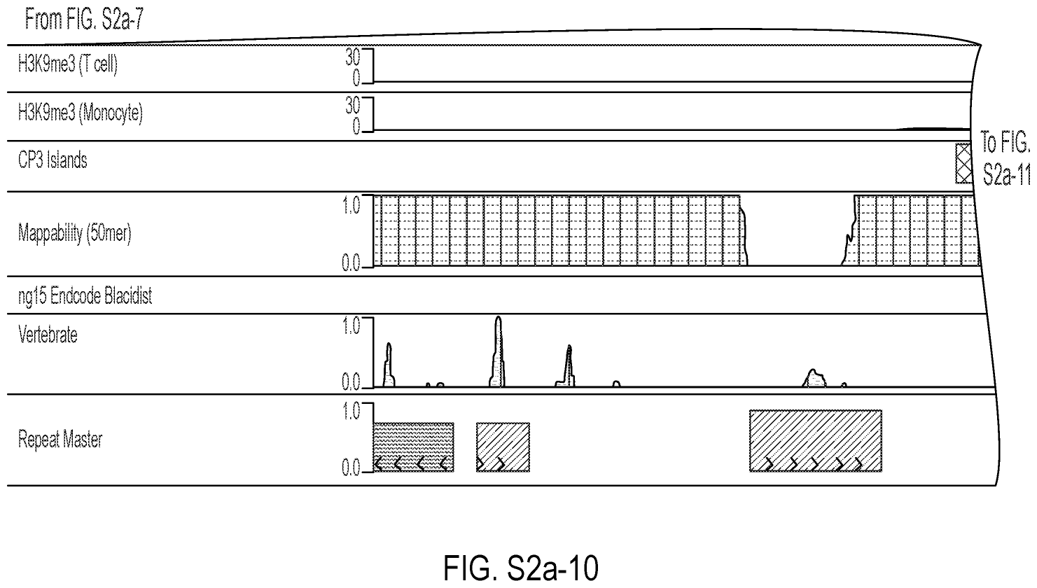

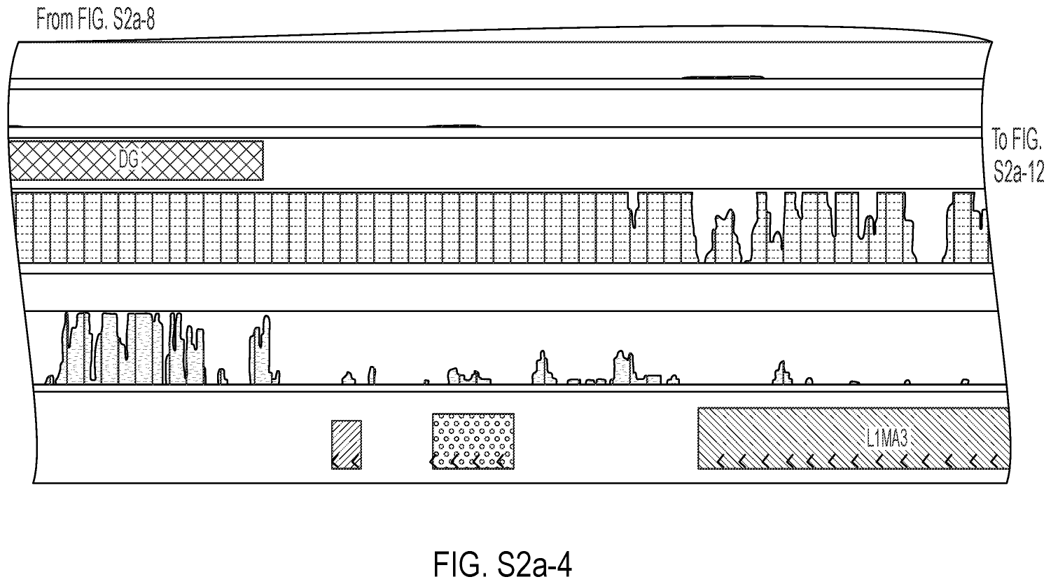

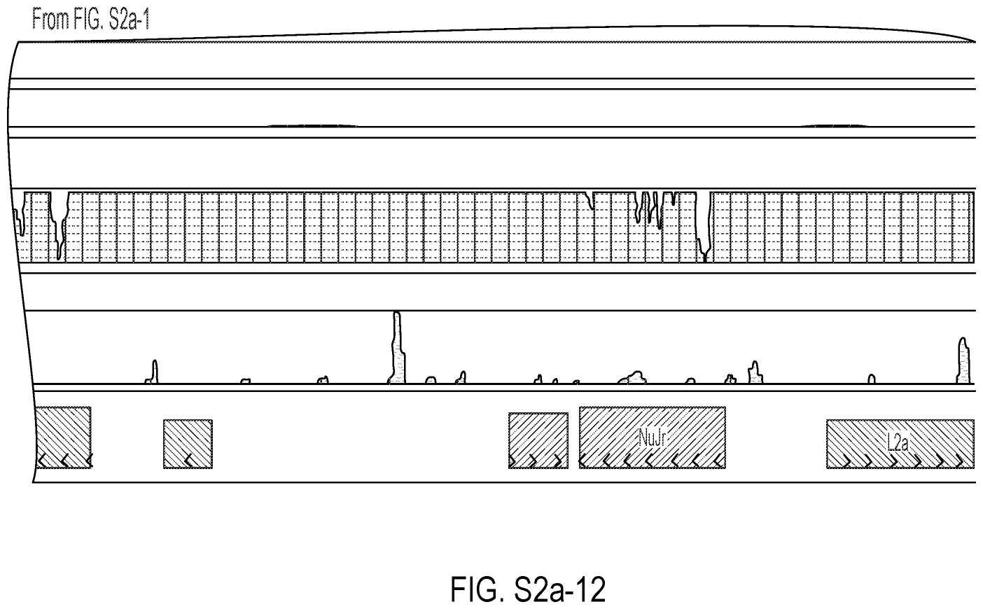

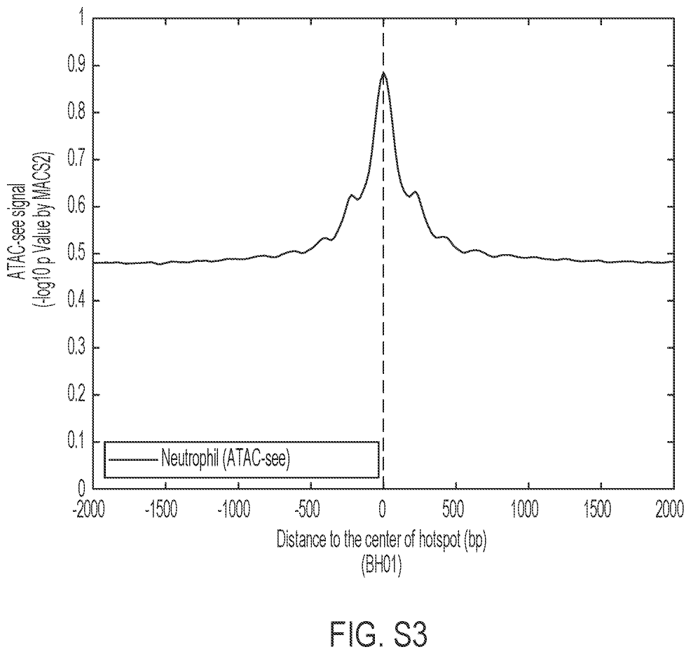

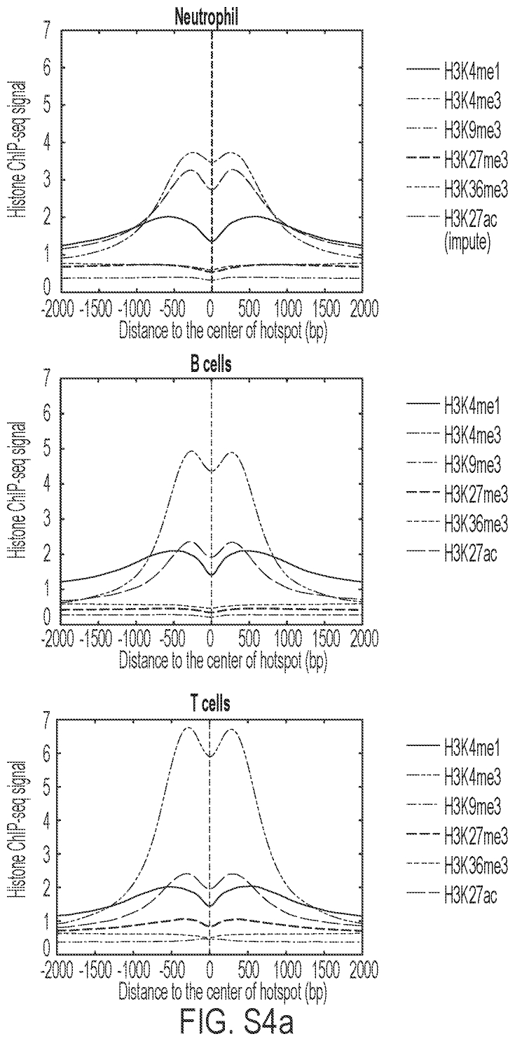

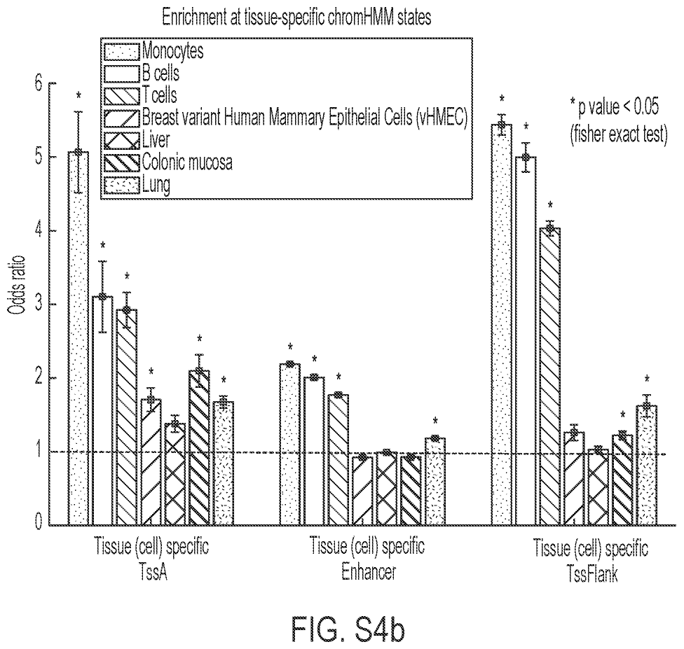

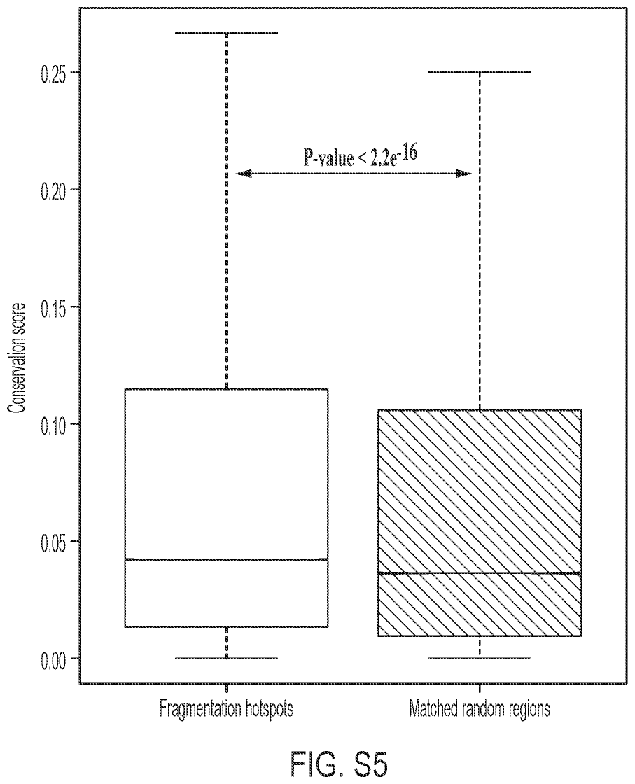

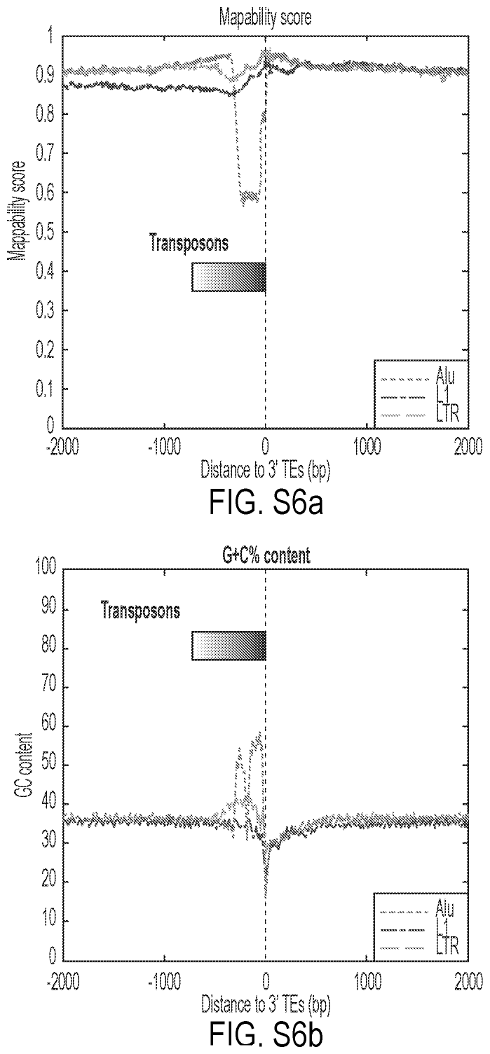

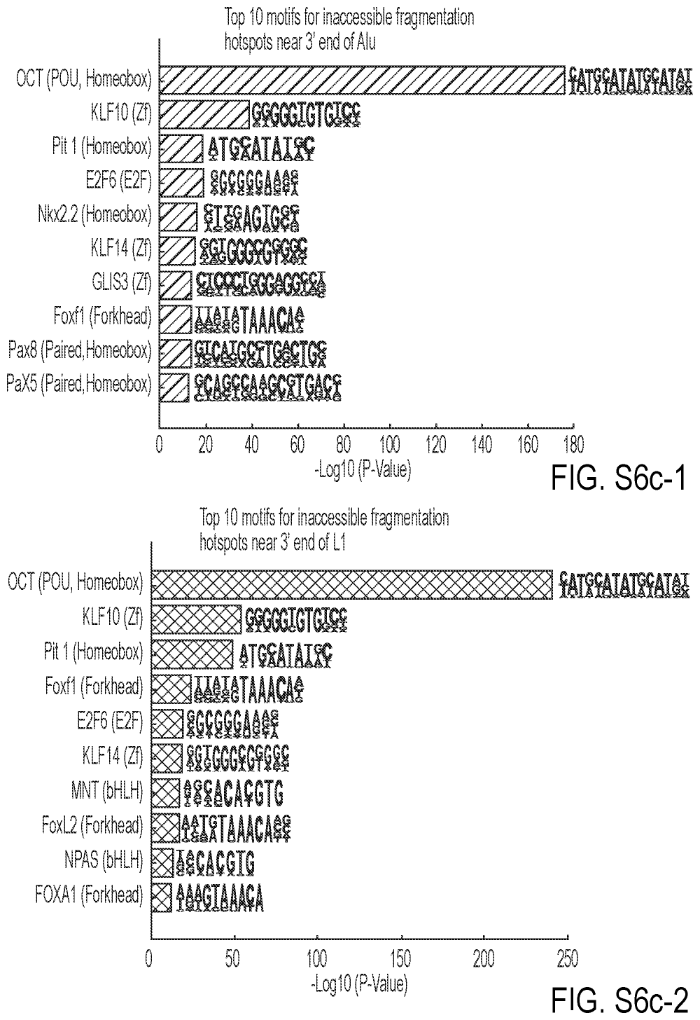

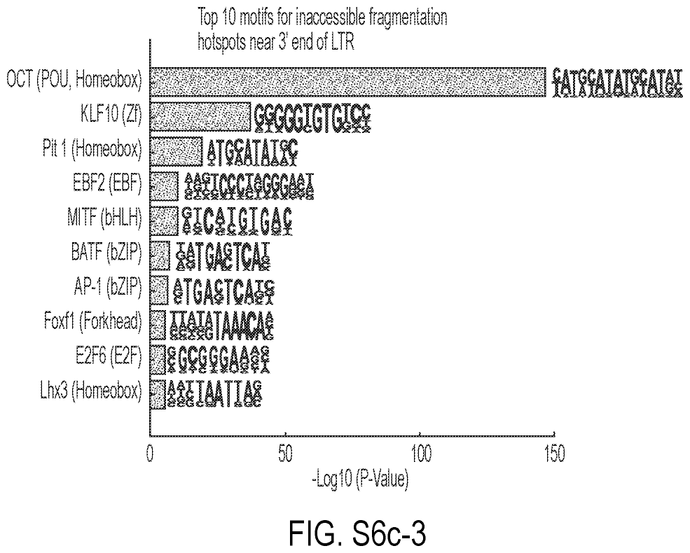

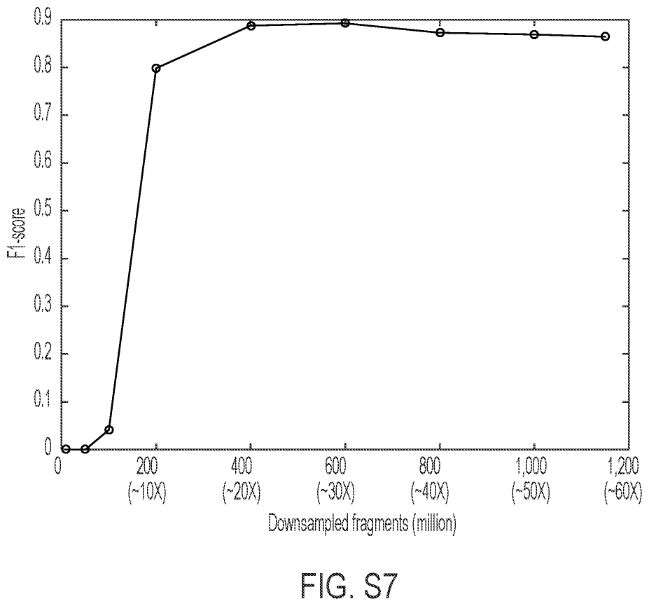

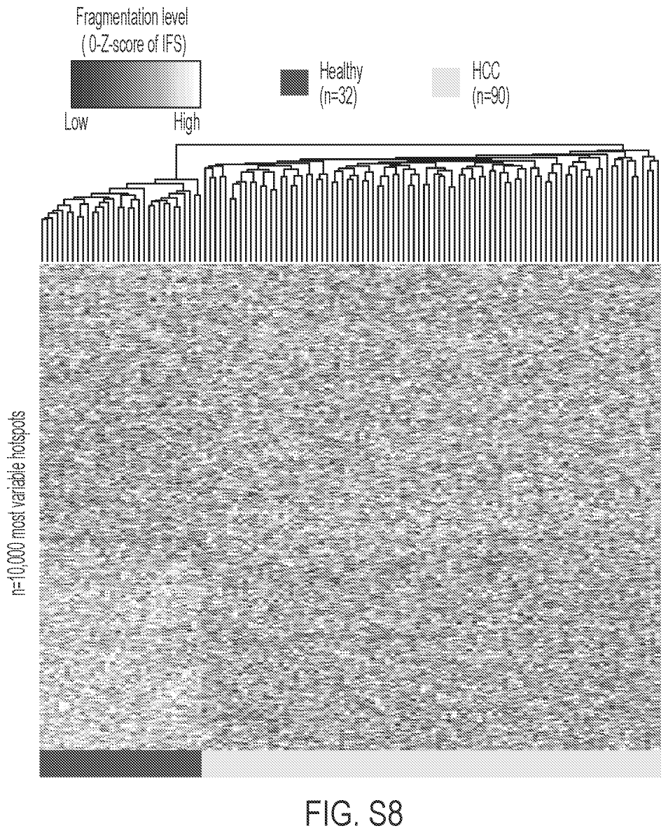

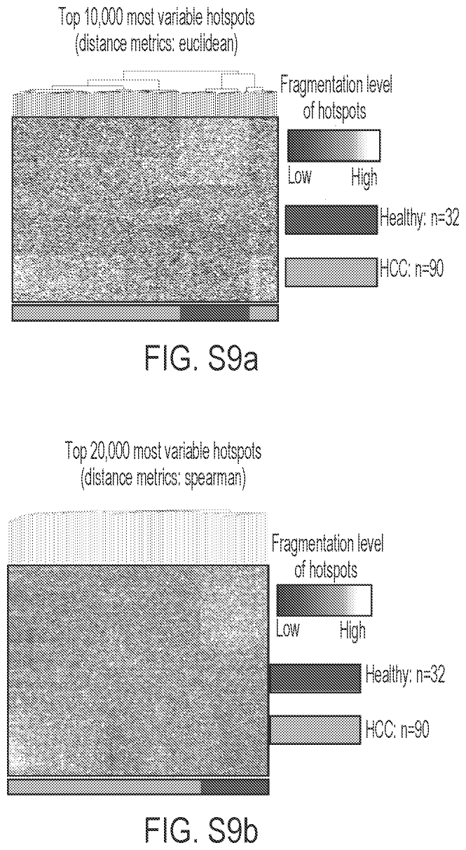

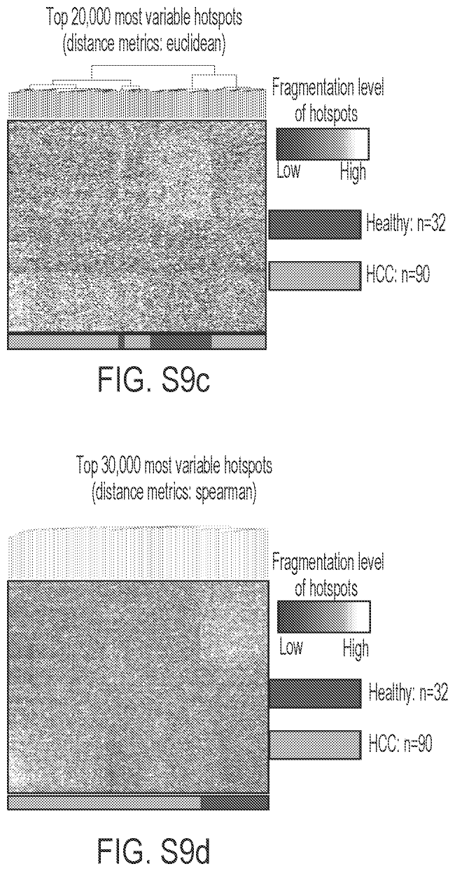

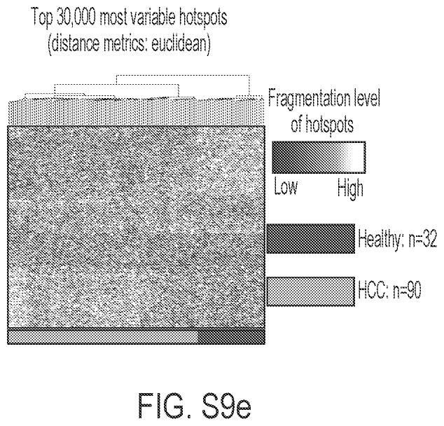

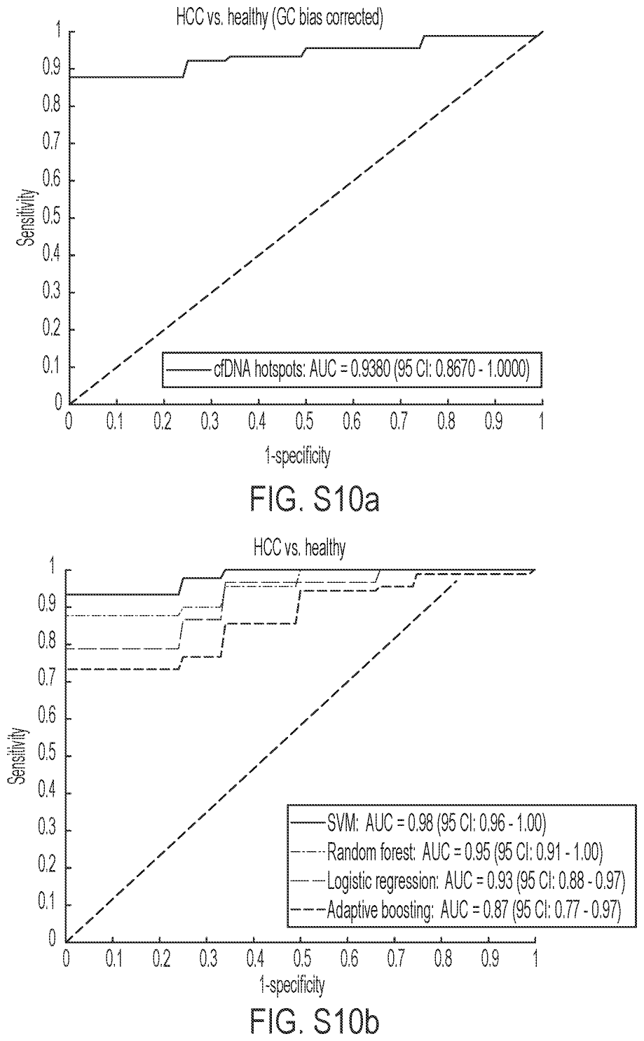

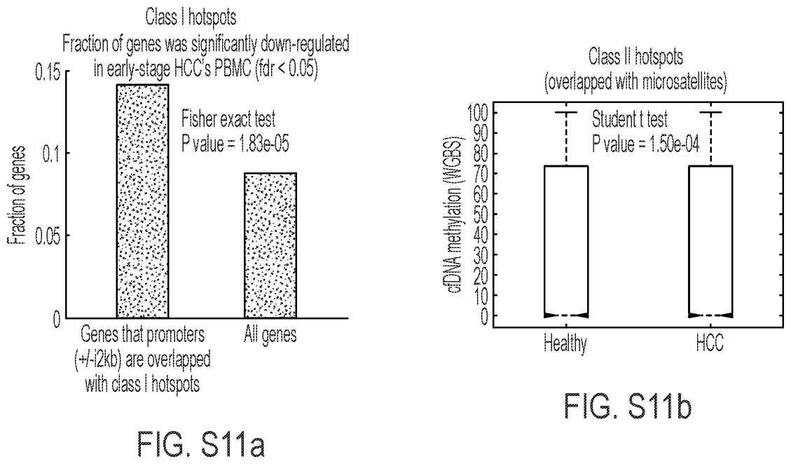

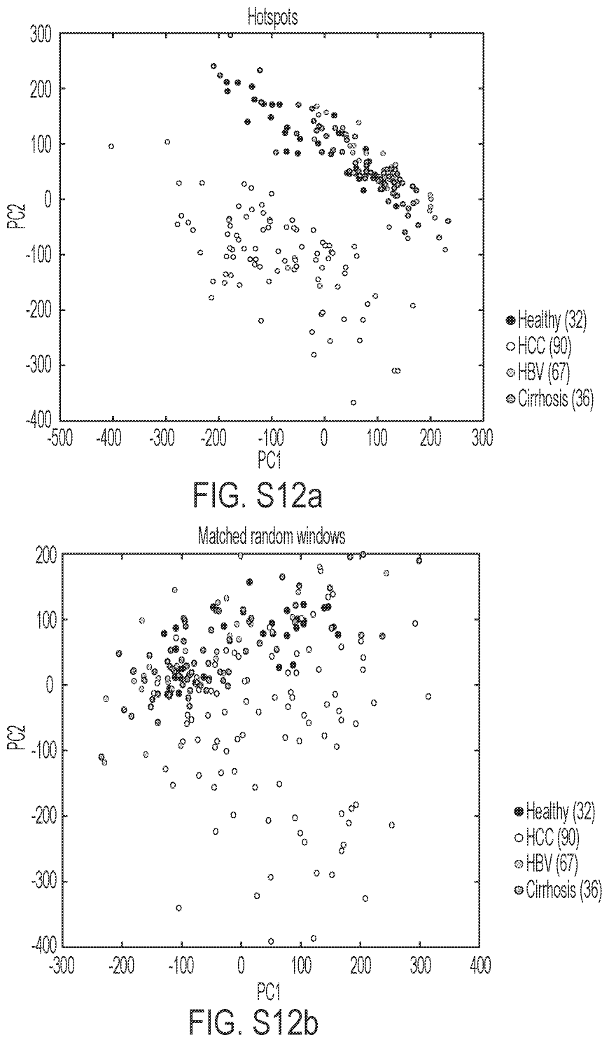

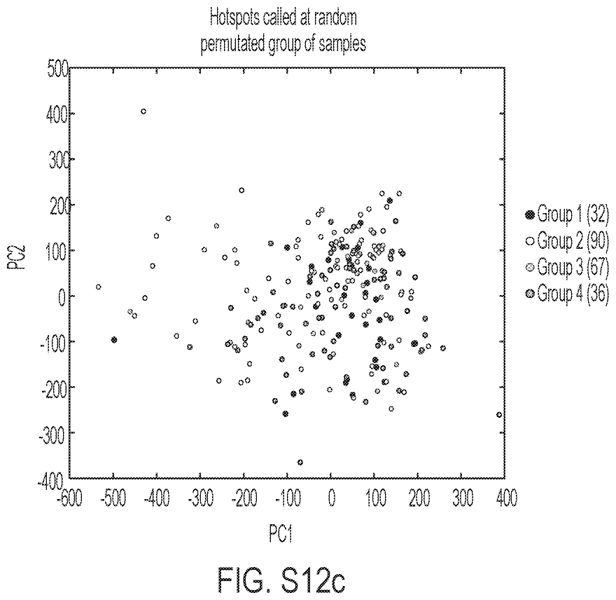

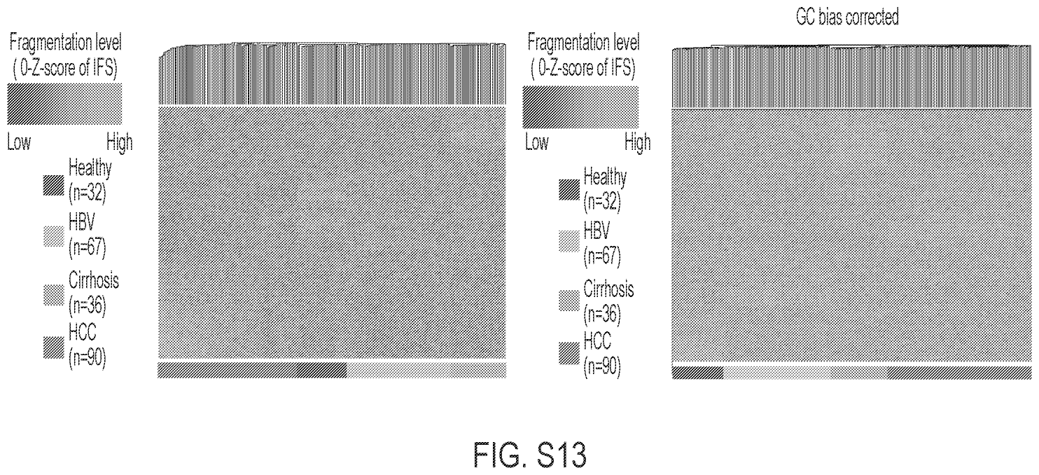

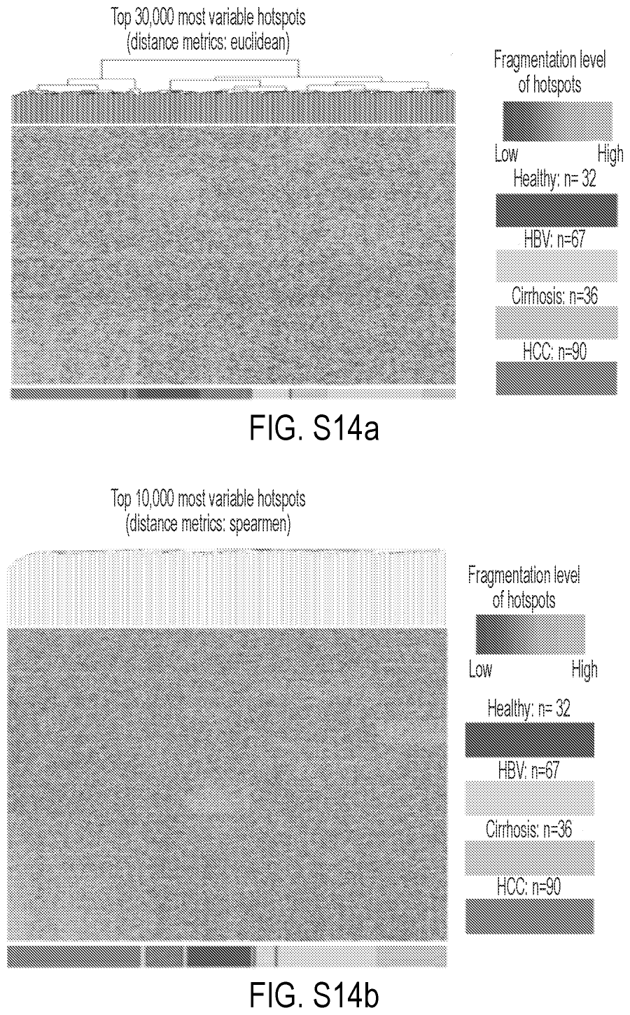

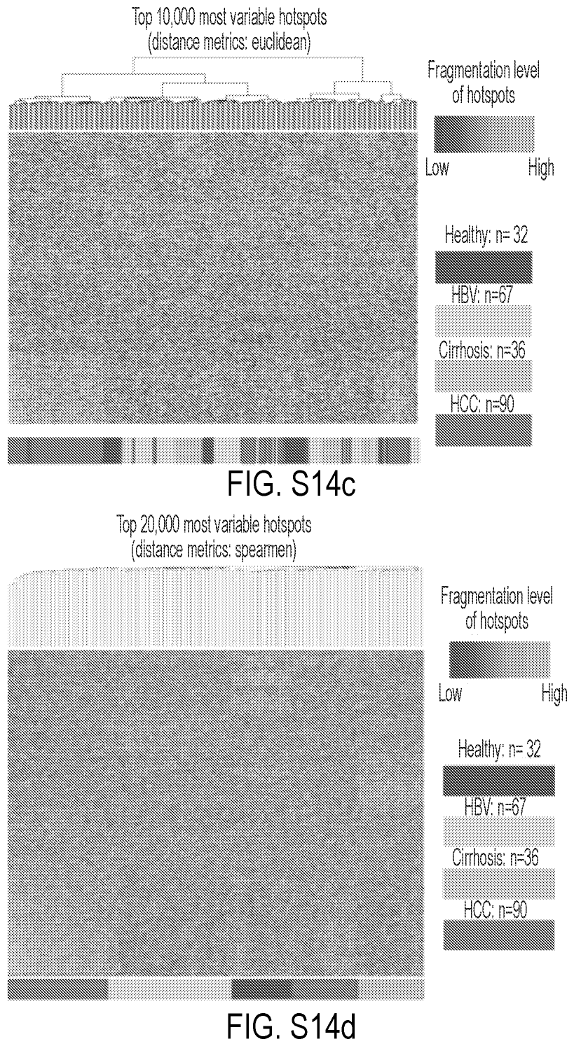

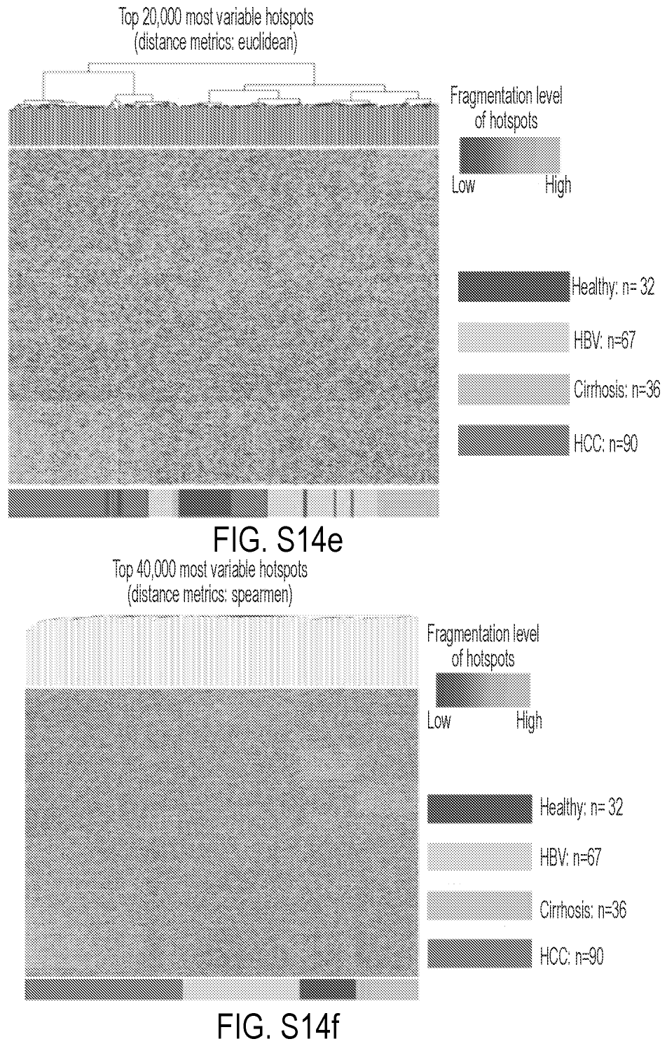

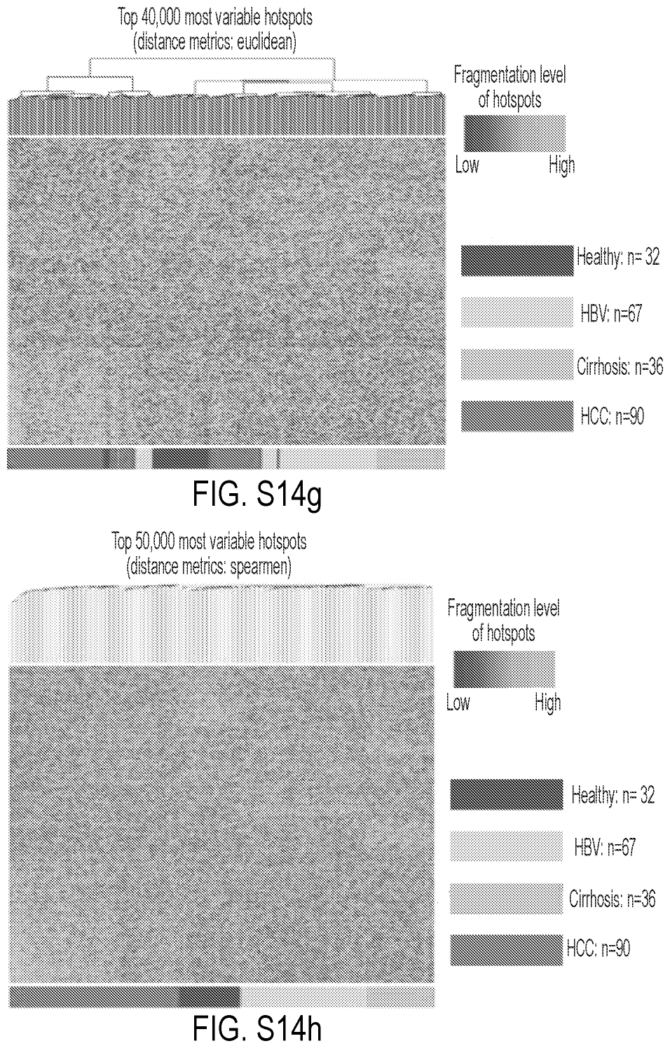

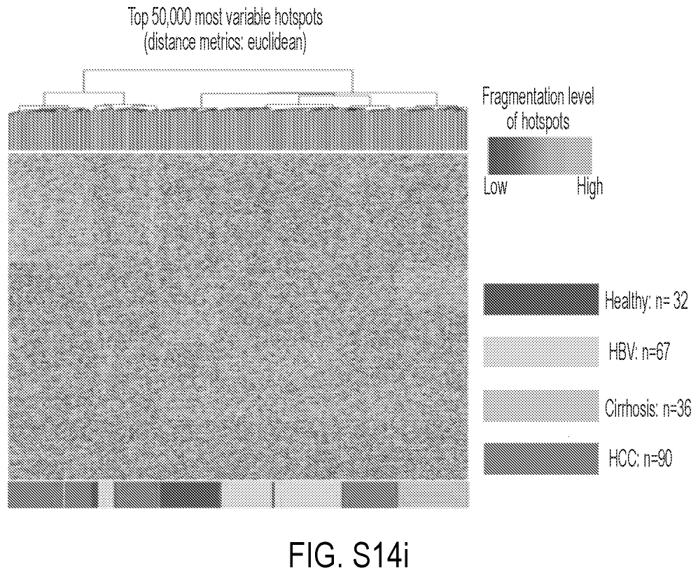

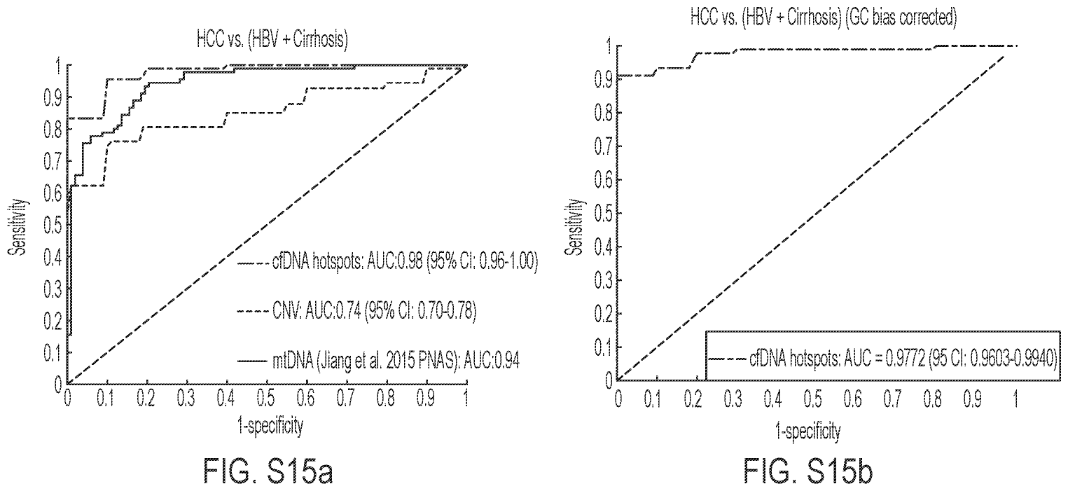

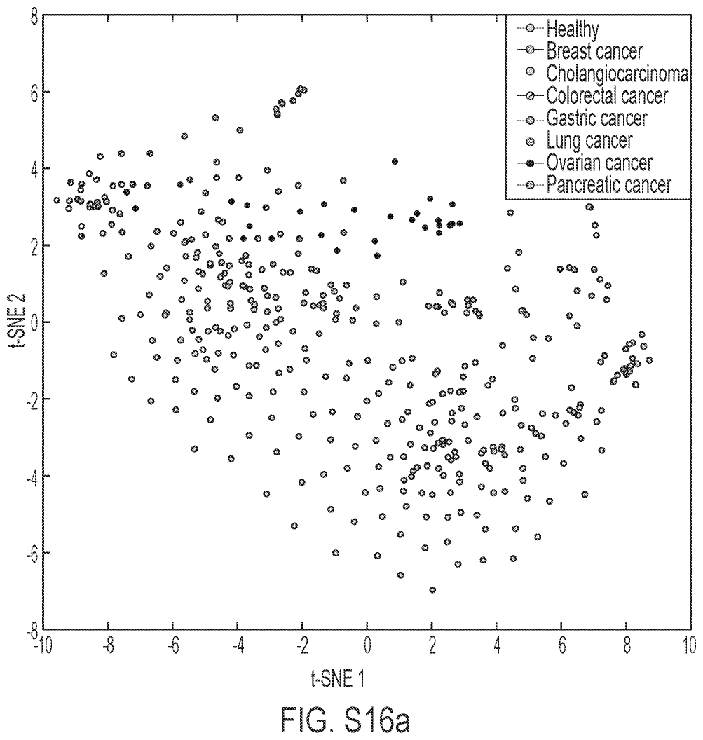

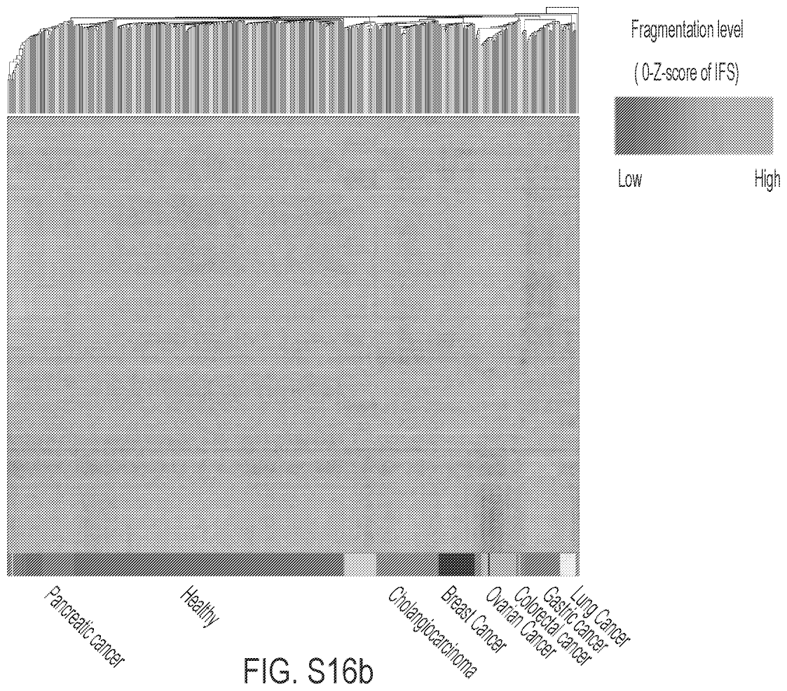

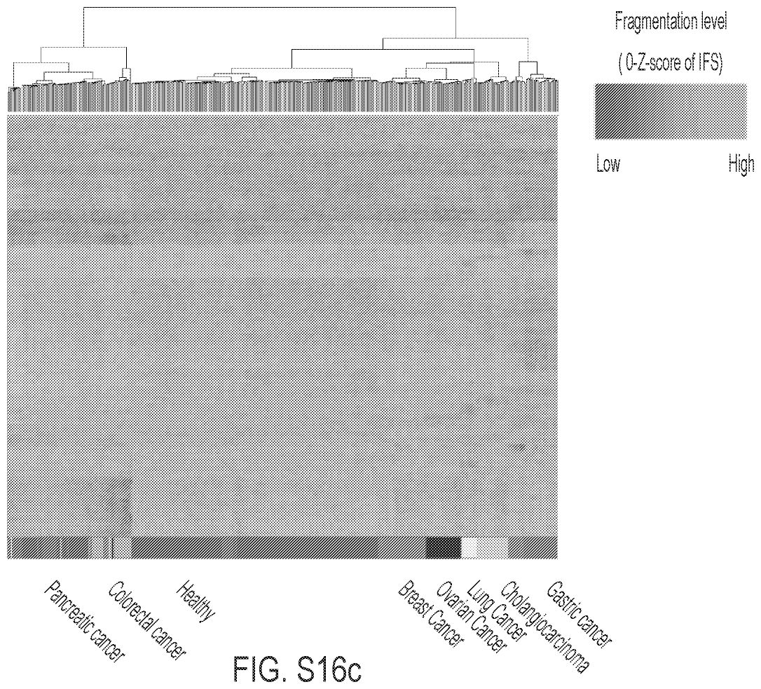

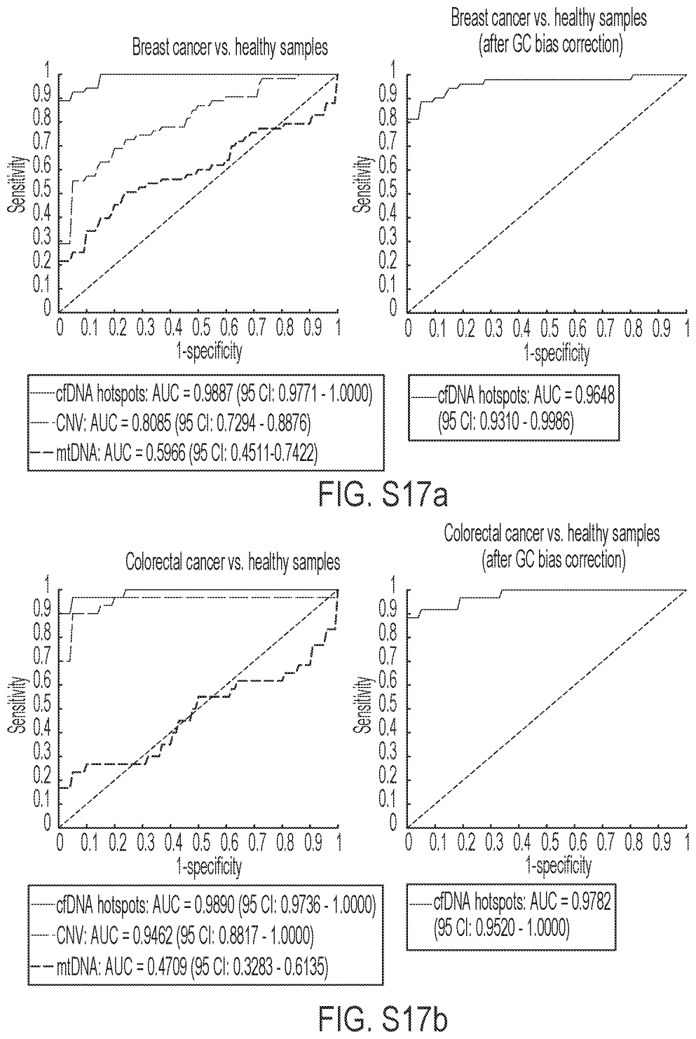

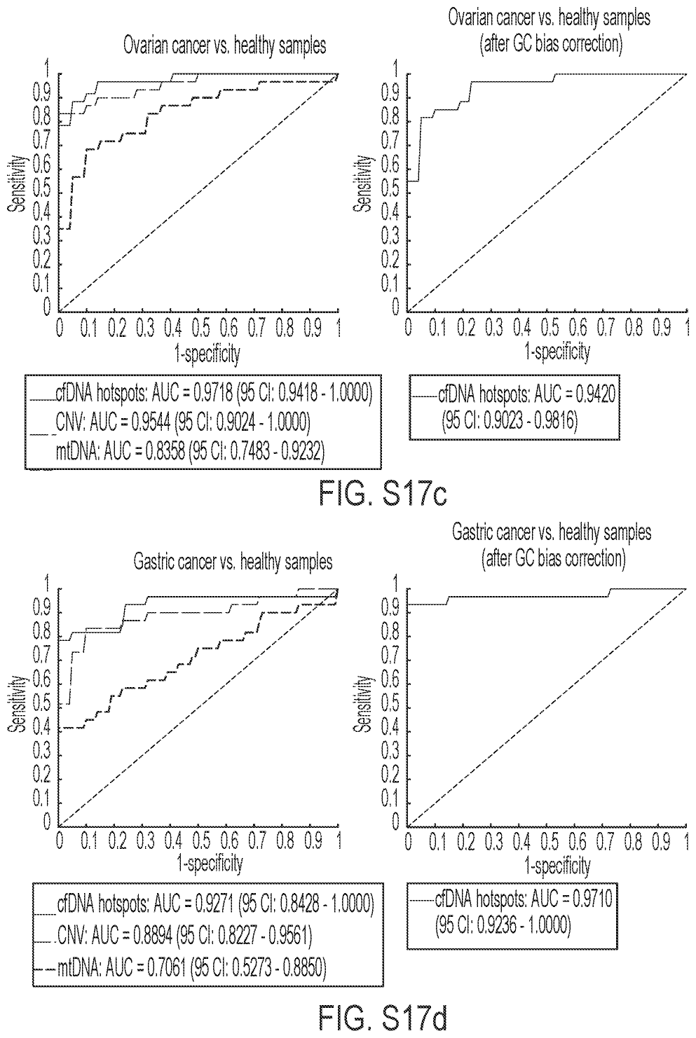

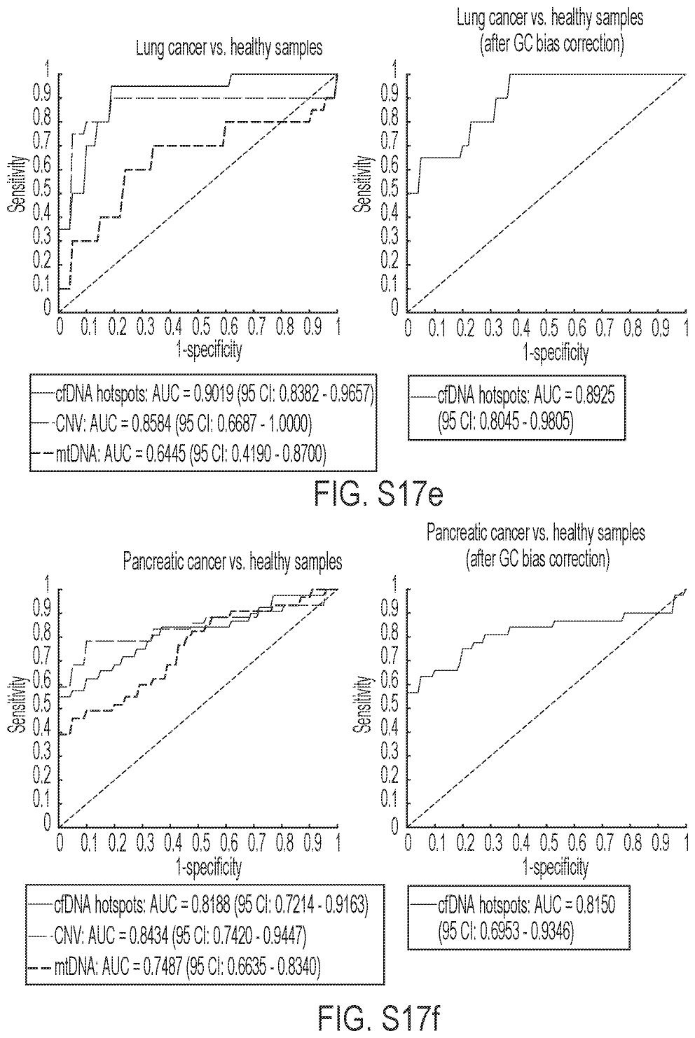

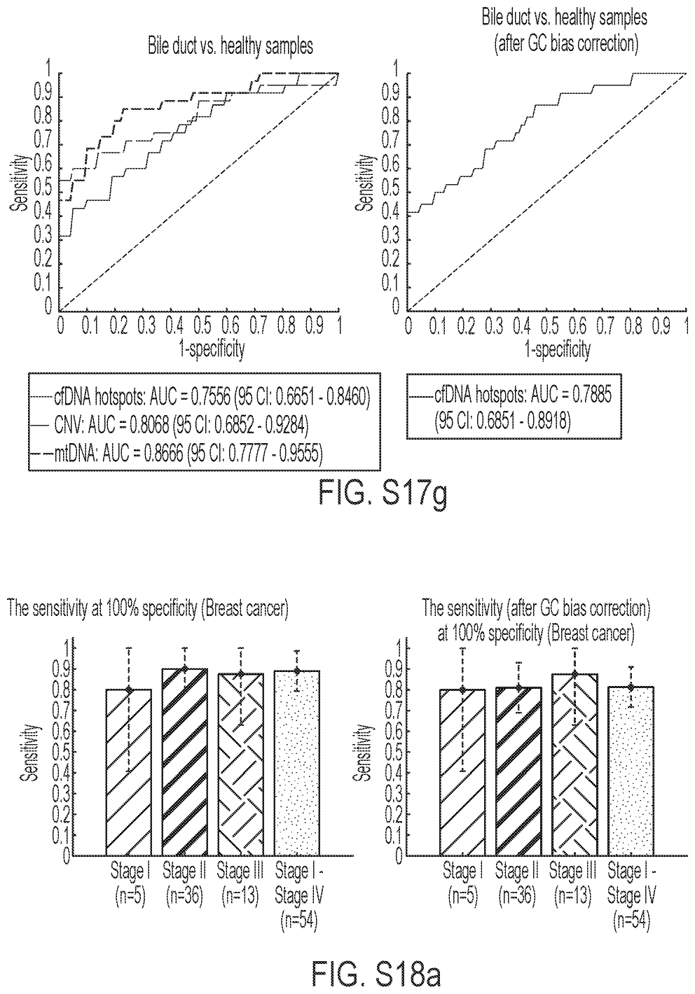

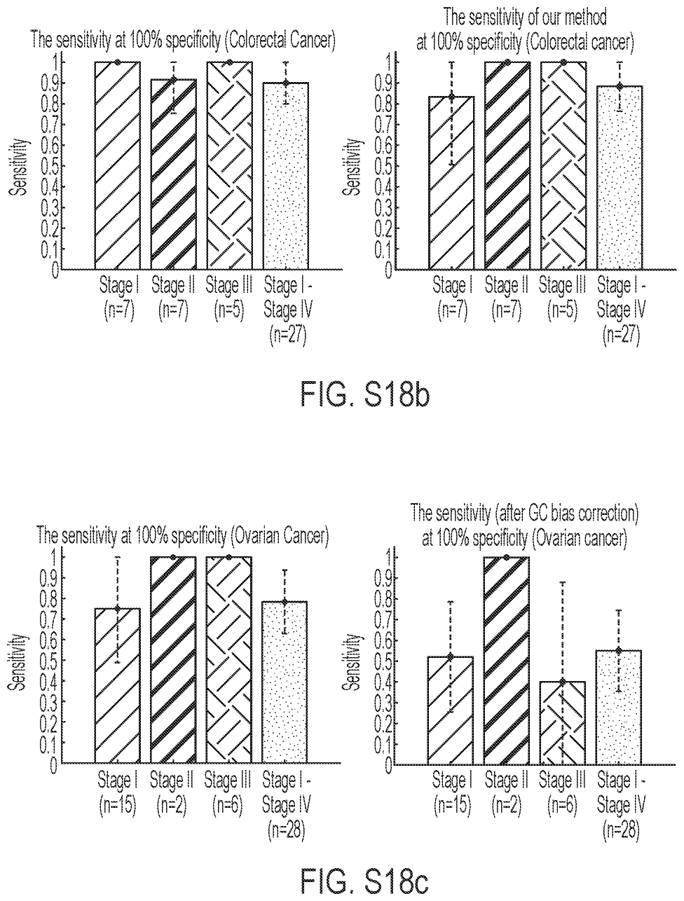

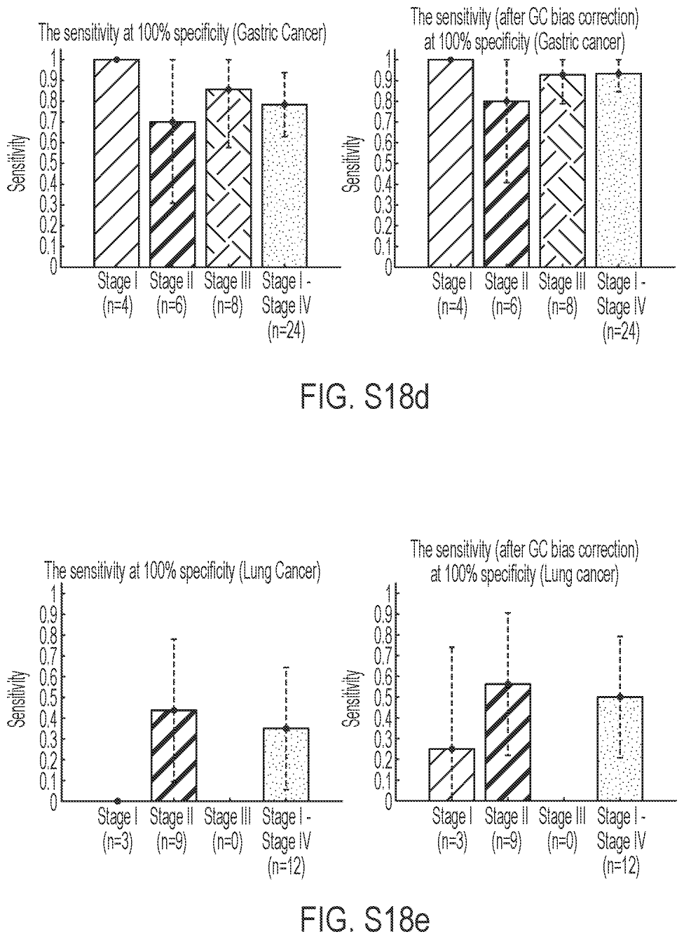

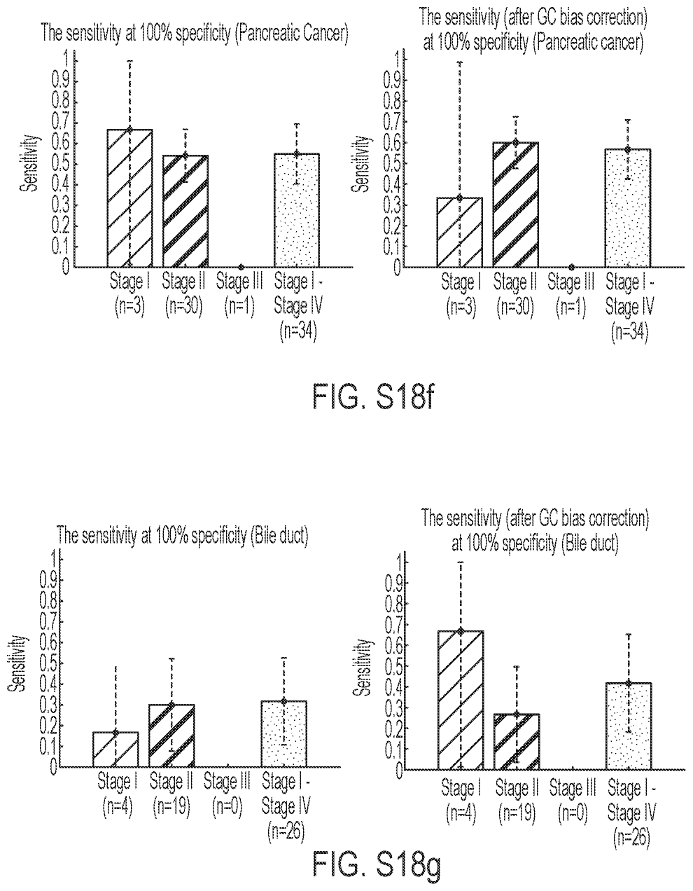

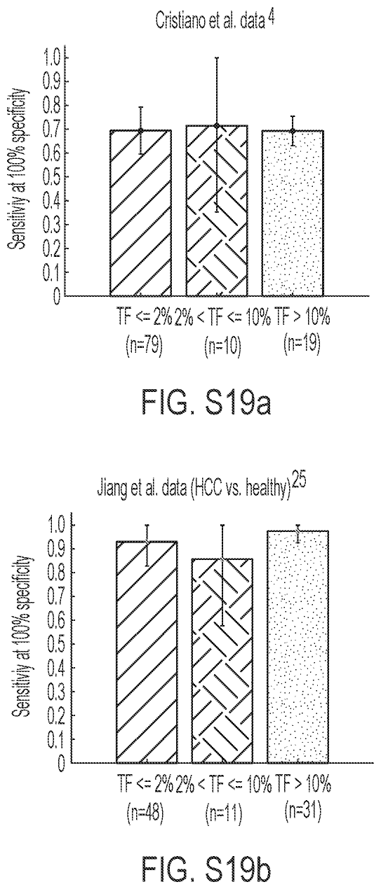

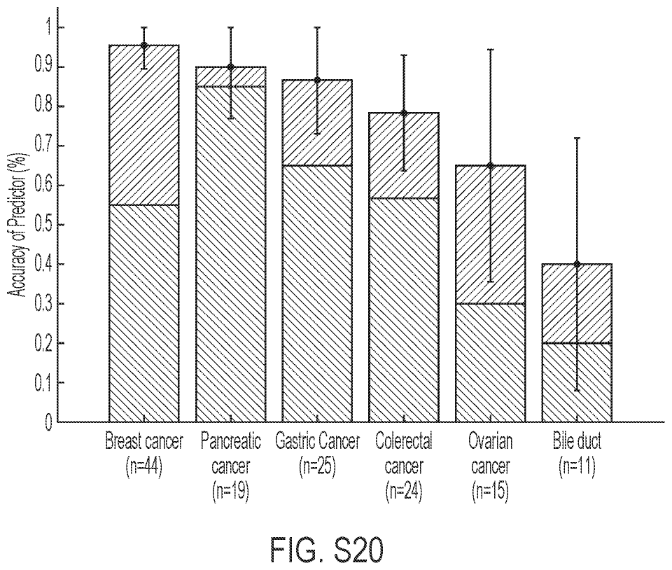

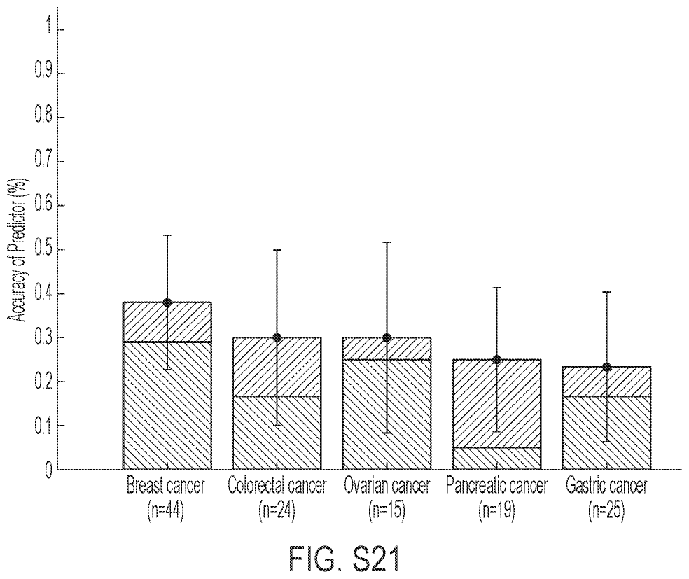

One of the biggest challenges for the detection of early-stage cancer is to obtain high accuracy across multiple types of cancer, which is not available in clinics yet. To further validate our method in a more comprehensive early-stage cancer dataset, we collected publicly available low-coverage cfDNA WGS data (˜1×/sample) from 208 patients across seven different kinds of cancer (88% in stage I-III, colon, breast, lung, gastric, bile duct, ovary, and pancreatic cancer) and matched 215 healthy controls in the same study[4]. We applied a similar strategy to the HCC study above for the hotspot calling (pool the samples to achieve enough coverage as stated in FIG. S7). Across seven different types of cancer and healthy conditions, the z-score of IFS signals in the most variable fragmentation hotspots showed clear cancer-specific fragmentation patterns in both t-SNE visualization and unsupervised hierarchical clustering (FIG. 4a-b, FIG. S16, Details in Supplementary Methods). The fragmentation patterns alone at these hotspots can separate the cancer types very well. By 10-fold cross-validation, the linear SVM model showed a consistent high classification performance across different stages for its high sensitivity at high specificity (64% sensitivity to 82% sensitivity at 100% specificity). Overall, the performance is complementary with large-scale fragmentation patterns and significantly higher in different stages than previously reported results by CNVs and mtDNA from the same dataset[4] (FIG. 4c, Table 1). For example, at 100% specificity, we achieved 93% sensitivity (95% CI: 85%-100%) in gastric cancer, 88% sensitivity (95% CI: 76%-100%) in colorectal cancer, and 81% sensitivity (95% CI: 76%-91%) in breast cancer, which of these are poorly detected at high specificity level by other liquid biopsy studies[4,29-32]. (FIG. S17-18, Table 1, Table S8). In the other cancer types, the performance is largely comparable to the previous results[4]. We also tested the performance before GC bias correction, and the results are largely the same (FIG. S17). Moreover, we estimated the tumor fractions in each sample by CNV based approach (ichorCNA)[33]. Our approach showed high performance even with a tumor fraction of less than 2%, and the performance is robust across samples with different tumor fractions (FIG. S19).

TABLE 1
CRAG Performance for the Detection of Early Stage Cancers.

[symbol]

| | | | 100% specificity | | |
|---|---|---|---|---|---|
| | | Indivi- | Indi- | Sensi- | 95% |
| | | duals | viduals | tivity | |
| | | analysed | detected | (%) | Cl (%) |
| Cancer | Breast | 54 | 44 | 0.8133 | 0.7168-0.9098 |
| Type | Bile duct | 26 | 11 | 0.4167 | 0.1819-0.6515 |
| | Colorectal | 27 | 24 | 0.8833 | 0.7635-1.0000 |
| | Gastric | 27 | 25 | 0.9333 | 0.8462-1.0000 |
| | Lung | 12 | 6 | 0.5000 | 0.2078-0.7922 |
| | Ovarian | 28 | 15 | 0.5500 | 0.3519-0.7451 |
| | Pancreatic | 34 | 19 | 0.5667 | 0.4251-0.7082 |
| Cancer | I | 41 | 28 | 0.6350 | 0.4428-0.8272 |
| Stage | II | 109 | 72 | 0.6667 | 0.5816-0.7518 |
| | III | 33 | 25 | 0.8200 | 0.6813-0.9587 |
| | I-IV | 205 | 141 | 0.6858 | 0.6098-0.7617 |
| 95% specificity | | | 85% specificity | | |
| Indi- | Sensi- | | Indi- | Sensi- | |
| viduals | tivity | 95% | viduals | tivity | 95% |
| detected | (%) | Cl (%) | detected | (%) | Cl (%) |
| 48 | 0.8867 | 0.7892-0.9842 | 51 | 0.9433 | 0.8865-1.0000 |
| 12 | 0.4500 | 0.2320-0.6680 | 14 | 0.5333 | 0.3166-0.7500 |
| 25 | 0.9162 | 0.8051-1.0000 | 25 | 0.9167 | 0.8061-1.0000 |
| 25 | 0.9333 | 0.8462-1.0000 | 26 | 0.9667 | 0.9013-1.0000 |
| 8 | 0.6500 | 0.3560-0.9400 | 8 | 0.6500 | 0.3660-0.9400 |
| 23 | 0.8167 | 0.6692-0.9741 | 24 | 0.8500 | 0.7263-0.9737 |
| 21 | 0.6333 | 0.4790-0.7877 | 22 | 0.6583 | 0.4889-0.8278 |
| 34 | 0 8290 | 0.7046-0.9454 | 34 | 0 8250 | 0.7046-0.9454 |
| 79 | 0.7297 | 0.6603-0.8092 | 85 | [symbol] | 0.7018-0.8607 |
| 28 | 0.8892 | 0.7796-0.9904 | 30 | 0.9360 | 0.8698-1.0000 |
| 169 | 0.7742 | 0.7229-0.8255 | 167 | [symbol] | 0.7596-0.8669 |

[symbol]

indicates data missing or illegible when filed

TABLE S8
Performance in seven types of cancer vs. healthy samples (before GC bias correction)

| | | | Sensitivity | Sensitivity | Sensitivity |
|---|---|---|---|---|---|
| | | Sensitivity @100% | @98% | @95% | @85% |
| AUC | | specificity | specificity | specificity | specificity |
| Breast cancer | 0.9887 (95 | 0.8900 (95 CI: | 0.8900 (95 CI: | 0.9267 (95 CI: | 1.0000 (95 CI: |
| vs. healthy | CI: 0.9771- | 0.7936- | 0.7936- | 0.8436- | 1.0000- |
| | 1.0000) | 0.9864) | 0.9864) | 1.0000) | 1.0000) |
| Bile duct vs. | 0.7556 (95 | 0.3167 (95 CI: | 0.3167 (95 CI: | 0.4333 (95 CI: | 0.4667 (95 CI: |
| healthy | CI: 0.6651- | 0.1075- | 0.1075- | 0.2200- | 0.2731- |
| | 0.8460) | 0.5258) | 0.5258) | 0.6467) | 0.6602) |
| Colorectal vs. | 0.9890 (95 | 0.9000 (95 CI: | 0.9000 (95 CI: | 0.9667 (95 CI: | 0.9667 (95 CI: |
| healthy | CI: 0.9736- | 0.8002- | 0.8002- | 0.9013- | 0.9013- |
| | 1.0000) | 0.9998) | 0.9998) | 1.0000) | 1.0000) |
| Gastric vs. | 0.9271 (95 | 0.7833 (95 CI: | 0.7833 (95 CI: | 0.8167 (95 CI: | 0.8167 (95 CI: |
| healthy | CI: 0.8428- | 0.6290- | 0.6290- | 0.6592- | 0.6592- |
| | 1.0000) | 0.9377) | 0.9377) | 0.9471) | 0.9471) |
| Lung vs. | 0.9019 (95 | 0.3500 (95 CI: | 0.3500 (95 CI: | 0.5000 (95 CI: | 0.8000 (95 CI: |
| healthy | CI: 0.8382- | 0.0560- | 0.0560- | 0.2078- | 0.5833- |
| | 0.9657) | 0.6440) | 0.6440) | 0.7922) | 1.0000) |
| Ovarian vs. | 0.9718 (95 | 0.7833 (95 CI: | 0.7833 (95 CI: | 0.8833 (95 CI: | 0.9667 (95 CI: |
| healthy | CI: 0.9418- | 0.6290- | 0.6290- | 0.7636- | 0.9013- |
| | 1.0000) | 0.9377) | 0.9377) | 1.0000) | 1.0000) |
| Pancreatic vs. | 0.8188 (95 | 0.5500 (95 CI: | 0.5500 (95 CI: | 0.5750 (95 CI: | 0.6583 (95 CI: |
| healthy | CI: 0.7214- | 0.4055- | 0.4055- | 0.4451- | 0.4889- |
| | 0.9163) | 0.6945) | 0.6945) | 0.7049) | 0.8278) |

Another big challenge for the diagnosis of early-stage cancer is identifying the cancer types for the most appropriate follow-up treatment choices. Here, we asked whether we can identify the tissues-of-origin of cancer samples by using the fragmentation levels alone. In the cancer positive samples identified above by machine learning algorithm, without any clinical information about the patients, we further localized the sources of cancer to one or two anatomic sites in a median of 85% of these patients across five different cancer types and 82.5% accuracy across six different cancer types. Furthermore, we were able to localize the source of the positive test to a single organ in a median of 65% of these patients across five different cancer types and 56% accuracy across six different cancer types. Our performance is similar to the previous reports using the combination of mutations and proteins[29] or DNA methylation[30] but superior to any other fragmentation approach (FIG. 4d, Table S9, FIG. S20) (Details in Methods). The prediction accuracy varies among tumor types, from 70% (95% CI: 44%-96%) in ovarian cancer to 98% (95% CI: 94%-100%) in breast cancer (FIG. 4d and Table S9), but significantly higher than random choices by the sample frequency in each cancer type (FIG. S21).

TABLE S9
Performance in multi-cancer classification (after GC bias correction)

| | Number of | | |
|---|---|---|---|
| Cancer type | individuals | Top 1 accuracy | Top 2 accuracy |
| Breast cancer | 44 | 0.6550 (95 CI: 0.4800- | 0.9800 (95 CI: 0.9408- |
| | | 0.8300) | 1.0000) |
| Colorectal | 24 | 0.6833 (95 CI: 0.4742- | 0.7833 (95 CI: 0.6290- |
| cancer | | 0.8925) | 0.9377) |
| Gastric | 25 | 0.5333 (95 CI: 0.2958- | 0.8667 (95 CI: 0.7307- |
| cancer | | 0.7709) | 1.0000) |
| Ovarian | 15 | 0.4000 (95 CI: 0.1152- | 0.7000 (95 CI: 0.4387- |
| cancer | | 0.6848) | 0.9613) |
| Pancreatic | 19 | 0.6500 (95 CI: 0.4408- | 0.8500 (95 CI: 0.6408- |
| cancer | | 0.8592) | 1.0000) |
| Breast | 44 | 0.5500 (95 CI: 0.4084- | 0.9550 (95 CI: 0.8957- |
| cancer | | 0.6916) | 1.0000) |
| Colorectal | 24 | 0.5667 (95 CI: 0.3589- | 0.7833 (95 CI: 0.6368- |
| cancer | | 0.7744) | 0.9298) |
| Gastric | 25 | 0.6500 (95 CI: 0.4298- | 0.8667 (95 CI: 0.7307- |
| cancer | | 0.8702) | 1.0000) |
| Ovarian | 15 | 0.3000 (95 CI: 0.0387- | 0.6550 (95 CI: 0.3560- |
| cancer | | 0.5613) | 0.9440) |
| Pancreatic | 19 | 0.8500 (95 CI: 0.6408- | 0.9000 (95 CI: 0.7693- |
| cancer | | 1.0000) | 1.0000) |
| Bile duct | 11 | 0.2000 (95 CI: 0.0000- | 0.4000 (95 CI: 0.0799- |
| cancer | | 0.4613) | 0.7201) |

### Discussion

In summary, the current disclosure provides a computational approach, named CRAG, to de novo identify the cfDNA fragmentation hotspots by weighting fragment coverages with the size information. Similar to the previous studies on the open chromatin regions within the cells, cfDNA fragmentation hotspots are highly enriched at known gene-regulatory elements. The in vivo fragmentation process, however, is complicated. A previous study suggested the co-existence of fragmentation coldspots and nucleosome protection but did not characterize the fragmentation hotspots, due to some computational challenges[3]. Genomic regions with a higher fragmentation rate do not always indicate the open chromatin regions. Besides nucleosomes, both biological issues (e.g., DNA methylation and histone modifications)[2,27] and technical artifacts (e.g., G+C %, k-mer, and mappability)[34,35] can affect the measurements of fragmentation level. After excluding the known effects of open chromatin regions and technical artifacts, our genome-wide analysis here revealed the enrichment of hotspots after the 3′ end of transposable elements and potentially associated with local DNA methylation level, which suggested the unknown origin of the cfDNA fragmentation processes.

Further, in early-stage cancer, we found the increases in fragmentation levels at the hotspots near microsatellites and the DNA methylation aberrations from another dataset at the same regions, which indicated the importance of exploring the fragmentation aberrations at the de novo characterized regions. More importantly, the hypo-fragmented hotspots in early-stage cancer are mostly located at promoters of genes enriched in the immune-related GO terms from the peripheral immune cells. Many recent efforts on the detection of early-stage cancer, however, focused on how to enrich the circulating tumor DNA signals from tumor cells[29,32], which ignored the critical role of peripheral immune aberrations during the cancer initiations[36]. In addition, the CTCF motif is highly enriched at these hypo-fragmented hotspots, which indicates the potential three-dimensional chromatin organization changes during the initiation of early-stage cancer, which has been reported before but not characterized by the cfDNA approaches[37]. Overall, our results suggested that the de novo characterization of fine-scale cfDNA fragmentation hotspots is critical to reveal the unknown gene-regulatory aberrations in pathological conditions.

Previous efforts had been made to characterize the nucleosome-free regions by using the depletion of coverages from MNase-seq/ChIP-seq assay[38]. The measurement of cfDNA fragmentation here, however, involves information from both fragment coverages and sizes. CRAG can be further improved by better integrating the fragment coverages and sizes, or even with more dimensions, such as the fragment orientation, jagged ends, and endpoint, to fully capture the spectrum of fragmentation. Also, G+C % bias is known to affect the peak calling result in ChIP-seq/ATAC-seq[39]. A better statistical model with the incorporation of GC normalization on both of the fragment coverages and sizes will improve our method's performance. PCR-free library preparation for WGS will also mitigate the concerns of GC bias and other sequencing artifacts[40].

For the detection and localization of early-stage cancer, we also identify several areas for further development. First, due to the limited availability of public cfDNA WGS datasets from early-stage cancer patients, the classification performance here is evaluated by multi-fold cross-validation on a relatively small sample-size cohort in each cancer type, similar to other cfDNA WGS studies[4]. Multiple independent large-scale prospective cohorts with similar cancer types will be a better way to assess the power of our approach for the diagnosis of early-stage cancer. Second, we pooled the low-coverage WGS samples from the same condition for the hotspot calling, which may cause the problem with a small number of samples. Due to the random drop out of the fragment coverages and many genomic windows in the genome, the number of falsely discovered hotspots without any biological interpretations will increase. Our current strategy by filtering low mappability regions and correcting GC bias is helpful to reduce the false positive rate for the hotspot detection. However, the accuracy of IFS signals at individual hotspots from each sample are still affected by the low-coverage data. Recent effort showed the possibility to integrate genome-wide mutational patterns at low-coverage WGS to enable the ultra-sensitive detection of cancer samples with limited cfDNA abundance[41], which is similar to our strategy for the IFS signals here. Since we narrow down the regions of interest, even with missing values at part of the loci, many other hotspots from the same sample will still provide informative signals rather than noises for the model to make the classifications. In the future, appropriate statistical models for the imputation of missing fragmentation patterns may be useful to mitigate the missing data problem. Third, the proportion of cancer types and the ratio between cancer and healthy is not an unbiased representation of the average-risk population in the US. The sensitivity and specificity here may not represent the actual performance in the large-scale screening. Fourth, the proof-of-concept study on HCC here suggested the distinguished cfDNA fragmentation patterns between early-stage cancer and non-malignant liver disease controls. More cfDNA studies on non-malignant cancer, diseases, and benign status may be performed to minimize the overdiagnosis in the population-level screening. Lastly, in some cancer types, our fine-scale study here showed complementary classification performance compared with that in the previous large-scale fragmentation study at the same dataset[4]. For example, our results on gastric, breast, and colorectal cancer outperformed previous large-scale fragmentation studies, while at bile duct and lung cancer, the performance is reversed. Future combinations of the fragmentation patterns at multi-scales and information from other modalities or clinical meta-data may further improve the performance.

Our study here lays the foundation to non-invasively detect multiple early-stage cancers simultaneously on an existing matured high-throughput platform in a cost-effective way. It also paves the road to further elucidate the unknown gene-regulatory mechanisms in pathological conditions through the cfDNA fragmentation hotspots.

### Materials and Methods

### Public Datasets.

Public datasets used in this study were listed in Table S1.

### Preprocess of Whole-Genome Sequencing Data.

The adapter was trimmed by Trimmomatic (v0.36)[42] in paired-end mode with the following parameters: ILLUMINACLIP:TruSeq3-PE.fa:2:30:10:2:keepBothReads MINLEN:36. After adapter trimming, reads were aligned to the human genome (GRCh37, human_glk_v37.fa) using BWA-MEM 0.7.15[43] with default parameters. PCR-duplicate fragments were removed by samblaster (v0.1.24)[44]. Only high-quality autosomal reads were used for all downstream analyses (both ends uniquely mapped, either end with mapping quality score of 30 or greater, properly paired, and not a PCR duplicate).

### Preprocess of Whole-Genome Bisulfite Sequencing Data.

DNA methylation levels measured by WGBS in cfDNA from HCC patients and healthy individuals were obtained from previous publications (Details in Table S1) [28,45]. Single-end WGBS from cfDNA was processed by the following internal pipeline. Based on FastQC results on the distribution of four nucleotides along the sequencing cycle, the adapter was trimmed by Trim Galore! (v0.6.0) with cutadapt (v2.1.0) and with parameters “ --clip_R1 10” and “ --clip_R1 10 --three_prime_clip_R1 13”. After the adapter trimming, reads were aligned to the human genome (GRCh37, human_glk_v37.fa) by Biscuit (v0.3.10.20190402) with default parameters. PCR-duplicate reads were marked by samtools (v1.9)[46]. Only high-quality reads were used for all the downstream analyses (uniquely mapped, mapping quality score of 30 or greater, and not a PCR duplicate). Methylation level at each CpG was called by Bis-SNP with default parameters in bissnp_easy_usage.pl[47].

Identification of cfDNA Fragmentation Hotspots by CRAG.

Fragment coverages and sizes are both essential parts of the cfDNA fragmentation patterns. However, popular peak calling tools, such as MACS2[48], cannot address the signals from two different dimensions. Thus, we created an integrated fragmentation score (IFS) by weighting the fragment coverage based on the ratio of average fragment size in the window versus that in the whole chromosome. Specifically, we utilized a 200 bp sliding window with a 20 bp step to scan each chromosome (autosome only). In the iₜₕ window:

$${IFS}_{i} = {n}_{i} * ( 1 + \frac{{l}_{i}}{L} ) \qquad ( Eq . 1 )$$

$${C}_{i} = ⌊ {IFS}_{i} ⌋ \qquad ( Eq . 2 )$$

where Cᵢ is the IFS score round down to the nearest integer in the iₜₕ window, nᵢ is the number of fragments whose mid-points are located within the iₜₕ window, lᵢ is the average fragment size in the iₜₕ window, L is the average fragment size in the whole chromosome. Windows overlapped with dark regions or with average mappability scores smaller than 0.9 were removed. Dark regions were defined by the merged DAC blacklist and Duke Excluded from the UCSC table browser. Mappability score was generated by the GEM mappability program on the human reference genome (GRCh37, human_glk_v37.fa, 51mer)[49]. We assumed the background Cᵢ following the negative binomial (NB) distribution.

$${C}_{i} ∼ NB ( n , p ) \qquad ( Eq . 3 )$$

We denoted the sample mean and sample variance as u and v. Thus, we can estimate NB parameters as follows:

$$p = \frac{μ}{v} \qquad ( Eq . 4 )$$

$$n = \frac{{μ}^{2}}{v} \frac{1}{1 - p} \qquad ( Eq . 5 )$$

We utilize the NB model to test whether the Cᵢ in the iₜₕ window was significantly smaller than the local background (20 kb and 40 kb) and global background (the whole chromosome). In R, we can calculate p-values using the following function:

$$pnbinom ( q , prob = p , size = n ) \qquad ( Eq . 6 )$$

Only windows with a p-value smaller than a cut-off (p-value<=1.0e-05) in both the local and global background were kept for further analysis. P-value from the comparison with the global background was utilized for the multiple hypothesis corrections (Benjamini and Hochberg method). Windows with a false discovery rate (FDR) of more than 0.25 were filtered. Finally, significant windows with a distance of less than 200 bp to each other were merged as the final hotspots.

To remove the possible sequence composition bias caused by G+C % content, similar as previous study[4], locally weighted smoothing linear regression (loess, span=0.75) was utilized to regress out the GC covariates from the raw IFS score in each window. The mean IFS score in each chromosome was added back to the residual value after the correction. The hotspots were called based on the corrected IFS finally.

To check the possible fragmentation bias caused by k-mer, we first calculated the expected IFS by using the average IFS at each possible type of dimer (16 types) across the genome. Then at each location, the adjusted IFS was calculated by dividing the original IFS with the expected IFS based on the dimer composition at that location. Finally, the adjusted IFS at each location was multiplied by the ratios between the average adjusted IFS and average expected IFS in the same chromosome.

### Benchmark the Accuracy to Predict the Open Chromatin Regions.

To benchmark the performance of our method on the open chromatin region calling, we utilized the 15-states chromHMM segmentation result across different cell types from NIH Epigenome Roadmap Consortium. We generated a balanced fragmentation-positive and fragmentation-negative group randomly sampled from two types of regions: (1) constitutively open regions: we used the −150 bp to 50 bp regions around the transcription starting sites which are overlapped with TssA chromHMM states shared across all cell types; (2) constitutively closed regions: we used the Quies chromHMM states shared across all cell types, then we randomly sampled the intervals from these regions with matched GC content and mappability as the constitutively open regions. We utilized the IFS score and k-mer (k=2) composition within these two types of regions as the features and applied the linear SVM with default parameters in the ten-fold cross-validation.

Cancer Early Detection by cfDNA Fragmentation Hotspots.

Here, we took the classification of liver cancer vs. healthy controls as an example. Ten-fold cross-validation was applied to evaluate the performance. At the training data set, all the liver cancer samples and healthy samples were pooled to identify the hotspots, respectively. We kept the top 30,000 most stable hotspots (ranked by the sum of variances in cancer and control group) as the feature for the classification. It is well known that the sequencing depths will largely affect the number of peaks called in ChIP-seq and ATAC-seq[19]. In Cristiano et al. dataset[4], the sample size in the healthy group is ten times larger than that in any cancer type, which will lead to the uneven sequencing depths between healthy controls and cancers. Thus, by following the similar procedures in the previous publication[19], we downsampled the number of healthy controls to the same size as cancer before hotspot calling in each comparison (e.g., Breast cancer vs. Healthy). IFS before and after GC bias correction have both been tested. IFS after GC bias correction was shown in the main figure for the classification. Only genomic regions at +/−100 bp of the hotspot center were used to retrieve the IFS in each sample (the same strategy was used in PCA and unsupervised clustering analysis). The IFS at each corresponding hotspot was z-score transformed based on the mean and standard deviation at each chromosome of each sample. Finally, a support vector machine (SVM) classifier with linear kernel and default parameters (fitcsvm function at Matlab 2019b) was applied. At the testing dataset, the z-score transformed IFS in each sample was calculated at the hotspot regions identified from the training set in that particular fold. The average AUC and 95% Confidence Interval (95% CI) of the AUC was calculated based on the classification results at the testing dataset across the ten folds. To avoid the randomness of the data split, we repeated the cross-validation randomly ten times.

Tissues-of-Origin Predictions by cfDNA Fragmentation Hotspots.

Only samples predicted as cancer were kept for the tissues-of-origin analysis. The saturation analysis of the fragment number needed for hotspot calling suggested that 400 million fragments are required to achieve the saturated performance (FIG. S7, Details in Supplementary Methods). Thus, pathological conditions with less than 400 million fragments in total were not used for the tissues-of-origin analysis (e.g., lung cancer). Bile duct cancer was at the boundary condition with 380 million fragments. Therefore, we performed the analysis with or without bile duct cancer. By 10-fold cross-validation, similar to that in the cancer early detection part, hotspots for each cancer type in the training set were identified. The z-score transformed IFS after GC bias correction in each sample was obtained as the feature. Since the total number of fragments in breast cancer is much larger than that in the other cancer types, we downsampled breast cancer to the median sample size across all the cancer types. The centroid in each cancer type was then calculated by the z-score transformed IFS across all the hotspots in the training set. In the testing dataset, each sample was assigned to the top two candidate cancers based on their distance to the centroids in each cancer type identified at the training set. The distance was calculated by corr function with ‘Type’ of ‘Spearman’ at Matlab 2019b. To further narrow down the best candidate cancer type, decision tree models (fitctree function at Matlab 2019b) were learned to identify the better candidate by the top 100,000 most stable hotspots in each possible pair of cancer types at the training set. Finally, we applied the corresponding decision tree model on the top two candidates to further characterize the best candidate at the testing set.

### Supplementary Methods

The Saturation Analysis of the Fragment Number Needed for the Hotspot Calling of cfDNA Fragmentation Hotspots.

A group of fragmentation-positive regions and fragmentation-negative regions were generated for the benchmark. For fragmentation-positive regions, we chose the CGI TSS that are overlapped with conserved TssA chromHMM states (15-state chromHMM) shared across the cell types from NIH Epigenome Roadmap. Regions that are −50 bp to +150 bp around these active TSS were defined as the fragmentation-positive regions. For fragmentation-negative regions, we chose the same number of random genomic regions from conserved Quies chromHMM states shared across the cell types but with the same chromosome, region size, G+C % content, and mappability score as that in fragmentation-positive regions.

We downsampled the high-qualify fragments in the BH01 dataset from 1.2 billion to 10 million. We identified the hotspots at these downsampled datasets and calculated TP (true positive), FP (false positive), TN (true negative), FN (false negative) based on their overlaps with the benchmark regions generated above. F-score was calculated:

$$Fscore = 2 * \frac{Precision * Recall}{Precision + Recall} \qquad ( Eq . S1 )$$

in which, Precision and Recall were calculated using equation (S2) and equation (S3), respectively:

$$Precision = \frac{TP}{TP + FP} \qquad ( Eq S2 )$$

$$Recall = \frac{TP}{TP + FN} \qquad ( Eq . S3 )$$

The performance is saturated at ˜0.9 F1-score with 400 million fragments. Even with 200 million fragments, we can still achieve good performance (˜0.8 F1-score) (FIG. S7).

The Enrichment Analysis of the cfDNA Fragmentation Hotspots in Gene-Regulatory Elements.

The number of hotspots that overlapped with the regulatory element was counted by bedtools v2 [50]. After filtering out the dark regions and low mappability regions (mappability less than 0.9), random genomic regions were generated with matched chromosomes and sizes. Fisher exact test (two-tail) was performed to calculate the enrichment of hotspots over the matched random regions.

The Principal Component Analysis of the cfDNA Fragmentation Hotspots Across Different Diseases.

The cfDNA fragmentation hotspots were called at each pathological condition as described in the Methods. Principal Component Analysis (PCA) was performed on the z-score transformed IFS across all the fragmentation hotspots (pca function at Matlab 2019b).

Unsupervised Hierarchical Clustering Analysis of the cfDNA Fragmentation Hotspots Across Different Diseases.

The cfDNA fragmentation hotspots were called at each pathological condition as described in the Methods. Top N most variable hotspots were kept for the clustering (ranked by the variation across all the samples). Spearman's rank correlation was utilized to evaluate the distance among the samples. Also, weighted average distance (WPGMA, with ‘weighted’ as the parameter in clustergram function at Matlab 2019b) was applied together with the linkage method. In the Cristiano et al. dataset, one-way ANOVA (p-value<=0.01) was applied to select the hotspots that showed the group-specific fragmentation patterns. Further, hotspots are ranked by the z-score difference between the samples within the group and outside the group. The top 5,000 hotspots in each group were finally visualized in the figure.

The t-SNE Visualization of the cfDNA Fragmentation Hotspots Across Different Diseases.

T-SNE (tsne function at Matlab 2019b) was utilized for the dimensionality reduction and visualization of the fragmentation dynamics in the hotspots across multiple cancer and healthy conditions. Hotspots with a p-value<=0.01 (one-way ANOVA) were used for the analysis. Distance similarity was calculated by the Spearman correlation together with default parameters (tsne function at Matlab 2019b).

The Gene Ontology Analysis of the cfDNA Fragmentation Hotspots.

Gene Ontology (GO) analysis of the cfDNA fragmentation hotspots was performed by GREAT (v4.04) [51]. The GO Biological Processes (GO BP) with a q-value of less than 0.01 (binomial test) were selected. Only the top ten GO BPs were shown in the main figure.

The Motif Analysis of the cfDNA Fragmentation Hotspots.

Motif analysis of cfDNA fragmentation hotspots was performed by HOMER (v4.11) with the command ‘findMotifsGenome.pl hotspots_file hg19 output_file-size given’[52]. Only motifs with a q-value of less than 0.01 were kept. Only the top 10 enriched motifs were shown in the figures.

The Estimation of Tumor Fractions by ichorCNA.

The ichorCNA v0.2.0[33] was run at 1 Mb resolution with the normalization by the normal panel provided in the package together with G+C %, mappability, and the following parameters: --normal “c (0.75)” --ploidy “c(2)” --maxCN 5 --estimateScPrevalence FALSE --scStates “c(1,3)” --chrs “c(1:22)”

### Application to Non-Malignant Diseases

In non-malignant diseases such as multiple sclerosis (MS), the changes in IFS in cfDNA fragmentation hotspots showed distinct patterns across MS disease subtypes indicating the potentially generalizable application to fine-scale fragmentation patterns to monitor the progression of complex diseases.

### Example Computing Environments

The current disclosure provides methods and systems for identifying DNA fragmentation hotspots as part of diagnosing early stage cancer. The computing engines, modules, machine learning modules, machine learning engines, deep learning modules/engines, training systems, architectures and other disclosed functions are embodied as computer instructions that may be installed for running on one or more computer devices and/or computer servers. In some instances, a local user can connect directly to the system; in other instances, a remote user can connect to the system via a network.

Example networks can include one or more types of communication networks. For example communication networks can include (without limitation), the Internet, a local area network (LAN), a wide area network (WAN), various types of telephone networks, and other suitable mobile or cellular network technologies, or any combination thereof. Communication within the network can be realized through any suitable connection (including wired or wireless) and communication technology or standard (wireless fidelity (WiFi®), 4G, 5G, long-term evolution (LTE™)), and the like as the standards develop.

The computer device(s) and/or computer server(s) can be configured with one or more computer processors and a computer memory (including transitory computer memory and/or non-transitory computer memory), configured to perform various data processing operations. The computer device(s) and/or computer server(s) also include a network communication interface to connect to the network(s) and other suitable electronic components.

Example local and/or remote user devices can include a personal computer, portable computer, smartphone, tablet, notepad, dedicated server computer devices, any type of communication device, and/or other suitable compute devices.

The computer device(s) and/or computer server(s) can include one or more computer processors and computer memories (including transitory computer memory and/or non-transitory computer memory), which are configured to perform various data processing and communication operations associated with diagnosing liver disease as disclosed herein based upon information obtained/provided over the network, from a user and/or from a storage device. In some implementations, storage device can be physically integrated to the computer device(s) and/or computer server(s); in other implementations, storage device can be a repository such as a Network-Attached Storage (NAS) device, an array of hard-disks, a storage server or other suitable repository separate from the computer device(s) and/or computer server(s).

In some instances, storage device can include the machine-learning models/engines and other software engines or modules as described herein. Storage device can also include sets of computer executable instructions to perform some or all the operations described herein.

The following list of reference has been cited herein by their number. Each reference below is incorporated herein by reference:
1. Heitzer, E., Haque, I. S., Roberts, C. E. S. & Speicher, M. R. Current and future perspectives of liquid biopsies in genomics-driven oncology. Nat. Rev. Genet. 20, 71-88 (2019).
2. Ivanov, M., Baranova, A., Butler, T., Spellman, P. & Mileyko, V. Non-random fragmentation patterns in circulating cell-free DNA reflect epigenetic regulation. BMC Genomics 16 Suppl 13, S1 (2015).
3. Snyder, M. W., Kircher, M., Hill, A. J., Daza, R. M. & Shendure, J. Cell-free DNA Comprises an In Vivo Nucleosome Footprint that Informs Its Tissues-Of-Origin. Cell 164, 57-68 (2016).
4. Cristiano, S. et al. Genome-wide cell-free DNA fragmentation in patients with cancer. Nature 570, 385-389 (2019).
5. Chabon, J. J. et al. Integrating genomic features for non-invasive early lung cancer detection. Nature 580, 245-251 (2020).
6. Ulz, P. et al. Inferring expressed genes by whole-genome sequencing of plasma DNA. Nat. Genet. 48, 1273-1278 (2016).
7. Jiang, P. et al. Preferred end coordinates and somatic variants as signatures of circulating tumor DNA associated with hepatocellular carcinoma. Proc. Natl. Acad. Sci. U.S.A. 115, E10925-E10933 (2018).
8. Sun, K. et al. Orientation-aware plasma cell-free DNA fragmentation analysis in open chromatin regions informs tissue of origin. Genome Res. 29, 418-427 (2019).
9. Ulz, P. et al. Inference of transcription factor binding from cell-free DNA enables tumor subtype prediction and early detection. Nat. Commun. 10, 4666 (2019).
10. Jiang, P. et al. Plasma DNA End-Motif Profiling as a Fragmentomic Marker in Cancer, Pregnancy, and Transplantation. Cancer Discov. 10, 664-673 (2020).
11. Corces, M. R. et al. The chromatin accessibility landscape of primary human cancers. Science 362, (2018).
12. Calderon, D. et al. Landscape of stimulation-responsive chromatin across diverse human immune cells. Nat. Genet. 51, 1494-1505 (2019).
13. Lambert, S. A. et al. The Human Transcription Factors. Cell 172, 650-665 (2018).
14. Tak, Y. G. & Farnham, P. J. Making sense of GWAS: using epigenomics and genome engineering to understand the functional relevance of SNPs in non-coding regions of the human genome. Epigenetics Chromatin 8, 57 (2015).
15. Guo, M. et al. Epigenetic profiling of growth plate chondrocytes sheds insight into regulatory genetic variation influencing height. Elife 6, (2017).
16. Hook, P. W. & McCallion, A. S. Leveraging mouse chromatin data for heritability enrichment informs common disease architecture and reveals cortical layer contributions to schizophrenia. Genome Res. 30, 528-539 (2020).
17. Buenrostro, J. D., Giresi, P. G., Zaba, L. C., Chang, H. Y. & Greenleaf, W. J. Transposition of native chromatin for fast and sensitive epigenomic profiling of open chromatin, DNA-binding proteins and nucleosome position. Nat. Methods 10, 1213-1218 (2013).
18. Lui, Y. Y. N. et al. Predominant hematopoietic origin of cell-free DNA in plasma and serum after sex-mismatched bone marrow transplantation. Clin. Chem. 48, 421-427 (2002).
19. Roadmap Epigenomics Consortium et al. Integrative analysis of 111 reference human epigenomes. Nature 518, 317-330 (2015).
20. Siepel, A. et al. Evolutionarily conserved elements in vertebrate, insect, worm, and yeast genomes. Genome Res. 15, 1034-1050 (2005).
21. ENCODE Project Consortium. An integrated encyclopedia of DNA elements in the human genome. Nature 489, 57-74 (2012).
22. Stunnenberg, H. G., International Human Epigenome Consortium & Hirst, M. The International Human Epigenome Consortium: A Blueprint for Scientific Collaboration and Discovery. Cell 167, 1145-1149 (2016).
23. Corces, M. R. et al. Lineage-specific and single-cell chromatin accessibility charts human hematopoiesis and leukemia evolution. Nat. Genet. 48, 1193-1203 (2016).
24. Soufi, A. et al. Pioneer transcription factors target partial DNA motifs on nucleosomes to initiate reprogramming. Cell 161, 555-568 (2015).
25. Jiang, P. et al. Lengthening and shortening of plasma DNA in hepatocellular carcinoma patients. Proc. Natl. Acad. Sci. U.S.A. 112, E1317-25 (2015).
26. Shi, M. et al. A blood-based three-gene signature for the non-invasive detection of early human hepatocellular carcinoma. Eur. J. Cancer 50, 928-936 (2014).
27. Jensen, T. J. et al. Whole genome bisulfite sequencing of cell-free DNA and its cellular contributors uncovers placenta hypomethylated domains. Genome Biol. 16, 78 (2015).
28. Chan, K. C. A. et al. Noninvasive detection of cancer-associated genome-wide hypomethylation and copy number aberrations by plasma DNA bisulfite sequencing. Proc. Natl. Acad. Sci. U.S.A. 110, 18761-18768 (2013).
29. Cohen, J. D. et al. Detection and localization of surgically resectable cancers with a multi-analyte blood test. Science 359, 926-930 (2018).
30. Liu, M. C. et al. Sensitive and specific multi-cancer detection and localization using methylation signatures in cell-free DNA. Ann. Oncol. (2020) doi:10.1016/j.annonc.2020.02.011.
31. Wan, N. et al. Machine learning enables detection of early-stage colorectal cancer by whole-genome sequencing of plasma cell-free DNA. BMC Cancer 19, 832 (2019).
32. Shen, S. Y. et al. Sensitive tumour detection and classification using plasma cell-free DNA methylomes. Nature 563, 579-583 (2018).
33. Adalsteinsson, V. A. et al. Scalable whole-exome sequencing of cell-free DNA reveals high concordance with metastatic tumors. Nat. Commun. 8, 1324 (2017).
34. Cheung, M.-S., Down, T. A., Latorre, I. & Ahringer, J. Systematic bias in high-throughput sequencing data and its correction by BEADS. Nucleic Acids Res. 39, e103 (2011).
35. Benjamini, Y. & Speed, T. P. Summarizing and correcting the GC content bias in high-throughput sequencing. Nucleic Acids Res. 40, e72 (2012).
36. Gonzalez, H., Hagerling, C. & Werb, Z. Roles of the immune system in cancer: from tumor initiation to metastatic progression. Genes Dev. 32, 1267-1284 (2018).
37. Liu, E. M. et al. Identification of Cancer Drivers at CTCF Insulators in 1,962 Whole Genomes. Cell Syst 8, 446-455.e8 (2019).
38. Mammana, A., Vingron, M. & Chung, H.-R. Inferring nucleosome positions with their histone mark annotation from ChIP data. Bioinformatics 29, 2547-2554 (2013).
39. Teng, M. & Irizarry, R. A. Accounting for GC-content bias reduces systematic errors and batch effects in ChIP-seq data. Genome Res. 27, 1930-1938 (2017).
40. Aird, D. et al. Analyzing and minimizing PCR amplification bias in Illumina sequencing libraries. Genome Biol. 12, R18 (2011).
41. Zviran, A. et al. Genome-wide cell-free DNA mutational integration enables ultra-sensitive cancer monitoring. Nat. Med. 26, 1114-1124 (2020).
42. Bolger, A. M., Lohse, M. & Usadel, B. Trimmomatic: a flexible trimmer for Illumina sequence data. Bioinformatics 30, 2114-2120 (2014).
43. Li, H. & Durbin, R. Fast and accurate short read alignment with Burrows-Wheeler transform. Bioinformatics 25, 1754-1760 (2009).
44. Faust, G. G. & Hall, I. M. SAMBLASTER: fast duplicate marking and structural variant read extraction. Bioinformatics 30, 2503-2505 (2014).
45. Sun, K. et al. Plasma DNA tissue mapping by genome-wide methylation sequencing for noninvasive prenatal, cancer, and transplantation assessments. Proc. Natl. Acad. Sci. U.S.A. 112, E5503-12 (2015).
46. Li, H. et al. The Sequence Alignment/Map format and SAMtools. Bioinformatics 25, 2078-2079 (2009).
47. Liu, Y., Siegmund, K. D., Laird, P. W. & Berman, B. P. Bis-SNP: combined DNA methylation and SNP calling for Bisulfite-seq data. Genome Biol. 13, R61 (2012).
48. Zhang, Y. et al. Model-based analysis of ChIP-Seq (MACS). Genome Biol. 9, R137 (2008).
49. Derrien, T. et al. Fast Computation and Applications of Genome Mappability. PLoS One 7, e30377 (2012).
50. Quinlan, A. R. & Hall, I. M. BEDTools: a flexible suite of utilities for comparing genomic features. Bioinformatics 26, 841-842 (2010).
51. McLean, C. Y. et al. GREAT improves functional interpretation of cis-regulatory regions. Nat. Biotechnol. 28, 495-501 (2010).
52. Heinz, S. et al. Simple combinations of lineage-determining transcription factors prime cis-regulatory elements required for macrophage and B cell identities. Mol. Cell 38, 576-589 (2010).

What is claimed is:

1. A method for identifying DNA fragmentation hotspots as part of diagnosing early stage cancer, comprising:
de-novo characterizing genome-wide cell-free DNA fragmentation hotspots from whole-genome sequencing by integrating fragment size and coverage into a score; and
identifying DNA fragmentation hotspots of interest based upon the score being below a threshold.

2. The method of claim 1, wherein the score identifies regions with lower fragment coverage and smaller fragment size.

3. The method of claim 1, further comprising a step of scanning a chromosome with a sliding window of a first size and a step with a second size.

4. The method of claim 3, wherein the score is calculated by weighting fragment coverage based on a ratio of average fragment size in the sliding window versus that in the whole chromosome

5. The method of claim 4, wherein the score is calculated based upon the following equation wherein, in the iₜₕ window:
$${IFS}_{i} = {n}_{i} * ( 1 + \frac{{l}_{i}}{L} ) \qquad ( 1 )$$
$${C}_{i} = ⌊ {IFS}_{i} ⌋ \qquad ( 2 )$$
wherein Cᵢ is the IFS score round down to the nearest integer in the iₜₕ window, nᵢ is the number of fragments whose mid-points are located within the iₜₕ window, lᵢ is the average fragment size in the iₜₕ window, L is the average fragment size in the whole chromosome.

6. The method of claim 4, further comprising utilize identified DNA fragmentation hotspots for the detection and localization of multiple early-stage cancers.

7. The method of claim 3, wherein the first size is 200 bp and the second size is 20 bp.

8. The method of claim 1, further comprising utilize identified DNA fragmentation hotspots for the detection of early-stage cancer.

9. The method of claim 8, wherein the detection step includes one or more steps taken from the group comprising:
performing Gene Ontology (GO) analysis of the identified DNA fragmentation hotspots; or
performing Motif analysis of the identified DNA fragmentation hotspots.

10. The method of claim 1, wherein integrating step weighs fragment coverages with size information.

11. The method of claim 10, wherein the integrating step weighs the fragment coverage based on a ratio of fragment size in a window versus that in the whole chromosome.

12. The method of claim 1, further comprising filtering out dark regions and low mappability regions.

13. A method for identifying genomic regions with higher fragmentation rates than the local and global backgrounds as part of diagnosing early stage cancer, comprising:
de-novo characterizing genome-wide cell-free DNA fragmentation regions with higher fragmentation rates than the local and global backgrounds from whole-genome sequencing by weighing the fragment coverages in each region by a ratio of average fragment sizes in the region versus that in the whole chromosome to generate a score; and
identifying DNA fragmentation regions of interest based upon comparing the score with a threshold.

14. The method of claim 13, further comprising a step of scanning a chromosome with a sliding window of a first size and a step with a second size.

15. The method of claim 14, wherein the score is calculated by weighting fragment coverage based on a ratio of average fragment size in the sliding window versus that in the whole chromosome

16. The method of claim 14, wherein the first size is 200 bp and the second size is 20 bp.

17. The method of claim 13, further comprising utilize identified DNA fragmentation hotspots for the detection of early-stage cancer.

18. The method of claim 17, wherein the detection step includes one or more steps taken from the group comprising:
performing Gene Ontology (GO) analysis of the identified DNA fragmentation hotspots; or
performing Motif analysis of the identified DNA fragmentation hotspots.

19. The method of claim 13, further comprising filtering out dark regions and low mappability regions.

20. A non-transitory computer memory including computer instructions for performing a method for identifying genomic regions with higher fragmentation rates than the local and global backgrounds as part of diagnosing early stage cancer, the computer instructions configured to perform steps of:
de-novo characterizing genome-wide cell-free DNA fragmentation regions with higher fragmentation rates than the local and global backgrounds from whole-genome sequencing by weighing the fragment coverages in each region by a ratio of average fragment sizes in the region versus that in the whole chromosome to generate a score; and
identifying DNA fragmentation regions of interest based upon comparing the score with a threshold.

21. The non-transitory computer memory of claim 20, wherein the computer instructions are further configured to perform a step of scanning a chromosome with a sliding window of a first size and a step with a second size.

22. The non-transitory computer memory of claim 21, wherein the score is calculated by weighting fragment coverage based on a ratio of average fragment size in the sliding window versus that in the whole chromosome

23. The non-transitory computer memory of claim 21, wherein the first size is 200 bp and the second size is 20 bp.

24. The non-transitory computer memory of claim 20, wherein the computer instructions are further configured to utilize identified DNA fragmentation hotspots for the detection of early-stage cancer.

25. The non-transitory computer memory of claim 24, wherein the detection step includes one or more steps taken from the group comprising:
performing Gene Ontology (GO) analysis of the identified DNA fragmentation hotspots; or
performing Motif analysis of the identified DNA fragmentation hotspots.

26. The non-transitory computer memory of claim 20, wherein the computer instructions are further configured to filter out dark regions and low mappability regions.
